SEQUENCE LISTING (1059 sequences)

SEQ ID NO: 1 - DNA, 250 bp, Homo sapiens
tgagcgtggaggtggagtgggaagatgggtcaacttgcagcagttctccygggactgtatgaagagggcatcccgtttctcagagtctttgtgcaggccagttacctttccaagctgatgggggccatccactgtcttcccatcgtcatctggtccccagctgttgagaaataaatacctagaggggacagccagagccaggctgcggctacccacccagcgcctcccagtctcagctggctgccctttg

SEQ ID NO: 2 - DNA, 170 bp, Homo sapiens
gccgggcagagacagcggctagagaccccgaggtgggggacacaaatgarctccttgcccgtccccccaatttctcctaccctacaaagggagggaatcccaaaaggacccttctacccctgtgactggttttcatgaaggactatgattcccatcatccctcaggggca

SEQ ID NO: 3 - DNA, 189 bp, Homo sapiens
caaatccctgaaagagtaagcattatgctaatgggtctgaaaatataaaygaagaatcaaaatctattctgctttaaagaaggcaccagtttaggaggcaggctctaagtacttgaagcaataaaaggaactcccctttgcctaacaagtcccatatagcagcctccttcatacattcagtttaacgtc

SEQ ID NO: 4 - DNA, 110 bp, Homo sapiens
aggaatgatgcctggatgcctgggggtgtgaagaaaagagccttgggttygactagggaacctggggccactccttcctctgctactaaatcaccaagtgatcttgttct

SEQ ID NO: 5 - DNA, 170 bp, Homo sapiens
cttccttctatgacccctacctgttgtgggtctccaggcaagcagtgcayaggtagagccatccttaggtagcctttagacttaatattaggtgagctctccccacagatagcctctcctttatttgaatggaattatattttaagtttggaaatatttttcagcttatt

SEQ ID NO: 6 - DNA, 170 bp, Homo sapiens
ctctcaaacattagcttttacccttttaccttcaggttcgtaccccaccrtatttgcataattgtattacttccctaagaaccaattttttgttgttgttgtttgctagtacttttgtgccacctgacaaacttacaagctatttttaaatgcttgcttttaaatagagg

SEQ ID NO: 7 - DNA, 126 bp, Homo sapiens
ccagtaggtgtcagctacgcacagcctttctctttctaaatccatatttrtaagaaaacggcctttcatccataattttccctagttaaagggatcaatttcttctgatctgaagcacgcaaaaca

SEQ ID NO: 8 - DNA, 170 bp, Homo sapiens
gaaacgccatggctgttctcataccaagtgctatttaataagacagatcrctcaaactgggatcccaagagggttcaattaagagcataatgtaattccaggctaccagtgctgccctcagcccaagtatctcatacctgctctatgaagtctgtcctgatgtccccaga

SEQ ID NO: 9 - DNA, 170 bp, Homo sapiens
tggcctgtgaagtccagaagctctttccgcgctcggctctttgagcctcrgtttccccatctgtcaggcgtgtgtatgtgggaggggggtacggagtagatttacctagatcactcggggttgtcccttccagttttgagggtgatgtcggggtgtctgtgaggaggagg

SEQ ID NO: 10 - DNA, 170 bp, Homo sapiens
gtcgctgtcagttgaaatagattacaagagggatctcctggcagaatttrtttagtttagctcaaaggagaatgttatagtcaagggtacatttggttgcaaggacaagagagtcactcgagttggcttaagtaaaagggtacttgtcccaaaaacataacagccagtcc

SEQ ID NO: 11 - DNA, 170 bp, Homo sapiens
aatctcttttccagaagtctaagtgaaaatctcactttgtctctgtactrgttaggcaaggctaagttgctatgacaaatagacctcaaattctgctgtaacagctcagactataacactgcattttgctgtcatgtaatatcagtggtgggcctttctggtcatttctc

SEQ ID NO: 12 - DNA, 170 bp, Homo sapiens
ctccatgctgcatgtaggtttataaaccaataccctgctcagacaccacrttcttctaacaccctaatgatggggtcacatttctctctttaggtactacaatctggttttgaagttgcccaaagaaacttgaatggcttcctctttcattcccacaaggctctgaaaac

SEQ ID NO: 13 - DNA, 170 bp, Homo sapiens
gacactcccctactccaggcctgcagcactcacctgcatctcacactgcratgcaaaatttgtgtgattaatgggttcaaatgtgcccccctcccctggactttacagggtgctggagctgctggcttgctgttcccagtgccaagtcccatatgggtgctcagccatat

SEQ ID NO: 14 - DNA, 311 bp, Homo sapiens
ttaatatggtgtcctgctaaaagattatccttgtcttcttcttttccccrtagatgatcttagtagccatattttcagaaacgggatttttcgattattgtgctgtaaaggtaggtatgatgttgcatttaataattctatcctgattaatttatatatgtatttttctgacattatatatttaggaaacaaacatttaaaacaaacattgaaaaattccatccttcttttaaaggttgcttctgcagggcagggtatacttgctatgttaagttgtatggctctgagcagcactttcagctgctcagtaa

SEQ ID NO: 15 - DNA, 242 bp, Homo sapiens
cctcttcacgcctgtgaccataaggtacgcaaagcacctctgccgtgggrgttgcggccaggttctggcaggcaggggctctgcctgcactgcctggctccaggttccattctcaggtgcatgaaaaggtgggggcrgttgagcccacagctcactgcattccagtccagctcgtgtctgctttgtgtgactgcagtacatgctacaagcagtggggcctcagaagctggtggcagaaatgc

SEQ ID NO: 16 - DNA, 110 bp, Homo sapiens
gcctgtaaagacccttctcttgctttggaaacaccctccctctcctgccrtgatcaaccctgatgttgggtagttctgcagtgggcacgtctgtacatgcctacccccaa

SEQ ID NO: 17 - DNA, 95 bp, Homo sapiens
tctgagtgcaagataaaaaggaaatagcaattcaaggcacaaagctaagyacatgcaacaatatagatgatttgggggtgggacagtacagaatt

SEQ ID NO: 18 - DNA, 110 bp, Homo sapiens
ttcttctgaatctagatgcagcatttagcaactctatactattcaccaayattatcttgctttatgttttcaacctataaaattcaactcagtttctgaaaaaggtgtag

SEQ ID NO: 19 - DNA, 110 bp, Homo sapiens
gaatctgctgagactgacgctggcgtcccaaccagacacttggaggcatrgggcatgcccttcaagtctagaaccctgccaggtacctatccctcctgcccaggccaaag

SEQ ID NO: 20 - DNA, 73 bp, Homo sapiens
catatggtgcaataaaattgtggtacagcaaagacaaagagaaaaaccayagaaagaaccgcagtaaaaacat

SEQ ID NO: 21 - DNA, 110 bp, Homo sapiens
ccctcccctcttgggtcttgtagtcggctgtgggtggcaagtacagcagygatggcagacaggtgtgatgagttcaaggacagggaaataacatccacagccccatccca

SEQ ID NO: 22 - DNA, 64 bp, Homo sapiens
tgtcacctcggatcacccgggaacgtagactccaaactcccaatggcttyagcagaaattgagc

SEQ ID NO: 23 - DNA, 110 bp, Homo sapiens
gggtgaatggatggatgagtgctgggaggctgtattgtaaataacaaaartgggatctgaaggaaggtatgggactctgaagtctgcattccttttaccttttgctatat

SEQ ID NO: 24 - DNA, 110 bp, Homo sapiens
gataaattgaattaaacgtggtattttatttggatttcttgatttttaaygtaagcatgaattatagtaactcttccttgctatctgaaatactgcaattcttgttctag

SEQ ID NO: 25 - DNA, 110 bp, Homo sapiens
gcgctgatgtggttgtggaggtcactgtggagaccacagaggggtagttrtcatgtccaggtcccaactgttaagcatgaagggaagtatagttttaaaggcttttgtgt

SEQ ID NO: 26 - DNA, 110 bp, Homo sapiens
gaagcgtccctgtgggcccccagaaataactgtcagaggcatgactggcrggggagatgggcaagggtcctgccattatgaagtgggttccaagagtcagcagagttgag

SEQ ID NO: 27 - DNA, 110 bp, Homo sapiens
cattgttctgtcttggagagggttcagtgctgggtggggagacatctgaraacagattattgcaattaagtgagaaaagctgaaagttaaattgcctagagtgtgcattc

SEQ ID NO: 28 - DNA, 110 bp, Homo sapiens
gccaggggcttttgctcccctacccacctgactctactgaggctcacctrggcgccccctctacctatccccagaaccgggccattgccacacccaatcagggtgtctgg

SEQ ID NO: 29 - DNA, 110 bp, Homo sapiens
tccccactctctggctgcatagaggttttaatgaaacaaggggataaccrtctccctcctgctggctctgttgacactgccagtgggttttgcaggcagagcaccctgcc

SEQ ID NO: 30 - DNA, 110 bp, Homo sapiens
gcccgcagacagaagggctgggacggctgtcagcgcctgtgcttccaagrtaagagccaaggactaatgagggccatcaggttctgggcagggacagggggcatcaggaa

SEQ ID NO: 31 - DNA, 110 bp, Homo sapiens
tatgcaagagtgctgagagacgagggatgatggctccactgttgtgctgsccacattgtatttggtgagtccaggctcctgctcttatcagaacagcaatggtagctgct

SEQ ID NO: 32 - DNA, 110 bp, Homo sapiens
ggttacatctctgatgtaaagttattgtataaaattcaagagaacatgcrttatggttgcgcttcagggtacaaaaccactggagggaaggatgaagaagtggttcaatg

SEQ ID NO: 33 - DNA, 110 bp, Homo sapiens
catctgaaaacacattgtaaagaatggcctctcgatgagtaaatatgggycaaatgaagtaaacagactatgcgtaggtatcatcaaatatagagagaatgaacagaccg

SEQ ID NO: 34 - DNA, 110 bp, Homo sapiens
cagggccccatattgaaaagagccatccatgctcattagcatggctctcycctccctccttatgtgtggtaatcatgtgagaaacagtcagctgagcttgtgacatgaaa

SEQ ID NO: 35 - DNA, 116 bp, Homo sapiens
gctgtcgggacccctactgagagaatagagaccttgtgttttatctcttcttgccrgagagctctgcctatggcctcgcagacagcaatcagtcaaggctcagccacccacacttc

SEQ ID NO: 36 - DNA, 110 bp, Homo sapiens
agattgcaagcaaggcattctcgagtttcaattctggaagagaaagcacrttggagcatttggtaggacgaactagctccagggcaaagatactgtggccggcactcact

SEQ ID NO: 37 - DNA, 110 bp, Homo sapiens
gatgtttaattcaacatgcagttcagaaaaatgacagatttgtcttgtasaaaaagacctaacacaagctaagcctttaagaaaaccaacctcaactgcatgaggcactg

SEQ ID NO: 38 - DNA, 110 bp, Homo sapiens
attataatccatcctccaacacacacacaaataagcagctaatggcaatrctagtggtcttcccaattcacaagacctgtgcttcaaattgttttcctgataatgtggag

SEQ ID NO: 39 - DNA, 110 bp, Homo sapiens
gtagtgtagtctttattcttggtgaaaggaaagaaatgtgcaaatgcttstttgcctgttgcacagtttcaaggatgaatttatccacacatccacttttgaaggtgcct

SEQ ID NO: 40 - DNA, 110 bp, Homo sapiens
ccaggtccctgtggctggacggggcacagcttgggagcagggcttcatgytacctttgctctgtgcagcaaagccctgcatggtcactggtgtctggctctctcctcctc

SEQ ID NO: 41 - DNA, 110 bp, Homo sapiens
ttgcctatcctagtaccaccctcactgcttctggtgctcacccatttctrgaacaaagctctgcattttttatatcaaatgtgcagacgtgtgccacaagcacacctcct

SEQ ID NO: 42 - DNA, 110 bp, Homo sapiens
actagctcccagaaagtagtttatcacggctcaacaaaagatcaataacrttgattgaattccattcatactagaaaacatatctatttaactgtaccctcagaaaaatt

SEQ ID NO: 43 - DNA, 110 bp, Homo sapiens
aactttaaacctattgtctaccacatccaaccaccacattgctttggctrcagagatcagtttaaagagcagatgattaaaagaattaaccacactttagaggtgaagaa

SEQ ID NO: 44 - DNA, 110 bp, Homo sapiens
cagtggtgacttgttagttatgtgactacaaacaaggcacagaaaactgrttgagcttcaggtttttcatatccttgagaattatagcatctgtatgtcagggttattaa

SEQ ID NO: 45 - DNA, 110 bp, Homo sapiens
aatagaaaaaccaccagaattagctacacggaatacacggatcactgccrtctcctcagagggatcaatgcgtttaagaaaaacacttggctcagcccaaggagaaccag

SEQ ID NO: 46 - DNA, 110 bp, Homo sapiens
ccagcgtaaccaccttctcctgactttagccctgacatccctgctttctrgtaatccccagctccaggcaagcccgaaggccagcaccctacactcacccttcctgggtg

SEQ ID NO: 47 - DNA, 110 bp, Homo sapiens; identical to SEQ ID NO: 46

SEQ ID NO: 48 - DNA, 110 bp, Homo sapiens
ggccctaagccagagctggagctcaggcccccagagttccgtccagggcrctccaggactcttcttaacaaagctatagaaattccgagctgaaaagacccataaagact

SEQ ID NO: 49 - DNA, 110 bp, Homo sapiens
atttacatctatttatttccattcagtgtatcacatcttcaggataggtrataacagtgtgaagggtgtgctcattttcttcagctgtgagtagaggagtcttcccgaga

SEQ ID NO: 50 - DNA, 104 bp, Homo sapiens
gaaatcgaatacgtccatttctttgtaaaataacaataacgttraaggcaaaagcaagattctgtaaaccaacattggaaaaggggacacagggaggggcagag

SEQ ID NO: 51 - DNA, 98 bp, Homo sapiens
agggaaagggaagtaggcatgtaccattttaatgcacrccttggttttcagaaaatttaagaagaaagactttctgcttttctctgactattctgtat

SEQ ID NO: 52 - DNA, 110 bp, Homo sapiens
ctgtccctatcttcaccagcctgccctcaaatcattggttcaaatcgctstcgagaagtttgggagctcccagggcgggggctttctaacagctctgcctagctcgggcc

SEQ ID NO: 53 - DNA, 110 bp, Homo sapiens
ggaaaagagtgtggaagttaactaaatctgcagctaataccacagaaccrtggagatgatctgaatgaagaaacttagaaaatgtggtgggtcagaacaagagtttactc

SEQ ID NO: 54 - DNA, 110 bp, Homo sapiens
aaaaaatcctgataagcactgttaggcatattaactttaatgattacaayttttaggacactctgtggcctagacttagaaacacaactaatgtccagaaaaagattcct

SEQ ID NO: 55 - DNA, 110 bp, Homo sapiens
tcccctccctcggcacccactcccaggccaagagcgtcctatgaatcatrcatttgttcctgttattgctgttcacagagtggcaactcttgcaaagggaggggtacaaa

SEQ ID NO: 56 - DNA, 51 bp, Homo sapiens
ttttacccaataaggtgagtggatgrtacatggagaaggagggaggaggtg

SEQ ID NO: 57 - DNA, 110 bp, Homo sapiens
atccctgcaacttggcacgtcttgctgtaaatggaagcagtggcatgtcygtgtcaggcatgaaagccgttcataaaaccaccttgctgagcacttcctgtctgtcaggc

SEQ ID NO: 58 - DNA, 110 bp, Homo sapiens
ctgcaagggttttgcttaattctcaattcaatgtctcttcatcttttagyagctgtggggttttgttgttgttcttctgtttttgcttagtatctgactactttttaatt

SEQ ID NO: 59 - DNA, 110 bp, Homo sapiens
acagaagtattttaactcctattttcattttattccctttctaaaactarctcagatcatagtcaggcgattgcttcattctcagctttgtggagagtgatatacacaga

SEQ ID NO: 60 - DNA, 79 bp, Homo sapiens
ggagtagctcttgtcatcatctaaaaaacttgaactgcattttgagagayttattggccttgcttctgcaggctcataa

SEQ ID NO: 61 - DNA, 110 bp, Homo sapiens
agaatcccccttgccattcccatccctgtatggtccttcctctagggctrtttgggaatagtacttatggtctaactggttctgcagctgcccttcgtgtggttataaat

SEQ ID NO: 62 - DNA, 110 bp, Homo sapiens
ctttcactgtgttttgtcgtgtatgtaggatttacccagtagcaaaactrtaaattcagggtcttcaagctaaaacttcctaaagcaataccctaagactcagttgtgaa

SEQ ID NO: 63 - DNA, 110 bp, Homo sapiens
attctcagtctgagcatctgaaggaggatggggactttatcccttcatcycaggtctgtaaatgtcgtgtgtatgaatgaatccacaccagtcccttcccatagacaaca

SEQ ID NO: 64 - DNA, 110 bp, Homo sapiens
agaagttcactctccttcccagatcatagacagaagacctgggttcaagycacgactctgctacagacctgcctgtgatttcaggctaaccgctgagggtcacagagata

SEQ ID NO: 65 - DNA, 110 bp, Homo sapiens
atcagagggagagaataaatgaccctgatactcttacttaggaaggcatsctcccccctccctcccctccctcgagaatcatggctttaaagacacagagatgagaaaga

SEQ ID NO: 66 - DNA, 82 bp, Homo sapiens
agcatactgaagactattcctaaatggctatttgtgttgggtggtcaagrctattcagaaaatctcagaggaggacaaatga

SEQ ID NO: 67 - DNA, 110 bp, Homo sapiens
ttctttttatttatgtctaactcctcagcaaaggaaggccttacttaacrtgataaacattcacattttattttagttaagcatttctttaaatgtaagaaattggaggc

SEQ ID NO: 68 - DNA, 110 bp, Homo sapiens
ctaaaggggacactactgtgtccaggagcatctcctctctcccaacacayacacacacaacaaaataagatcaaacctgctgatttaatatcaatttttcaacaaaaatt

SEQ ID NO: 69 - DNA, 110 bp, Homo sapiens
catgttttttatgagtcctctggcgcctttttgccttggctaccgaagcrtcttgaaagcagagatttttgaaagccccattgatttttgtgtcatccatctcagatcct

SEQ ID NO: 70 - DNA, 110 bp, Homo sapiens
aaaaccagtgcctcttgtaagcagccgcctcaacactgaacgcagaaaasaggacaaaaccatctgcgtgtcagcctgtcagcatatcactatataaattaaaagtcaaa

SEQ ID NO: 71 - DNA, 110 bp, Homo sapiens
gccagaccttagtggggaaagcgtttcagacccagacagtatgtctcccrcatggcatagagaatggcttgatgacttatttcctaaaagacggagcggcagtcccacta

SEQ ID NO: 72 - DNA, 100 bp, Homo sapiens
gcgcacgcaccagagccagtggcaaagcaacttaagctgcaggctcctcytgcccacccccagccgcgacgcccctgggggcccttagcagactgccaag

SEQ ID NO: 73 - DNA, 100 bp, Homo sapiens
atagtatttcttttcttcagtagaatggtcaggtctctaagtgctgcagyggcttgaactcttgatgacaaacatgtaggaaaacttcatgcagccagtt

SEQ ID NO: 74 - DNA, 100 bp, Homo sapiens
cttcaaaggccagaggcttggagctggcacaaggagtccttgtgcagaaragaacagaaggtggttttgctgccctacatccatccccggaaccttctca

SEQ ID NO: 75 - DNA, 100 bp, Homo sapiens
tccctataaggtatactgtttaaagactaggtccttggctttgccttcargtaacttgaagatgtttttgttgaaatatcttggagactaaaaaatagtc

SEQ ID NO: 76 - DNA, 100 bp, Homo sapiens
aagctcccccctactcagttgccttgaataggaattatactgaccaaaarctagatttaaaatgagaaactttaacatacccagttagacccacctacac

SEQ ID NO: 77 - DNA, 100 bp, Homo sapiens
tgagaagagagagcaactggaagggtgaggggagatactgagttttagayggttttcgaaaaagcaagtcactgcccaacaagtgatgttcagtagacaa

SEQ ID NO: 78 - DNA, 100 bp, Homo sapiens
caaaaaggcagctaaaaagacaaataaatataactggtggcacttacttyagtgtctgagctttaaagtcaaatttggctctggcaggtctcatctgaac

SEQ ID NO: 79 - DNA, 100 bp, Homo sapiens
gttaacatatctgaaattcataggtaacacctttcccaagataatttagytctggtattagatttagaatcttcatcctggtttttttggtctagtgttt

SEQ ID NO: 80 - DNA, 100 bp, Homo sapiens
tcaatagataggttgatcactctataaaatagtagtctgttggcacttaygtgcgtaaccagaggccatcatgttctgagactgccccagggctttttgg

SEQ ID NO: 81 - DNA, 100 bp, Homo sapiens
agagaagtcctgcagaggggaaatcataactcggagtacctcctaaatayacttgctataatctcacctccttctattttttatttttttttaattttca

SEQ ID NO: 82 - DNA, 100 bp, Homo sapiens
ttttattagtatagtggatgtctcttttgtttccctttcttgtcatttcwctttctttcccctgaggcaaatactattatgaatttagtgtcttcatttt

SEQ ID NO: 83 - DNA, 100 bp, Homo sapiens
cttccccgttgtggactttgggctgggccttgcaatttctctggccaagrgatgttggcctcctgcctgctgaggacatgccctgaagaacgtgtgcccg

SEQ ID NO: 84 - DNA, 100 bp, Homo sapiens
tccattccattctgtgccatcccatgccactcccttccctgccactccawgctattagaattggagacagagaccccaattacttttaatcctcagaatt

SEQ ID NO: 85 - DNA, 100 bp, Homo sapiens
ccttacccttgttagtggatagtggggatttaaaatagcagataatattrtatttagtaatagttaatattttttaaggatttattatgtgtgaagtact

SEQ ID NO: 86 - DNA, 100 bp, Homo sapiens
gtctcctttgaacatctggcctggactcatccccatgtctgtccatctgytgtcatcccaggcctggcctgcccacagcccgggccgcagtcaccactca

SEQ ID NO: 87 - DNA, 100 bp, Homo sapiens
tgattcacgtgtggcagggagggccgtcattaatgaaaatgcgagggcgkcgcacgagtgttgatgactcagcaggcctttctacttctgtatgagtcag

SEQ ID NO: 88 - DNA, 100 bp, Homo sapiens
cttctcatcaaataagtttgagtgttaagcatattgtggaatttggagartggcactgagtactattagaagcagtactgacgcaaatacattgcatact

SEQ ID NO: 89 - DNA, 100 bp, Homo sapiens
tctctccacagggccccagccaccgaaggctcaaaaaaaaaaaaaaaaamaaaaaaaacaaaaccctgcatggggagttgtgagaatctgaagcctgata

SEQ ID NO: 90 - DNA, 100 bp, Homo sapiens
tagagaagaatattctaaatcttcttgtgtcttctccttgttggtcttcrttctaatcattggaatcatgaagaagtttagtttctcttctgtgtgactg

SEQ ID NO: 91 - DNA, 100 bp, Homo sapiens
tgaaaaatgcttcccagaaatatgctcatatcattcatccattgacaatmatcatttgttggacatcaactgcttgtactacattagcttatgacatgaa

SEQ ID NO: 92 - DNA, 100 bp, Homo sapiens
gtacttattcccacagccaacttcaaatgaccccctccctggcccccccmcaagaaaaaaacagggcagctgaaaagtttgcttaaacttcttaatactt

SEQ ID NO: 93 - DNA, 100 bp, Homo sapiens
ctttaagatccatgtaaaaatattgcaaatgagggattttagtagtaagraaaatttgccaatttttactcgattagtttggctatttttatatattctt

SEQ ID NO: 94 - DNA, 100 bp, Homo sapiens
ccatctggcctaccctaggttaacgctagcttgcattttgctttgctttytgattttaatacatatgtcagtattactaaacaatatattgtttagtttt

SEQ ID NO: 95 - DNA, 100 bp, Homo sapiens
tcaagcgattcttgtgcctgagcctcttgagtagctgggattacaggcawgtgccactgtgcttggctaattcatatatatatatatatatatatatata

SEQ ID NO: 96 - DNA, 100 bp, Homo sapiens
tttaaaatgttactttattttagaataaggtagagttgaaaattgtcagraatacaaacattatttaactatttaatcctttatatatgtgaagttttaa

SEQ ID NO: 97 - DNA, 100 bp, Homo sapiens
ataatacttaattttttggctatgaagaggagaaaaatcatcattttctyatttttctcatcaggggactctgaagggcttgggaggagtgatctgccca

SEQ ID NO: 98 - DNA, 100 bp, Homo sapiens
atttagtcccagaagggcgtgaggacatcatgtagacacaaaagagcatrcaacagcaaattattgggggtaaatatttaatatcttgcgtctggggaac

SEQ ID NO: 99 - DNA, 89 bp, Homo sapiens
caccactttacattctctcaacaacagcgcacaagggtwccagtttctctgcatccttgacaacacttgggattttctgttattttgat

SEQ ID NO: 100 - DNA, 100 bp, Homo sapiens
ggttgtggggttctccaggtgctctgtcgcccaggctggagtgcagtggygcgatctcggctcactgcaacctcctcctcccgggtttaagtgattcttg

SEQ ID NO: 101 - DNA, 100 bp, Homo sapiens; identical to SEQ ID NO: 100

SEQ ID NO: 102 - DNA, 100 bp, Homo sapiens
aactaacttagagaagccactaaaggacaattcattctaattttcacgtraatcaaagacttatctgactaatgctgaataagttggggagagatttcat

SEQ ID NO: 103 - DNA, 100 bp, Homo sapiens
aaaaagaatggcatttggcaagatgagactttggttcctaactgctctargacctaggctggcttgaggttcaaagtgccggttactctccatttgactg

SEQ ID NO: 104 - DNA, 100 bp, Homo sapiens
agcagtggtgagaggggaagagatttttctatgccatgtttcctgacagkagcagaacctgtcttcagacctgaaacacttctccttttatagtcactat

SEQ ID NO: 105 - DNA, 100 bp, Homo sapiens
tactgcatgaaggaccaaaccaacaagccagtcaggacaccaaaagcacmcaaggaagcaaggaacaagatgctgcagttcttaaaataaactgtaggtg

SEQ ID NO: 106 - DNA, 97 bp, Homo sapiens
attgacatcaacaaaagttatcacagggttaaactgacacacgtttmatgatatgcgtagtcattgctaagtaagtaggcataagaacaaggtaatt

SEQ ID NO: 107 - DNA, 100 bp, Homo sapiens
aggtgccccggcccagagaacctgccagtgaaattgcgtcctacccacarcccctcgttcccatagctatggccttacttggggccctctttgcctctga

SEQ ID NO: 108 - DNA, 100 bp, Homo sapiens
tattaaccacctccaaccttctgtctagagatcatccttattatcactawactctagtttggctataaagtatataaagaagtagatgtcaaaatggaca

SEQ ID NO: 109 - DNA, 100 bp, Homo sapiens
taagacccctgtcatttgttcaacagatgttcccctccaaggctcaccayaatctgccccaaatccatctctctggttttatcttctaaaattccccacc

SEQ ID NO: 110 - DNA, 100 bp, Homo sapiens
gttcctcttcaggtcggtcaagaccaggtttggaactcagcttgttcacrtctgtttccaagtcatcatcctaaaatggaatagtcatagaagagattac

SEQ ID NO: 111 - DNA, 100 bp, Homo sapiens
tttttcccattctgtgggtctgttcactttgttgtttttgtttttttgtktgtttttgtttttgttttgctatgcataaactttttagttttatgcaatc

SEQ ID NO: 112 - DNA, 100 bp, Homo sapiens
cttttggggctgaggtaatagtggaatgctcccttacaggaagctacccrggacttcacaggccctgtgccagtgacttccttgaaaccaggaatattga

SEQ ID NO: 113 - DNA, 100 bp, Homo sapiens
ttgcaaacgtggagtgagggaaggggattaaaaagggagaaggtgaatargtgactttgaatccaacctacagtggttaattttcatttaactatctggc

SEQ ID NO: 114 - DNA, 100 bp, Homo sapiens
attgttaagtataacagtttgctaagtaaggtgagtggtataatcatatrtttgtaaaaagcaaaacaaaataaagccaaaactcgaacctatctttgtg

SEQ ID NO: 115 - DNA, 100 bp, Homo sapiens
tgtcaaaggggtctttatttaagcctcagaacacatctagtagaagacargtaatatgaggaaagaggttgaggtaggacaagactgaaaactttgtaaa

SEQ ID NO: 116 - DNA, 100 bp, Homo sapiens
gtccacagggcccctgccctgcctttctgtctgtcatagagcaggaacgrctgaccatccagatgtccctcagcgagaaaccctgactatacaaatccat

SEQ ID NO: 117 - DNA, 100 bp, Homo sapiens
atctccatctataaaatgggaatcacagtagtacccacctcatatgaccrttgtgaggattagatgacattttatatggaaaatgtctgcagtaaaccgt

SEQ ID NO: 118 - DNA, 100 bp, Homo sapiens
ttgagaaattttgaggaagtacaaccaaaagcaattggagactggttatyggaaggtgcggaattgggtgaaggcacgatgcctgggtagctggtgctgc

SEQ ID NO: 119 - DNA, 100 bp, Homo sapiens
acaatctgtcttgcactaaaactaggcgttaaagcaccatctaagaattwtttttttgagctagaagatggacccatttaagaaaggtcacagttccttc

SEQ ID NO: 120 - DNA, 100 bp, Homo sapiens
agatgagcagccagttaaggttaaagaatcagtggatttggaataaggayggaggtttggaggattctgggcttgggaaaaataagagcaaaggtactga

SEQ ID NO: 121 - DNA, 100 bp, Homo sapiens
ggtcaggggttgggctgactcatccagacctctaaacattatctgaggcyacctagggggtcagggggagttaaggcagtggcctctggctgcctcggtt

SEQ ID NO: 122 - DNA, 100 bp, Homo sapiens
cttccttccttccttccttccttccttccttccttccttccttccttccytccctctctctctctttctttctttctttcttttatttttgttttgggtg

SEQ ID NO: 123 - DNA, 100 bp, Homo sapiens
agagcagaaacccaaaagctgttagctgaaaaacaaacaaaaaatacctwtggtgaagcgtgatttttgggtgaagttaaaggcaggaagctggtagagt

SEQ ID NO: 124 - DNA, 100 bp, Homo sapiens
ccaagtacttacaggatttctccaaatgtgttgtattgataataatctgracaagctagaaaacaacaaacacatgtaaaagtcatttaactactcaccc

SEQ ID NO: 125 - DNA, 100 bp, Homo sapiens
tttcagaagctaaccacctgacttgagactagaattgcacatgttgcatrccattcttgcctttgactctttcagacattgtatccaatatcatctatca

SEQ ID NO: 126 - DNA, 100 bp, Homo sapiens
taaggtactataattgatatacttgtatacagggtccttataaaatacarctcatacctaaatccagatatttaataattgggcccattaagtccaatca

SEQ ID NO: 127 - DNA, 100 bp, Homo sapiens
gtcatgaaaaggttaatctacaatgcacttagacagctaataataagtaraacatttccaaagaaataggaacctgggattgtctctttttaataaatta

SEQ ID NO: 128 - DNA, 100 bp, Homo sapiens
cccacattaggcataaaacataaatgtataccttaataaatttcggttaygtaaaagagcgcctgggtaacctcacccaggttgagatggaaaatgtgcc

SEQ ID NO: 129 - DNA, 100 bp, Homo sapiens
tatacaggatggggattccagacaatcagagtatgatgttctcttgtcaytgatcacattcactagtgacaactaattagcacatccgagtaatcaaaaa

SEQ ID NO: 130 - DNA, 100 bp, Homo sapiens
gcaccgcctgtctataccagtgctgatcccacctctcccctcccccttcygccctcctgcctttgcaattggctgtttccctccagctttgtccatcctc

SEQ ID NO: 131 - DNA, 100 bp, Homo sapiens
gcgggtggagccctcctctagctatcagcatgactgctgtgttttctgaytcaccagttccctgtccaaggccatccctaagctctgttttcttccagtt

SEQ ID NO: 132 - DNA, 100 bp, Homo sapiens
atggtactatgctgtaataaaaacctgaagtgtgggagaagtttcagaaycaagaactgagaggaagctgaaaggactttgaggagagtgtaagcagaat

SEQ ID NO: 133 - DNA, 100 bp, Homo sapiens
ttttatacaaattcatcttcttatagttgggatgttattacaaaagtttsgatatcttaaaaatccctaatgttttagagtttcatatttattgacctga

SEQ ID NO: 134 - DNA, 100 bp, Homo sapiens
ctcccttttcaaaggtgtcatttatgtatacatattatttttatttttawttcacatacagatacctaaactattccatttttaagagtagaaatcatgc

SEQ ID NO: 135 - DNA, 100 bp, Homo sapiens
aggggagtgaacagaggatgaaagagagaagaaagggcagtctcagctcrctgtgccagagtgcttagctcagcgtataacagggactccataaatatgg

SEQ ID NO: 136 - DNA, 100 bp, Homo sapiens
tgcaggcaatttagtagacactgaagcatatttttacctttccaaattgraagtggataacaaggtaacaatagcactgtccctggagccatcctgtcct

SEQ ID NO: 137 - DNA, 100 bp, Homo sapiens
gatactcatccctgggtcccacgcccagagagtctgatgtaatcagtgtsgggtgtggacagggcattgggatttttaaaagctccccaggtgattgcag

SEQ ID NO: 138 - DNA, 100 bp, Homo sapiens
ctatattttacgtaagttcaaggtatgacaatgcatatgtggcaaagatrtatgtagtctgctttataaaaagtgtctaaggaaacaaaattttctttcc

SEQ ID NO: 139 - DNA, 100 bp, Homo sapiens
atgtaacaaatacaattaaatggagatgcaatttgatttattgaaaatarcatgattttaatgcagattaactgtaggcagtaccttaccagaatataat

SEQ ID NO: 140 - DNA, 100 bp, Homo sapiens
attctgatagagaccatatgacctccaagtggattagctttcaatgaatkctacaaaccagaagagtctttcccccatcttcatcttcattctctaactt

SEQ ID NO: 141 - DNA, 100 bp, Homo sapiens
taaggtgagagaggcagaaggagggtggaaagaatggcgtttttggtgcrgtgcaaactaatttgcccaaactgaaaaagctggtgctctcaccattatg

SEQ ID NO: 142 - DNA, 100 bp, Homo sapiens
cagtccttccgccgccggggctaacacaatagtgaaagctgcgctcctgrtcgtcggcaggctggcactgttatacaggaaaagggtcgtgtgcgacctt

SEQ ID NO: 143 - DNA, 100 bp, Homo sapiens
gcttttaggtctttaaagcagcacatgattttcaagtgagttattcaatyaatcgtttctgtatcactctgatgcccctttggactgtctgcttattgaa

SEQ ID NO: 144 - DNA, 100 bp, Homo sapiens
ctgagctcagccagggaccaggaaccagggaccagagtcaggggagccgkctagaacagaagggcacgtcggtgctaggctgggggaggcgcagagatgg

SEQ ID NO: 145 - DNA, 100 bp, Homo sapiens
atcaggaaggggaggccacaggagttacagaggactctggaaatacagaragatctgtgatttactcagtgggtatttgatagactaagaaggcaaattc

SEQ ID NO: 146 - DNA, 100 bp, Homo sapiens
cctgcagttttccacatggaattcctcttcttgcactttggcctaagcaygcattttttctaatggtggaaaatattaaacagacttttgctttagaagg

SEQ ID NO: 147 - DNA, 100 bp, Homo sapiens
tcagcccagagaaagagtcagtcccataatcccagatctccaccacatcygcacagaggcctgttaggccagggggctgccctgctccccgctgccccca

SEQ ID NO: 148 - DNA, 100 bp, Homo sapiens
attaaaagggaactttttatctcatatttgaaacataagtcacaagaatractaacatttatgctttaaattattaatcctagttttcagaattttatta

SEQ ID NO: 149 - DNA, 100 bp, Homo sapiens
aaaacttttaccaataattttcatggaggattttatgaaataaccaaaakctattaaaaatgcttatgctgaaaaatataaaatattcagttttctatta

SEQ ID NO: 150 - DNA, 100 bp, Homo sapiens
tgcaaaagagaaggccacaccttgctcagaaagacctgatgtgtggggcyagtgcaaggggtcaaaaactgaggagaggccagaaggccaaagcagcttg

SEQ ID NO: 151 - DNA, 100 bp, Homo sapiens
tcacataatggaagaatggcccggcctagaccagaggttcctaattaggrgtccatgagccacgttcaattatatatattttttcttctggtatttttct

SEQ ID NO: 152 - DNA, 100 bp, Homo sapiens
cagtttaaggtggaaagggggtaggaggaaggaggaagtgtatttctttmgtcacattagccagattctctaccttgacattgtatctcccagatctatt

SEQ ID NO: 153 - DNA, 100 bp, Homo sapiens
tctttcctcttttatgtttccttggctacatgctgggataggggctcatrgtaagtttgccagattcaaccaaaaaacgccacaaaaacaagaaaacagg

SEQ ID NO: 154 - DNA, 100 bp, Homo sapiens; identical to SEQ ID NO: 73

SEQ ID NO: 155 - DNA, 100 bp, Homo sapiens
tttaacctccctccctgcgggcgaatacccagttgtcccagtacattttrttgatgagtccttttttgtttttgttttttgagacacagtctcgctttgt

SEQ ID NO: 156 - DNA, 100 bp, Homo sapiens
cataattgatgcggccttacaaactatttgatgtccaaaccccatccttyaccgggcctaccaggcgaggctagtggaggtgagaagcctcacaatccat

SEQ ID NO: 157 - DNA, 100 bp, Homo sapiens
acagtgccaccttcacgccagggcctgggaggtctgtatgcaggttaccrggaagaataagtcagtttagtgagcattcggcccataaaaccaccgtgca

SEQ ID NO: 158 - DNA, 100 bp, Homo sapiens
agtgtgtcactcctaactcttcttacctcctttctttggtttaaggtctmttagtgtctgtactgggtagtgttttcacagctttctcctctgacatggc

SEQ ID NO: 159 - DNA, 100 bp, Homo sapiens
cctcacccacgatggcggccagtctcccgaagcgcgaggccccgcccagytctcggcctctcccgcggccggtccctgcaccaagctcgcgagctgactg

SEQ ID NO: 160 - DNA, 100 bp, Homo sapiens
tgggcctggggggttcatgtccaatgttgtccaatcactggataattctracatctaactaaacctcttttataggaaaaagagatgctttaaaattgtt

SEQ ID NO: 161 - DNA, 100 bp, Homo sapiens
ctgcaggcagactcagatcagcagagaagcatctcgcctctggctgtgcygtccaggttgagctgcgtgtggcagtgagcacagaggagaaatgcaggga

SEQ ID NO: 162 - DNA, 100 bp, Homo sapiens
tgggaactttaccactttttattattttgttccttttctctgctggaaaygcgtttgctgtcaatattatgccatatatcgctgtgccaacttaagttgc

SEQ ID NO: 163 - DNA, 90 bp, Homo sapiens
tctctatcagtataacaaaggagtcagacttgatggcctstaagggtccttccaagtactttaacattctagaaatttaccggacagaag

SEQ ID NO: 164 - DNA, 100 bp, Homo sapiens; identical to SEQ ID NO: 76

SEQ ID NO: 165 - DNA, 100 bp, Homo sapiens
atgctgtcctagccaatatctgctcaccctcttcagccattccctgtacygcacattcgctccagccatgactgccatccacggcccactcatcacatac

SEQ ID NO: 166 - DNA, 100 bp, Homo sapiens
acatatttgatgacagagacacaatccctgtcctcaaggagctttcattytagtagagaagatgaaaaccagtacagtttggtaagttagatgatattgg

SEQ ID NO: 167 - DNA, 100 bp, Homo sapiens; identical to SEQ ID NO: 77

SEQ ID NO: 168 - DNA, 100 bp, Homo sapiens
aagaggaagggggatgtgaaacatatacagtacattactctctgcgatcytcctctgtgctatcaacaagcaagaaaaaagagatctggagaaaacagat

SEQ ID NO: 169 - DNA, 100 bp, Homo sapiens
gtcactgggcagtccatgactgattgccgtgctagttgtgtgggcactamtccctttagatctttagaggaggctgagttcactttagacagagaggttt

SEQ ID NO: 170 - DNA, 100 bp, Homo sapiens; identical to SEQ ID NO: 78

SEQ ID NO: 171 - DNA, 100 bp, Homo sapiens
atgtttttgagtttgttttgggaaatctactttgcacattttgttaattrtctttgaactcaagctttccacaggttcaactgctattcttcccacttag

SEQ ID NO: 172 - DNA, 100 bp, Homo sapiens
taggaactgaaagtacctgagctgtaaaactagagaaacctttcatatgkaaagggcaagggaaaaaggagttaacatgtataagcccgaggaagttgaa

SEQ ID NO: 173 - DNA, 100 bp, Homo sapiens
agagagctgtactgggagcctggaaaaggaaggtctaatcggcaattttyctttctctacccatgtatttggaaaaaataatgaattatctggttgtggg

SEQ ID NO: 174 - DNA, 100 bp, Homo sapiens
ccagtaggtgtcagctacgcacagcctttctctttctaaatccatatttrtaagaaaacggcctttcatccataattttccctagttaaagggatcaatt

SEQ ID NO: 175 - DNA, 100 bp, Homo sapiens
ttattttacttttttgtatttgaggttttgatcttgctctgttttgtgtragaaataaattagaaattcagcattatttccttctcaaatgtttagccat

SEQ ID NO: 176 - DNA, 100 bp, Homo sapiens
cgtgaagcaaaatctcattggtaagtctgagttgcccaatgagggtttayaagaatacattcacgttttctaacttcattaccttttcttacttgaattt

SEQ ID NO: 177 - DNA, 100 bp, Homo sapiens; identical to SEQ ID NO: 79

SEQ ID NO: 178 - DNA, 100 bp, Homo sapiens; identical to SEQ ID NO: 80

SEQ ID NO: 179 - DNA, 100 bp, Homo sapiens; identical to SEQ ID NO: 80

SEQ ID NO: 180 - DNA, 100 bp, Homo sapiens
cttccttaatgatcagaacagagataaaaatagtgtttagaatgtcctgraagcctttgggaatgttaaattagaagacatctattttgatacagatgtg

SEQ ID NO: 181 - DNA, 100 bp, Homo sapiens
aaacatttccttctgttccacccctggtattcttgtatgtgtgttctgcrtaggagggcctgggagttcctgttgcttctctgtgaaggggggaggactt

SEQ ID NO: 182 - DNA, 100 bp, Homo sapiens
tcaggttcacatgtgcatttggttcattatgtgtctttagtgttttaatytggaacagttctcttcccacctttggatgtaagtggcacatttgtttccc

SEQ ID NO: 183 - DNA, 100 bp, Homo sapiens
aagtaggcaagaccctaaaaagaagccacctgcctctcccagtccctccrggaggactccaagagggcaaatctgcaggcagtgagctcagggccagcgg

SEQ ID NO: 184 - DNA, 100 bp, Homo sapiens; identical to SEQ ID NO: 81

SEQ ID NO: 185 - DNA, 100 bp, Homo sapiens
agctccttttgatattccctgccccagagctcatgaccagaacccagagytgatttaaaatattttgaaaaatggaggaggcagactgctcccagcagcc

SEQ ID NO: 186 - DNA, 100 bp, Homo sapiens
acacaggtccatctgtggacattgtatatttaaaaaggcctgtgggaacratccatgacagggccactcatagaagaagctgcaaatttttagacggtgg

SEQ ID NO: 187 - DNA, 100 bp, Homo sapiens
tccctctagctgctggggggctcacttggatggtcctaaaggcacccccyttcttccaatggtggggtgccctccagtgttcacgggatgtgggctcata

SEQ ID NO: 188 - DNA, 100 bp, Homo sapiens; identical to SEQ ID NO: 83

SEQ ID NO: 189 - DNA, 100 bp, Homo sapiens
gctatggttttttgtttgtttgtttagcaccagcaggcctgatagcaatrgcaatttttaagaaaagaaaaaattagttgggagaaaggtagtgtatgag

SEQ ID NO: 190 - DNA, 100 bp, Homo sapiens; identical to SEQ ID NO: 84

SEQ ID NO: 191 - DNA, 100 bp, Homo sapiens
caaggaagggctattataattctatttgaaactggctgcatttatcttcwtctctctcagaagtcacatttcccttgcccttggatgaggactggaatgc

SEQ ID NO: 192 - DNA, 100 bp, Homo sapiens; identical to SEQ ID NO: 85

SEQ ID NO: 193 - DNA, 100 bp, Homo sapiens
aatttggaaactatagtacgtatcaggtatcgagaaggaagtgaatatawctcctataagctaactttctagtctttttttcttgagcatttttttttca

SEQ ID NO: 194 - DNA, 100 bp, Homo sapiens
ttgagtattcctcagctttagaagaaaatgtttttctaaaagggaacaayactctgaattcctttttttctctaaaatggaatcactttttacaaatctc

SEQ ID NO: 195 - DNA, 100 bp, Homo sapiens
taatgaacaggatgcacctgttccatgaaccctctctctcttccctagarggagacacccagatcctcatcagtggcagcagacacctccaagtctagga

SEQ ID NO: 196 - DNA, 100 bp, Homo sapiens
ggctttgggcataaagcagccggctggctgttggatctacagagtcaaaygtgaacagggaggccttacgaagcaagccttgttttgctgagagctctgt

SEQ ID NO: 197 - DNA, 100 bp, Homo sapiens
tctagtgaaaagactaattattttttgtattatcctgtttccaattttgyggatcacttcatactaaactgaagacaaactcctatataatccttatgtc

SEQ ID NO: 198 - DNA, 100 bp, Homo sapiens
tagacttatcttccaggctcgtgaagagtgcagtaatgctaatgttgagrgatgttaggccagatcacatagcatattcctctcatcatcctgtcaggaa

SEQ ID NO: 199 - DNA, 100 bp, Homo sapiens
tacattgaagaccaagccattctcagtctgctcttcacttctaattctamggttattctgattgctcaggacaatgctgtctgggaagactgccccacag

SEQ ID NO: 200 - DNA, 100 bp, Homo sapiens
catgaagcagtaaatggagcaattaacctggggcctgaatgggaagaagkttctggaggcaacagggagtctggagcaaaatatcaccagatgacaatat

SEQ ID NO: 201 - DNA, 100 bp, Homo sapiens; identical to SEQ ID NO: 88

SEQ ID NO: 202 - DNA, 100 bp, Homo sapiens
tcctagtttttgactttactgcctatattacagaggaagtacggtatgtrttgaagaaattagaagtccacccatagctcaaatatgcttcagtatcctt

SEQ ID NO: 203 - DNA, 100 bp, Homo sapiens
tccatgtgcccaacttggaattctcttctctgattttcccataacttaaytgaagtgagaactttggggagttcaagcactgtttcacttatttcctttt

SEQ ID NO: 204 - DNA, 100 bp, Homo sapiens
ggcctcttatggctgtattctattagctctgggtttactgtgatattatrtatatttgtcttgggccaaacagcttgcttgcctcagttgaagtctaaaa

SEQ ID NO: 205 - DNA, 100 bp, Homo sapiens
ggcatgaatatatatcaaagtttcttttttcccaaggataaacaaattgycccagcaccattaattgatgtactaatgtgctcaaaacacaaaataaatg

SEQ ID NO: 206 - DNA, 100 bp, Homo sapiens
atgcacaaatgaaaggttccctgtgctctatccctgcacaacatccttcrtctgctgtgggagaaatcatcttaaatacttaaatgtagaattctgcagc

SEQ ID NO: 207 - DNA, 100 bp, Homo sapiens
agtctatctattcatttatttttcctccaaacctgcttattttttttaartaccaatacctgaaaattgtattttattgaactgcatattaatgcattct

SEQ ID NO: 208 - DNA, 100 bp, Homo sapiens
tgttttactaaagttgattgaaattagttagttgatattcatcctaccakattttgagttagagaaatttagatagcaatatttgcacctaaactatttt

SEQ ID NO: 209 - DNA, 100 bp, Homo sapiens
actacaacagaaacacactgtcacacttgctagaatataactgtacttgrgcttctcctttcctgtgaagtagtgctgggctttctagagtttaattctc

SEQ ID NO: 210 - DNA, 100 bp, Homo sapiens
ctgctgaaaggcaatacagtggccccatctagagatggtcacccaggactatgaatcactatgaatttggcaggggaatggaaagatcaygagcagctca

SEQ ID NO: 211 - DNA, 100 bp, Homo sapiens
ggacttccaggcaggatgctgaaggataaatgtggacacgggtgagggargcacctgtggaatctgcagggaggctggactccagtcctgatgtcggccc

SEQ ID NO: 212 - DNA, 100 bp, Homo sapiens
ggcattatcatctcaaagggcaaaagaaggatggctaaaccattggaaaygtggattcgctatttttgctatggtcccatttagcccctacccatgtctg

SEQ ID NO: 213 - DNA, 100 bp, Homo sapiens; identical to SEQ ID NO: 91

SEQ ID NO: 214 - DNA, 100 bp, Homo sapiens; identical to SEQ ID NO: 92

SEQ ID NO: 215 - DNA, 100 bp, Homo sapiens; identical to SEQ ID NO: 93

SEQ ID NO: 216 - DNA, 100 bp, Homo sapiens
ctaaagttcccttgtcctcagagtgagagttcatagggctcatttcaaarctctcagccaatgaagaaattaaactggactgtcccaaagaagatgaaaa

SEQ ID NO: 217 - DNA, 100 bp, Homo sapiens
ctccatgttattcctgacaaacagttgtcccatgaacaacatcccaacaratcatacttttcaagtagcaattatcagagggtgctaataattccaggtt

SEQ ID NO: 218 - DNA, 100 bp, Homo sapiens
gctcttaagtcaagtcacttgtggaacttgccatataaacaatggtttgyaatgtaatgtgttgaagtataaaatagagttgtgttcaaagtactggggt

SEQ ID NO: 219 - DNA, 100 bp, Homo sapiens
acgggcgagtatccatggtgctgattcttccagttgtcccaaaacaaatyttcaatgaattctgaggccccaaggcttcccttgttggccaagtcactga

SEQ ID NO: 220 - DNA, 100 bp, Homo sapiens
cagaagttatttgctcttgtgcataattgaaaacaaatgttctgtgttgkgtacaattattttgatcttaaaggctatgcttcgtagaaaaagagaaggt

SEQ ID NO: 221 - DNA, 100 bp, Homo sapiens
cagcttgtccccgttaggtgacaataagcagacagaagagccaagcagcyatggaggaaaatcaggtacctaaatttagctcaactcctgcctcattcag

SEQ ID NO: 222 - DNA, 100 bp, Homo sapiens
ttttagcttcttttctactctctcaatcttatccgggcttgtttgggggygaactctttatcttttatagaagataaagtttggttttattaaatttttt

SEQ ID NO: 223 - DNA, 100 bp, Homo sapiens; identical to SEQ ID NO: 96

SEQ ID NO: 224 - DNA, 100 bp, Homo sapiens; identical to SEQ ID NO: 97

SEQ ID NO: 225 - DNA, 100 bp, Homo sapiens
ataaatgtctggtttaaaggagaaatcaatactgttattcctctccaaarattaagcccagtctaggaagagaagattggagttttagacttgattagtt

SEQ ID NO: 226 - DNA, 100 bp, Homo sapiens
acattaggcaagttatattacttccttgctccttactatcttcatctgtmaaatggggaaagtatttaacatagggccaactacaagcaaattggctttt

SEQ ID NO: 227 - DNA, 100 bp, Homo sapiens
gaaacagtggtatatactgccaaaaatgtatcttcctaattgtcagtctyaaaaaaggtagtttccaaactacctcttttttaaacatctatcagttttt

SEQ ID NO: 228 - DNA, 100 bp, Homo sapiens
tcctagctactcgggaggctgaggcaggataatcgtttgaaccctggagrtggaggttgcagtgagctgagattgtgccactgcactccagcctggacta

SEQ ID NO: 229 - DNA, 100 bp, Homo sapiens; identical to SEQ ID NO: 98

SEQ ID NO: 230 - DNA, 100 bp, Homo sapiens
agaaccagaggctgggaaggacaagtcacctgcttgtgagtggcaggggmaggattgggagcagtggactcggtctcaggactgcggccctcaggaccaa

SEQ ID NO: 231 - DNA, 100 bp, Homo sapiens; identical to SEQ ID NO: 100

SEQ ID NO: 232 - DNA, 100 bp, Homo sapiens
aatggttgcatattattgtcaagtgggtgttaccacatgaatatttacakttgttcagctcatctgtgaatttcttctcttcattcccaggactcaccat

SEQ ID NO: 233 - DNA, 100 bp, Homo sapiens
atgcaaatgtgttcatggtcatgggtggtacactggctagttttaaataygagccctcagagctgcaggagaaggccacatgaatgcctccaccacagac

SEQ ID NO: 234 - DNA, 100 bp, Homo sapiens
ggataatgttatatataccttgtggatatacaccttgtggtgtgtgtatrtatatatatatatatatatacatacacacacaccttgtggagtgcagtgt

SEQ ID NO: 235 - DNA, 100 bp, Homo sapiens; identical to SEQ ID NO: 103

SEQ ID NO: 236 - DNA, 100 bp, Homo sapiens
gggatgatacactctttggtcctaccaaacagctttcatcttgagatcgycctccatcttcattgtggcccttcctctcagttctcttttgtgtggtccc

SEQ ID NO: 237 - DNA, 100 bp, Homo sapiens; identical to SEQ ID NO: 104

SEQ ID NO: 238 - DNA, 100 bp, Homo sapiens
gctggacacctgggaggggcagcctgtctgtcttctgctctgccctttcytgctgtgttctggaagcataggcaagcattccacagtatcctgcctgcag

SEQ ID NO: 239 - DNA, 100 bp, Homo sapiens; identical to SEQ ID NO: 151

SEQ ID NO: 240 - DNA, 100 bp, Homo sapiens; identical to SEQ ID NO: 158

SEQ ID NO: 241 - DNA, 100 bp, Homo sapiens
ccaaacagcactcagaaaatcataaaatggcaagcaaaatgagcaattcygcacgcagcacgtgacatggagtatgagctcgcatttgccgaaggagcaa

SEQ ID NO: 242 - DNA, 100 bp, Homo sapiens; identical to SEQ ID NO: 166

SEQ ID NO: 243 - DNA, 100 bp, Homo sapiens; identical to SEQ ID NO: 168

SEQ ID NO: 244 - DNA, 100 bp, Homo sapiens; identical to SEQ ID NO: 169

SEQ ID NO: 245 - DNA, 100 bp, Homo sapiens; identical to SEQ ID NO: 171

SEQ ID NO: 246 - DNA, 100 bp, Homo sapiens; identical to SEQ ID NO: 173

SEQ ID NO: 247 - DNA, 100 bp, Homo sapiens; identical to SEQ ID NO: 176

SEQ ID NO: 248 - DNA, 100 bp, Homo sapiens
ctggtaataacttggttggtctgaacccatgcacagctcagaagtgtgartgagttaatacccccaaggaaaacttttgactaattgggggaagaaagca

SEQ ID NO: 249 - DNA, 100 bp, Homo sapiens; identical to SEQ ID NO: 180

SEQ ID NO: 250 - DNA, 100 bp, Homo sapiens; identical to SEQ ID NO: 181

SEQ ID NO: 251 - DNA, 100 bp, Homo sapiens; identical to SEQ ID NO: 182

SEQ ID NO: 252 - DNA, 100 bp, Homo sapiens
gcaggaccgcgcacagagtaggcagcatttgtgtgtgcctcgcacgtgtrtggtggcactgtatgcacttgtagatactcaaatacgtagggaaagatga

SEQ ID NO: 253 - DNA, 100 bp, Homo sapiens
acccagtggttctgagctctggcccttctactgcctcttgccagctctgkgtctcagccttcctatctgtgagttagacaccaggtagctggaggggaaa

SEQ ID NO: 254 - DNA, 100 bp, Homo sapiens; identical to SEQ ID NO: 193

SEQ ID NO: 255 - DNA, 100 bp, Homo sapiens; identical to SEQ ID NO: 196

SEQ ID NO: 256 - DNA, 100 bp, Homo sapiens
cggctgcgtctatggtcatgacgtctgacagagcgtccacccgtcttcgmcaggactctatggttcttacgcgcgcagacagaccgcctatataagccat

SEQ ID NO: 257 - DNA, 100 bp, Homo sapiens; identical to SEQ ID NO: 197

SEQ ID NO: 258 - DNA, 100 bp, Homo sapiens; identical to SEQ ID NO: 199

SEQ ID NO: 259 - DNA, 100 bp, Homo sapiens; identical to SEQ ID NO: 203

SEQ ID NO: 260 - DNA, 100 bp, Homo sapiens; identical to SEQ ID NO: 204

SEQ ID NO: 261 - DNA, 100 bp, Homo sapiens; identical to SEQ ID NO: 205

SEQ ID NO: 262 - DNA, 100 bp, Homo sapiens; identical to SEQ ID NO: 207

SEQ ID NO: 263 - DNA, 100 bp, Homo sapiens; identical to SEQ ID NO: 208

SEQ ID NO: 264 - DNA, 100 bp, Homo sapiens; identical to SEQ ID NO: 209

SEQ ID NO: 265 - DNA, 100 bp, Homo sapiens
tcaagtgtattttaccacaaaggcctcttcagggacatttataacatatmccagatgtagttttccaaggagagtgctttgagaaacactgaccaagcct

SEQ ID NO: 266 - DNA, 100 bp, Homo sapiens; identical to SEQ ID NO: 211

SEQ ID NO: 267 - DNA, 100 bp, Homo sapiens
aataagcattctcaattctgcaattcagtggcattgcatatatcttaggyggtgtttatgacttcatatttataatttggcattcatgtgtttatcttct

SEQ ID NO: 268 - DNA, 100 bp, Homo sapiens; identical to SEQ ID NO: 216

SEQ ID NO: 269 - DNA, 100 bp, Homo sapiens
acatgaaagaatttttggccaggcaatggagatagacagtaccattagcygaaaacaataagacaatgacatgccatactagaaatattaaatcattctt

SEQ ID NO: 270 - DNA, 100 bp, Homo sapiens
tgctctgtattggctgtggggtgggggtggcggtggaactgacccctggsgtctgcatttctcaggctcccagggctgtggctgactttggccaatggga

SEQ ID NO: 271 - DNA, 84 bp, Homo sapiens
ctccaggcgattcagatgcaggcttaagtctgagaaccactgttgtaggyggttgtggttggccccgttcttagagtagaattc

SEQ ID NO: 272 - DNA, 100 bp, Homo sapiens
agcattgctttgagaatagaaatagtcaggaatggttggctcatgcctgyaatcccaacactttgacaagctgagactatcacttgagaccacgagttca

SEQ ID NO: 273 - DNA, 100 bp, Homo sapiens
cctcatgatttgttctgccacttttagaatactaaatgctcacttatctytaagatcttatctagagctatcatattaagtttaggactattcctctacc

SEQ ID NO: 274 - DNA, 100 bp, Homo sapiens
ggtaaaatactgacttgggtcacatgatctacctcaccatagggagaggkatactcactgacctggtcagagccatacacccaaggagataacctttgcc

SEQ ID NO: 275 - DNA, 100 bp, Homo sapiens
aagccaatattctatgttctatttctcagtttctgtcatcaatagatatmtaggcccttctcccacattcactgaatatctacagccaacctatccaata

SEQ ID NO: 276 - DNA, 100 bp, Homo sapiens
aaattcatcatgatcttaggtcttctgatatttctaacttcaatcctcawtgctgttatggattaaatcttttaacccacttgctattatattgatggga

SEQ ID NO: 277 - DNA, 40 bp, Homo sapiens
tgatccctgcctacaagagacagtggctgggattcgggaa

SEQ ID NO: 278 - DNA, 100 bp, Homo sapiens
cttgacaagggtggcagagattaaaaaaccccacgctttaaacaaacatmtttgggggtggctatggcaccttgaaaaacccacaaccagcttgacatga

SEQ ID NO: 279 - DNA, 100 bp, Homo sapiens
ccctggctaaggacgccaccggcctccaggacagggccctgcctcacagkgtgctgcctccccgctggctccgcagcagaagcttcagcaggccattcca

SEQ ID NO: 280 - DNA, 100 bp, Homo sapiens
tccacctataccatgctttaaaatgagtatttgggcaggctgctctagcrtctatgtacagtgacagcaaaaccttcatattatgaagggggccactcgg

SEQ ID NO: 281 - DNA, 100 bp, Homo sapiens
ccttcccccaaaacaacatattatctggtctgtcatagaaactgaaattygcatattattcttccagttggattagaagctatgcattaattaatatgca

SEQ ID NO: 282 - DNA, 100 bp, Homo sapiens
ttcactcttttgggaaatttacttacctcttagcttttggttccaggatraatatcactaataggcgttagtctgctcaattaaattcaataaatatgaa

SEQ ID NO: 283 - DNA, 100 bp, Homo sapiens
tagaacagcgtgcacatctgtaagcatctgaagactacactcaagtgccrgatatatagttattctgaaaacatgtaacatgtagcaaatgggtttttct

SEQ ID NO: 284 - DNA, 100 bp, Homo sapiens
tctctaaaatcaatgacctaaattgagagaatactgattattaaaagaarataatgtcaacaaagatgtcagaaatgttattaaggtgaaattgtaacca

SEQ ID NO: 285 - DNA, 100 bp, Homo sapiens
ccacatttctcctagttttgtagcaattgcaacacagatatgttaagtgwgtataaaggaaaacgttatggctatttcatcagcaagtgttttttgccca

SEQ ID NO: 286 - DNA, 100 bp, Homo sapiens
ggagagaatggagggaaacaaaggatgtgaaaacccagagggaatgaggrtaagtggctcactgtacaatccacaagtcagaaggtatagggatgacagg

SEQ ID NO: 287 - DNA, 100 bp, Homo sapiens
aatttcttgaagtcttggaaatgaaaattttcagaggtctttttatgacmttatagtccttttggttaaagcaaaagaatagactgttttattttgcaaa

SEQ ID NO: 288 - DNA, 100 bp, Homo sapiens; identical to SEQ ID NO: 122

SEQ ID NO: 289 - DNA, 100 bp, Homo sapiens
  misc_feature (100)..(100): n is a, c, g, or t
tttttagctgcaatcttttcacggaaaaacaatgtaaaaggcataacatrgagaatagagatggaaaagaaagatacagacttgtaattgcctgacattn

SEQ ID NO: 290 - DNA, 100 bp, Homo sapiens
tcacttctttcaagtctctgttcaaatgtccccttattagcaaggccttmtttgacctccctatgtagaacagtaaaagcccaccccctttttcctccta

SEQ ID NO: 291 - DNA, 100 bp, Homo sapiens
gtgaggacctttttactttttgaaaaatggcaagccaaggacttaacaaytgccaactaaaacacaaatccttcacttcacaaaataaaattagatgatc

SEQ ID NO: 292 - DNA, 100 bp, Homo sapiens
gtcacagttaagccacagtatcaaactggtgaggcagaaaggggagagtmatgtaactggtagtcaagaaacctggattctatgattaatactaaaacta

SEQ ID NO: 293 - DNA, 100 bp, Homo sapiens
aataccatcactttccctactttgatggcataccttatagtctttcttcycttcacatgtagtttttcttattagtatgtaagaagcagcataaggtagt

SEQ ID NO: 294 - DNA, 100 bp, Homo sapiens; identical to SEQ ID NO: 126

SEQ ID NO: 295 - DNA, 100 bp, Homo sapiens
gtgtgtatgcttttgtatatgtatgtgtatatatatatatatataatttwaaaaatcttctctatggctttgataatgctgtttccccatcatgtctata

SEQ ID NO: 296 - DNA, 100 bp, Homo sapiens; identical to SEQ ID NO: 127

SEQ ID NO: 297 - DNA, 100 bp, Homo sapiens
atagttcactgctaaattatctttggtgcaaagtagggtacaaaacaaaraaagtcaaaaccacaaatgatcaattcctaattatgcagaatagccacca

SEQ ID NO: 298 - DNA, 100 bp, Homo sapiens
gaacccaggcaaggggcatttgctgactacagtgtgtctttcagcaaacrcagccggaggccccccacccatggaagggacagccttgccaagaaaactg

SEQ ID NO: 299 - DNA, 100 bp, Homo sapiens; identical to SEQ ID NO: 129

SEQ ID NO: 300 - DNA, 100 bp, Homo sapiens; identical to SEQ ID NO: 130

SEQ ID NO: 301 - DNA, 100 bp, Homo sapiens
agatttgggaatcactgaattgaactgccggccattggtggctggtgagrctgtcctggcagtgatgcagtattctggtccctgtgccagcactgatggg

SEQ ID NO: 302 - DNA, 100 bp, Homo sapiens
atagtttgacaggaggaaccatccaggctgtaattgtggtaaggaatacmtggttgaatttcactttttatgtctttattttggaattctgtagctaggt

SEQ ID NO: 303 - DNA, 100 bp, Homo sapiens
ataggttattattctataagaacaagattcagatttaggataaagctctstgcatatagaagctactgaatggtaaattgataaagttaatctttaatat

SEQ ID NO: 304 - DNA, 100 bp, Homo sapiens
aaaagaagattaaatcacattcagaagaaaacaacatttgggagaagacrtctagagacaaaaattgtcaaaagtttattgtaaggctatgaaaaatgag

SEQ ID NO: 305 - DNA, 100 bp, Homo sapiens
acattacttaaactatcaaatctttgatgaggtatttctttgactaagtrtaaacacaaatctcttagactagaatgcatcaatcctcaactgtccaccc

SEQ ID NO: 306 - DNA, 100 bp, Homo sapiens
agcagtggcaagatcagattacctcaaggtcacattactgggcaaagagytagacttctgcagggccagaagaggtttgggagctcatttcatccattcc

SEQ ID NO: 307 - DNA, 100 bp, Homo sapiens
ttttttttttttgagatggagtctcagtctgtcacccaggctggagtgcrgtggcatgatcttggctcactgcaacctctgcatcccagcttcaagcaat

SEQ ID NO: 308 - DNA, 100 bp, Homo sapiens
caatataaatccatggcagaaagatttttaaaaagaactatttttataartataaaataaaacaattagaaaacattgttctaacttaattggcagcatt

SEQ ID NO: 309 - DNA, 100 bp, Homo sapiens; identical to SEQ ID NO: 140

SEQ ID NO: 310 - DNA, 100 bp, Homo sapiens
tggtagcaaacctaaagtcaaatttttggctcaattacttcaaccaacayagatctaaatagcttattttttcttttattcgggcctgagctcttattct

SEQ ID NO: 311 - DNA, 100 bp, Homo sapiens
ttatgttaaggacatttatgatatattatatataatataccataggtcamtagatacaaaaacatatatgtcaaggatatatataacatacaatacatgt

SEQ ID NO: 312 - DNA, 100 bp, Homo sapiens
tatatgtaacgacctatttgcagacacacaagtctcaaagaggggtttayatgcagtgacttttggtacaatccctgtggttagtggaagcatttatacc

SEQ ID NO: 313 - DNA, 100 bp, Homo sapiens; identical to SEQ ID NO: 141

SEQ ID NO: 314 - DNA, 100 bp, Homo sapiens; identical to SEQ ID NO: 142

SEQ ID NO: 315 - DNA, 100 bp, Homo sapiens
tcaacatatttggtattccatggagagttctcttagtggagatagttcasgaaggggtttattttcttaatttgctttctcagttataaaatcttcccaa

SEQ ID NO: 316 - DNA, 100 bp, Homo sapiens; identical to SEQ ID NO: 143

SEQ ID NO: 317 - DNA, 100 bp, Homo sapiens; identical to SEQ ID NO: 144

SEQ ID NO: 318 - DNA, 100 bp, Homo sapiens
tctgagctgcatcctgaaggccacagacaaggcacagaaagtcactccaraaagagggaacaggatgtacagaatcatggaacatctactgtgtgccagg

SEQ ID NO: 319 - DNA, 100 bp, Homo sapiens
attgaaaacacctcctggcaaagaaggtgtgatttccacgggacaatgckgatgactttttaattaagattcttgtcttcagcctgctgccttaccaggt

SEQ ID NO: 320 - DNA, 100 bp, Homo sapiens; identical to SEQ ID NO: 145

SEQ ID NO: 321 - DNA, 100 bp, Homo sapiens; identical to SEQ ID NO: 146

SEQ ID NO: 322 - DNA, 100 bp, Homo sapiens
tataattacccttacctaattttctaatacagtcctaatattttctagarccctctccaaaaggtcagtacagactatcactgcatttgtttaagtccac

SEQ ID NO: 323 - DNA, 100 bp, Homo sapiens; identical to SEQ ID NO: 147

SEQ ID NO: 324 - DNA, 100 bp, Homo sapiens; identical to SEQ ID NO: 148

SEQ ID NO: 325 - DNA, 100 bp, Homo sapiens
aaaagtgggtttttggatgctcttaaatgagaaatcatttcttaccagtwaaagagaaaaaagtaccaagtagagaataatggggatactacctaagaca

SEQ ID NO: 326 - DNA, 40 bp, Homo sapiens
tgcttatgctgaaaaatataaaatattcagttttctatta

SEQ ID NO: 327 - DNA, 100 bp, Homo sapiens; identical to SEQ ID NO: 150

SEQ ID NO: 328 - DNA, 100 bp, Homo sapiens
caggctgtggatgccgaagagttcattctacctgcttcattaaataactratgtgtttctgggcaatcaggcccaatctcagccctgccacctataatta

SEQ ID NO: 329 - DNA, 100 bp, Homo sapiens
tgatgatgtatgccacgtgcttgcccctgtctgcagaaggacagcagcayagtcctgaattagccaccggggccttcagagaggatcccgggacagtgtg

SEQ ID NO: 330 - DNA, 100 bp, Homo sapiens
ggttattctttccaaaattccgcaaggctccaattaattccatgtgtatkgtatatttaacacgcatttcttcctgagaacaacacaagatggtgtactt

SEQ ID NO: 331 - DNA, 100 bp, Homo sapiens
ttactcttctcagccccagggagatgcattttaccagcagagccacccayacccaagaccagagctatgaccaaggcatatgagctcaaaaagccccagc

SEQ ID NO: 332 - DNA, 54 bp, Homo sapiens
catcatctgcggcatcacgtcygtggccttctccctcagtggccgcctactatt

SEQ ID NO: 333 - DNA, 82 bp, Homo sapiens
gcaggcgcctacttctgggcggggatcatgtcgtcratggactggcctttctccaacttcttctccatttccaccatgagct

SEQ ID NO: 334 - DNA, 60 bp, Homo sapiens
gcatacctcctatgggttgtcaccaagctaggtgctattkgccatctgctcctgttcaca

SEQ ID NO: 335 - DNA, 59 bp, Homo sapiens
tgctgtcagaatcacctattcaaaaggcgaatcygatcatgtggttcctgctgccctta

SEQ ID NO: 336 - DNA, 70 bp, Homo sapiens
tgggggtgtgaagaaaagagccttgggttygactagggaacctggggccactccttcctctgctactaaa

SEQ ID NO: 337 - DNA, 95 bp, Homo sapiens
ggattccctccctttgtagggtaggagaaattggggggacgggcaaggagytcatttgtgtcccccacctcggggtctctagcccgctgtctctg

SEQ ID NO: 338 - DNA, 97 bp, Homo sapiens
cctgagtaatggttacatctctgatgtaaagttattgtataaaattcaagagaacatgcrttatggttgcgcttcagggtacaaaaccactggaggg

SEQ ID NO: 339 - DNA, 63 bp, Homo sapiens
gcccagaacctgatggccctcattagtgcttggctcttaycttggaagcacaggcgctgacag

SEQ ID NO: 340 - DNA, 70 bp, Homo sapiens
tgcaagggttttgcttaattctcaattcaatgtctcttcatcttttagyagctgtggggttttgttgttg

SEQ ID NO: 341 - DNA, 100 bp, Homo sapiens
tcatctacgccttccacagccaggagctccgcaggacgctcaaggaggtgctgacrtgctcctggtgagcgcggtgcacgcgctttaagtgtgctgggca

SEQ ID NO: 342 - DNA, 75 bp, Homo sapiens
gcggtaatttcctgtgcttctttccagatcgtgcacagaactctggcrgccatgctgggttcccttgcagcactg

SEQ ID NO: 343 - DNA, 62 bp, Homo sapiens
gcctctcgatgagtaaatatgggycaaatgaagtaaacagactatgcgtaggtatcatcaaa

SEQ ID NO: 344 - DNA, 69 bp, Homo sapiens
ttatgtgtcaatggatgcactgcttgggggatmtgaaatctggagagacattgattttgcccatgaagc

SEQ ID NO: 345 - DNA, 79 bp, Homo sapiens
ccatcctccaacacacacacaaataagcagctaatggcaatrctagtggtcttcccaattcacaagacctgtgcttcaa

SEQ ID NO: 346 - DNA, 52 bp, Homo sapiens
cttcaaattgctttaagtactctasgaagaccttgactgttggatttttgag

SEQ ID NO: 347 - DNA, 69 bp, Homo sapiens
ccatttctttgtaaaataacaataactttraaggcaaaagcaagattctgtaaaccaacattggaaaag

SEQ ID NO: 348 - DNA, 66 bp, Homo sapiens
gtgaccctgtgaggtcaggtcccccagattgasgtctgagtgtgggcaagtgtgtcaaaaggggct

SEQ ID NO: 349 - DNA, 62 bp, Homo sapiens
caggccaagagcgtcctatgaatcatrcatttgttcctgttattgctgttcacagagtggca

SEQ ID NO: 350 - DNA, 60 bp, Homo sapiens
atttgtgttgggtggtctagrctattcagaaaatctcagaggaggacaaatgatagtgca

SEQ ID NO: 351 - DNA, 84 bp, Homo sapiens
catctgagtgcaagataaaaaggaaatagcaattcaaggcacaaagctaagyacatgcaacaatatagatgatttgggggtggg

SEQ ID NO: 352 - DNA, 67 bp, Homo sapiens
aaaaaggcaggacattccaaggctctctaacaygagtgtctgcagccccattcgctttgagatgtga

SEQ ID NO: 353 - DNA, 80 bp, Homo sapiens
ctccttggcagggatttgtgacactgcattgctgggctgtgttccycgggctcttctggaccttgcaccgtggataccag

SEQ ID NO: 354 - DNA, 66 bp, Homo sapiens
gcctgagaatcccacacatttcagtccaagawaaccttcctcaaattttccatctcccatcagagg

SEQ ID NO: 355 - DNA, 60 bp, Homo sapiens
tccattcagtgtatcacatcttcaggataggtrataacagtgtgaagggtgtgctcattt

SEQ ID NO: 356 - DNA, 56 bp, Homo sapiens
  misc_feature (29)..(29): n is a, c, g, or t
caaaatctgctccatgtccaatctgctcnaagttcaaccaacctccctaacccttg

SEQ ID NO: 357 - DNA, 47 bp, Homo sapiens
  misc_feature (28)..(28): n is a, c, g, or t
atattcacctccagccaccagtcacccntatgggttgggacctctgg

SEQ ID NO: 358 - DNA, 63 bp, Homo sapiens
  misc_feature (41)..(41): n is a, c, g, or t
tctgcttctcttgagatacatagttaaagtagacacggccnagacttaggaggattgcctgtt

SEQ ID NO: 359 - DNA, 465 bp, Homo sapiens
  misc_feature (152)..(152): n is a, c, g, or t
ccacaggtgtaacattgtgttttccttgtctgtgccagggacaccttggcatcagatgcctgaaggtagcagcttgtccctctttgccttctctaattagatatttctctctctctctccctctctccataaagaaaactatgagagagggntttcctaaccaagtttgagggtatgaacatactctccttttccttttctataaagctgaggagaagagtgagggagtgtgggcaagagaggtggctcaggctttccctgggcctgattgaactttaaaacttctctactaattaaacaacactgggctctacactttgcttaaccctgggaactggtcatcagcctttgacctcagttccccctcctgaccagctctctgccccaccctgtcctctggaacctctgcgagatttagaggaaagaaccagttttcaggcggattgcctcagatcacactatctc

SEQ ID NO: 360 - DNA, 97 bp, Homo sapiens
  misc_feature (58)..(58): n is a, c, g, or t
aagtgctgtaggccatttagattagagaagaaaagagccttctcaagaaggccttctnaaaactcctctgagtctgtatacgctgtcatcgaagact

SEQ ID NO: 361 - DNA, 141 bp, Homo sapiens
cccgggtgtgcgtagggcgggcgccaaggcggctcggcggggatccgtcctcgccaggggccggrtcccggcgggaggaggcgccctccctgccccccgccacggcggagcgtctgcagaatggtgacaggattctgggtt

SEQ ID NO: 362 - DNA, 127 bp, Homo sapiens
  misc_feature (77)..(77): n is a, c, g, or t
aactgggaaaatttgaagagaaagtacacagatacatttgtaaggttgtctgttacttaggctacagaggaaagaanagaattccctctctctaaacacactctaacacacaggagttgagaactca

SEQ ID NO: 363 - DNA, 136 bp, Homo sapiens
  misc_feature (106)..(106): n is a, c, g, or t
tctaggcccagatttatggtaactgataattattgataatttctttagatttgttgatattctacttgatgaaacatccaaattttactttatgtaaatgtggccnaaaagtgtcagaaaataaccaattgtgctt

SEQ ID NO: 364 - DNA, 110 bp, Homo sapiens; identical to SEQ ID NO: 70

SEQ ID NO: 365 - DNA, 110 bp, Homo sapiens; identical to SEQ ID NO: 31

SEQ ID NO: 366 - DNA, 110 bp, Homo sapiens; identical to SEQ ID NO: 65

SEQ ID NO: 367 - DNA, 110 bp, Homo sapiens; identical to SEQ ID NO: 52

SEQ ID NO: 368 - DNA, 110 bp, Homo sapiens; identical to SEQ ID NO: 37

SEQ ID NO: 369 - DNA, 110 bp, Homo sapiens; identical to SEQ ID NO: 39

SEQ ID NO: 370 - DNA, 110 bp, Homo sapiens; identical to SEQ ID NO: 25

SEQ ID NO: 371 - DNA, 537 bp, Homo sapiens
  misc_feature (286)..(321): n is a, c, g, or t
  misc_feature (466)..(466): n is s
tctagactgcatttctacgaagcgctgattaggtaggaaccaggattcaattctttttcctttgtttactaattgggttaggcccctcccattttcagcctttcccaatgttctatgtttttacctgtttaaaaatagtaactatgttttaaataaacacttccagtccactatgaagactctatgtctatgctctgagaggtgagctgaatttgttttagcatgactattaaaaatgtgagattactaatgcatttcaaatcaatagtcccttgtaaatgcttannnnnnnnnnnnnnnnnnnnnnnnnnnnnnnnnnnntgtacgtggatcagtaagctatgactagagctgatttccagtatttggaagagtagttgctattttatacatttccttctgctgcagcatctccacagctagtcgctaggttttcttagctcattctcacctttgcaggtcctastgcattttgttggcatactcccccccccagtgccttatcacatacagttagcccagcagaagggatttcag

SEQ ID NO: 372 - DNA, 401 bp, Homo sapiens
  misc_feature (466)..(466): n is s
tctgggccaaggagctcagggattgaactacagacacaaggagggcagagaacagacatggaaggatcccacccttgaccagggtggttgtagatacttaaggggctgtggggtgtggcaccagtcctgctggatttcaacctgaacatctcctaagtagctgtgggcccctgaaacaattcagtgatcagtaacacatascaatcgtcaaaccctttgctgcctaactcaggccaggggactacacatcaactcagagcctcgggctcacgggagcctcacactaccctgacccagagatgccatcaccctgtgggcctggagggcagcggggccagcctgggctacccagttgaatacaccttggattcagctcagggtcagctgagaaatatctctgt

SEQ ID NO: 373 - DNA, 1023 bp, Homo sapiens
  misc_feature (309)..(684): n is a, c, g, or t
  misc_feature (758)..(923): n is a, c, g, or t
gctggtccttctccagagaaatatatttcccttagcttaaactccaccatcaaggtgtgtaatttggggctttttccttcatgagatctcagccagtattatagacctggcagctcctacttcatttcatttctaggtagagagaccagagcacgaaaacctctcactgttccaccccgcacatcaccttcagattcttcsgtcaaaaccataatgtcatcatttcccctgccccaagatacttcatgggctccccatggccaatgtgatcaaaatacattattggttctgccacccatgatgtttacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttaccgagcatggtactaaaagtctgatacgaagtctgtgaaaagcttaagccttgctaaaagatgtaatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtggaaactatattattaaataagatactctaatccttgtataagggctctttttaacatcttcattctctcaaggatcactgggtatcttggtgtcttt

SEQ ID NO: 374 - DNA, 392 bp, Homo sapiens
agcagaaaaccttggagcacttgtttctcatctcacatgaagtaggggagttaatgggaacctgggtgtgggaggggctgaaggatggggatcttgctttgtaatgtgagaatgtaasaagaacgtgaccttctactgttttcctaggttggaggggaccggccaaaccccaaggaaagtggagagaagaaaggcatggaggggttctgtggagggtgtggaaagggaggatgtgtaaatcccagcgtgggtttggagaacctaagtaggttggcttccctggggtttaaatcacagaggtgttgggaggtgagcccgaaatggaaataggccgcatcttgaagaatcccacgtgcggtgctgagaagtttgcatatggaacaatggactaa

SEQ ID NO: 375 - DNA, 487 bp, Homo sapiens
gaattcagagaggcaaagcattctgctccaaatacacagccctgcatggcggagsttggttttcccaccagtctgagtgtggagtctagaatctttccacacttcattgcatctcttcacaggaaagaatttcccactagatccctgcagataattatttcaaagaagctacagaaaatgagttctacaaggctgattggtttgggtcttggtgtaaggtaggtttaagggaagatcaaggatatttatttattcaatttcatgaaatttcatgagaagctcctttctgtccaactactgaacaacttaggggtaccttagcattctttttatctggtagctccaaaaattttacatcacagccctctaagtgttgtgtcagtctacccagttaaatcaaattgcccttggttactttttaaagattatttttcctaaggttacctgatgcaaaatatattgttcccgaagctggcttgcagaattc

SEQ ID NO: 376 - DNA, 466 bp, Homo sapiens
ccatcttggataccagggactctgaggccctagcccagaggcccctccaagcagaagagaacaacgagagtctcaaccacaagagaaggtgggggttgagtgggaagatcacaacccagtaacttggtccgtgcttcagggcaggactccagacccatgaatagagagggccttagaaaccagaggctgggtggagggaaagacgcaccaatggagtgcactcagaattgggagccaggggactggaggtgatgctsctgcagactcctcaagatgttcagaccagatgcgtctgataaaagaaaactgactcctcaggaactccctctcctgtgggaacaagttcctcttgggaatggttgcttccatgggttccgggtcagagggagattagactgtggctgaccagcctgagtagggctttgttcctgagctgggcttggcaaagttcttctgcaaagagtag

SEQ ID NO: 377 - DNA, 477 bp, Homo sapiens
tgtggcctgcgtgggcagaactggaaccttatcagagccttgggttgtgtgcgttagaggtcaaacaggcctccgtgttttctttgcctatsagcgccttttggcagccggcactgcttacctcctagatctggaaacgcctgcggttggacaggccaagccgaagcggcttttccacaattagaaacacgtgctcagttccttccacaagcgggggaggcggccaaggaactcggtgcagccaatcacagagccgagcgtagccacacccacgcccccacaggcccggcttccctgcccagcggctccaacctccagcctgggcacccagacccagagggctgctgggagggacatgcctcaccccttttgctccagctggaggggagggagtctgtgggagctggccagagcgtggcagaaggcagagaagtgaacgccccagcaaaaggtgaggctgagaagccaacccttctg

SEQ ID NO: 378 - DNA, 1192 bp, Homo sapiens
atgtcataatcttttctttgttttttaaatcattagattgtctaactcattgatatttttgaacatgggctttctgttgcactcctcatcgaagtaattttcattatgtgttgttttttctaagtttatttatttttattgcaagtggccaaatgataggccctttctagggaactatgtctacacaaaataaaataaggttaaaattatgcaatgaaactggcacttcggtgagctgtaaaccttctctgtagacattgtccttagatcatcctcacacctgtaatgttgaattattccctccctaggtttcattcctagcatttctgatccttaatctctgcttatccagaaagtcattttcagttccagggctaacttcctgaaatcctcacagattgattcaggctaaatcctgtattcaggttcatgttgaatccccaacgaaaattaatgggatacgtgcagctgcatctctggacagaccacacctcatcttgctttctgcaatccccgtccctcacttgcctaaagtgctttgagaactgttgtgactacttcagaatgaactatgtttctgtgactctttcttgctggctcatttcacaggaagcccgaatttacttgtacatggctcacagatcaatttctgcagtgtggatgtttgtaatatacataaatgtgttggatgaaggcattgaaataaaatttttgagtctgcaaattaagctggaattgaaatcagagaaaataatgaaaggcaatgaaaactgattcaaggagaagttttgagtaagagtgccaacaggcaccaaatcattttaaatgaaggcaaatattaaataatttaagagcgaagaaccaagctggagactatttsttaaatagaataatgaatcagcagttcaatcattatatggtttgaaagttattatggaaaaatcactaaagctatcaactcaaataaaacctaacaagaaattaatagaaatataataatagtgtgtattttgaaagtgatgaaatacctatcaaaacattaaaaaattataatacaggactctgattatgttactgtagaaatattctcatttgcttaaatttgtgagagggtttcttgagttggaaagcccacaaagatgcaaactctgatttattttataatgaatcttctttctgcaaattcatttca

SEQ ID NO: 379 - DNA, 608 bp, Homo sapiens
aagtcccataaaataactaccgaacaaacacctgttgtgtacaaggcagggcatgtattaaagactggcatcagggcattgcaaaaagaaaaacaagccttgcagagacatccacaagccaatctggtcaatagatttcacgactgggtaaaacaacagtgcgtgcgaagaacacagcttctggcacaaccttcttgtgaatggsagctgacttctaaacaaggattgaaaaatgccttagcacaaactgactgtaattaataagaataatttattaaagaaacaaagaacactgtctaattgtctttcctgactgccatgcactttcccccattgtacttcctctttgcagaataccagcctcgtagccctaactgtaagaacattatctatgggatgcaaaaccaaaacacttctagaaatcaaatattccttacaacatttatttttaggttagcgacacaagccaaatctgcttaatttccattaagcacatggtcacatgctcattgaaataaactacaaacgtcccaggaaaaaaaaaaaaggaaaaaaactatcatgcagaagataccccaaatttcgccaggtttgcccgtttcaggaac

SEQ ID NO: 380 - DNA, 427 bp, Homo sapiens
tcctgtttttaaatgcaaatgtgttttgaaatgtatctgttccatcatttctccctcaaaatcatactatagctgaattactcaaaaggaattctagcaaagcacasaacattttcgagcataagcctgtgatgcttttcatctaaaaaggtgactaacagcttgcataagcttataaccagacttaaagcttaaaatccctgtggagtttgcaaagggcattgcgaaagccagccaaacctgggagacataaatgtctttgcatgctgacttatctaccagtcacgaaaccagtctggtttaaaattgctgttattcatttcagctccacaacctttattttcatgacagtgagttgaaatagcactcttacctctgttaagaaaaacaagaaatgacttctatctaacaaatgttaattagggca

SEQ ID NO: 381 - DNA, 637 bp, Homo sapiens
ttttaaccaggaaaacagggtgggggggatccaaaaacaaaaacagaagatatctactgtatgtgtatgactttcctcaccaggaggaaaaggagtttgtgaatttaaaatatgaagtctcaaactaaatttgtaccccaactgcaagacagtaatcaaatcactaaaaacaagaagcatgtgtgtagtaaattggagaggaaagacagaagagagatacaaatgatgacttaaatggtataaattacagctaaacccaatcgcatctgcttcccatatcctaactccctccktttgggactccttctgaattccaccctcatgcccttccagatgctgttcaaagataataatactgttatccaaatgtaaaaatattattgaaaaatgtaaaccaacttatttgtgggatcccttcctttgtctctaaagctgctgttttctaacacacattttacttctcaggcattttctaaaagaagccacccagttcatacctaaaaaccaatcgctcagtggctactgtttctcagcattctacaaagtgctcaacattcttttcagcatatgtgagagcaacatttttatcactgattacattttcacattcccctaaataaataaacactagattatg

SEQ ID NO: 382 - DNA, 608 bp, Homo sapiens
ttgcaacccacccaaggagtcatagtatgaactcttcctcagggttatttcaccctggaattgatgactgttacattcgacaaatatgtgagcacattttaaaaatttatcctttccactttacttggtagcacgaacgtgttctttacggcatattcacagctgaacaagctatcatgcatctgtatkaggagcatggttacactcacaggacgaccggcttttgtctttgaagactcagggctcatttagttgccatgggttgtcaccatcaccattcacatgccaaacccctgtttctgccagtctctctagaaagagacactatcttgccagggcttttaaagtttcactgttcttaattcttagtgggagccactaaaaaaccttagggcagtggctgatgaggtctgcaggaccatgcaagttcaactaaaagaaacacagagtgagagagagtgacgtggttctgagtactgggactaattagagaacactactgtgggggaaagtgttttgggaaatgtgacaaaagcataagtctctatatctttcatccttttcccaaccacagttatacagtagattgaaaccgacagcagacaatc

SEQ ID NO: 383 - DNA, 667 bp, Homo sapiens
  misc_feature (159)..(176): n is a, c, g, or t
  misc_feature (272)..(295): n is a, c, g, or t
  misc_feature (427)..(480): n is a, c, g, or t
cttcctccctggtgctgtcggtctttccgtatacccagtcactggtccgtctggacctctccctctttgacattgtcacagtgtcagagccctcagcttcctgcccctcgtcccctctgctccatcctggctcaggcctcgtctgctctccttggcccnnnnnnnnnnnnnnnnnnagcctccccacctccagtcttccccacctcagcacatctccaccatggccccaggggagtctttccacaccctgcccctccctgccacagcccctnnnnnnnnnnnnnnnnnnnnnnnntgagtcccagccctgaagcctggccttggaggccctgcagagtctccgcttccctcactcttccaatttcatctctcctcactcacccctgcctcccccacactctccttctgccaccaaacccaggtaccnnnnnnnnnnnnnnnnnnnnnnnnnnnnnnnnnnnnnnnnnnnnnnnnnnnnnntgcagtcgtgattattctctctgcctctcagctcttccctctggggtgkttctgaggactaaacgagaggaggcacttagagcccttccctctgcgacttctgtctcttccctcctggcctcactaattcctattaatttcttaggttcatttgcaagatagtccaagcatacagaaaaatacgaag

SEQ ID NO: 384 - DNA, 576 bp, Homo sapiens
agatctgtttttctaaatacagcaagatacacatgtacatcaaatgaaagaatggaaaaccacacgtgcagcatctagcctgctaccactttgccattattctctgtgattctaaagcccatcatggttgggtttgctcaggcctgctacacctctttccctgtgattgactttctctctcttgttactcatgtaactgttttgcatcaggtctctgtgaacaaggaccatgccttgcttttgtktcccgtgtttccttcaagcaataggagtaggtggccactaattactgcagaatttaattgagggacgtgaacaaaaggccttgttatcagcattaagtgatattaagctgtaggctgattctccctctcttacaaaatagaaactgtgtctggcccataaagaggcttttaaactaaagttgtgttaacaaagacctctttccagagacctccctgcttgaggggagctggagtaaattcaggctttgtttcctctgtcttagggagccagctggaggtgaggggacaggaggtgtgcctgctgtccaaccgattttaatgcacaagatct

SEQ ID NO: 385 - DNA, 437 bp, Homo sapiens
agagatgtggctgaaataaaagtagtcaattgaatagccttcaatataaagcactgaatttagccacgcttatttttctagaactgtctatattagggtactttgctttccaatattgtctttttcttggtgttkgtgctggtagcactggtgttattctgcttgtttcaggtaacagattgacaatgtcttgaagatacaaagtctgctctactaaggcacaggtctgatgtccactttctgaagttccattcaggcagtgggttcagcacacattaaaatttgcttgtttggagtccaaactctcaagacgtggtttctgttggtttcttgttccttctacaaacaacccacaatgcatgacttaaggtattgagataaaccccaaaatctcattgccttaacacaacaaaattatcattattattattttggct

SEQ ID NO: 386 - DNA, 415 bp, Homo sapiens
acacactcttgtgtgtgcaggaagtctaggtcaacagaggggtagtgcaacctaggatacagtgtagactagacagccagggactggacaaaaatccacttctcagagaatgccagtctagaacatagcaatgaccaagaattagatgattgatgtgtttkcaccaatgtctggctgtctcatcatatcctcatgggaaaaaagaactctctctgggaaggcaggtatgtctgaaaaaattcttatgtgactaaatctacctggaattggcaagacaaaattttctcctactgagtagaagagagatctgaaatagaactcaaattctgttacaggaaaataaaggaggttccatttcttatacctgagtttgtggcctgtgaaaatgcatgaccattgtacaaatataaaattt

SEQ ID NO: 387 - DNA, 479 bp, Homo sapiens
cagaacaaaatcatctgattccactctctgtgctcttaaatacttcactagaataagtcttattttttataattgatacaactggtctaaatattaatgaagttttaagattcaatttacatatagcttctgtctctgtaagttaccagttattactacctaaaatacacttgkagtcaatagtaacattgaatattcctagattttattttataataccaaccagaataggaggcctcaaaacacctgtgatggaaaaaatgcagtgcctgagtgcatgcagctaagcctgatcactagaatacatttcatttatattttataagataccaaagacttcagaactacaaagagaactatagttgctgcaaccagaaacctattcacatatacgtacttttataaatcatgtgcatatgtgtgtgggtaccttcatgtgtgtgcatatataaatacatctaaaagctgagaaccacctt

SEQ ID NO: 388 - DNA, 401 bp, Homo sapiens
ttctttatcttttcctttcttcagactccgtccaaggagatgctctccccggtcttctgctgcaatttagattcctttcccttctctccagttctccttcccttaccaaggagaggggagcaaatggttttgggcaagggctttggccattcatgtcaagctggttgtgggtttttcaaggtgccatagccacccccaaakatgtttgtttaaagcgtggggttttttaatctctgccacccttgtcaagggagtcttgtaaagttgccgagggtaggttcatctccaggtttcgggattcccatccgtcctggcgatcctgccagcagtgggtgggcagcctgagctccctcgggctcgcctgccagcctggagttcttcctgtgctccttgatcacctg

SEQ ID NO: 389 - DNA, 608 bp, Homo sapiens; identical to SEQ ID NO: 382

SEQ ID NO: 390 - DNA, 581 bp, Homo sapiens
cagagacggagacaaaatgtggcagttggagctgccgcttggcctgctgcctgggacgcggccccttgtcccgcagaacgtcagccaagcgctttccgtgcaggctgataacagggtggtgccgtgcttcccttgggccgtggcaggatcctgctccaagggggggtcctgctccgagggggccggggtttctgttccaacagggctggtggggggggtcctgttcccagaggtccgggatcctgttctgagggggccagggtcctgttctgggccgggggtcctgctccgagggggccgggcgtcctgttcctaggggcggtgggctgcctgtgtgagagctgttcttcacctgccacgtgcagtctgggcattctggtgactggagaatttgggttgtgttacagtgttgttgctctgtcctgcagatgcctgaattcccaggggtcttgcaggtccccttgcccttgckctgtgtgggctggcgtgggtctcagtgggctgtgggttgagggtcgcttggttgggaagctctgcgcgcttcccagtggagggtccgcgacgggggttttctgatccct

SEQ ID NO: 391 - DNA, 585 bp, Homo sapiens
attcacagagacacccaggcccttaagaacaagttttaagcatgatcactagcttataggccaggctgtcctcagcttttcacacatgagtgagacctaccatcttaacagctttgggtaaactatggaaaaagggatttcttttcactgtgtctggtaaggtgtggtattagctttgtcccaagctccaagctcagaagtgaagagtactctataggaaaaatgtatgttgcccacaagaatggttcatkattttgctcatattacacagtgatttgggtaccaatctttgagtagacttgtaaaagtagataggaagggaggatgctaaaaactgtgtctcagaatgtcatgatcagtttaaattttagaatcaggatgtttcttgttattatgtcccattttgagaaatgttttgagctcattttgactctgggcttgcaattggagaagtttgtgggtgaattacttattttccttcttgtacaagtagtcttccactctggggcgctaaaatttgtaatttgaattcttccctgtattaaggaatgctagtgtttaccaaacactataccaaatatcttg

SEQ ID NO: 392 - DNA, 526 bp, Homo sapiens
tgagcagtcataggttgagcattttacaaagcatgcagataccctcctttgtgttttaataatcaaatgacaggcgtaagataactaagttcaacttgaatattaaaagctgtagtccatttcttctttatatgtttaatgactatatagttatttggctaaattaatttttttctgtgcttaagttaatttttgtacaatttatgtccatatttgcttggaaccttagcgcaatatttgagccagaagacttgcaaagcttgagctaaatgaaataaaattgttggcattaaakgaaagctaacaactttgaagttttaaaatctctgatggtttgagtataagaaatgactccattggatgaggtgaggtaattccaagatacagggtactgataatactaaacgtaccttgatgactaattaagcagtttctgagtataggtattatgctgtaaaaccaaactggaaatggactatccatggagcatagtgtatagctgagttatcaatgaaaatgttaattc

SEQ ID NO: 393 - DNA, 548 bp, Homo sapiens
taatgacacctttattgattgaattagtaaacttggtgtttggattttgtggcttggtgcatgaggacaactctatagagttcattagatatttattggcccaaagtgttttcttataaccaaatcagtaaggtgagacacaaaacatacaaaaccctatgcaaaacaattatagttagccttgagatgtatttgaatcagtcatttttgtgtgtatgtaaataattttatttatctacagaatatgttgcttcatatacttttcaatgactatttctcagatagatccttgggaattcttagatgcttgacttttatttaaaagcatgtctcttcatagctgtccaaaaatcgtggatagtatgacaatttggttgctaccaatttttcttgtataattaatgtataataaatttacttgtatacaaatctattattttctcakaaaaaaattacattcctgtaagttgaattaccaggtcaaatgatatgtgcataaatttaatttatgtacaaaatgtacttagccacaatgaaacttgcaaaaa

SEQ ID NO: 394 - DNA, 977 bp, Homo sapiens
gaaaatgctcaatagtttccttccatttttctattcattctgttgtttgtaaataagtaaactgagtcgagtgtccagcagcagactactttctttcgcatcttgtttggcatatatagcacattttacatcttttaatattcgtgttttcctatcaatcagtttaaaattacaacaaattgaactaaataatttggctttttaaaatgaagatgctactaaacatagtctcaatcaaacaaatagctcagaaacaacaatgcaggcaaagctatcagaaatataaaaataacaaaccakatctttactgagcaagagtctaaaaagcagcaacgctttctgtaaacggatctcacaattcaaagagcaattttatatctgaaaagtacagaaacgacagtgggatttcaataaacttagaattttctggtttttgagcctgtgctctaaaaatacagaaatcattgataaacctaaagctgagaaaaatttaaaaggcataaccagttcaaaaaacaaaacaccatctctgtggatcagagactgcatagaaacatactgtggtaggaagaaatgaagttgtaggcttctgggcttcagtctgaaaacaggcaaagcaattaggggctcatagtatatgggatatgaacggcagtaagtgtgcagaaaaggaggcatgaaccagagctaaattcattgaataaaaccagaagaagctacggcagggcttctcttgaccatgaaaggagggtaaagttttagtaagctgctacctagagaaaatgctcatttagagcattttccgcagctttgccccagaacctttgccaaactgttgttgggttcagtgactggatcttcaatacaatgaagcacagagctatgaatataaggcctacttctgaacaaaaaaacctagcaagtgagagaaagcagccactcaagcactaaataggaaaaagtaagcaaaacaaaca

SEQ ID NO: 395 - DNA, 494 bp, Homo sapiens
  misc_feature (370)..(407): n is a, c, g, or t
ttttttttgagattagttaatagaagcacgtacactaaatagaatgatgttgaccttctatggaagcacataattggttcgaaggtatttttgttttctaggatggtttctttttacatagtctaaatactgttgataaaaattttacttaagtaaacaacatgtatatttattcagtgtccaaatatcaccatagtttttccaggtgaatgctkcaataatgggcaacagattatacactaataagtgctttaaaattgaaaatgaaaaaactcagctactgtcagttcaatatttagtgccagatagtttacatttctgtggtatctgggtctatttgaagtgtttacttcttggttctatagctcannnnnnnnnnnnnnnnnnnnnnnnnnnnnnnnnnnnnntgagaaatagagccatcaatgttgaagacttttttttaagtttattttcaaagtaaaacaagctaattgaatctatttcattgactt

SEQ ID NO: 396 - DNA, 1006 bp, Homo sapiens
  misc_feature (35)..(100): n is a, c, g, or t
attctgagtttaaaaacctggcaagatactcactnnnnnnnnnnnnnnnnnnnnnnnnnnnnnnnnnnnnnnnnnnnnnnnnnnnnnnnnnnnnnnnnnnccaaatctttggactaagatgcccggaagtctttctaccatagattgttatcttcctaaacaacctgtctttctagattctctgctactatttcttggtttagaaacatacacttcacatactgaagactttactgcaacaggactgtggccagaactgtacttgtctcattaggcttaccttgctaaggtaagaaagtgatctcactcaaaggcattgcatctccactgaccttgaataggaaatacatattcatatcttgccaagagttcaagtgccaagtcaatacatactacgaagkctatggtttggaggagactatgaaatgatgacatttcccatgcattccaaatgtattaggatgtatgactgaattaagtgggacatgcacctggagccttctcaccttcacagttgacagccatgaatgaactggccatttgtctctggaaatctgcttccgaggaggaaagcaaatgattttttaaaatgcaaaacaggaaggaactaaaggcagcatggcatgagcctcattttttccccaacaggtcaacactgagacatcacccctccacttctacttgagactttgttactttccatggctatcagggtgtgtgtgtttaaatatttctacatttggtaagcaggatctgttacccccttttctcaaatatgccctaatgagacataggaaacatgctgtcactctcccaaaatgaaacacacatgaagactgtataacttttcctctaattaagagtaagctttacccatgtattactagcaatttttctatcactgtaatctcaagttacaatgtctagaagacagatgaagacctgctggccagactatgacagtattctttacagatattcttcatttctatcattattttattac

SEQ ID NO: 397 - DNA, 774 bp, Homo sapiens
catgcctgccagaaacaggtcctgggccacaggacacaaccagcatgaccaatctgagtcaatattatttctttcaaaaagtggggaaaggtagccttattgtaggtaaacaaatgacttctcttcgagtctttgaaatggaagcgagcgcttggccttccatgtggtcctacataggacctgcttccctgtgagagggagggagctcaagaatgtctgagggcttttgggatctacacccacctccccatcaggcagtgacacatcagctcctggcatgccctccctgctttcctcctaaaagagagaggagaagaatcagatccaaatggtctagacagggctgggctaaccttgcaccttccgtggcagaactggctttgagcagttggagttcaggagaccctgaccagcatgtgactgtgtcaagactgaatccatcaatgccactggaacacctgctgtttcgttgggtttctatcgcatccttgggttggagggagctattgacactttcctggctgttcaaaaatgggcacatgtggactatagagtgtagtaattgaacagcaccaaagccgtttgcactctgcttgtgcctgcaggcacacctggctccacagcccgggtctgaggatgctgaggtcacagcctcacaccagctcccagacgtcattggatctgtctcctttttcttagrctttcaaggcaggtgttctccctcctgtttttattttcctttagctcctgaccaactgcttttgtcaaaatcaa

SEQ ID NO: 398 - DNA, 479 bp, Homo sapiens
agaaggcagagagaaacgtcagagcaggcatacttacttctaactgactcctcccattccatcggcaagagctagtcacagtgccaccttcacgccagggcctgggaggtctgtatgcaggttaccrggaagaataagtcagtttagtgagcattcggcccataaaaccaccgtgcaggtgaggaaactgctgcaaagtatctactgctacaaagtgcctacgcgaatcaaccagtgtcacatagaagcagattccatattccactgcagcactctttgcctacaacattgatgcttcctaaaaagacgtgtcttgcttttatttcttatatcacacttactatatagatttaaaaaatacagaaaaacttaaagaaaataagtttgcactatacttttccaacatagtcgctctttatgttttgatgcccgctaacctcttccacgtgtgtacccttattatatatcacacagacaaa

SEQ ID NO: 399 - DNA, 110 bp, Homo sapiens; identical to SEQ ID NO: 41

SEQ ID NO: 400 - DNA, 475 bp, Homo sapiens
ctgcaggcagggccaacaaggggcagcaggaggggctggatgcttgggccacctcaggcgattgactgagcccggatgttccggccacctcaggtgactgactgagccacggttacatcacctagaaatgggttaataaccaaggtcctgcctcccagggctgtggccgggattacaggggatgrgttaggtaaggtgccccgtgcagccctggtgtggagtaggcgcccctcagggagccctcagcccccactgctagcagggtaagcgaagcgatggagggtgggtttgagaagacccctgattcctggaaggccacccgctgtttttcagggtgctgcctgaggatgacggacccatgctgccaatccaacaagacaaaccctcctgcttcagtctccccacctctggaaccgaggttgtcctcaaagccccagaatgcatgaaaactggcatttattggagacctgccatg

SEQ ID NO: 401 - DNA, 579 bp, Homo sapiens
  misc_feature (90)..(194): n is a, c, g, or t
ccctggcaattgttcatgctggggccccaagctccttttatatcttcagatggagggaaggtgtgcttttggcagggaggaagtagtcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatagtgtgtaaggtgcccctggcccctggggttacctggctttgctatgaggatctctggctgctctggagagagttaaaagtgtcttagcttttggaatgcttgctatagatttgcccatattaracctaaaaagagcaagcgtgtcagcgctggagcaatgagagtatgtagagttggcaggctggccagggggctgcatcacccctgcgagttcagaggcatttctcatcaatgcccgctcagcaaaaagacgctgaatctcaaccctggcacattgaaaatttctttgaaattctccagcccaaacagctcttagatatgcaaagcataattgacagaaacttgaagctagaagcctccttgaaaggccacctatgcca

SEQ ID NO: 402 - DNA, 587 bp, Homo sapiens
  misc_feature (106)..(106): n is a, c, g, or t
aaagtttgctttctctgctctgtgtcccttctcattcccaagcccaaacttggaggggatgctgaggatgctgaccgagcgcaaatgtggctgaattaaggcagtntgtgggacaacagaagtttgtagtttatgataattaaacagtgttctctcttcacatcataagactctatctttggcgatagaattgattgactttcaaatttggttgtttttctcctctctgccttaataagatgttctaaaggaaaaatggctgtttgttttgtggtaggatccttaagagaaggattcgaggccctgcaatcccaaaagatgctgtttcaagaaaggagtaarcggtcctttcacaggtctcctgggtctccgtgaaggattacaaagagttatagatctgggctccgctatgatattttactagttttggtcaaatgaccatcttttggcaacacgggaattgacaggagagttgcttcaacttcttaaaaggcttggactgatgggtgttgtccatttctcaggtctggtaacgtgtcaaactttattccttaggtgtccaccaaacaaatatggccatagaggtg

SEQ ID NO: 403 - DNA, 846 bp, Homo sapiens
  misc_feature (329)..(357): n is a, c, g, or t
cctgtcctgtgtatttgcgcagccggcgtccctgatgcatgataccctctgctgccacgtgctgtctccctctgctctgctgggtctgcaagtgagagatgcaggggagcctggaggagccagttgcaaggacggcccttggaatattgtcagtgatggcaggtggcactgggggatctccaaattctactgtcattcattctacagaaaggtcactcagttggagtcaagggtaaggaaacttgccttggagctccttagtctctgaataggaaggagctggggctgtttcatgcttgggtatcacacttggccttgaaagtcaagcnnnnnnnnnnnnnnnnnnnnnnnnnnnnntgtccctctgatgagactccacctagctctgcatgtccagcatgtgtgcatggggtgagggagagggtgtcttgccccagacccaggcatgtgtgctagaaacctgggtggagccaacagcctcttctaagaagggagggagtgtcagtcattgggtggtgatgtctgacgtctgctctctaggggaaatatccaaaggaaggcaggtcaaaaagacagaggcaccatgagcacaggcctggaaggggaccacactttggggctggcacatagaatagctgaactgaaatcgaggacctcagtggagcagtagtgaaagaagagtttaaagagagaaagtttgttgagactatcrtgatggtcaaggcttgggagactttgccttggagcagatgatggtgatttggggggaaatgagatgctggatgagtgctttaaggcagggactttcaggatccattgcagggggcagagttgaaagggaggagg

SEQ ID NO: 404 - DNA, 586 bp, Homo sapiens
agaaagtgactgttcaaaagctgagcgaatggtaaaggtacctgcagggcctgctgtgtgtgagcagtgactttgccctgcaattacagcataggaggtgtggggcccaggacactccttcccaggggccacttaggacgttgtaaccattttttagtggccctttgatggcactctgcctcaggcatcactgacacctgctattagatttgattcccaagaacctatgccacattcccctcaacacatctccatctcctccagcagtaactcacttttacagrctcccttctgaaagctataaggccctttgggaaagtagctcttctctaggacattctcacaaccaggcatctaagaaagcttgatcatgactgacagacacaaaatcccctcttcccagacactctggcagtgtctaacaggagaaaacaggtttcattttctactttttgatgattatcagagtcttacatgctgctgtcttcaaagggagactgcctcctcctaggtcagcagctgcctgttggtgagaggtctgatatctcaaagagactgttatgaaccaatgaaatgctccccagcc

SEQ ID NO: 405 - DNA, 812 bp, Homo sapiens
  misc_feature (230)..(361): n is a, c, g, or t
tgcttcctaggggctgctaagaaccacctgctgatgaccaggatctcgattttgggattggttagggactcctaatcctcctgttcacacttaccaaggaatagctcttcatggatgaatgaatgaagagttgaccgtatgtctggcctattgttgaagcagaaattgccacgctatcataaagtactacttatgcattttggtggatcctccttcattataaagtctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaataaaaatagaatcaagtttttctgtttgttttgttttgttttgctacccagctcctaatgctcagttactgtttgttaagagttaacttcacatctggatctggtcagcccctggccaagtgcaaaagccaatagtgaaccaaacartatcacggcaagggaggcagacatcaccaaagactgcattttgtcagaccataaataaaactgccaccccaaactccacagacgaaaccagaattgttcaattccatcttgctcttcatctactaaaacatgtctttcctctgatttccataccctgttaatatacacattttcagtcacttagtttcaaacctcaatcattttgcattcctttggaatcattccttagcatccagtcagtccctcagccttggggaattttttggtcctatactctcctttgctgaggctcttctt

SEQ ID NO: 406 - DNA, 490 bp, Homo sapiens
tcaagacacgaggacatcagaggcagtggaattgccctgtgttgagtggctgaggtcgacgccccatatttctgggttttgttcgttgagagacagttgctgattaccaagggttccacggtcttggatggrcgtgttccactcttggcacacttatcatgtgctggacatagcctctggtctgggtaaataccaccctgttttgcagggagccctgcccagaacttacctttttgggtattagttgcaagtatgcctacccagtttttaaaatcctctctgattttgttgtataaacgggtctcattctcccttacaacttcttctccttctactaacttttcgatgtcctgattaaacatcttgattttctgaaaatgatataaagtcaatagtctttgcggaaatcatatgaaagaactgggaccggcgttgcaaggagatgtccacccgcccacctcatctcatgggtgaagaaggggaagggcag

SEQ ID NO: 407 - DNA, 580 bp, Homo sapiens
  misc_feature (101)..(127): n is a, c, g, or t
ggatctgagggttattgatatatagaccgaaattgaaactgtaagcatgaattaggtaactgggagaagataatgggcaacggaccacacatcctttatgnnnnnnnnnnnnnnnnnnnnnnnnnnnctggaattgataatgccccattgttaatcccactgtgcagcaagcttttagaaagtctrtggcagtacttctcagattttatgtcatgcatgcctggaaaatgataccatttatagggcaccttgggataaacagagaaggctgcttgtattggaagcaactggtccagggcctctaggatgcccaggtcccacccagccacactgcatgatgaggggatcaatatcttggtcacacctgtaaaccagtgtgactcactgatttagaaggttctaattgtaaaactcacatggagctcaaggggcaggctactaaaaattaaggctaaggcatagaacagacttggtactacccagatgtgagttcacatgtggcctatcttggacaggaattgacaatcagcccccagattctgggcatggagtaatataagttcatagcagccatactaca

SEQ ID NO: 408 - DNA, 579 bp, Homo sapiens
gaccaaacactaacagggattcaaaagaagaaaagatggtgccatgtgacaacatatcttcaactgagttgagctacttttttccctgaagaaacrtaagtagcaggccatgtgagaccaaagcttgctcaatattcctagtgtgatctgcttgtaatgagatcccccggctcatttctggattctcattgtgcagacccaagggagacatggtaagtctcttgacaaattgatgcagtgactagagctagacagaagaacttctcctctgagagtctcttcctaatcacagagtttaagggggcagctcttctagagccaaagcaaaaagggccaaacggactctgtgaatgtcaccctgtgctttagccagagtccagtgaaatgtaagtgacccaataatctaataaggctgagaatcaaataaggctaaacttttaaagacatcaagagtctgtcaattatttttccagacaaacaataatcagatttcctagaattgtaatgatcttttgtaaattagtcacaattattctaaaactgaacacagattccacagactgcagaaggtggatggac

SEQ ID NO: 409 - DNA, 242 bp, Homo sapiens; identical to SEQ ID NO: 15

SEQ ID NO: 410 - DNA, 438 bp, Homo sapiens
  misc_feature (235)..(378): n is a, c, g, or t
gagttgcttattggagctcggagagagctagagttctggatgaggggacttgcctcctgggtctgagcctgagtggtgcagttcttacaagtcagcccgcatggtacagaacacagcataggagagtaaagaatggatgtggtggggtagggaggaaacagaggataaayaggacagtgataaatagaaatccacagtttaaaacttagtatctaataaacattagattcatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatagctttctggagaataatttgccattagttatttctgtttaaaatactatttaaacca

SEQ ID NO: 411 - DNA, 527 bp, Homo sapiens
agattcctggcctccaattcctctccctttggtaagccagcctacatacgctgacaggctgatctccctaaaacctaattctgattatgcattactatgcatcatccccctgctgaaatactgtcttcaaagtaagtccaagtttctcttcttggctttcaaggacttgcatatgatacagccccaaattcctgctcccaccttgtccccactattgtcccaccttggttgtcccacctgctcccactttgtcccacctagtccccagtatgtggactctggttccagttgaactgagttgtgggcttttatttatagayacaatttctcctgcattttctccatccaaaaatggccacccatctccacacacctgccctcaaattggcttgctgtcaaatattttcagtgacccagtactacttctctcatgaaacctttcctgccatccccaaagagatgtgagcaacttattcctttgaactcattttgcctttatttttaaaattatagtggtgttatgaaaa

SEQ ID NO: 412 - DNA, 395 bp, Homo sapiens
  misc_feature (70)..(171): n is a, c, g, or t
  misc_feature (208)..(234): n is a, c, g, or t
tggggcttgagagtggggtagtttcaaatgacactgacctcccaggagggaggaggccacagaggccatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcttcctgatgctgagtggacagagaggtgtgtttnnnnnnnnnnnnnnnnnnnnnnnnnnngagtcaggtctcttttccatctaccatttcccaccaggctgtggtccttgatcagtctaaggcagctgtgcttacagttggctgcgtgtacygaaggacagagcctcatcctccctttctaaaacctcccacattctttgaatttatgctgtgtacgcttg

SEQ ID NO: 413 - DNA, 494 bp, Homo sapiens
caaatggcactctcttctctcctttgcagtaacaggccctgccacacacacaagcatacccccacctctacatgtaaaccccacctgcgtacacaccatatatgacttacccatgtccccacatttatacatgcacattccatgcacatattcacataaccacacacgctcatctgtgcacatatacctaaacacccccccctccccaacacacacaaactgcccacatayacactcacagaagaccctggacatctgtaaggaaagcatctggagacctttgggaatcctcagacctgtgactgtcgtggagccggtgccaggtcctggctgtctcctccatacatttctggcaagtgccagaggctcccttgagctacctacctcagatgtcctaccttttccactcttacccatcttctctctcaacccctagtcacttcctacaaaggaaacggattccttccagagtgttactgttatgggaagcaaat

SEQ ID NO: 414 - DNA, 250 bp, Homo sapiens; identical to SEQ ID NO: 1

SEQ ID NO: 415 - DNA, 424 bp, Homo sapiens
  misc_feature (136)..(172): n is a, c, g, or t
tgtttacctgtgacttccctcaattcattgcccttcagtccttaaaggaaaattggatggcacagtacagtacccatgacaatacactcttttttttttttgcttgatccactgctttgtatgctttctgagaatnnnnnnnnnnnnnnnnnnnnnnnnnnnnnnnnnnnnntgaaactcagtagagtaaggttagtgagataaactacaacacctgatgctgcagttggccttcgaactgcaactgatactcatcttcttcctccaccaccatccatttcgtattctcctcayccttggctggctcttctgttggtctcagtggctcttctggtggtgtgacaaagaccttcatccttgaggggtctgaaaggcctctatgcctttctgatgtttctatgatttttataatcaaaattgtatc

SEQ ID NO: 416 - DNA, 110 bp, Homo sapiens; identical to SEQ ID NO: 4

SEQ ID NO: 417 - DNA, 110 bp, Homo sapiens; identical to SEQ ID NO: 30

SEQ ID NO: 418 - DNA, 482 bp, Homo sapiens
atgatcaagccagaaaacaaggctatgtattacctttcttttagtcaaggtgcaaaattatagtgatataccaaaaaataaaatcttcatcaagtttctaggaactggttttctaagctattttccatccttgtagtttttggtttcatgctagtcaccagaaatccagggataagagaagatacacagtagyagccaggagttagtgggaggctaatatgtcagtgctaaaatctcatctagatctggtacacaatagctagctgactggcaccagcaggcctgttagcaggtgtgagataaagaggattgggatcctaccacggctcaaaaagcaattacacagaaatgttgctgcaggacttaacattaaaaactctccatcagttctagttggctctactcaccagtccaaaggcaattttctcccaaactttaagcattatagattccctctttccccttgaacctgaaaatccagc

SEQ ID NO: 419 - DNA, 64 bp, Homo sapiens; identical to SEQ ID NO: 22

SEQ ID NO: 420 - DNA, 848 bp, Homo sapiens
  misc_feature (277)..(390): n is a, c, g, or t
cccaggtatccccagtcaccaccggcagtaatcagcttggtaaacacccctagtttacctctacttcctttcttatattactcttttcygattccactttccaaaataagtgacctgcatccacaccctgcctgaagctctgctttttgggctctactggctaagacagaggccacttgatactgaccttcatatttatgtgcagccttggcccctggcttatctgcagatacattccacgatttgcgtcacatacatcaaatctaacgaacaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgggcagttacagtggggaggaatccttcatgctgcaattcagtaaagatcatgttctttcatcctgagcaacagtaatttccactcacctctgtatgcggagaaagcctcagatgaggctatttgccaattagtcagttaaaatggagaaacagaacatctgaccagtctgcccttagacagtacccatccacgaggatatacagtgtcccccgccccgggctcacacaggcatgatactcttgtccaccaaggctccagggccctcagccacaccctttcgattagagtaaacaggaaactactatcaactgtcacattgcacattcacaatgaaacctattttcctgggtaaaaactcggcattatatagataagaaaccagcccagggaaggggagggacttccccaaggaaaactgccaattaataacgggtttagacaagaaccttggctac

SEQ ID NO: 421 - DNA, 95 bp, Homo sapiens; identical to SEQ ID NO: 337

SEQ ID NO: 422 - DNA, 402 bp, Homo sapiens
attcagtaagcaaaattctctggggagtttctggggaagaaaaagaggaataagtaggcaagaccctaaaaagaagccacctgcctctcccagtccctccrggaggactccaagagggcaaatctgcaggcagtgagctcagggccagcgggaggccctggagtttggccgtcacagagtggggcagcatgtgggggaccaagggagggctatcattgcagccccactcctaggaaggccaaaccatgggattaccactcagagcttttttctaggcttaaaaaatttttcccttatattttaaacactgtttatgaaattttttacctgcttttcccacttcccatgctaaaaaatttttctaagttattaagcactcataagcattaatgtctgcgttca

SEQ ID NO: 423 - DNA, 438 bp, Homo sapiens
ttcatctaaactgtgatgccctacattgatctgatttacctaaaatgtctttcctctcctttcagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggtgagtggatgrtacatggagaaggagggaggaggtgaaaccttagcaaaaatgcctcctcaccacttcccaggaraatttttataaaaagcataatcactgattctttcactgactctatgtaggaaggctctgaaaagaaaaagaaagaaacatagcaaatggttgctactggcagaagcgtaagatctttgtaaaacgtgctggctctg

SEQ ID NO: 424 - DNA, 170 bp, Homo sapiens; identical to SEQ ID NO: 13

SEQ ID NO: 425 - DNA, 401 bp, Homo sapiens
aagggaggaagatgttagagcgacaatgaagaggcctggaatcaattaggtcaaattataccctgtgttacttgtgtaactcccgttacagtcaaatgaaacagcagaaaagaacactaccctgaacttaggccccatttcagaacggaaagtgcttcataaacagtatctgaatcaagtcatttcaaagggtgggtcagraattgccgactgaaaatgcttttctccgggaacatacatttgcccgaactggggctcaaccccatagcaaccagtcgaaaggcggacatagcaactaccatgccagcgctacatccctccaaatcagaccttccttctagatagtctcccattttctcccccaagcaagtatatcccaacccaagacccccagaggagct

SEQ ID NO: 426 - DNA, 110 bp, Homo sapiens; identical to SEQ ID NO: 69

SEQ ID NO: 427 - DNA, 110 bp, Homo sapiens; identical to SEQ ID NO: 29

SEQ ID NO: 428 - DNA, 110 bp, Homo sapiens; identical to SEQ ID NO: 16

SEQ ID NO: 429 - DNA, 110 bp, Homo sapiens; identical to SEQ ID NO: 71

SEQ ID NO: 430 - DNA, 170 bp, Homo sapiens; identical to SEQ ID NO: 5

SEQ ID NO: 431 - DNA, 315 bp, Homo sapiens
catcctgaccttgccaagggaagtaggaatcaggaaatggtggcttctaacgtgatttcatccaggcccttatgcttgggtccatctcaggaatagtgtttggaattaaygcaggagccagagactaagcaaaccccgcctccaccccagtgcagatttcagttgaatgcagactagagcctttgaaaattatgcattggaaaacaattctggataaattaatttccatcatacataatgcatgaggggaaatccgtggtatttttaaacaatggaaatgagagaatctgccttttgggcattgatcccaattaa

SEQ ID NO: 432 - DNA, 848 bp, Homo sapiens; identical to SEQ ID NO: 420
  misc_feature (277)..(390): n is a, c, g, or t

SEQ ID NO: 433 - DNA, 720 bp, Homo sapiens
  misc_feature (346)..(441): n is a, c, g, or t
  misc_feature (511)..(567): n is a, c, g, or t
tttcttatcactccaagacccccatattgcattaagagaaacagacactcagaatgccctgcaccctcagcccagggccaccctagtggaaacctcatttaaggtgtgcccaggagtgaactgtctttgacagagtctcccactgtacctccccctgggaaaaatctggtgttgaccaaagtcctgaagctaaccaaagacctgactgttctccttgggccatggagagaaagagaccagggagtcttcagcccttgctttggcttctggaactgagaataagagtggagagggggagggaaaagacaccaggatgaactgtcaggagactgctctgactcattynnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacaaactgtcagttacctgtgaccacagccaggaagtatttaatgctccctaagtccagagagaatgannnnnnnnnnnnnnnnnnnnnnnnnnnnnnnnnnnnnnnnnnnnnnnnnnnnnnnnngcgtggaggagctggtttgaaggccagcctgcctgcgcctgcactgcggagactgctctggttctttatgtgggtgaagcagcgtctgtccctgtgggaggctctcacctttcaccacgacttccagggtgagcctgtctgcctcgaaccccg

SEQ ID NO: 434 - DNA, 110 bp, Homo sapiens; identical to SEQ ID NO: 63

SEQ ID NO: 435 - DNA, 110 bp, Homo sapiens; identical to SEQ ID NO: 64

SEQ ID NO: 436 - DNA, 450 bp, Homo sapiens
  misc_feature (14)..(33): n is a, c, g, or t
tgtgaagagactcnnnnnnnnnnnnnnnnnnnntttgcagtctctctctgccatggccttggcaggctgctggaaggcagcttgtggaggccgttggttccgcccactcattccttctcgtgcactgctttctccttcacagctaagatgccatgtgcaggtggattccatgccgcagacatgaaataaaagctttgcaaaggcacgaagcagcacgggtgtgtgcttgtgtgtggggtgggagggctaccgccgactcgtctgggctctatgctgtcctctggccaggtttygtgaggggccgtggtgtccctgggggtacactggtaagttggagctgaccaacccaagacatggtgctccatgggtcacctaacctcctcgagatggtttccacaggagagaaagactgctccaacactcatcaggctcctctaaaatgctcctcat

SEQ ID NO: 437 - DNA, 110 bp, Homo sapiens; identical to SEQ ID NO: 57

SEQ ID NO: 438 - DNA, 464 bp, Homo sapiens
aatttccccctcacccaatcacaccctgtttcttaccactacatttaacccttgtctgaggagcatcccaatggggaggaagcagatattgatatttgtcagaagtgggctaagaacagaytccccctctcctgagttttgggaggcttttttaagcctctcatcaatcatgttcacacacattcaaagtctgatcaaatcgaagcgtaaacaggagatttgtttccaggccaagaagaaagcacgggaaaagcataaagatatttctcaccctctgaataacatgtatgttttgaatactctattccactaccatctgaatagagagccctctctggaaagactaacagctcagagactgtccactctgaatgtactggccacacagacatgcttagaacataaactgacagtcagacttgtctcaggaagagtaacctttctgaaggatggttctctctaga

SEQ ID NO: 439 - DNA, 642 bp, Homo sapiens
agacagtgacaacacttagctggccacaggtcactgggcaggagtttattaaaatgagactgctactagtgagacaaccgcagcatgatcctgggtgacagaacgcattcactcacacgctgtgagaggcgggatgacagggcaccctctgcaatcacagatgcaagattcttggttttaaacatttgcygtattcaacatcttttgtttggtgtccatttactcctcctaaactctcttgctggcttctagtactatttcttcttgctctgtaccaaaggggtctactttgtatcaacagggctgcagagtagggatagagaggatattatgtcaggtagactccaggaagcaatttcacaacaggagaaaaggttcaataaccctggatcaggatgcttcaaagcaggcaaggcaaaaatgtctacggcagtcagagagctaaggaaagcttcagaaagctcatttctatgagaagcgttgaaaatctttgaagatatttctctctcttactcagggtttgctattaggttatgttccttgaacaggtcttttgaaaacattcaagaattttccctgtccaggcattgctcaactctaaaggtaagtgatctctgtgttatataagcccaggaactttct

SEQ ID NO: 440 - DNA, 412 bp, Homo sapiens
aaatacctaatatttgtccttcttaaatttcatcattcatcaaatatcattcccctgctcagccagcctattaacttgttttgcttttttaatggaaaaatttaatttggtactctgactccttttcccagaaaggggggcttgtggggaggggtctggagtagagcagaaatgatcaatggctacaggacttggagcaaaagggaaacagatataggatccaagggaaaggtttgttcagaaaccaaagagacagaccagataaagcaayagatacagtatttggccagagaggcttaaaccaagtctgacagacaacctctggggtcaggaatgagaagcaaaggcaataagaggagaagaccagttgatgtgttacagagagatcccctgagacagtggtgtgtacagc

SEQ ID NO: 441 - DNA, 400 bp, Homo sapiens
tttttaagtgtggggtaggatgatgtaatgagcatcattggtatttgggggcccagccagtcctggcaccaaccttgtccatccttcagcctagagcggtaggaggacttgtcgctggtggtgattctgggctgctatttatgtgacatccatctatgatgggtcagttccttctaccttatcctgtcactcatccaccctaygctcactgctgtcctcctgtaggcctcaagaatctccagtagagatcccacaccagcttgttgatcttcccttattctctgctcttcccatccttggacagaccaggctgagaagggaaggagcaacgtcccacagaaactatggctctgaccccaaaatgcttagtgactctgtgcaggtcacctgctctctctgg

SEQ ID NO: 442 - DNA, 116 bp, Homo sapiens; identical to SEQ ID NO: 35

SEQ ID NO: 443 - DNA, 491 bp, Homo sapiens
ctgaaaatgtttgggaacactgcaaatgcaaatgacaacacagatgctcttataattcacagtgatagtagcaggtgggattccagcccacttggggcgacaggccagctagagagcacgtgtgggatcactctttccctgcatattcctggttggtttcagcagcccaagatcctctgtggagaggacggtgctcaccttggtcacagtggatgccgtacttgccctcggtgcaggtctcgcaggctgtgccgctgaagccctccccacactcacacacacctgtgccattcactccatccaaacagatgccattaccaaagcagacattctgggcattccctggrcagggctggcattgggggccaaagaagccggcacagcattctctcgtctgaaacagaaggacaaagcttgacaacgcacttctatcctctggtgttgtccagcctcggtgtagaaatcctgctgtgggtgacttcttgtgcacc

SEQ ID NO: 444 - DNA, 110 bp, Homo sapiens; identical to SEQ ID NO: 19

SEQ ID NO: 445 - DNA, 110 bp, Homo sapiens; identical to SEQ ID NO: 36

SEQ ID NO: 446 - DNA, 110 bp, Homo sapiens; identical to SEQ ID NO: 55

SEQ ID NO: 447 - DNA, 110 bp, Homo sapiens; identical to SEQ ID NO: 61

SEQ ID NO: 448 - DNA, 110 bp, Homo sapiens; identical to SEQ ID NO: 26

SEQ ID NO: 449 - DNA, 110 bp, Homo sapiens; identical to SEQ ID NO: 28

SEQ ID NO: 450 - DNA, 534 bp, Homo sapiens
agcattgcaattctttttcccagttttttttttttttgagacttttcctgagaaagttttataaacacttcaaataatatgatataggaagtaattcctccttgcctggaagtggggctgagactggagtgctagggctggtgtggaaccaaggccagtgacaaagcagacactgttgcttctatgtggctgtattaaaatgcacccrcccctctgcactgccccacagaccaggatttcaacacagccctgtcgacttgaaagcccaagttcctaaccctgactttgtttcactctgctggggtaaaaccttaccgcagaggctggcatttgctgaaatgaggtcgaggaagtgggaaaggtggttgggagtccaatccaagtctcaccaatgattggataaacacagccaagtgcttcacctccctgcactgcaatctcacagctgtaaaatcacagctgggtcatcagtgatgacaccagcctagaagtgtgggtagcaccattagatgagtctgtgggggaggaaagcac

SEQ ID NO: 451 - DNA, 584 bp, Homo sapiens
  misc_feature (252)..(434): n is a, c, g, or t
aaaagagtttctttaaaaactctattctcccctaatgaaagaaagaatgtggtggacattgtcctagcctgctaagcgtgcctgccttgtgtgtttccccagacagactcacctgcctcactgatcaaagggttgggcactgacaaactggtcacactccttccccagaatctttcaaaagttttaaaaattagtttgacrgtatatttgagctgaatttgagggacagagctcacattcattgtgtagatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagtgcaaactaacccagatacataactctttcatcatatgaaatttctctattgaagaaaaataatggaacccgtggtatatccaaatatttgtagctctaccacaacaaagtcctctactacttgctcatacatatgatttattaaac

SEQ ID NO: 452 - DNA, 571 bp, Homo sapiens
taggcaacattttgtcaagggttctgcctatccagtctcaccaattctggatctcaggatggtaaccaggttcagatagcactgcttaataatgtggttcgattaaacagaagtggtttaattaaaactgactgagactttaaaaggagagtggatgctattgactatgtgatctctgttctattggccaccgtgatgtgcacaactgtttggtcatggactatrttgagaaacattttctctgattaataagagaagaaccaagtaggcttggagctagctgggggaccagctagagagatggaaatccttgatttcccggtgaacaagaacatttataaagacagaaatatgcagaatctgttgttgttgttgtttttgagtttgtggggactaaatcccagatctgtttaagcatttgtgagcactaatgcttttgtgagaatgaatttttctgaaggaacttacaaaagtgttgagtttcatgtttgggttaaagcaatcaagcatgaagacatatatatggatgaagggccaggatggagagagaacttctccaggccatctgagt

SEQ ID NO: 453 - DNA, 82 bp, Homo sapiens; identical to SEQ ID NO: 66

SEQ ID NO: 454 - DNA, 110 bp, Homo sapiens; identical to SEQ ID NO: 21

SEQ ID NO: 455 - DNA, 857 bp, Homo sapiens
  misc_feature (294)..(649): n is a, c, g, or t
cacattccgatctgagaattatcttatggtgacacaaagcgagattttgaggggtaagaggctyggaagaggtttgaggggtggccactggaagccttctgagagggttgaagtgtgtgggatacaggaaggtggtaagtgtgtgtaggtgggaaagtagaagaagagacaccccgagtttctttattgatggagcatattagctcctggctcacaatctttcttcctgtcttcccttttcatcattctgggtggcttacaagctcatgcaagtgactggtcatttactggagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntagcttaacattagtgaaggaaaatcaaacaagcagaccaactagtttcacttaaaattcgtgaccacagacctcacgtgcatactcaatactgctctgacatctgacagtgcttttccaggcagattaccttctatcttcagaaggaaattttaaaattcttactcttttaaaccttgcattctgctttcctaattcttactctcaa

SEQ ID NO: 456 - DNA, 558 bp, Homo sapiens
ggacctccccgtgtaaactactttgggcagtgccttgggtttttcaagagtttgtcaggcacttgaggttgaggctcgaagaaagaatattatttgattcttagccatggtgggaaccgacatttatctaagtgtttttcatagacaggaagtggtaattctcaccctccctgatctgggctctgactcactggggtgccccagcctctgggcctgggctcactagtggtagccagctgacctgagcatcagagtaagatgcgtgcctgaaaaattcccctggtggggccggtcatgaaaagtgccgttctcctcattcatgtggttttgtcaggatgccyggattccgcaggcccgggccagttattttgagcacacaagtcactttaaaagacccctaaaggtcactggtcccaaggattatttcaccccctcaccacagttaatgtcgtctccccacctctgtttttctgtattcccataacacattctgagtgtggctatgtgatgtaaagagctgttttcatgcaaatgttcagggtaacatcatgcatagtg

SEQ ID NO: 457 - DNA, 110 bp, Homo sapiens; identical to SEQ ID NO: 24

SEQ ID NO: 458 - DNA, 104 bp, Homo sapiens; identical to SEQ ID NO: 50

SEQ ID NO: 459 - DNA, 110 bp, Homo sapiens; identical to SEQ ID NO: 68

SEQ ID NO: 460 - DNA, 785 bp, Homo sapiens
acccaaaaggtatcactaattgtccttaaaactcttctcattgccttacytatgatgtatttttaaactggcaaatatataaatgccaacttacacctattgctctgcagcctctattggtgctggccacaagacaaccttctgttgttgccattggaacctgaaattctttttcatctaagcaaaggggtcctgccactccaacagggatgggcatatatscaataacattctcacaacaagctcccatcacctaaaaggtaaagtcaggcaccaaatgaaaatctatatagtaaatgcacaaaattttatctcagcttgtcagtataactatcttcaaacttaatcctttagtatgtattctttttaaacaaaatgtttaattcacctttaaaaagtgtttaaaatactaaaatctttgagtaatgactataaaagcaggaaatatatttttaatttctatgacctacatcataagcagaataaaaaattaggataaaatgatttaaaaggaaaatgttttataaaactatgtacatatatgaaactaatcaagaaaaaagattaaaaacatacaaagcaaatctctcttaatcgagaaaataacatmccaaggagtaattataatccctgtaaggtaggtactggagagaagaaggttctgaaagcttcttggaaagtaactgtcggcgaatagatacaccacgctcatgagtttccatcagagtttccaacttgtaggctgggatatgcttagcattgactaactggatgatctcagcatc

SEQ ID NO: 461 - DNA, 320 bp, Homo sapiens
  misc_feature (3)..(3): n is a, c, g, or t
gantctagaagtaaacacagagctcctctaattttttttgttttctaagcagctgagatgaggtttytctgaatactcaatgttatttcacatggtgtccaatccctgcactttaagaaaccaaatgggaatgtgaaatgcttgttgtttataagataaacaacactcaaagtgacaaattcaaaaatgccatttactcactctgtgggtatccatcatgctgcacgttgggaaccaggacagtaacaatgactaagccgaggcccagttttccatgagcccacagccaaccacagttgagactgagttccaggtcctat

SEQ ID NO: 462 - DNA, 636 bp, Homo sapiens
tttaaaactgggagagataccagcaaaacctatttcaatcacttttcacgttgactttctgagggtaattaaagaaaattcttcctctattatattctttttttttttaaccctctgggccaaattctttgtctagattacactaccatctgttttcatttgtttggtatttagttacaattgtttcacagggtatatttttgaataataattaatagaaataaatcaaacctttcagagaaataattctcatatgtggaatagaaaaaccagatgaatatatatctggaaatatcaagttaattttcaaagtaaggactagggattttgccttgtgrccttgatcaaggtcgataggactcatgtgggtaagtcttggcacagtgctctgaaaatgctatctcctaccaaaagctaaggaaactggaaccaggtaaaagacatcaagaaaacacaggggcataaaaggaagaaaacctagtaacccatattttgtagagctagaataagttgttgtgttaaatcatttagtatttttggcgttatattggaggtaaaattttatactccaaagcaaccatgactaattcttctacctgctgttccctgggtttctatattatttcactgctgttt

SEQ ID NO: 463 - DNA, 629 bp, Homo sapiens
ctgtgaacaattatgttagtgtgctacatttttattttgtaagttctgttatcagtgttttaaatttgggctctcagttgtcatttacatgtgttctatccattctgactgacatttttagatgatgagatagatggttagtccctctgtctttaactcargtatagtaatagatgaagcataatagatggagtgtgtggttgcaattcacaattggtagatttccaaccagctttttctcccctgtaacctgatctcagatacagaagcaggcctgttgatcatttgcacctgaagtacctggaaggtgtccctaatggaaatctacctgctccctggtttccttaagttttcacagtgtcagtatcatggaagtcttgatcaaatcatttggttcctgtgggcattttggggtccagggaattagttaagcttgttatacatagggttattgtcaaatggtattctttccctccttcctgaagaggtgctctctatatacctcagagtgtcagggtcttcccaggatatgctacttctggcttctaggttaaggctcccaaataatgtctccaggtacttgcctggaccaccatgaagaaaggcctgctccagttatctgtaatgag

SEQ ID NO: 464 - DNA, 110 bp, Homo sapiens; identical to SEQ ID NO: 59

SEQ ID NO: 465 - DNA, 126 bp, Homo sapiens; identical to SEQ ID NO: 7

SEQ ID NO: 466 - DNA, 110 bp, Homo sapiens; identical to SEQ ID NO: 62

SEQ ID NO: 467 - DNA, 1282 bp, Homo sapiens
  misc_feature (1253)..(1253): n is a, c, g, or t
  misc_feature (1258)..(1258): n is a, c, g, or t
  misc_feature (1267)..(1267): n is a, c, g, or t
tgtgatgactagcctttggtgttcattaactgactttagtcaaacatcgacctagtgtgagtcagtaaatgccagttttgaggccagatgaagttccaaatcgtcacattctgtaacttacagaattttcatgactatgtcaagcatattaccaatgtatatactttgtgtgtgccttttgttttttaaaaagcacactcccttaatttttggtgaaagaattccagttacattatttrcgcatatttacatggatgtgcatactcaagaaaaaaataacaaatccagaaaaaacaatgaggttcttagaatgaaactttgtttaaccatattgggtgaatatttcccaaaactagcattatcacctttttccattttattgaatccatacataccacgactctaaaaacctgctctactttagtaaacatataacaaaaaaagaatcttatatttagtaaaagcaggaagttggaagagttgtagggttttaatgctcagtatcttaaacaagtatagccccacaatatttactagcatctagccctctcttatgcacccttactaagaaaattaaagaagaagacatttctatacatgttttatttttgaaactgcagctgctgcgggcagagatctcataacagaacattgctttgtcttagaaaaagggaagatgctgagtgtagccggcagcttgcccctggcaagaataagaagagaaggcatcatcttctccctcctgctcatgattcctggctcaggcactctaccagctctttcctgattccctagagtgaagagcaatggctgtgagtgcttcaaactccgggagcagttttgaggcttcaaagcacagcccctgacagctgtccttctcttttccttctccactaaagcagtatctacagtaaatatgaaacattgaagaaaagggaattgcaaatgcaaatacccctctaccatttcatagtaaaccttcttccttccatgtatctctctctctccctctcgtctcctgtcacatcctgctctctctacctcttattcacttcatcaaatgttttcatcaaatgttctaaacaggccattttaggaagttgaataattgctctagacatgaaataaaacaggagctttgctccttattccaacaaccctgttctttggaaattggtataaaaatccaagatggaaagtatctgccaagagtggccgacaagcagaatgcagctcttaaaagaatgccttcntcctnaacaaagtnattgaacctactact

SEQ ID NO: 468 - DNA, 217 bp, Homo sapiens
tctagaggagaattttaaaaaatggaggaaggagttgaaatacatggcccaaaagttttcatatttgaaaatagttccttgctcacttatrttataaactagtgcatcaaatgaccatttctaaccattgtctcatcaaatgagacatttctcagggcaggattagtagtttatacttaaccacaatcattggaggcaagttagtgggataatcatg

SEQ ID NO: 469 - DNA, 552 bp, Homo sapiens
  misc_feature (113)..(178): n is a, c, g, or t
agttctgctttatgtaactctcaattagaccagactttatttgaaacatgttcttatggcaaaatgcaggagcttagaaaagtaaacacagctgcgcagccattttaagcaannnnnnnnnnnnnnnnnnnnnnnnnnnnnnnnnnnnnnnnnnnnnnnnnnnnnnnnnnnnnnnnnncactgtggtcaacagggaggtgaatatttgctatatcataatagaattagcatgcatgtgtgcacacaaatacataccrccgtcacccaatcccatagccatatattgcatatgagtgtcttcttcctttctaatcaaagatttctgttattatttaactaaatccactgaatcttccactgtttctctggcatttagttgggttcattgagtatgagattcctgttccagcaagcatcatattggtaactgcaattcaaagagaggattcttgctgcttgctggacttaactgcacaactatatttttgaaggagaaacaacttgttatcactgttcaatgacttagtcttataatttggggaaaaaaaagta

SEQ ID NO: 470 - DNA, 511 bp, Homo sapiens
  misc_feature (51)..(75): n is a, c, g, or t
  misc_feature (450)..(473): n is a, c, g, or t
actcactagctaatggattcactcccatacactaaccttgctgtccacagnnnnnnnnnnnnnnnnnnnnnnnnnatcactgtgtggaatggatcctcagtcactaattccatgactgccaactttacagatgtcaaggctgccaaatgtcaaggctctgttatagttcaaggtagcaggttttaatctcagctgcatcttaattcattgtgagattaaaagtaatatttttttaaaatgtacccacargtgcaaataattcccattaaaaataagtaagagtgagggtggttgactaaagattatccctacaataacatgattttcttactcaaattatgattttttttggaaggaaaaaatagtgctgtctaaatataattgccaaagacccacagcctcacttacctaagaccaaatatacaacaagtgatcattatgacgttatcnnnnnnnnnnnnnnnnnnnnnnnnttattggttaaaacttcctgagaagcttttgaggtttc

SEQ ID NO: 471 - DNA, 595 bp, Homo sapiens
ggatctaagaacgtagagtttggtttttttcatccgtttattcatgccaagaaattctctttaagattgacttagtttggcatcaacctttaacacatcaactgcataactggcttgggtctacctgcctttgtactctgcagacacacaaaccatgtatagggcatgtttctccaccagattttctttttcagaatttgcactgccaaacagacattaagctactctaaattcttgacaagtatgtaactttataggaacacagtagtgtatttaaacattgaagagaactttctattcatcccactcttctaacagagccatttaatcctatccagaaagatragtccattttcagactctcctttgatcctacagattagagggacagagaagtcaccactgaaaattgaaaaaggacgcaagaagaaatggagatccatgaccatggccccaagaaagcaaaagcatgttactcaggtttcccaagatccaagttagagaggggagaagagattagagctagaaaacaaatacttttaaaaacagcattttggaatttatcaaagtaggctttaatgtcagattcataaaa

SEQ ID NO: 472 - DNA, 431 bp, Homo sapiens
tttatttttcttcagttttgtacttgtttttaaccaaattccatttcttaaagttatgtaataatttctctttttaccatgcatttaaaatcttggctttaaaaatgaaaaaatagtctctgaggtgttcrtatacttttcacccatattggcttgtattctttatttttctttgaattaccacctcaaagccctttcttaatgatcatctttttcttgcattcacaccttttccaaaatccatgcctcatttgtaaaaacaaccctttacaacccttttctattgggtttgtctgacaagcaacaattcattgacaagtgagttatgcagaggggaatggcataaataactctctcaatcagcctaagacaatcattgaggaagtaaacactttttccgaaattctaagtcaagaatcttatctgtatta

SEQ ID NO: 473 - DNA, 705 bp, Homo sapiens
  misc_feature (447)..(465): n is a, c, g, or t
tatttcatcactatgaaagcccaaataccttattataatcactgcaatggacatgacaagaactgggagttctatttcacagtccctattagcgattatgacagaaagtctacacgtgccttttaatttcatcacttaacgtttcttgggctgaggtaatatcaaactcatctttacattcatccactgtaatttcaagtagaatgcctgaaaagtggtagacatatataaaacatctaccgaactgaattaaagcaaaataaaacatgtaatgtaaaacagaacactcaggccttgtgtcttacaaaatccttgaaacttcacaatcgagatgtgaccattgccaagtctttccaatgaattaaaaagcaaacaaaatttctactatacttatgccacgtgaaattgctgcttttgtagrtcaagaatgattggatatcaacaatnnnnnnnnnnnnnnnnnnncagaatataatcaatcacacaaactaaggtagaaaatctggatatatctccaacaacaatgttccacttaccacaatattcatatacaaaggatataatcacaaggtatataccaaattagtgtgaaaacagcatgtttcaggacatctaaggaaatcgtataatcaaaacttattttttaaattaacaagatataataaaggcattttacgtgtaataaaattcaccaattttaaacgt

SEQ ID NO: 474 - DNA, 642 bp, Homo sapiens
  misc_feature (261)..(409): n is a, c, g, or t
cacagattatattgaaatcctgattgaataattactgtcatgattgttattttactatcagaaagaaacttagaggtgtcctcaaaaacatatattattttctttcataaatttgatatgtatgtgtatatatacagatatgaatgcacaatattggggaaatatttcatttttcaatgcatcttttacatctactgttttacttgttcctcaaaaaaattctgtgagaagctgaggtattttattgtgaggaaattcaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctaaagccacactgcaagctagtttcaaagatagaattcattactgaattttataatcaagagtgatgattaagatggtgatgacagtgtgtgggtgttgttgaaaagactgcctctagacacccttccttttgtgttggygtgtaaggatgggtctttaatttttgtactgtctttcatgatttgtttgtgataacaaatcgatgaccgctatgagaagtctttcattgct

SEQ ID NO: 475 - DNA, 664 bp, Homo sapiens
gacccaatggttcccttctcgtaagtcactgctggtgctggtgccttcactccctccttttacccagtgcagagtctcccaaaggtgcacagtggctgaatgcggagattcctggagattcagagtcccagggggaggggctgggaaagtgtgtattaggaaacactcgaggctccaggctgacgacctcagggctcccgggctcacccccaagcctatttggccttggaggctgactggagctaaggcctggaacagagccagcaggggtcaagccaaagaatcacagtcaccctcatgcttcccatgccccttccatgactggcagagaaggctccagtattaacacagcacttttaaggcacttgatcctgtcagaacagaccactgcagccccctgaccaaaaggctgggtggtaactaaggctcaaacaggtgactccctcgcagataggtcctgatgggagtaggcaaactcaggacttctctggaagctgcatacatttctgaaacggacaccataaggaattaggagagttttgyttgttttttgtcggtaatcatgaaattataccaccagctcacaagagctaggaggacttgggacaagattttagagatgttaaagcacgccatccacatgatctcattaaatcctgacaat

SEQ ID NO: 476 - DNA, 110 bp, Homo sapiens; identical to SEQ ID NO: 54

SEQ ID NO: 477 - DNA, 495 bp, Homo sapiens
aggagactagacctctgtgatggtactgagaggttcccaagggccagaggtccccagggatgtaaatatacacagataaaatttttctagataaatatgctatttggtttgctatggctgtggaactatgaggttttcctagctttctcttctgtggttatgtaactttataaaaaaaggactttatggttaaaaatggaacagattattctagactttaagaaataattgaccaaagttaattaccatgtttataacagccacacctgctgtatcatgtggttgccaagaatgatcttaaaaatataaagatgcatctgctacyatgacttgatagaagtggcttggaagcctgcaggttagtgacatcactccacttcgtgagctcttcgatgagaggtgggttggctggaggtgggtttattccctggaggaactttcgtgctgagatgaggactaaacgccttttcttgctccttcttgatcaccagatcc

SEQ ID NO: 478 - DNA, 401 bp, Homo sapiens
gttctttatcccattccagtgtaaggtggccccacttcttcccagccttctccatgtctattagtattaggtatcttagaagtcaaggacagcattggtaacaagagtcccccaagatggagacatctccatcatgtgtagggcaggttggggtgtgtgtatgtggatgaaaacaaggttgaagccggctgttatagactyataaatatccatattggaactcaccaactgtgcatgttgggggaaaagaagcagagagatggttgcagaaggagaaaggagagtcatcaataatacccaaatgagctggttaatgtttccagtttccatccagaacttgacatttgagcagagccacagacagaagctccttactggctttgtttcagtgcctctcagtt

SEQ ID NO: 479 - DNA, 557 bp, Homo sapiens
tgctgttctgcaggcctcccatggcactaccaataccataacctatcttcattttgaaacttttcaggttaccacttctaatttctactactctggtctctcataaatttctttttcctttctagtttctccgtattttgaacttccatacataaaagctgcccttccacaggaaaaaaattattttccttgcggtacaaaatacaaccagagcttgggaaaacttctttactaccaaaactttaaaaaggatgattttttggttctgacccattccatcaacctaatggtttcgaggggctattcagatcttacctgactgtattcataccccttagttggagaaacaaaaacaagaacacaccaaacttattagaataaagagtgcattctggaattctgttatttaactatcatcacagtaaytgaccagtttcaataaatgggtaaaaatctaatcaacgagaaagaaagtcactagaagaactggtaattccatcttaagaaaggaaaaaaggcctccaatcaaaaattatgcttgaacatatctaaaagaa

SEQ ID NO: 480 - DNA, 504 bp, Homo sapiens
ggatctcacagtcagtaaaacgcagtgttcttaactacctggctagatgacttctcttaagagatgttcagtaacattagttgaatgaatgagaaatttagtgaggactcctaaggccagggcaggtgaccagaaagccaccatacccgtgcttctcaaaagttgtctctccataatattctctagtaaggcccgcgtaagccagagggaatttaagaatgtagagaattttctgaggaacacattatgagggttacaagaatactcyattttcactggccatagagactgagttatgatatagggtaacaaaccaaagccagagtccacagaggcttaggattgtacagggtgatataggcgactacatttttacctacagaagtgagtttccccaaaatgttggcttttatttcctatcttaccacctaacttcccacctgatgttatagaagactttttatccctgacagtttccaggctttgttgtctgcattaggccaa

SEQ ID NO: 481 - DNA, 1148 bp, Homo sapiens
  misc_feature (109)..(388): n is a, c, g, or t
tcactgtaagtatcaatttgctaagcttctagacattctttctacctacaataactttttttacaaaactaatatttttgtagttttcttcacctttatttctagcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttaattgatttgcaattaaaaaaatgctgacacactggaacattctcccttacgtagttccttgccagaaaaagaaaaaaaaatctattattgaaagcaatcacttgaggccaggtttaacaaccattttaaaagatgcaaaagtttccaaatttgtactctcctgtctctgaatctattttaatggtcctatattccttaatatgttataaatgaaaattcagtataaacaaaaagctggaagattttttcaaaagataacattcaattataaagttcctaatgtttcttagaattgaggtgaggcaatggaaactaaaatttccagaactgtttttgtacctggccccacccttggcaatcttccccttctcaagaattattgacattggtgtttccaggaactcaatttatattatgtatgtcagcttccaytttcccctcacattacagagtttataaacagttccacttgttaatccaactcatgagtgcttctcaaaagcactacagaacataaaacgtcataagtgaaagctctcaagtactacatcaacaattacccaaccatgtctacatagtaatcaatattttggatacgttggaaaaattgacattaatttctgagatattttcaaatacccttaggtttgaaatcaagttaccaatcatttgggtggataagtaattttaaaagttttatactggataactagatatgtttcaatctattttagacacacagtctcaacaaaatta

SEQ ID NO: 482 - DNA, 563 bp, Homo sapiens
gcacccaggcatgtgcctgggctcacttagctgtcgtccgccttctctccttccctctcttcccctgccatgagccctctctcttttcccataatcactttccatctcttccagaatgcttaatggagtctagagacagaggaaggaggatttyatctcaaagcaggaaccagcatttgcacttcttttcctgttattttgaaagtggatttggcattgggagtgagtgtttcttgggggagggagaggaacttctttccactgcccctttctcccttctggaaaaggcttttttttccccatcccctgctctgttgtcactggtcccacgtagctgtccaagcccagtgctgtctgtggtcatggaccaacttcagacggagaggaacagaatgtatggagaaggatgattggagggaaagaatgctgatagctaatgggtaccctggcaatgtccagggagagggaccccaatcctgtacttcaaatagcttgtctcctaatgcttgttgttgggaagtggttgtcttcccctcctagcctgtctgccaagtgaagtgcaa

SEQ ID NO: 483 - DNA, 350 bp, Homo sapiens
  misc_feature (282)..(302): n is a, c, g, or t
tcagattcattctccatcgtgactcttgctggacacagcctaatgatgtgcaaatgatgatcagtacctgtctctttgctaccgcaacggaaaaaaaatcatagcttggttcactaacaaaattaaataaataaaacttataagaacactaaacattggatttttactgctcatgtaaaaatggcacaaaacaaacatacyacatcaataacaagaacatcacctgttttcatatccctggggttttattacctaaagtgtgttatagtgaaactggatagnnnnnnnnnnnnnnnnnnnnncttttgttacgctcattttattttgaatttgaggggcctgtttcagac

SEQ ID NO: 484 - DNA, 973 bp, Homo sapiens
  misc_feature (350)..(377): n is a, c, g, or t
  misc_feature (387)..(479): n is a, c, g, or t
gcaagatattggtgggataaaatcctagatgtggacctttcacttgaagctttatgtaattaaagtcattgaatagacacatagcatctattcaattttgcttcctggatatctcatattggatattacacacagtgaacattttccgtggtgttgtacagcctccaagattaaagttttaatggtttattatttcttattatgcggatacactcaacctttcctctattgtgagacagttcaactgtttctaattatttgcctcataaagaatgcaatgactatccacatcccctgtgtcttacccttggcccatttgcgctgtcttctcactgcatttttcttcacannnnnnnnnnnnnnnnnnnnnnnnnnnnggagaggcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcatgccaggggtggctgtttggggaggtgcagagggatgtggaactagggctgccacctgggtctccacttgcatgcaaacaaggcctatgtccagtaggacataagctaggagtggcaggagaagcagggagcagtccctggacctccatttatactagcttacccaggccccctaaatgttggaatggttctgctatgacacccttaccttcttttcccagattcaatagagttctctaaagtagaccgctggattaaaartaaaaccgatcaaccaaccaacaacaacaacaaaaaacccaaaacttcattttccaaagttatttgtaaacttacctcttttgcacacctgttatatattgccaaaatttatcatttgcgaaatttaaagacctgtgccatgagagatgatttgaaattgcaaaaaggacagtgtcagggggagcagttgacttcacttctgatctgtactatcaaaatcctgctctag

SEQ ID NO: 485 - DNA, 217 bp, Homo sapiens
tatgccttattacagatgctattaagggcacactttaatactatgcatgctaattttattcactttaaagaaaaaagcatctaaaactcaaccagaagctttccatttaatataatattttcctttaggagtaagtrtatgagaaggtagtttggacacacttctgaggcttaaaagactttatatatgaactttgcattagcaaaaataattttta

SEQ ID NO: 486 - DNA, 170 bp, Homo sapiens; identical to SEQ ID NO: 11

SEQ ID NO: 487 - DNA, 576 bp, Homo sapiens
  misc_feature (90)..(183): n is a, c, g, or t
  misc_feature (205)..(294): n is a, c, g, or t
tctttcagtttcagcatctcagctcatacaatgaccatatgtaaagactgaatttgtcagtgctacctgacaataatcttacagaaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactcgtctggactatagagttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatatcacataatttcaaacataattctgaggtgtagtaaaattacgaatagcaacttataaactacagaaatctcagccaatttctcaagtatacaaatagattagtacaargatgatctttctgtaacattagtaaggcagcaggaaaaataaggtaagataatgtacaattttttagcctgaacattttttaactttacacctaaggtagtgttataaaagcactgaataggtgaagatagaaggcttaggaaaaaaataatactaactatcagaaaat

SEQ ID NO: 488 - DNA, 110 bp, Homo sapiens; identical to SEQ ID NO: 27

SEQ ID NO: 489 - DNA, 170 bp, Homo sapiens; identical to SEQ ID NO: 12

SEQ ID NO: 490 - DNA, 110 bp, Homo sapiens; identical to SEQ ID NO: 23

SEQ ID NO: 491 - DNA, 110 bp, Homo sapiens; identical to SEQ ID NO: 44

SEQ ID NO: 492 - DNA, 170 bp, Homo sapiens; identical to SEQ ID NO: 10

SEQ ID NO: 493 - DNA, 110 bp, Homo sapiens; identical to SEQ ID NO: 42

SEQ ID NO: 494 - DNA, 110 bp, Homo sapiens; identical to SEQ ID NO: 67

SEQ ID NO: 495 - DNA, 679 bp, Homo sapiens
gtgcctaagcactgctcttctgcatttttgtaggaggaaattagctcattatttaggatctgctgtgtcttcctaactctctgatctccttggagcctttgccttggccacctgtagagtctttaggttgatgcttacatgttggtggagtttctaggcattctgcttctccctctgataggtcactacagttcttaaagttgaaggrgaggcagaggctattggcaaaggcaagatactgtgtggatttcagagttccttgtcatttgttgtattttcaatctgaacagaaaccaaactgaagtctccaaatcctaggattcgagagggagtcatttttgcaaggagtctatgattccctatgtttgtggggaagcattttatgtggaaaaccaaaaacccatggcctggtatatacagagctttcaggaaatggcagctgtaggcccatgactgagaagtttggcttcccacaaaagctgggccttgtgctgaatgtgactgatactgatggagtttcagaaggattgcagtggagacgagtgtatggtcagaattacattttctagccagattatcagagtctattatgcatttatatggccaagaaggcaggcagaaagagcagttgggaagcaatttgtgatagtgtaggagagattttgggggctattgtgtt

SEQ ID NO: 496 - DNA, 631 bp, Homo sapiens
gagagtatctctcttgctctgagaagtggcagcggccctcagatgctaaagataataggccacttcccacataattattgctgtagggagcaaaagggaagtagagaaatagcaagcagtaatgccaggatcctatttggcacagagagccagagaaatgtggctgcaggctagatggatttgagaaattctatttgtgtgacttaattggatcaatttattttaaaactccaagtaccttagggtaaataattctatgtggcttgttggagggcagagcctgcagcgtgatttccaaaacatgagctacctgctagtggagcatccattctgaatgaggtgccttatcttcccacccgaaatgaacagagtgcaaatcttttccaccagctttgaaacacagagttctgtatgaattattttgcatatgatcaattctaagagggagtataaagatattaaccatttttgtatggattcacttrtgcaaccccactcggtcaattaagaaagtgggagacctcagagaatattctagcattgggccccttgggaggaataaggggtccccaatagtcttagctgattctaaagagcttctgctaaggctcagcccgggttttatgtgcat

SEQ ID NO: 497 - DNA, 501 bp, Homo sapiens
ggctcctcatatggtctgatctttgttgcattgcccatttttgcagattctgacatctggaatgtaaattagaactacatatttaatatttctgtttaatgtttattattgggctcccctgaagattgttatttggaattggctaccaagggcctgttattctaattgacagagctctcatccagcttactagttattcctttctctccctcagtacactaagaaagaggtgrtgagctttttcaattttcactgaaaagagtaagtgagcagagatctgtaagttgacttttttgcctggcagttgataaatgttcactgaatgaataattctagagtcttctaggacatttctgtgtagaaacttcttatttacattctagaaactttggagaaccaatgttggttctgctaaacagtattctggtcctcaatgcctagattcaccaatttagaccccagacacaccaagttatgcaccttgctttactaaattctata

SEQ ID NO: 498 - DNA, 317 bp, Homo sapiens
caggaaaaagaaaaaaaacctagtaaggtgaaactaaatagaatggcttaagtaaatatatgtataagttggtgtgaaacattgtaatctgaggttgcatgaatttcttggacagcrtttatctctatctgccagttatggtggtgggacggggtggttcaagttggatatcgatggattattttcattatcagcctatccataggaaatatgttggtgcattaatattttccaagttgaagttcctaaggagatgtaagaaaactgaatttgcccaaagcaagaggaagaaaaataaggaatatattttagtgtga

SEQ ID NO: 499 - DNA, 110 bp, Homo sapiens; identical to SEQ ID NO: 53

SEQ ID NO: 500 - DNA, 170 bp, Homo sapiens; identical to SEQ ID NO: 8

SEQ ID NO: 501 - DNA, 110 bp, Homo sapiens; identical to SEQ ID NO: 32

SEQ ID NO: 502 - DNA, 649 bp, Homo sapiens
  misc_feature (440)..(461): n is a, c, g, or t
cctatacatattaaaacaagcatttgaaagtttcaacaaagctaattattacaagagactctcctgtcaagcacatgtccctactaagagtrcatattcgtcttacctttgcctcattaatggaatattaatgcaattagcagcagctacggcagcaaagggaacaaaacgtcctatcagtggtgagacatgctgcagaggagaaaatttcaagagctttatcttatttttcctgtactagtaaatggaaataacttatttcttcttgaaaactctgaaaaatgcagagtcccatttgcatagaaactcaggccaagaatgctttcaaaacttttcctagcagtatttgtatatttcccacatgggtgtgctgggaccatgcaagcttctaaggccttgtagctaagtgtggttgcaggagtatgggctttggagtgggnnnnnnnnnnnnnnnnnnnnnntgctcttgcctctcttcagagctgtggcctgaggcacactgccctctgcaatgcctctggtgggctgtcaggatgggtgaacatttagcacttcgtacacagagtcaggcacagaccacagtcctggtaggtgttactggtgacaactcttcccaaacttattggaagatataactaaaatatgcagc

SEQ ID NO: 503 - DNA, 640 bp, Homo sapiens
  misc_feature (122)..(145): n is a, c, g, or t
ctcctctcttggactataatctctttgaaagtaatgacgtgtcttttgcatatccaccaaacatccccctccattccccagcatccatgtatttactcactaaatgataatggaatgaatgnnnnnnnnnnnnnnnnnnnnnnnngtgcggacactttgttaattgtctgtggagatataaaaaccccaagaagaggggaacatactgctgctaagaaatgtataacgtctgtaaagagacaaaacaacctagggatggtctgacaaatgccacattgtgtaataaaaatcaaacacctgagtgctgaggaggaattagatgtggtagaatctaagggttcttttgagagtaaaactttgaatgagcactctctgtctcatactccagggtttccagtcctgaactgggctttctagtcttcatgtcctcactgtcgtctccagtacaaaatgraaatatgtgacagctgttatttactttgtatgtgtgttttgataataaaataattgcatcggcagttgagtggctgtgtaggaaatgccatttgaccctatcttccatctcagttacccatgtttgtttatgggctcagccctagttccagggctcagttctcatgataactagtctctttctaga

SEQ ID NO: 504 - DNA, 533 bp, Homo sapiens
agaattcaataataaaatcttgactgacttttcttcaaacacatatgtaatacatagcaagaaagtatttcaggtgaccttattttatcttctaaaatggttattgggacagttcacctaacactcttttgccaatttacataatgatgagaagaatcaacaaatacttagatgggattactagtcaatgtaagaaaggaggagacagcttcattcatacrgaaatgactcatcaggtgtttctctataaacttgcagaatttaccgtctgctaaatggaatggtggccggaatgtcctgactgattagctgttgcccatagaaaaatctctggttttttcattttaccatttgttttatttttataagtcattatcaattaaaggagtaaaaaaattactttttaacccctctaaaaaaacctctagtataccaggttaaagcaggtaatattttccatgatatttattacccccgagaagatccagttttcattgcccaataaatagtacagcagggcttaaaaaatatgt

SEQ ID NO: 505 - DNA, 110 bp, Homo sapiens; identical to SEQ ID NO: 45

SEQ ID NO: 506 - DNA, 591 bp, Homo sapiens
taaaattgagggtctccatatatagattttcaagtgtggaatcaatggcatcattgaggtaaaggcttaagtggaatatcaccactataatccttgctatgaattagattgccagatgaaaaacatggaattcactgcctgtcacaagggacacctccccactccaaacagaagaaacacacctcttacagactcatttttacacccctcaagagcaaatagtccatgcttattattatttaaaaaacctagagtccttccaaaatgtaatgcattataaaagccttcacatcatggctaatattgagaaaagattatttatattttcaactcagaggcagagcttggtaaaatgctatagttgtaaaattttgtggctatgttattctttttcaagccraatctcaggcagaatctacgtatatagaaatatgacagaaaataaagagttgctctagttgaagttggaggcttggccataactgtttggctttttcttgctgccttggacatcctggctttatcacacccgagtcacacctcagatagtgagagtccacagtacatagtagagtcctaccagtagagtag

SEQ ID NO: 507 - DNA, 311 bp, Homo sapiens; identical to SEQ ID NO: 14

SEQ ID NO: 508 - DNA, 500 bp, Homo sapiens
tgagaaagttgttctttatattaaatagctgtgtatcttgctgaaaattgacacagaaaaatttaaaaatatatgtttattttcaagattccaatattatgagaatttaacttctaagaagaaactgaaattaccaccacacccaaaaatcttgtatggcatctaatggtcaggtggcctatgagcttatgaagtttaatttgcatcactattgtactagctacgttcaatatcaaccaggcagcaagtgtttactaatcttttacctctagtaattctactcatgatatcccttcaaggtggtaaatatatgaaatgtagttacacagtaataaatttttaaatacagatactcagaaagttcactttggaaaactagccattgattttagaaagagctttgtaatctatcgtttttyttaattttctttggttgacacaaaggtcagagctacagttttagtgccggtataattgccttagttcattcagatcttcca

SEQ ID NO: 509 - DNA, 542 bp, Homo sapiens
tatccttttatcatcacttcttttaatccttctctagttggatgaggggcagtatcaaaaggcgtattttcctatatgattaaaacatttttaaatctttttttttctctttttgttcttgggattgtcttttttaaagggtatcctttcaagytactaatctgctaacagtcctccataaaaatgtgatcccatttctacttttgatggaagacagctctgataccatctgtcagttctgcatttatacttgcaactggacacctttgttgagtaccccaccaacttaaacaattctgtcaggtgttttacatagaggcagacatatagtagtggctcaataaaatatctgcttctgagtgaaataaatgaatttattcatcttcttagaggtgaatcagtaaatgacatttctgtttgttcacttctgtttttagcctgattaaataaataacttttctgaaaagcagcagaaatatcaatacttaaaagtaccatataaggttaggagattatggcatggacacttggaatcaaactca

SEQ ID NO: 510 - DNA, 597 bp, Homo sapiens
aacttattttagcaatataagttcacaggtacacaaagatttatataaatagatcatctttacacaaaaactcaggatatacaaacacactagacacctcttgtaagccacttcatatttattaagtctaatgtagacgttgtaccattcaagcaggtgcatgatgcacagaggaattaatgccccataagcaaactttggccaatgggagacaggagacagcagataaattactctcccttccttccttgtcctgagacgttgtttatgttgcctttgaagatatgattctgaaagactgagcaatctatyatgcttgatactgagcagtggtcagctcagtacctcattctcatttttgctttcctgacatccttacctcattttccttttcctaaatgtgaggccattttccaaaaggagaagggaaatgaggcaagaaagccacttaccctctaggattgaactaactcacaaaattatgacacaaaagcctttaccttacacattgttatctgaaaaattcttagctaaaagaaatgcagaaattctaaatgtccatcaataggaatttatgcagaaattaaaaatgataag

SEQ ID NO: 511 - DNA, 436 bp, Homo sapiens
  misc_feature (280)..(373): n is a, c, g, or t
aactttactaatgatcttctagatatacagataacacaaagtgtggtctgggtcttacagtgctattctcaggcttttcttgttgcatcaggatgcagactgtttcgtggtaacatgaatttttccagcaatgtgtgtgtccacatattactcacacagaaaggaatcctttaaatcaygaaaagcccacttggtgagtaaaatagagcaatttcccttactttttaatgccttgcattgagagactaaatgtttcacaagaattcacacatttagtccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatcagtatcttggcctttaactgctaaactataatgccactttcaggctggcgaaggagatg

SEQ ID NO: 512 - DNA, 339 bp, Homo sapiens
tgactaataccaagctgagtaatcctgagctgaataatactcaacttctaggctagttcataaaagcatgaataaatactcaacttctaggccaacttctaggcctagttcataaaagcatgaataaatactcaacttctaggcctagttcataaaagcatgccatgcctaattgactgtgttctttccttggaatgtgtgtgtggaagctaatagaatcctggaaycactatctgaaggagacatattgttcatagtcacctgttttggaatttacgagatgcattgacttatacttattaatccacttagattttggggtgtatctattatagtacc

SEQ ID NO: 513 - DNA, 95 bp, Homo sapiens; identical to SEQ ID NO: 17

SEQ ID NO: 514 - DNA, 538 bp, Homo sapiens
agaggtaggctatgtgtttgcctaagggaggagaatatgtaccatctgtaagttctttacagagagtccctttgtctagaaattatgtttgtttgctttatttcccctaaagaactggggagacacatgtctctttgtatctcaaataaagacaaaaataaaatataattggatgctacttacaagccacttatttcagttctgacagaaggaagaataagggtcagaacacctgctcttctctgttgctgaagggttgacctggcagtggcttgttttaggacatcagataatatccttatgtgttttctgtccccrgcaaggtacatactcaggatcaagggcagttatgaagatcagaaataccaccttaggtgaaagatttcagcagaaaggagaatatgttggcattttatgtattaggaaataaaagcagaaggaaaccatgaagctgtctcttgatatcctagaagttataccttattgcaattaaaataaatcatagggtaatcttggacatataatatagatagcccta

SEQ ID NO: 515 - DNA, 110 bp, Homo sapiens; identical to SEQ ID NO: 49

SEQ ID NO: 516 - DNA, 609 bp, Homo sapiens
  misc_feature (383)..(406): n is a, c, g, or t
aaaggatttgaacaccacttacaatagagcaaatttccagatagctccaggagcttaatctttggaagcaggagtttctttgtttacaaacaagcaggcgtttgttagccagtgcaacaataaacctctgctctctggtcagccattttcattaaattattcttaaagaccaccaataaaaggtgccartcattctacttaactatgtggctaaccatctattattaaccagagcacatacttctggaataacaaatggggtcgcaaagatctgagctcgataagccctataatgagataactcccgggcctggtatcatgtgataaatgggatgattgatcagaactgcacaggagagactgacgtcctctctgcgctctcnnnnnnnnnnnnnnnnnnnnnnnnggtttagctctactaaaaaatgctaaatagagatatatatatttcatataagaaatacgcaaataagccttttttattaaatgcatatttgctagaggttaaaccctggtatcctgcaaaaatcaagagaagaaaacttgctgagtgtttctgccatgtcattttccagccatatttaacagagagagatttctggcaggact

SEQ ID NO: 517 - DNA, 110 bp, Homo sapiens; identical to SEQ ID NO: 43

SEQ ID NO: 518 - DNA, 110 bp, Homo sapiens; identical to SEQ ID NO: 46

SEQ ID NO: 519 - DNA, 315 bp, Homo sapiens
catcctgaccttgccaagggaagtaggaatcaggaaatggtggcttctaacgtgatttcatccaggcccttatgcttgggtccatctcaggaatagtrtttggaattaacgcaggagccagagactaagcaaaccccgcctccaccccagtgcagatttcagttgaatgcagactagagcctttgaaaattatgcattggaaaacaattctggataaattaatttccatcatacataatgcatgaggggaaatccgtggtatttttaaacaatggaaatgagagaatctgccttttgggcattgatcccaattaa

SEQ ID NO: 520 - DNA, 748 bp, Homo sapiens
agccccttccttctgcttccctaaatgccacgtcagtgggaacatgctggggcgggaacgcagaatgtccctttctcaggaaacgcctgaccaggccaggcccacgggtcagaccttggtgcgaatgacattcttgtcggaatcgatgtcggtcaatgtgtcgtagtcatcctcctccacagagctcatggagtccactcggggccgagtacctacaggttcaaaggagcggcctggaaagggcagggagggaggggttgcaccggtggacacaaaacccacagtggccctcgcccatctgaggcaattrtaacaattattaggcagtagggaagaggcaaattgtgggctgggcctcagagttggggtggaagtgggctcaggagctgggaggagacatcaggtgctcctaagtcctgggggatgatggtagactctagaagaggccaccacacaggggccgctctagctttgctctgttcctggcacccctcatgggctagggcttcccaggccaggacactgagcagaccccaggcagcagacacaaacaacaggattgaactcggcagtcatgctgattcaagggtgcccagccctgtgggttctgagaaaacactgccttggctgccactgggttaaagaccattgggcctggggtgcagaaagagcatttctcctttatccaagaggcagatgggacccctatgcctggagccctccccaacagccagggagtgagggcgct

SEQ ID NO: 521 - DNA, 597 bp, Homo sapiens
gggtaacccagcaaggccatacccaccagccatgcgaagacccctaagccaacccttctcctgctctccctctggctccccacagctctcccatcatcccatgctgctctccggctgcttcctgactttctcacatgtctcttccaaggcactgtgcacacagaagatgctcaataaacacaatttgtcccttcccatcccttttctaagttagctttgctttctctatgtgttcactgcactcttacctgactgtgctctcatttcagcatttcctaaagacatacaaagggctaaaaacacacctctcctgaaattccatatctatcccaaatgtaggatgactcctcacattctcctctccttctaccatatggtttttattctaagctcccaggaaagttatttgcaggcttggtgagtacaaagggcatgtacgtgtcttgtactacagcacactgtgatctagactccagccatgcccrgtccctacgacatccatttgacacaattcctacctatttccagcactccccgtcataaccagtgagccaatatggggcagagaaaagtacactgatgggaatttatgtttca

SEQ ID NO: 522 - DNA, 79 bp, Homo sapiens; identical to SEQ ID NO: 345

SEQ ID NO: 523 - DNA, 170 bp, Homo sapiens; identical to SEQ ID NO: 6

SEQ ID NO: 524 - DNA, 110 bp, Homo sapiens; identical to SEQ ID NO: 48

SEQ ID NO: 525 - DNA, 98 bp, Homo sapiens; identical to SEQ ID NO: 51

SEQ ID NO: 526 - DNA, 419 bp, Homo sapiens
aggttatatcccaaatgttcacttactaccagcctgtgggcacagatgacccctgaaaagcaacaataattcaaatttcactggactgttgtccctaccaatgtaaagcattctctctctacccacccaggtatgcctatgtatcttagtggatattttacacgtattcatgaacccatctaccagtagatttaatgttctatcacttcyccattggatacaatcatggcattattgcctcacctgctacttatctggcccacctttcctgacagtggccaccatcactccactaggcaacacggtccatggcagctcttccatgccacccagtaggatgtagcatcttcctgtcacagcctgccatcaacccactgagctagcaatcccctgacctagttgctgctcattcaccactc

SEQ ID NO: 527 - DNA, 73 bp, Homo sapiens; identical to SEQ ID NO: 20

SEQ ID NO: 528 - DNA, 110 bp, Homo sapiens; identical to SEQ ID NO: 58

SEQ ID NO: 529 - DNA, 792 bp, Homo sapiens
tagctggaaatgtcacccaagagtataataaaaccagctaactccacagtcattcaagatggctctctgacttcttccaggtaatctcctgctatcagtactgaaacatgcctatgaccgagggttaagctcatgctttcayctccccaacacttaaaactccctctgaaagtgacccctctttatctttacgtctctcttatcatgtaaaaatctatcagtgacaaagaggaacaactgcaaagctgcagaaattaaaaacagaccttttgacttaggtacaaaatgtttgcactttggacataaagggaatttgctgagttactgaatcaagtatttatagcaccacataattgattttctgccataataatttcttctgtaatttagcagctcctccacaaatccccatcaaatctatgacttctcaaaaatgctaacaatgctggctaatacaaaggaaacagtgccacctaagcaaaattaaactagaataataatctattcataaagttctttttagggacacatagttattaaaatctttgcttttgttattcacgagtcatgagttttgcattttccattcttttctcaattatgttattaggctgtatcttatcaaaagtattctaattcctatcttcaggctacaaagtccttgccctaaatgaaaatattagagtccactaaaaaccaacataatcaagtatgccctgtgacttccaaccccaaaagtggaggatgctgacatgcatcatcatcggttttgccatttgccagctggaggac

SEQ ID NO: 530 - DNA, 189 bp, Homo sapiens; identical to SEQ ID NO: 3

SEQ ID NO: 531 - DNA, 110 bp, Homo sapiens; identical to SEQ ID NO: 33

SEQ ID NO: 532 - DNA, 79 bp, Homo sapiens; identical to SEQ ID NO: 60

SEQ ID NO: 533 - DNA, 369 bp, Homo sapiens
  misc_feature (152)..(192): n is a, c, g, or t
attgtctgcttaccttatgaagacaccataaggaggtataacgtgtggtgagcacaggtgaagagctagaggcctgaaactagatggggagggagaagccagccatgcctgcatccagggctgcactcagcctcccagcatctgtgcagagnnnnnnnnnnnnnnnnnnnnnnnnnnnnnnnnnnnnnnnnntatgtggaacatcccagtcatcccagatcatcccagtcaaccaagatcataaaattgttttcagctattaagtttaaataygtgaaataaggagataaaaatactgacttggactctgtctgaatgaaaggattagtagattagattagtgattcttaaaagactttcttggatgca

SEQ ID NO: 534 - DNA, 534 bp, Homo sapiens
  misc_feature (236)..(329): n is a, c, g, or t
ggctggatcaccaggggtgttggtttatactcatcaggcaaattgcagagaaggaatattatgacctaataggctgattttcaaacactgctagaagcctcatatatcaaatagactaagacagaacttctctgaatcatctacatgggcccatatctacccatgaggccccttaagcaactccacagacactgtgggagagacacgttagagaataactagccaatcatcagtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggaaataagttttgtttcagttcagtaatactatagctccttatattacatttttaaatctacgaactctattattatgcctgggccatcagagaaagtgatctctctaaccayggtaccaatggaaatatttggtggagagaggggagatgtttctaaatgatgtagcagtcttgacagcccaaagtataattctttgtacaac

SEQ ID NO: 535 - DNA, 426 bp, Homo sapiens
ttcttcatgacacactttttcaccgggtaattttgtaggagcaatattaatctgaaattagataggatggtgacttatagatcagttttcagcactcattgctgaaatttgctgtcacttgaaatagytgattttgaaaaacaagcccaaaagatttgttttttcttcccagatagtttgcctatattggtaaatgtttccatcaaatgaagagtggaatattagtagtgtttacatcaaatgaagagtggatctaaagtttaaacatgaagatttcatccggctatgaatgaaagatgagagagtaacgtattttccttcctttttcgttctcatgcacaattcattattataaaaataattatgcactaatactcaattctgcatttattttacaagcatagtcaagatccactaaggctgtcc

SEQ ID NO: 536 - DNA, 557 bp, Homo sapiens
gaaacatttatccagaatactctgttgttgaagaaagcactataaagctcagtcaggatatttgagccgttggctgtaacagttacaagctgtgaaaacgtgagcaaagcgcttgcataacagacagattacgggatcaggttaaaagaaatactgaattaaatttatttcaacaaacatattcagctaaaaatcaaagagctcctcaaatatcactgtgaacacactgatcaacagattgaaacttcagctttgtactggtcttgaaaatgtgtcccgctcaccagcaaaatgtgggatatacagtttttgccaaatctgaaatttacttgtcacttyagagttgtctttgaacggaaagattgagattttcgcaagatatgtggagaacagatgatttaatatgcttctttaattttgtgctgtttgtctttctcttccttttttctttggtacatttggacagtgatatattctgactttatttcaaaatactttggctaagattggaagaaaaagagaagggtgcaaaatgaataatgatgagacatttcagt

SEQ ID NO: 537 - DNA, 430 bp, Homo sapiens
atagtgtaaaatgttatcagatacaaaccaacaaggatggaatagaggattcctggcctatctgatggcggttgaatattccggtcctgatcaatggcttggattggtggcgtaacaataatggggttcagttcttcctgaaaaaaaaattaagagagtttcattcagccgcattactaatacttcatgaaggattgagacagatgytttagtttggaaatctatataacgtctgagacagtaaactcaagtttcaacatttatgatctaataccttttactagttaattttgaatgttaagggggagcaaagtattctcaaagtcaaaatggtaagacatagtgattatgcacatagacatttatatcagtaactactcctaaaaggaaatcaaggaagactgataatgcacttttaatcttctctaga

SEQ ID NO: 538 - DNA, 40 bp, Artificial sequence
  Primer
ttttttttttttttttttttttaacactgaacgcagaaaa

SEQ ID NO: 539 - DNA, 20 bp, Artificial sequence
  Primer
ttttctccactgttgtgctg

SEQ ID NO: 540 - DNA, 35 bp, Artificial sequence
  Primer
ttttttttttttttttttcttacttaggaaggcat

SEQ ID NO: 541 - DNA, 45 bp, Artificial sequence
  Primer
ttttttttttttttttttttttttttttcattggttcaaatcgct

SEQ ID NO: 542 - DNA, 30 bp, Artificial sequence
  Primer
tttttttttttatgacagatttgtcttgta

SEQ ID NO: 543 - DNA, 25 bp, Artificial sequence
  Primer
ttttttaagaaatgtgcaaatgctt

SEQ ID NO: 544 - DNA, 50 bp, Artificial sequence
  Primer
ttttttttttttttttttttttttttttttttttccacagaggggtagtt

SEQ ID NO: 545 - DNA, 58 bp, Artificial sequence
  Primer
tttttttttttttttttttttttttttttttttttttttttcacctttgcaggtccta

SEQ ID NO: 546 - DNA, 30 bp, Artificial sequence
  Primer
tttttttttttcagtgatcagtaacacata

SEQ ID NO: 547 - DNA, 35 bp, Artificial sequence
  Primer
tttttttttttttttttatcaccttcagattcttc

SEQ ID NO: 548 - DNA, 25 bp, Artificial sequence
  Primer
ttttttttgtaatgtgagaatgtaa

SEQ ID NO: 549 - DNA, 70 bp, Artificial sequence
  Primer
tttttttttttttttttttttttttttttttttttttttttttttttttttttttccctgcatggcggag

SEQ ID NO: 550 - DNA, 62 bp, Artificial sequence
  Primer
ttttttttttttttttttttttttttttttttttttttttttttttgactggaggtgatgct

SEQ ID NO: 551 - DNA, 40 bp, Artificial sequence
  Primer
ttttttttttttttttttttttcgtgttttctttgcctat

SEQ ID NO: 552 - DNA, 50 bp, Artificial sequence
  Primer
ttttttttttttttttttttttttttttttttccaagctggagactattt

SEQ ID NO: 553 - DNA, 54 bp, Artificial sequence
  Primer
ttttttttttttttttttttttttttttttttttttcaaccttcttgtgaatgg

SEQ ID NO: 554 - DNA, 45 bp, Artificial sequence
  Primer
tttttttttttttttttttttttttttgaattctagcaaagcaca

SEQ ID NO: 555 - DNA, 54 bp, Artificial sequence
  Primer
tttttttttttttttttttttttttttttttttttttatatcctaactccctcc

SEQ ID NO: 556 - DNA, 45 bp, Artificial sequence
  Primer
ttttttttttttttttttttttttttagctatcatgcatctgtat

SEQ ID NO: 557 - DNA, 20 bp, Artificial sequence
  Primer
tttttcttccctctggggtg

SEQ ID NO: 558 - DNA, 50 bp, Artificial sequence
  Primer
ttttttttttttttttttttttttttttttttaccatgccttgcttttgt

SEQ ID NO: 559 - DNA, 30 bp, Artificial sequence
  Primer
ttttttttttattgtctttttcttggtgtt

SEQ ID NO: 560 - DNA, 25 bp, Artificial sequence
  Primer
ttttaattagatgattgatgtgttt

SEQ ID NO: 561 - DNA, 40 bp, Artificial sequence
  Primer
tttttttttttttttttttttactacctaaaatacacttg

SEQ ID NO: 562 - DNA, 58 bp, Artificial sequence
  Primer
ttttttttttttttttttttttttttttttttttttttttttcatagccacccccaaa

SEQ ID NO: 563 - DNA, 45 bp, Artificial sequence
  Primer
tttttttttttttttttttttttttttgctatcatgcatctgtat

SEQ ID NO: 564 - DNA, 20 bp, Artificial sequence
  Primer
ttttttccccttgcccttgc

SEQ ID NO: 565 - DNA, 54 bp, Artificial sequence
  Primer
ttttttttttttttttttttttttttttttttttttcccacaagaatggttcat

SEQ ID NO: 566 - DNA, 25 bp, Artificial sequence
  Primer
tttaaataaaattgttggcattaaa

SEQ ID NO: 567 - DNA, 30 bp, Artificial sequence
  Primer
ttttttttatacaaatctattattttctca

SEQ ID NO: 568 - DNA, 35 bp, Artificial sequence
  Primer
tttttttttttttgaaatataaaaataacaaacca

SEQ ID NO: 569 - DNA, 50 bp, Artificial sequence
  Primer
tttttttttttttttttttttttttttttttttttttccaggtgaatgct

SEQ ID NO: 570 - DNA, 40 bp, Artificial sequence
  Primer
tttttttttttttttttttttgtcaatacatactacgaag

SEQ ID NO: 571 - DNA, 54 bp, Artificial sequence
  Primer
ttttttttttttttttttttttttttttttttttttctgtctcctttttcttag

SEQ ID NO: 572 - DNA, 62 bp, Artificial sequence
  Primer
ttttttttttttttttttttttttttttttttttttttttttttttctgtatgcaggttacc

SEQ ID NO: 573 - DNA, 66 bp, Artificial sequence
  Primer′
tttttttttttttttttttttttttttttttttttttttttttttttttggtgctcacccatttct

SEQ ID NO: 574 - DNA, 70 bp, Artificial sequence
  Primer
ttttttttttttttttttttttttttttttttttttttttttttttttttttttgggattacaggggatg

SEQ ID NO: 575 - DNA, 25 bp, Artificial sequence
  Primer
tttttctatagatttgcccatatta

SEQ ID NO: 576 - DNA, 30 bp, Artificial sequence
  Primer
ttttttttttttgtttcaagaaaggagtaa

SEQ ID NO: 577 - DNA, 35 bp, Artificial sequence
  Primer
ttttttttttttttttaaagtttgttgagactatc

SEQ ID NO: 578 - DNA, 40 bp, Artificial sequence
  Primer
tttttttttttttttttttttcagtaactcacttttacag

SEQ ID NO: 579 - DNA, 58 bp, Artificial sequence
  Primer
ttttttttttttttttttttttttttttttttttttttttccaatagtgaaccaaaca

SEQ ID NO: 580 - DNA, 74 bp, Artificial sequence
  Primer
ttttttttttttttttttttttttttttttttttttttttttttttttttttttttttccacggtcttggatgg

SEQ ID NO: 581 - DNA, 45 bp, Artificial sequence
  Primer
ttttttttttttttttttttttttttgcaagcttttagaaagtct

SEQ ID NO: 582 - DNA, 50 bp, Artificial sequence
  Primer
tttttttttttttttttttttttttttttttcttttttccctgaagaaac

SEQ ID NO: 583 - DNA, 78 bp, Artificial sequence
  Primer
tttttttttttttttttttttttttttttttttttttttttttttttttttttttttttttttcacctctgccgtggg

SEQ ID NO: 584 - DNA, 40 bp, Artificial sequence
  Primer
ttttttttttttttttttttttgaggaaacagaggataaa

SEQ ID NO: 585 - DNA, 30 bp, Artificial sequence
  Primer
tttttttttttgtgggcttttatttataga

SEQ ID NO: 586 - DNA, 66 bp, Artificial sequence
  Primer
ttttttttttttttttttttttttttttttttttttttttttttttttttagttggctgcgtgtac

SEQ ID NO: 587 - DNA, 20 bp, Artificial sequence
  Primer
tttcacaaactgcccacata

SEQ ID NO: 588 - DNA, 70 bp, Artificial sequence
  Primer
ttttttttttttttttttttttttttttttttttttttttttttttttttttttcttgcagcagttctcc

SEQ ID NO: 589 - DNA, 45 bp, Artificial sequence
  Primer
tttttttttttttttttttttttttttcatttcgtattctcctca

SEQ ID NO: 590 - DNA, 62 bp, Artificial sequence
  Primer
tttttttttttttttttttttttttttttttttttttttttttttgaaaagagccttgggtt

SEQ ID NO: 591 - DNA, 54 bp, Artificial sequence
  Primer
ttttttttttttttttttttttttttttttttttttattagtccttggctctta

SEQ ID NO: 592 - DNA, 50 bp, Artificial sequence
  Primer
ttttttttttttttttttttttttttttttttagagaagatacacagtag

SEQ ID NO: 593 - DNA, 58 bp, Artificial sequence
  Primer
tttttttttttttttttttttttttttttttttttttttttcaaactcccaatggctt

SEQ ID NO: 594 - DNA, 25 bp, Artificial sequence
  Primer
ttttctttcttatattactcttttc

SEQ ID NO: 595 - DNA, 40 bp, Artificial sequence
  Primer
tttttttttttttttttttttttgtgggggacacaaatga

SEQ ID NO: 596 - DNA, 70 bp, Artificial sequence
  Primer
ttttttttttttttttttttttttttttttttttttttttttttttttttttttttctcccagtccctcc

SEQ ID NO: 597 - DNA, 25 bp, Artificial sequence
  Primer
tttttttcaataaggtgagtggatg

SEQ ID NO: 598 - DNA, 54 bp, Artificial sequence
  Primer
ttttttttttttttttttttttttttttttttttttttctgcatctcacactgc

SEQ ID NO: 599 - DNA, 58 bp, Artificial sequence
  Primer
tttttttttttttttttttttttttttttttttttttttttttcaaagggtgggtcag

SEQ ID NO: 600 - DNA, 66 bp, Artificial sequence
  Primer
ttttttttttttttttttttttttttttttttttttttttttttttttttccttggctaccgaagc

SEQ ID NO: 601 - DNA, 30 bp, Artificial sequence
  Primer
tttttttttttttgaaacaaggggataacc

SEQ ID NO: 602 - DNA, 20 bp, Artificial sequence
  Primer
tttttcctccctctcctgcc

SEQ ID NO: 603 - DNA, 50 bp, Artificial sequence
  Primer
tttttttttttttttttttttttttttttttttcagacagtatgtctccc

SEQ ID NO: 604 - DNA, 35 bp, Artificial sequence
  Primer
tttttttttttttttttttaaggatggctctacct

SEQ ID NO: 605 - DNA, 30 bp, Artificial sequence
  Primer
ttttttttttggaatagtgtttggaattaa

SEQ ID NO: 606 - DNA, 25 bp, Artificial sequence
  Primer
ttttctttcttatattactcttttc

SEQ ID NO: 607 - DNA, 50 bp, Artificial sequence
  Primer
tttttttttttttttttttttttttttttttttgactgctctgactcatt

SEQ ID NO: 608 - DNA, 45 bp, Artificial sequence
  Primer
tttttttttttttttttttttttttttggactttatcccttcatc

SEQ ID NO: 609 - DNA, 58 bp, Artificial sequence
  Primer
tttttttttttttttttttttttttttttttttttttttttgaagacctgggttcaag

SEQ ID NO: 610 - DNA, 66 bp, Artificial sequence
  Primer
tttttttttttttttttttttttttttttttttttttttttttttttttttcctctggccaggttt

SEQ ID NO: 611 - DNA, 70 bp, Artificial sequence
  Primer
ttttttttttttttttttttttttttttttttttttttttttttttttttttttgaagcagtggcatgtc

SEQ ID NO: 612 - DNA, 54 bp, Artificial sequence
  Primer
tttttttttttttttttttttttttttttttttttttagtgggctaagaacaga

SEQ ID NO: 613 - DNA, 35 bp, Artificial sequence
  Pirmer
ttttttttttttttttcttggttttaaacatttgc

SEQ ID NO: 614 - DNA, 40 bp, Artificial sequence
  Primer
ttttttttttttttttttttttacagaccagataaagcaa

SEQ ID NO: 615 - DNA, 62 bp, Artificial sequence
  Primer
tttttttttttttttttttttttttttttttttttttttttttttgtcactcatccacccta

SEQ ID NO: 616 - DNA, 30 bp, Artificial sequence
  Primer
ttttttttttttgttttatctcttcttgcc

SEQ ID NO: 617 - DNA, 20 bp, Artificial sequence
  Primer
tttttctgggcattccctgg

SEQ ID NO: 618 - DNA, 54 bp, Artificial sequence
  Primer
tttttttttttttttttttttttttttttttttttttcagacacttggaggcat

SEQ ID NO: 619 - DNA, 50 bp, Artificial sequence
  Primer
ttttttttttttttttttttttttttttttttttctggaagagaaagcac

SEQ ID NO: 620 - DNA, 45 bp, Artificial sequence
  Primer
tttttttttttttttttttttttttttgagcgtcctatgaatcat

SEQ ID NO: 621 - DNA, 58 bp, Artificial sequence
  Primer
tttttttttttttttttttttttttttttttttttttttttttccttcctctagggct

SEQ ID NO: 622 - DNA, 70 bp, Artificial sequence
  Primer
ttttttttttttttttttttttttttttttttttttttttttttttttttttttcagaggcatgactggc

SEQ ID NO: 623 - DNA, 62 bp, Artificial sequence
  Primer
ttttttttttttttttttttttttttttttttttttttttttttttctactgaggctcacct

SEQ ID NO: 624 - DNA, 35 bp, Artificial sequence
  Primer
tttttttttttttttttctgtattaaaatgcaccc

SEQ ID NO: 625 - DNA, 25 bp, Artificial sequence
  Primer
tttaagttttaaaaattagtttgac

SEQ ID NO: 626 - DNA, 40 bp, Artificial sequence
  Primer
tttttttttttttttttttttttgtttggtcatggactat

SEQ ID NO: 627 - DNA, 66 bp, Artificial sequence
  Primer
ttttttttttttttttttttttttttttttttttttttttttttttttttgtgttgggtggtcaag

SEQ ID NO: 628 - DNA, 20 bp, Artificial sequence
  Primer
ttttgtggcaagtacagcag

SEQ ID NO: 629 - DNA, 62 bp, Artificial sequence
  Primer
tttttttttttttttttttttttttttttttttttttttttttttttgaggggtaagaggct

SEQ ID NO: 630 - DNA, 58 bp, Artificial sequence
  Primer
tttttttttttttttttttttttttttttttttttttttttggttttgtcaggatgcc

SEQ ID NO: 631 - DNA, 25 bp, Artificial sequence
  Primer
tttttttggatttcttgatttttaa

SEQ ID NO: 632 - DNA, 35 bp, Artificial sequence
  Primer
tttttttttttttttttgaatcttgcttttgcctt

SEQ ID NO: 633 - DNA, 70 bp, Artificial sequence
  Primer
ttttttttttttttttttttttttttttttttttttttttttttttttttttttcctctctcccaacaca

SEQ ID NO: 634 - DNA, 40 bp, Artificial sequence
  Primer
ttttttttttttttttttttttctcttctcattgccttac

SEQ ID NO: 635 - DNA, 54 bp, Artificial sequence
  Primer
tttttttttttttttttttttttttttttttttttttcagctgagatgaggttt

SEQ ID NO: 636 - DNA, 70 bp, Artificial sequence
  Primer
tttttttttttttttttttttttttttttttttttttttttttttttttttttagggattttgccttgtg

SEQ ID NO: 637 - DNA, 66 bp, Artificial sequence
  Primer
ttttttttttttttttttttttttttttttttttttttttttttttttccctctgtctttaactca

SEQ ID NO: 638 - DNA, 25 bp, Artificial sequence
  Primer
tttttttattccctttctaaaacta

SEQ ID NO: 639 - DNA, 30 bp, Artificial sequence
  Primer
ttttttttttctctttctaaatccatattt

SEQ ID NO: 640 - DNA, 54 bp, Artificial sequence
  Primer
ttttttttttttttttttttttttttttttttttttttacccagtagcaaaact

SEQ ID NO: 641 - DNA, 35 bp, Artificial sequence
  Primer
tttttttttttttttgaattccagttacattattt

SEQ ID NO: 642 - DNA, 58 bp, Artificial sequence
  Primer
ttttttttttttttttttttttttttttttttttttttttagttccttgctcacttat

SEQ ID NO: 643 - DNA, 62 bp, Artificial sequence
  Primer
tttttttttttttttttttttttttttttttttttttttttttttgcacacaaatacatacc

SEQ ID NO: 644 - DNA, 40 bp, Artificial sequence
  Primer
ttttttttttttttttttttttttttaaaatgtacccaca

SEQ ID NO: 645 - DNA, 45 bp, Artificial sequence
  Primer
tttttttttttttttttttttttttttaatcctatccagaaagat

SEQ ID NO: 646 - DNA, 20 bp, Artificial sequence
  Primer
ttttagtctctgaggtgttc

SEQ ID NO: 647 - DNA, 74 bp, Artificial sequence
  Primer
tttttttttttttttttttttttttttttttttttttttttttttttttttttttgaaattgctgcttttgtag

SEQ ID NO: 648 - DNA, 62 bp, Artificial sequence
  Primer
tttttttttttttttttttttttttttttttttttttttttttttccttccttttgtgttgg

SEQ ID NO: 649 - DNA, 35 bp, Artificial sequence
  Primer
tttttttttttttttttggaattaggagagttttg

SEQ ID NO: 650 - DNA, 25 bp, Artificial sequence
  Pirmer
tttatattaactttaatgattacaa

SEQ ID NO: 651 - DNA, 40 bp, Artificial sequence
  Primer
tttttttttttttttttttttttaaagatgcatctgctac

SEQ ID NO: 652 - DNA, 20 bp, Artificial sequence
  Primer
tttgccggctgttatagact

SEQ ID NO: 653 - DNA, 30 bp, Artificial sequence
  Primer
tttttttttttttaactatcatcacagtaa

SEQ ID NO: 654 - DNA, 45 bp, Artificial sequence
  Primer
tttttttttttttttttttttttttttagggttacaagaatactc

SEQ ID NO: 655 - DNA, 50 bp, Artificial sequence
  Primer
tttttttttttttttttttttttttttttttttatgtatgtcagcttcca

SEQ ID NO: 656 - DNA, 58 bp, Artificial sequence
  Primer
ttttttttttttttttttttttttttttttttttttttttacagaggaaggaggattt

SEQ ID NO: 657 - DNA, 54 bp, Artificial sequence
  Primer
ttttttttttttttttttttttttttttttttttttggcacaaaacaaacatac

SEQ ID NO: 658 - DNA, 50 bp, Artificial sequence
  Primer
cccccccccccccccccccccccccccccccccagaccgctggattaaaa

SEQ ID NO: 659 - DNA, 40 bp, Artificial sequence
  Primer
tttttttttttttttttttttttttcctttaggagtaagt

SEQ ID NO: 660 - DNA, 54 bp, Artificial sequence
  Primer
tttttttttttttttttttttttttttttttttttttcactttgtctctgtact

SEQ ID NO: 661 - DNA, 25 bp, Artificial sequence
  Primer
tttttatacaaatagattagtacaa

SEQ ID NO: 662 - DNA, 70 bp, Artificial sequence
  Primer
ttttttttttttttttttttttttttttttttttttttttttttttttttttttgtggggagacatctga

SEQ ID NO: 663 - DNA, 20 bp, Artificial sequence
  Primer
ttttcctgctcagacaccac

SEQ ID NO: 664 - DNA, 30 bp, Artificial sequence
  Primer
tttttttttgctgtattgtaaataacaaaa

SEQ ID NO: 665 - DNA, 62 bp, Artificial sequence
  Primer
tttttttttttttttttttttttttttttttttttttttttttttcaaggcacagaaaactg

SEQ ID NO: 666 - DNA, 58 bp, Artificial sequence
  Primer
ttttttttttttttttttttttttttttttttttttttttgatctcctggcagaattt

SEQ ID NO: 667 - DNA, 35 bp, Artificial sequence
  Primer
tttttttttttttttctcaacaaaagatcaataac

SEQ ID NO: 668 - DNA, 66 bp, Artificial sequence
  Primer
tttttttttttttttttttttttttttttttttttttttttttttttttggaaggccttacttaac

SEQ ID NO: 669 - DNA, 45 bp, Artificial sequence
  Primer
ttttttttttttttttttttttttttcagttcttaaagttgaagg

SEQ ID NO: 670 - DNA, 25 bp, Artificial sequence
  Primer
tttttatttttgtatggattcactt

SEQ ID NO: 671 - DNA, 50 bp, Artificial sequence
  Primer
tttttttttttttttttttttttttttttttttacactaagaaagaggtg

SEQ ID NO: 672 - DNA, 54 bp, Artificial sequence
  Primer
ttttttttttttttttttttttttttttttttttttatgaatttcttggacagc

SEQ ID NO: 673 - DNA, 70 bp, Artificial sequence
  Primer
tttttttttttttttttttttttttttttttttttttttttttttttttttttgctaataccacagaacc

SEQ ID NO: 674 - DNA, 30 bp, Artificial sequence
  Primer
ttttttttttgctatttaataagacagatc

SEQ ID NO: 675 - DNA, 40 bp, Artificial sequence
  Primer
tttttttttttttttttttttaaaattcaagagaacatgc

SEQ ID NO: 676 - DNA, 62 bp, Artificial sequence
  Primer
ttttttttttttttttttttttttttttttttttttttttttttcatgtccctactaagagt

SEQ ID NO: 677 - DNA, 66 bp, Artificial sequence
  Primer
ttttttttttttttttttttttttttttttttttttttttttttttttcgtctccagtacaaaatg

SEQ ID NO: 678 - DNA, 58 bp, Artificial sequence
  Primer
ttttttttttttttttttttttttttttttttttttttttagacagcttcattcatac

SEQ ID NO: 679 - DNA, 20 bp, Artificial sequence
  Primer
tttttacacggatcactgcc

SEQ ID NO: 680 - DNA, 45 bp, Artificial sequence
  Primer
tttttttttttttttttttttttttttgttattctttttcaagcc

SEQ ID NO: 681 - DNA, 35 bp, Artificial sequence
  Primer
ttttttttttttttttttgtcttcttcttttcccc

SEQ ID NO: 682 - DNA, 25 bp, Artificial sequence
  Primer
tttttctttgtaatctatcgttttt

SEQ ID NO: 683 - DNA, 40 bp, Artificial sequence
  Primer
ttttttttttttttttttttttaaagggtatcctttcaag

SEQ ID NO: 684 - DNA, 35 bp, Artificial sequence
  Primer
ttttttttttttttttgaaagactgagcaatctat

SEQ ID NO: 685 - DNA, 30 bp, Artificial sequence
  Primer
ttttttttttgaaaggaatcctttaaatca

SEQ ID NO: 686 - DNA, 45 bp, Artificial sequence
  Primer
tttttttttttttttttttttttttttgctaatagaatcctggaa

SEQ ID NO: 687 - DNA, 20 bp, Artificial sequence
  Primer
tttcaaggcacaaagctaag

SEQ ID NO: 688 - DNA, 35 bp, Artificial sequence
  Primer
ttttttttttttttttttatgtgttttctgtcccc

SEQ ID NO: 689 - DNA, 25 bp, Artificial sequence
  Primer
tttttttcacatcttcaggataggt

SEQ ID NO: 690 - DNA, 30 bp, Artificial sequence
  Primer
ttttttttttttcaccaataaaaggtgcca

SEQ ID NO: 691 - DNA, 45 bp, Artificial sequence
  Primer
ttttttttttttttttttttttttttttaccacattgctttggct

SEQ ID NO: 692 - DNA, 40 bp, Artificial sequence
  Primer
ttttttttttttttttttttttttgacatccctgctttct

SEQ ID NO: 693 - DNA, 35 bp, Artificial sequence
  Primer
tttttttttttttttttgtccatctcaggaatagt

SEQ ID NO: 694 - DNA, 45 bp, Artificial sequence
  Primer
ttttttttttttttttttttttttttttgcccatctgaggcaatt

SEQ ID NO: 695 - DNA, 50 bp, Artificial sequence
  Primer
tttttttttttttttttttttttttttttttttttactccagccatgccc

SEQ ID NO: 696 - DNA, 30 bp, Artificial sequence
  Primer
tttttttttttttaagcagctaatggcaat

SEQ ID NO: 697 - DNA, 54 bp, Artificial sequence
  Primer
tttttttttttttttttttttttttttttttttttttttggttcgtaccccacc

SEQ ID NO: 698 - DNA, 20 bp, Artificial sequence
  Primer
tttttagttccgtccagggc

SEQ ID NO: 699 - DNA, 25 bp, Artificial sequence
  Primer
ttttttatgtaccattttaatgcac

SEQ ID NO: 700 - DNA, 30 bp, Artificial sequence
  Primer
ttttttttttatttaatgttctatcacttc

SEQ ID NO: 701 - DNA, 45 bp, Artificial sequence
  Primer
ttttttttttttttttttttttttttaagacaaagagaaaaacca

SEQ ID NO: 702 - DNA, 50 bp, Artificial sequence
  Primer
tttttttttttttttttttttttttttttttatgtctcttcatcttttag

SEQ ID NO: 703 - DNA, 54 bp, Artificial sequence
  Primer
ttttttttttttttttttttttttttttttttttttgttaagctcatgctttca

SEQ ID NO: 704 - DNA, 35 bp, Artificial sequence
  Primer
tttttttttttttttaatgggtctgaaaatataaa

SEQ ID NO: 705 - DNA, 58 bp, Artificial sequence
  Primer
tttttttttttttttttttttttttttttttttttttttttcgatgagtaaatatggg

SEQ ID NO: 706 - DNA, 62 bp, Artificial sequence
  Primer
ttttttttttttttttttttttttttttttttttttttttttttgaactgcattttgagaga

SEQ ID NO: 707 - DNA, 25 bp, Artificial sequence
  Primer
ttttttcagctattaagtttaaata

SEQ ID NO: 708 - DNA, 66 bp, Artificial sequence
  Primer
ttttttttttttttttttttttttttttttttttttttttttttttttaagtgatctctctaacca

SEQ ID NO: 709 - DNA, 70 bp, Artificial sequence
  Primer
tttttttttttttttttttttttttttttttttttttttttttttttttttttgctgtcacttgaaatag

SEQ ID NO: 710 - DNA, 40 bp, Artificial sequence
  Primer
ttttttttttttttttttttctgaaatttacttgtcactt

SEQ ID NO: 711 - DNA, 20 bp, Artificial sequence
  Primer
ttgaaggattgagacagatg

SEQ ID NO: 712 - DNA, 22 bp, Artificial sequence
  Primer
tatagtgatatgctgacaggct

SEQ ID NO: 713 - DNA, 25 bp, Artificial sequence
  Primer
tttatatacaaaaccagtgcctctt

SEQ ID NO: 714 - DNA, 21 bp, Artificial sequence
  Primer
tgttctgataagagcaggagc

SEQ ID NO: 715 - DNA, 22 bp, Artificial sequence
  Primer
tctcaatatgcaagagtgctga

SEQ ID NO: 716 - DNA, 21 bp, Artificial sequence
  Primer
tctttaaagccatgattctcg

SEQ ID NO: 717 - DNA, 23 bp, Artificial sequence
  Primer
atcagagggagagaataaatgac

SEQ ID NO: 718 - DNA, 18 bp, Artificial sequence
  Primre
gctgttagaaagcccccg

SEQ ID NO: 719 - DNA, 19 bp, Artificial sequence
  Primer
tccctatcttcaccagcct

SEQ ID NO: 720 - DNA, 22 bp, Artificial sequence
  Primer
ttgaggttggttttcttaaagg

SEQ ID NO: 721 - DNA, 21 bp, Artificial sequence
  Primer
tgtttaattcaacatgcagtt

SEQ ID NO: 722 - DNA, 23 bp, Artificial sequence
  Primer
aaagtggatgtgtggataaattc

SEQ ID NO: 723 - DNA, 24 bp, Artificial sequence
  Primer
ggtagtgtagtctttattcttggt

SEQ ID NO: 724 - DNA, 19 bp, Artificial sequence
  Primer
attggtgccacaccactta

SEQ ID NO: 725 - DNA, 19 bp, Artificial sequence
  Primer
tgatgtggttgtggaggtc

SEQ ID NO: 726 - DNA, 20 bp, Artificial sequence
  Primer
tatgtgataaggcactgggg

SEQ ID NO: 727 - DNA, 25 bp, Artificial sequence
  Primer
tattttatacatttccttctgctgc

SEQ ID NO: 728 - DNA, 19 bp, Artificial sequence
  Primer
ttgatgtgtagtcccctgg

SEQ ID NO: 729 - DNA, 23 bp, Artificial sequence
  Primer
ttgtagatacttaaggggctgtg

SEQ ID NO: 730 - DNA, 20 bp, Artificial sequence
  Primer
atgaagtatcttggggcagg

SEQ ID NO: 731 - DNA, 20 bp, Artificial sequence
  Primer
cagagcacgaaaacctctca

SEQ ID NO: 732 - DNA, 18 bp, Artificial sequence
  Primer
tttggccggtcccctcca

SEQ ID NO: 733 - DNA, 18 bp, Artificial sequence
  Primer
aggggctgaaggatgggg

SEQ ID NO: 734 - DNA, 23 bp, Artificial sequence
  Primer
atctagtgggaaattctttcctg

SEQ ID NO: 735 - DNA, 21 bp, Artificial sequence
  Primer
aattcagagaggcaaagcatt

SEQ ID NO: 736 - DNA, 23 bp, Artificial sequence
  Primer
ttcctgaggagtcagttttcttt

SEQ ID NO: 737 - DNA, 19 bp, Artificial sequence
  Primer
aaagacgcaccaatggagt

SEQ ID NO: 738 - DNA, 22 bp, Artificial sequence
  Primer
tccagatctaggaggtaagcag

SEQ ID NO: 739 - DNA, 20 bp, Artificial sequence
  Primer
tgtgtgcgttagaggtcaaa

SEQ ID NO: 740 - DNA, 25 bp, Artificial sequence
  Priemer
aaaccatataatgattgaactgctg

SEQ ID NO: 741 - DNA, 22 bp, Artificial sequence
  Primer
aacaggcaccaaatcattttaa

SEQ ID NO: 742 - DNA, 22 bp, Artificial sequence
  Primer
gtttgtgctaaggcatttttca

SEQ ID NO: 743 - DNA, 19 bp, Artificial sequence
  Primer
taaaacaacagtgcgtgcg

SEQ ID NO: 744 - DNA, 24 bp, Artificial sequence
  Primer
ctttttagatgaaaagcatcacag

SEQ ID NO: 745 - DNA, 25 bp, Artificial sequence
  Primer
tccctcaaaatcatactatagctga

SEQ ID NO: 746 - DNA, 18 bp, Artificial sequence
  Primer
catctggaagggcatgag

SEQ ID NO: 747 - DNA, 19 bp, Artificial sequence
  Primer
acagctaaacccaatcgca

SEQ ID NO: 748 - DNA, 22 bp, Artificial sequence
  Primer
tgagtcttcaaagacaaaagcc

SEQ ID NO: 749 - DNA, 28 bp, Artificial sequence
  Primer
taaaaatttatcctttccactttacttg

SEQ ID NO: 750 - DNA, 19 bp, Artificial sequence
  Prirmer
agaagtcgcagagggaagg

SEQ ID NO: 751 - DNA, 22 bp, Artificial sequence
  Primer
tgcagtcgtgattattctctct

SEQ ID NO: 752 - DNA, 24 bp, Artificial sequence
  Primer
taaattctgcagtaattagtggcc

SEQ ID NO: 753 - DNA, 26 bp, Artificial sequence
  Primer
tcttgttactcatgtaactgttttgc

SEQ ID NO: 754 - DNA, 23 bp, Artificial sequence
  Primer
aatctgttacctgaaacaagcag

SEQ ID NO: 755 - DNA, 26 bp, Artificial sequence
  Primer
aattgaatagccttcaatataaagca

SEQ ID NO: 756 - DNA, 26 bp, Artificial sequence
  Primer
ataagaattttttcagacatacctgc

SEQ ID NO: 757 - DNA, 23 bp, Artificial sequence
  Primer
aatgccagtctagaacatagcaa

SEQ ID NO: 758 - DNA, 26 bp, Artificial sequence
  Primer
cctcctattctggttggtattataaa

SEQ ID NO: 759 - DNA, 30 bp, Artificial sequence
  Primer
ttaagattcaatttacatatagcttctgtc

SEQ ID NO: 760 - DNA, 21 bp, Artificial sequence
  Primer
aaacctggagatgaacctacc

SEQ ID NO: 761 - DNA, 19 bp, Artificial sequence
  Primer
tttggccattcatgtcaag

SEQ ID NO: 762 - DNA, 22 bp, Artificial sequence
  Primer
tgagtcttcaaagacaaaagcc

SEQ ID NO: 763 - DNA, 28 bp, Artificial sequence
  Primer
taaaaatttatcctttccactttacttg

SEQ ID NO: 764 - DNA, 18 bp, Artificial sequence
  Primer
accctcaacccacagccc

SEQ ID NO: 765 - DNA, 18 bp, Artificial sequence
  Primer
agatgcctgaattcccag

SEQ ID NO: 766 - DNA, 20 bp, Artificial sequence
  Primer
tttttagcatcctcccttcc

SEQ ID NO: 767 - DNA, 28 bp, Artificial sequence
  Primer
tcagaagtgaagagtactctataggaaa

SEQ ID NO: 768 - DNA, 22 bp, Artificial sequence
  Primer
tcatccaatggagtcatttctt

SEQ ID NO: 769 - DNA, 23 bp, Artificial sequence
  Primer
aatatttgagccagaagacttgc

SEQ ID NO: 770 - DNA, 26 bp, Artificial sequence
  Primer
atatcatttgacctggtaattcaact

SEQ ID NO: 771 - DNA, 28 bp, Artificial sequence
  Primer
gttgctaccaatttttcttgtataatta

SEQ ID NO: 772 - DNA, 18 bp, Artificial sequence
  Primer
acagaaagcgttgctgct

SEQ ID NO: 773 - DNA, 19 bp, Artificial sequence
  Primer
acaacaatgcaggcaaagc

SEQ ID NO: 774 - DNA, 25 bp, Artificial sequence
  Primer
acagtagctgagttttttcattttc

SEQ ID NO: 775 - DNA, 29 bp, Artificial sequence
  Primer
taaacaacatgtatatttattcagtgtcc

SEQ ID NO: 776 - DNA, 29 bp, Artificial sequence
  Primer
taattcagtcatacatcctaatacatttg

SEQ ID NO: 777 - DNA, 26 bp, Artificial sequence
  Primer
cactgaccttgaataggaaatacata

SEQ ID NO: 778 - DNA, 20 bp, Artificial sequence
  Primer
ttggtcaggagctaaaggaa

SEQ ID NO: 779 - DNA, 18 bp, Artificial sequence
  Pimer
tcacaccagctcccagac

SEQ ID NO: 780 - DNA, 18 bp, Artificial sequence
  Primer
tggttttatgggccgaat

SEQ ID NO: 781 - DNA, 18 bp, Artificial sequence
  Primer
attccatcggcaagagct

SEQ ID NO: 782 - DNA, 21 bp, Artificial sequence
  Primer
ttaaagaaggtgtgcttgtgg

SEQ ID NO: 783 - DNA, 26 bp, Artificial sequence
  Primer
ttatagagttaatacccccaaagttg

SEQ ID NO: 784 - DNA, 18 bp, Artificial sequence
  Primer
gcgcctactccacaccag

SEQ ID NO: 785 - DNA, 21 bp, Artificial sequence
  Primer
ttaataaccaaggtcctgcct

SEQ ID NO: 786 - DNA, 24 bp, Artificial sequence
  Primer
tgccaactctacatactctcattg

SEQ ID NO: 787 - DNA, 21 bp, Artificial sequence
  Primer
ttacctggctttgctatgagg

SEQ ID NO: 788 - DNA, 21 bp, Artificial sequence
  Primer
ttgtaatccttcacggagacc

SEQ ID NO: 789 - DNA, 18 bp, Artificial sequence
  Primer
tcgaggccctgcaatccc

SEQ ID NO: 790 - DNA, 19 bp, Artificial sequence
  Primer
atctcatttccccccaaat

SEQ ID NO: 791 - DNA, 25 bp, Artificial sequence
  Primer
atagaatagctgaactgaaatcgag

SEQ ID NO: 792 - DNA, 21 bp, Artificial sequence
  Primer
tttcttagatgcctggttgtg

SEQ ID NO: 793 - DNA, 24 bp, Artificial sequence
  Primer
tattagatttgattcccaagaacc

SEQ ID NO: 794 - DNA, 25 bp, Artificial sequence
  Primer
ttttatttatggtctgacaaaatgc

SEQ ID NO: 795 - DNA, 18 bp, Artificial sequence
  Primer
tggatctggtcagcccct

SEQ ID NO: 796 - DNA, 18 bp, Artificial sequence
  Primer
agggctccctgcaaaaca

SEQ ID NO: 797 - DNA, 22 bp, Artificial sequence
  Primer
atatttctgggttttgttcgtt

SEQ ID NO: 798 - DNA, 20 bp, Artificial sequence
  Primer
ttccaatacaagcagccttc

SEQ ID NO: 799 - DNA, 21 bp, Artificial sequence
  Primer
aattgataatgccccattgtt

SEQ ID NO: 800 - DNA, 27 bp, Artificial sequence
  Primer
attacaagcagatcacactaggaatat

SEQ ID NO: 801 - DNA, 24 bp, Artificial sequence
  Primer
aaacactaacagggattcaaaaga

SEQ ID NO: 802 - DNA, 18 bp, Artificial sequence
  Primer
aggcagtgcaggcagagc

SEQ ID NO: 803 - DNA, 18 bp, Artificial sequence
  Primer
catgctcctcttcacgcc

SEQ ID NO: 804 - DNA, 31 bp, Artificial sequence
  Primer
agatactaagttttaaactgtggatttctat

SEQ ID NO: 805 - DNA, 22 bp, Artificial sequence
  Primer
tggtgcagttcttacaagtcag

SEQ ID NO: 806 - DNA, 24 bp, Artificial sequence
  Primer
tactgggtcactgaaaatatttga

SEQ ID NO: 807 - DNA, 22 bp, Artificial sequence
  Primer
tatgtggactctggttccagtt

SEQ ID NO: 808 - DNA, 23 bp, Artificial sequence
  Primer
ataaattcaaagaatgtgggagg

SEQ ID NO: 809 - DNA, 21 bp, Artificial sequence
  Primer
ttttccatctaccatttccca

SEQ ID NO: 810 - DNA, 22 bp, Artificial sequence
  Primer
tccagatgctttccttacagat

SEQ ID NO: 811 - DNA, 23 bp, Artificial sequence
  Primer
atattcacataaccacacacgct

SEQ ID NO: 812 - DNA, 21 bp, Artificial sequence
  Primer
cacaaagactctgagaaacgg

SEQ ID NO: 813 - DNA, 19 bp, Artificial sequence
  Primer
aaaggagactgagcgtgga

SEQ ID NO: 814 - DNA, 21 bp, Artificial sequence
  Primer
atgaaggtctttgtcacacca

SEQ ID NO: 815 - DNA, 18 bp, Artificial sequence
  Primer
agttggccttcgaactgc

SEQ ID NO: 816 - DNA, 24 bp, Artificial sequence
  Primer
tgatttagtagcagaggaaggagt

SEQ ID NO: 817 - DNA, 22 bp, Artificial sequence
  Primer
aagagcattaaggaatgatgcc

SEQ ID NO: 818 - DNA, 18 bp, Artificial sequence
  Primer
agacagaagggctgggac

SEQ ID NO: 819 - DNA, 18 bp, Artificial sequence
  Primer
tgtccctgcccagaacct

SEQ ID NO: 820 - DNA, 27 bp, Artificial sequence
  Primer
atctagatgagattttagcactgacat

SEQ ID NO: 821 - DNA, 25 bp, Artificial sequence
  Primer
aactggttttctaagctattttcca

SEQ ID NO: 822 - DNA, 20 bp, Artificial sequence
  Primer
tctccctctcttttaccccc

SEQ ID NO: 823 - DNA, 20 bp, Artificial sequence
  Primer
tgaatcttcctgtcacctcg

SEQ ID NO: 824 - DNA, 21 bp, Artificial sequence
  Primer
agtagagcccaaaaagcagag

SEQ ID NO: 825 - DNA, 23 bp, Artificial sequence
  Primer
agtaatcagcttggtaaacaccc

SEQ ID NO: 826 - DNA, 21 bp, Artificial sequence
  Primer
ctccctttgtagggtaggaga

SEQ ID NO: 827 - DNA, 18 bp, Artificial sequence
  Primer
ggcagagacagcggctag

SEQ ID NO: 828 - DNA, 18 bp, Artificial sequence
  Primer
ccctgagctcactgcctg

SEQ ID NO: 829 - DNA, 25 bp, Artificial sequence
  Primer
aataagtaggcaagaccctaaaaag

SEQ ID NO: 830 - DNA, 23 bp, Artificial sequence
  Primer
ataaaaattytcctgggaagtgg

SEQ ID NO: 831 - DNA, 19 bp, Artificial sequence
  Primer
tgcaggaggaaattgatgc

SEQ ID NO: 832 - DNA, 18 bp, Artificial sequence
  Primer
taaagtccaggggagggg

SEQ ID NO: 833 - DNA, 18 bp, Artificial sequence
  Primer
actcccctactccaggcc

SEQ ID NO: 834 - DNA, 18 bp, Artificial sequence
  Primer
gcaaatgtatgttcccgg

SEQ ID NO: 835 - DNA, 23 bp, Artificial sequence
  Primer
aatgaaacagcagaaaagaacac

SEQ ID NO: 836 - DNA, 19 bp, Artificial sequence
  Primer
tcctccaaaattctgagcc

SEQ ID NO: 837 - DNA, 23 bp, Artificial sequence
  Primer
aagcaaatgcatgttttttatga

SEQ ID NO: 838 - DNA, 18 bp, Artificial sequence
  Primer
tgcaaaacccactggcag

SEQ ID NO: 839 - DNA, 19 bp, Artificial sequence
  Primer
aatttccccactctctggc

SEQ ID NO: 840 - DNA, 19 bp, Artificial sequence
  Primer
tttgggggtaggcatgtac

SEQ ID NO: 841 - DNA, 46 bp, Artificial sequence
  Primer
cgtcttttaggaaataagtcatcaatgcctgtaaagacccttctct

SEQ ID NO: 842 - DNA, 25 bp, Artificial sequence
  Primer
cgtcttttaggaaataagtcatcaa

SEQ ID NO: 843 - DNA, 21 bp, Artificial sequence
  Primer
aacaactgcagaacaagcact

SEQ ID NO: 844 - DNA, 22 bp, Artificial sequence
  Primer
ttctatgacccctacctgttgt

SEQ ID NO: 845 - DNA, 21 bp, Artificial sequence
  Primer
tggggagagctcacctaatat

SEQ ID NO: 846 - DNA, 18 bp, Artificial sequence
  Primer
aaatctgcactggggtgg

SEQ ID NO: 847 - DNA, 18 bp, Artificial sequence
  Primer
aggcccttatgcttgggt

SEQ ID NO: 848 - DNA, 21 bp, Artificial sequence
  Primer
agtagagcccaaaaagcagag

SEQ ID NO: 849 - DNA, 23 bp, Artificial sequence
  Primer
agtaatcagcttggtaaacaccc

SEQ ID NO: 850 - DNA, 21 bp, Artificial sequence
  Primer
tgtggtcacaggtaactgaca

SEQ ID NO: 851 - DNA, 19 bp, Artificial sequence
  Primer
gaaaagacaccaggatgaa

SEQ ID NO: 852 - DNA, 23 bp, Artificial sequence
  Primer
tttctatgttgtctatgggaagg

SEQ ID NO: 853 - DNA, 22 bp, Artificial sequence
  Primer
ctgcatgatgttctgatttctc

SEQ ID NO: 854 - DNA, 19 bp, Artificial sequence
  Primer
agcggttagcctgaaatca

SEQ ID NO: 855 - DNA, 21 bp, Artificial sequence
  Primer
atgacctaagttttgggcaga

SEQ ID NO: 856 - DNA, 21 bp, Artificial sequence
  Primer
tccaacttaccagtgtacccc

SEQ ID NO: 857 - DNA, 18 bp, Artificial sequence
  Primer
taccgccgactcgtctgg

SEQ ID NO: 858 - DNA, 23 bp, Artificial sequence
  Primer
ttcatttctgtgtcttcagacct

SEQ ID NO: 859 - DNA, 18 bp, Artificial sequence
  Primer
aaatccctgcaacttggc

SEQ ID NO: 860 - DNA, 23 bp, Artificial sequence
  Primer
aaacaaatctcctgtttacgctt

SEQ ID NO: 861 - DNA, 20 bp, Artificial sequence
  Primer
aatggggaggaagcagatat

SEQ ID NO: 862 - DNA, 23 bp, Artificial sequence
  Primer
aaagtagacccctttggtacaga

SEQ ID NO: 863 - DNA, 20 bp, Artificial sequence
  Primer
accctctgcaatcacagatg

SEQ ID NO: 864 - DNA, 22 bp, Artificial sequence
  Primer
tctgtcagacttggtttaagcc

SEQ ID NO: 865 - DNA, 22 bp, Artificial sequence
  Primer
aaatgatcaatggctacaggac

SEQ ID NO: 866 - DNA, 24 bp, Artificial sequence
  Primer
atctctactggagattcttgaggc

SEQ ID NO: 867 - DNA, 22 bp, Artificial sequence
  Primer
attctgggctgctatttatgtg

SEQ ID NO: 868 - DNA, 20 bp, Artificial sequence
  Primer
agccttgactgattgctgtc

SEQ ID NO: 869 - DNA, 19 bp, Artificial sequence
  Primer
agcagagagaagctgtcgg

SEQ ID NO: 870 - DNA, 21 bp, Artificial sequence
  Primer
tttgtccttctgtttcagacg

SEQ ID NO: 871 - DNA, 21 bp, Artificial sequence
  Primer
atccaaacagatgccattacc

SEQ ID NO: 872 - DNA, 19 bp, Artificial sequence
  Primer
agggataggtacctggcag

SEQ ID NO: 873 - DNA, 20 bp, Artificial sequence
  Primer
aatctgctgagactgacgct

SEQ ID NO: 874 - DNA, 20 bp, Artificial sequence
  Primer
agtatctttgccctggagct

SEQ ID NO: 875 - DNA, 19 bp, Artificial sequence
  Primer
tttttggaaattgggctca

SEQ ID NO: 876 - DNA, 18 bp, Artificial sequence
  Primer
cctttgcaagagttgcca

SEQ ID NO: 877 - DNA, 18 bp, Artificial sequence
  Primer
tcatggtcccctccctcg

SEQ ID NO: 878 - DNA, 21 bp, Artificial sequence
  Primer
agagctgggttttctttttcc

SEQ ID NO: 879 - DNA, 18 bp, Artificial sequence
  Primer
agaatcccccttgccatt

SEQ ID NO: 880 - DNA, 23 bp, Artificial sequence
  Primer
aaaactttaatgcctcaactctg

SEQ ID NO: 881 - DNA, 18 bp, Artificial sequence
  Primer
aaacaggaagcgtccctg

SEQ ID NO: 882 - DNA, 18 bp, Artificial sequence
  Primer
tgattgggtgtggcaatg

SEQ ID NO: 883 - DNA, 19 bp, Artificial sequence
  Primer
ttgggttgtatagccaggg

SEQ ID NO: 884 - DNA, 19 bp, Artificial sequence
  Primer
aacttgggctttcaagtcg

SEQ ID NO: 885 - DNA, 22 bp, Artificial sequence
  Primer
agtgacaaagcagacactgttg

SEQ ID NO: 886 - DNA, 22 bp, Artificial sequence
  Primer
acaatgaatgtgagctctgtcc

SEQ ID NO: 887 - DNA, 20 bp, Artificial sequence
  Primer
agactcacctgcctcactga

SEQ ID NO: 888 - DNA, 27 bp, Artificial sequence
  Primer
cctacttggttcttctcttattaatca

SEQ ID NO: 889 - DNA, 18 bp, Artificial sequence
  Primer
tctattggccaccgtgat

SEQ ID NO: 890 - DNA, 21 bp, Artificial sequence
  Primer
tgcagtgcactatcatttgtc

SEQ ID NO: 891 - DNA, 23 bp, Artificial sequence
  Primer
ttttttgcagttctctgaaagac

SEQ ID NO: 892 - DNA, 22 bp, Artificial sequence
  Primer
tgttatttccctgtccttgaac

SEQ ID NO: 893 - DNA, 18 bp, Artificial sequence
  Primer
tcccctcttgggtcttgt

SEQ ID NO: 894 - DNA, 23 bp, Artificial sequence
  Primer
tacacacacttaccaccttcctg

SEQ ID NO: 895 - DNA, 25 bp, Artificial sequence
  Primer
attccgatctgagaattatcttatg

SEQ ID NO: 896 - DNA, 20 bp, Artificial sequence
  Primer
attaactgtggtgagggggt

SEQ ID NO: 897 - DNA, 19 bp, Artificial sequence
  Primer
atgaaaagtgccgttctcc

SEQ ID NO: 898 - DNA, 27 bp, Artificial sequence
  Primer
aaactagaacaagaattgcagtatttc

SEQ ID NO: 899 - DNA, 24 bp, Artificial sequence
  Primer
tttgtaacttcttggacgtgataa

SEQ ID NO: 900 - DNA, 23 bp, Artificial sequence
  Primer
gaaatcgaatacgtccatttctt

SEQ ID NO: 901 - DNA, 19 bp, Artificial sequence
  Primer
tgtccccttttccaatgtt

SEQ ID NO: 902 - DNA, 26 bp, Artificial sequence
  Primer
gaaaaattgatattaaatcagcaggt

SEQ ID NO: 903 - DNA, 23 bp, Artificial sequence
  Primer
cactaaaggggacactactgtgt

SEQ ID NO: 904 - DNA, 29 bp, Artificial sequence
  Primer
ataggtgtaagttggcatttatatatttg

SEQ ID NO: 905 - DNA, 25 bp, Artificial sequence
  Primer
tatatgttagagaagcaagcacatg

SEQ ID NO: 906 - DNA, 21 bp, Artificial sequence
  Primer
tttcttaaagtgcagggattg

SEQ ID NO: 907 - DNA, 26 bp, Artificial sequence
  Primer
aagtaaacacagagctcctctaattt

SEQ ID NO: 908 - DNA, 19 bp, Artificial sequence
  Primer
tgccaagacttacccacat

SEQ ID NO: 909 - DNA, 30 bp, Artificial sequence
  Primer
cagatgaatatatatctggaaatatcaagt

SEQ ID NO: 910 - DNA, 19 bp, Artificial sequence
  Primer
aattgcaaccacacactcc

SEQ ID NO: 911 - DNA, 27 bp, Artificial sequence
  Primer
actgacatttttagatgatgagataga

SEQ ID NO: 912 - DNA, 25 bp, Artificial sequence
  Primer
aattagaagaatggattggaatctg

SEQ ID NO: 913 - DNA, 29 bp, Artificial sequence
  Primer
agtaccatttgacagaagtattttaactc

SEQ ID NO: 914 - DNA, 24 bp, Artificial sequence
  Primer
aaattgatccctttaactagggaa

SEQ ID NO: 915 - DNA, 25 bp, Artificial sequence
  Primer
tttgtgttttaactatcacaagcag

SEQ ID NO: 916 - DNA, 25 bp, Artificial sequence
  Primer
ttagggtattgctttaggaagtttt

SEQ ID NO: 917 - DNA, 30 bp, Artificial sequence
  Primer
ctattttatttatctcatgttctcttacca

SEQ ID NO: 918 - DNA, 25 bp, Artificial sequence
  Primer
tttcattctaagaacctcattgttt

SEQ ID NO: 919 - DNA, 24 bp, Artificial sequence
  Primer
ttttaaaaagcacactcccttaat

SEQ ID NO: 920 - DNA, 30 bp, Artificial sequence
  Primer
attgtggttaagtataaactactaatcctg

SEQ ID NO: 921 - DNA, 19 bp, Artificial sequence
  Primer
ttgaaatacatggcccaaa

SEQ ID NO: 922 - DNA, 25 bp, Artificial sequence
  Primer
gaagacactcatatgcaatatatgg

SEQ ID NO: 923 - DNA, 24 bp, Artificial sequence
  Primer
agggaggtgaatatttgctatatc

SEQ ID NO: 924 - DNA, 24 bp, Artificial sequence
  Primer
ataatctttagtcaaccaccctca

SEQ ID NO: 925 - DNA, 24 bp, Artificial sequence
  Primer
ctgcatcttaattcattgtgagat

SEQ ID NO: 926 - DNA, 24 bp, Artificial sequence
  Primer
ttctctgtccctctaatctgtagg

SEQ ID NO: 927 - DNA, 25 bp, Artificial sequence
  Primer
aagagaactttctattcatcccact

SEQ ID NO: 928 - DNA, 26 bp, Artificial sequence
  Primer
tcaaagaaaaataaagaatacaagcc

SEQ ID NO: 929 - DNA, 24 bp, Artificial sequence
  Primer
atgcatttaaaatcttggctttaa

SEQ ID NO: 930 - DNA, 27 bp, Artificial sequence
  Primer
attttctaccttagtttgtgtgattga

SEQ ID NO: 931 - DNA, 23 bp, Artificial sequence
  Primer
ttccaatgaattaaaaagcaaac

SEQ ID NO: 932 - DNA, 25 bp, Artificial sequence
  Primer
ttatcacaaacaaatcatgaaagac

SEQ ID NO: 933 - DNA, 20 bp, Artificial sequence
  Primer
agtgtgtgggtgttgttgaa

SEQ ID NO: 934 - DNA, 20 bp, Artificial sequence
  Primer
tagctcttgtgagctggtgg

SEQ ID NO: 935 - DNA, 22 bp, Artificial sequence
  Primer
acttctctggaagctgcataca

SEQ ID NO: 936 - DNA, 27 bp, Artificial sequence
  Primer
acattagttgtgtttctaagtctaggc

SEQ ID NO: 937 - DNA, 24 bp, Artificial sequence
  Primer
aaaaaaaatcctgataagcactgt

SEQ ID NO: 938 - DNA, 22 bp, Artificial sequence
  Primer
gagtgatgtcactaacctgcag

SEQ ID NO: 939 - DNA, 19 bp, Artificial sequence
  Primer
tggttgccaagaatgatct

SEQ ID NO: 940 - DNA, 18 bp, Artificial sequence
  Primer
ttcttttcccccaacatg

SEQ ID NO: 941 - DNA, 21 bp, Artificial sequence
  Primer
tgtgtgtatgtggatgaaaac

SEQ ID NO: 942 - DNA, 28 bp, Artificial sequence
  Primer
aattaccagttcttctagtgactttctt

SEQ ID NO: 943 - DNA, 23 bp, Artificial sequence
  Primer
aaacaaaaacaagaacacaccaa

SEQ ID NO: 944 - DNA, 29 bp, Artificial sequence
  Primer
tttgttaccctatatcataactcagtctc

SEQ ID NO: 945 - DNA, 30 bp, Artificial sequence
  Primer
aaaagttgtctctccataatattctctagt

SEQ ID NO: 946 - DNA, 23 bp, Artificial sequence
  Primer
ttggattaacaagtggaactgtt

SEQ ID NO: 947 - DNA, 21 bp, Artificial sequence
  Primer
attggtgtttccaggaactca

SEQ ID NO: 948 - DNA, 21 bp, Artificial sequence
  Primer
aagaaacactcactcccaatg

SEQ ID NO: 949 - DNA, 22 bp, Artificial sequence
  Primer
ttttcccataatcactttccat

SEQ ID NO: 950 - DNA, 22 bp, Artificial sequence
  Primer
ataaaaccccagggatatgaaa

SEQ ID NO: 951 - DNA, 24 bp, Artificial sequence
  Primer
taaacattggatttttactgctca

SEQ ID NO: 952 - DNA, 27 bp, Artificial sequence
  Primer
gtttacaaataactttggaaaatgaag

SEQ ID NO: 953 - DNA, 24 bp, Artificial sequence
  Primer
tcttttcccagattcaatagagtt

SEQ ID NO: 954 - DNA, 30 bp, Artificial sequence
  Primer
atatataaagtcttttaagcctcagaagtg

SEQ ID NO: 955 - DNA, 21 bp, Artificial sequence
  Primer
aaccagaagctttccatttaa

SEQ ID NO: 956 - DNA, 26 bp, Artificial sequence
  Primer
agaatttgaggtctatttgtcatagc

SEQ ID NO: 957 - DNA, 28 bp, Artificial sequence
  Primer
aaaaataatctcttttccagaagtctaa

SEQ ID NO: 958 - DNA, 23 bp, Artificial sequence
  Primer
cttatttttcctgctgccttact

SEQ ID NO: 959 - DNA, 27 bp, Artificial sequence
  Primer
ttataaactacagaaatctcagccaat

SEQ ID NO: 960 - DNA, 27 bp, Artificial sequence
  Primer
aatttaactttcagcttttctcactta

SEQ ID NO: 961 - DNA, 20 bp, Artificial sequence
  Primer
tgttctgtcttggagagggt

SEQ ID NO: 962 - DNA, 21 bp, Artificial sequence
  Primer
attcaagtttctttgggcaac

SEQ ID NO: 963 - DNA, 24 bp, Artificial sequence
  Primer
atatagcttttggaattctccatg

SEQ ID NO: 964 - DNA, 23 bp, Artificial sequence
  Primer
taaaaggaatgcagacttcagag

SEQ ID NO: 965 - DNA, 19 bp, Artificial sequence
  Primer
tgaatggatggatgagtgc

SEQ ID NO: 966 - DNA, 29 bp, Artificial sequence
  Primer
atacagatgctataattctcaaggatatg

SEQ ID NO: 967 - DNA, 24 bp, Artificial sequence
  Primer
aataggcagtggtgacttgttagt

SEQ ID NO: 968 - DNA, 23 bp, Artificial sequence
  Primer
tttacttaagccaactcgagtga

SEQ ID NO: 969 - DNA, 27 bp, Artificial sequence
  Primer
taataaatcagaaaaaatagtcgctgt

SEQ ID NO: 970 - DNA, 35 bp, Artificial sequence
  Primer
gtacagttaaatagatatgttttctagtatgaatg

SEQ ID NO: 971 - DNA, 25 bp, Artificial sequence
  Primer
agaacactagctcccagaaagtagt

SEQ ID NO: 972 - DNA, 24 bp, Artificial sequence
  Primer
agcctccaatttcttacatttaaa

SEQ ID NO: 973 - DNA, 28 bp, Artificial sequence
  Primer
cctgtgattttctttttatttatgtcta

SEQ ID NO: 974 - DNA, 22 bp, Artificial sequence
  Primer
tgaaatccacacagtatcttgc

SEQ ID NO: 975 - DNA, 22 bp, Artificial sequence
  Primer
attctgcttctccctctgatag

SEQ ID NO: 976 - DNA, 23 bp, Artificial sequence
  Primer
aatattctctgaggtctcccact

SEQ ID NO: 977 - DNA, 29 bp, Artificial sequence
  Primer
atatgatcaattctaagagggagtataaa

SEQ ID NO: 978 - DNA, 26 bp, Artificial sequence
  Primer
aaaaaagtcaacttacagatctctgc

SEQ ID NO: 979 - DNA, 26 bp, Artificial sequence
  Primer
tattctaattgacagagctctcatcc

SEQ ID NO: 980 - DNA, 18 bp, Artificial sequence
  Primer
ttgaaccaccccgtccca

SEQ ID NO: 981 - DNA, 27 bp, Artificial sequence
  Primer
tataagttggtgtgaaacattgtaatc

SEQ ID NO: 982 - DNA, 21 bp, Artificial sequence
  Primer
tgacccaccacattttctaag

SEQ ID NO: 983 - DNA, 21 bp, Artificial sequence
  Primer
attgggaaaagagtgtggaag

SEQ ID NO: 984 - DNA, 27 bp, Artificial sequence
  Primer
aattacattatgctcttaattgaaccc

SEQ ID NO: 985 - DNA, 19 bp, Artificial sequence
  Primer
tgtaaaggaaacgccatgg

SEQ ID NO: 986 - DNA, 19 bp, Artificial sequence
  Primer
atccttccctccagtggtt

SEQ ID NO: 987 - DNA, 25 bp, Artificial sequence
  Primer
acctgagtaatggttacatctctga

SEQ ID NO: 988 - DNA, 26 bp, Artificial sequence
  Primer
attgcattaatattccattaatgagg

SEQ ID NO: 989 - DNA, 29 bp, Artificial sequence
  Primer
acaaagctaattattacaagagactctcc

SEQ ID NO: 990 - DNA, 23 bp, Artificial sequence
  Primer
tgccgatgcaattattttattat

SEQ ID NO: 991 - DNA, 22 bp, Artificial sequence
  Primer
tgggctttctagtcttcatgtc

SEQ ID NO: 992 - DNA, 28 bp, Artificial sequence
  Primer
taaattctgcaagtttatagagaaacac

SEQ ID NO: 993 - DNA, 21 bp, Artificial sequence
  Primer
ttcacctaacactcttttgcc

SEQ ID NO: 994 - DNA, 23 bp, Artificial sequence
  Primer
tgagccaagtgtttttcttaaac

SEQ ID NO: 995 - DNA, 24 bp, Artificial sequence
  Primer
aaaaccaaaaactcataaccagaa

SEQ ID NO: 996 - DNA, 32 bp, Artificial sequence
  Primer
attttctgtcatatttctatatacgtagattc

SEQ ID NO: 997 - DNA, 27 bp, Artificial sequence
  Primer
agcttggtaaaatgctatagttgtaaa

SEQ ID NO: 998 - DNA, 28 bp, Artificial sequence
  Primer
agaattattaaatgcaacatcataccta

SEQ ID NO: 999 - DNA, 25 bp, Artificial sequence
  Primer
ttttaatatggtgtcctgctaaaag

SEQ ID NO: 1000 - DNA, 23 bp, Artificial sequence
  Primer
caattataccggcactaaaactg

SEQ ID NO: 1001 - DNA, 30 bp, Artificial sequence
  Primer
tttttaaatacagatactcagaaagttcac

SEQ ID NO: 1002 - DNA, 22 bp, Artificial sequence
  Primer
atggtatcagagctgtcttcca

SEQ ID NO: 1003 - DNA, 24 bp, Artificial sequence
  Primer
ttttttttctctttttgttcttgg

SEQ ID NO: 1004 - DNA, 22 bp, Artificial sequence
  Primer
atgtcaggaaagcaaaaatgag

SEQ ID NO: 1005 - DNA, 24 bp, Artificial sequence
  Primer
ttgtttatgttgcctttgaagata

SEQ ID NO: 1006 - DNA, 27 bp, Artificial sequence
  Primer
aattcttgtgaaacatttagtctctca

SEQ ID NO: 1007 - DNA, 21 bp, Artificial sequence
  Primer
atgaatttttccagcaatgtg

SEQ ID NO: 1008 - DNA, 24 bp, Artificial sequence
  Primer
taaattccaaaacaggtgactatg

SEQ ID NO: 1009 - DNA, 24 bp, Artificial sequence
  Primer
taattgactgtgttctttccttgg

SEQ ID NO: 1010 - DNA, 23 bp, Artificial sequence
  Primer
ttctgaattctgtactgtcccac

SEQ ID NO: 1011 - DNA, 27 bp, Artificial sequence
  Primer
actttatcttgttttgattgaagaaac

SEQ ID NO: 1012 - DNA, 22 bp, Artificial sequence
  Primer
tgctgaaatctttcacctaagg

SEQ ID NO: 1013 - DNA, 21 bp, Artificial sequence
  Primer
tggcttgttttaggacatcag

SEQ ID NO: 1014 - DNA, 23 bp, Artificial sequence
  Primer
tactcacagctgaagaaaatgag

SEQ ID NO: 1015 - DNA, 19 bp, Artificial sequence
  Primer
ttaggaaaaaagccccaca

SEQ ID NO: 1016 - DNA, 25 bp, Artificial sequence
  Primer
atttgttattccagaagtatgtgct

SEQ ID NO: 1017 - DNA, 22 bp, Artificial sequence
  Primer
ttagccagtgcaacaataaacc

SEQ ID NO: 1018 - DNA, 28 bp, Artificial sequence
  Primer
tctaaagtgtggttaattcttttaatca

SEQ ID NO: 1019 - DNA, 23 bp, Artificial sequence
  Primer
taagtcaatgcaaatgattgatg

SEQ ID NO: 1020 - DNA, 18 bp, Artificial sequence
  Primer
tgtagggtgctggccttc

SEQ ID NO: 1021 - DNA, 20 bp, Artificial sequence
  Primer
agcgtaaccaccttctcctg

SEQ ID NO: 1022 - DNA, 25 bp, Artificial sequence
  Primer
ataattttcaaaggctctagtctgc

SEQ ID NO: 1023 - DNA, 18 bp, Artificial sequence
  Primer
tgatttcatccaggccct

SEQ ID NO: 1024 - DNA, 19 bp, Artificial sequence
  Primer
acttccaccccaactctga

SEQ ID NO: 1025 - DNA, 18 bp, Artificial sequence
  Primer
ggtggacacaaaacccac

SEQ ID NO: 1026 - DNA, 24 bp, Artificial sequence
  Primer
ctggaaataggtaggaattgtgtc

SEQ ID NO: 1027 - DNA, 23 bp, Artificial sequence
  Primer
ttttattctaagctcccaggaaa

SEQ ID NO: 1028 - DNA, 22 bp, Artificial sequence
  Primer
aaaacaatttgaagcacaggtc

SEQ ID NO: 1029 - DNA, 23 bp, Artificial sequence
  Primer
attataatccatcctccaacaca

SEQ ID NO: 1030 - DNA, 23 bp, Artificial sequence
  Primer
aacaaaaaattggttcttaggga

SEQ ID NO: 1031 - DNA, 32 bp, Artificial sequence
  Primer
aaagtatgaactcagtaagtttttctagagta

SEQ ID NO: 1032 - DNA, 21 bp, Artificial sequence
  Primer
aaaaaatgttcgcggtagagt

SEQ ID NO: 1033 - DNA, 18 bp, Artificial sequence
  Primer
atttggccctaagccaga

SEQ ID NO: 1034 - DNA, 26 bp, Artificial sequence
  Primer
atagtcagagaaaagcagaaagtctt

SEQ ID NO: 1035 - DNA, 19 bp, Artificial sequence
  Primer
aaagggtaaagggaaaggg

SEQ ID NO: 1036 - DNA, 24 bp, Artificial sequence
  Primer
ataagtagcaggtgaggcaataat

SEQ ID NO: 1037 - DNA, 25 bp, Artificial sequence
  Primer
aatgtaaagcattctctctctaccc

SEQ ID NO: 1038 - DNA, 31 bp, Artificial sequence
  Primer
ctatctgtgtgattatcatttctttataatg

SEQ ID NO: 1039 - DNA, 22 bp, Artificial sequence
  Primer
atatggtgcaataaaattgtgg

SEQ ID NO: 1040 - DNA, 31 bp, Artificial sequence
  Primer
attaaaaagtagtcagatactaagcaaaaac

SEQ ID NO: 1041 - DNA, 18 bp, Artificial sequence
  Primer
ttcagctctggcctgtcc

SEQ ID NO: 1042 - DNA, 24 bp, Artificial sequence
  Primer
taaagataaagaggggtcactttc

SEQ ID NO: 1043 - DNA, 23 bp, Artificial sequence
  Primer
tgctatcagtactgaaacatgcc

SEQ ID NO: 1044 - DNA, 21 bp, Artificial sequence
  Primer
tcctaaactggtgccttcttt

SEQ ID NO: 1045 - DNA, 26 bp, Artificial sequence
  Primer
ataaaattagctgcctaaagacaaag

SEQ ID NO: 1046 - DNA, 28 bp, Artificial sequence
  Primer
tctatatttgatgatacctacgcatagt

SEQ ID NO: 1047 - DNA, 25 bp, Artificial sequence
  Primer
aacatctgaaaacacattgtaaaga

SEQ ID NO: 1048 - DNA, 25 bp, Artificial sequence
  Primer
ttttaaacgtctatctgctttttcc

SEQ ID NO: 1049 - DNA, 23 bp, Artificial sequence
  Primer
tatgggagtagctcttgtcatca

SEQ ID NO: 1050 - DNA, 23 bp, Artificial sequence
  Primer
caagaaagtcttttaagaatcac

SEQ ID NO: 1051 - DNA, 19 bp, Artificial sequence
  Primer
atcccagatcatcccagtc

SEQ ID NO: 1052 - DNA, 21 bp, Artificial sequence
  Primer
agaaacatctcccctctctcc

SEQ ID NO: 1053 - DNA, 20 bp, Artificial sequence
  Primer
tctattattatgcctgggcc

SEQ ID NO: 1054 - DNA, 27 bp, Artificial sequence
  Primer
atttaccaatataggcaaactatctgg

SEQ ID NO: 1055 - DNA, 30 bp, Artificial sequence
  Primer
aatattaatctgaaattagataggatggtg

SEQ ID NO: 1056 - DNA, 24 bp, Artificial sequence
  Primer
atatcttgcgaaaatctcaatctt

SEQ ID NO: 1057 - DNA, 24 bp, Artificial sequence
  Primer
aaaatgtgggatatacagtttttg

SEQ ID NO: 1058 - DNA, 29 bp, Artificial sequence
  Primer
ttgagtttactgtctcagacgttatatag

SEQ ID NO: 1059 - DNA, 20 bp, Artificial sequence
  Primer
tcattcagccgcattactaa

(19) United States
(12) Patent Application Publication
(10) Pub. No.: US 2007/0037182 A1
(43) Pub. Date: Feb. 15, 2007

(54) Multiplex assays for inferring ancestry

(76) Inventors: James Z. Gaskin, Sarasota, FL (US); Matthew J. Thomas, Bradenton, FL (US); Tony N. Frudakis, Bradenton, FL (US); Mark D. Shriver, State College, PA (US)

Correspondence Address: DLA PIPER US LLP, 4365 EXECUTIVE DRIVE, SUITE 1100, SAN DIEGO, CA, 92121-2133, US

(21) Appl. No.: 11/357,729

(22) Filed: Feb. 17, 2006

Related U.S. Application Data
(63) Continuation-in-part of application No. 10/644,594, filed on Aug. 19, 2003.
(63) Continuation-in-part of application No. 10/156,995, filed on May 28, 2002, now abandoned.
(63) Continuation-in-part of application No. 10/188,359, filed on Jul. 1, 2002.
(63) Continuation-in-part of application No. PCT/US02/38345, filed on Nov. 26, 2002.
(60) Provisional application No. 60/404,357, filed on Aug. 19, 2002.
(60) Provisional application No. 60/467,613, filed on May 2, 2003.

(51) Int. Cl. C12Q 1/68; G06F 19/00; C07H 21/04

(52) U.S. Cl. 435/6; 702/20; 536/24.3

(57) ABSTRACT

Oligonucleotide primers and probes for detecting Ancestry informative marker (AIMs) polynucleotides that contain a single nucleotide polymorphism or that contain an insertion or deletion, are provided, as are methods of using the oligonucleotide primers and probes to draw an inference as to a trait of an individual. The trait can be, for example, biogeographical ancestry, a pigmentation trait, responsiveness to a drug, or susceptibility to a disease. Also provided are methods of determining the proportional ancestry of an individual. Reagents and kits also are provided.

This application claims the benefit of priority under 35 U.S.C. § 119(e) of U.S. Ser. No. 60/654,672 filed Feb. 18, 2005 and is Continuation-in-Part of U.S. Ser. No. 10/644,594, filed Aug. 19, 2003, which in turn claims the benefit of priority under 35 U.S.C. § 119(e) of U.S. Ser. No. 60/404,357, filed Aug. 19, 2002, and U.S. Ser. No. 60/467,613, filed May 2, 2003, and is a continuation-in-part of U.S. Ser. No. 10/156,995, filed May 28, 2002; a continuation-in-part of U.S. Ser. No. 10/188,359, filed Jul. 1, 2002; and a continuation-in-part of International Application PCT/US02/38345, filed Nov. 26, 2002 (Intl. Publ. No. WO 03/045227A, Jun. 5, 2003), the entire content of each of which is incorporated herein by reference.

Each of the CD-ROM (compact disk-read only memory) and identical copy thereof, which are submitted herewith and contain a computer program listing, is incorporated herein by reference.

### BACKGROUND OF THE INVENTION

1. Field of the Invention

The invention relates generally to the identification of genetic markers predictive of an individual's biogeographical ancestry, and more specifically to combinations of single nucleotide polymorphisms useful as ancestry informative markers (AIMs), which allow an inference as to a trait of an individual, algorithms for identifying such AIMs, and methods of using such AIMs to infer a trait of an individual, including an individual's ancestry, responsiveness of an individual to a drug, and predisposition of an individual to a disease.

2. Background Information

The majority (80-90%) of the genetic variation among human individuals is inter-individual, and only a relatively small proportion (10-20%) is due to population differences (Nei, In Molecular Population Genetics (Columbia University Press, New York) 1987; Cavalli-Sforza et al., In The History and Geography of Human Genes (Princeton University Press, Princeton N.J.) 1994; Deka et al., Electrophoresis 16:1659-1664, 1995; Rosenberg et al., Science 298:2381-2385, 2002; Akey et al., BioTechniques 30:348-367, 2001; Akey et al., Hum. Genet. 108:516-520, 2002). Most populations share alleles and those alleles that are most frequent in one population are also frequent in others. There are very few classical markers (e.g., blood group, serum protein, and immunological markers) or DNA genetic markers that are population-specific or have large frequency differentials among geographically and ethnically defined populations (Roychodhury and Nei, In Human Polymorphic Genes: World Distribution (Oxford University Press, New York) 1988; Dean et al., Amer. J. Hum. Genet. 55:788-808, 1994; Cavalli-Sforza et al., supra, 1994, Akey et al., supra, 2001, 2002). Despite this apparent lack of unique genetic markers, there are marked physical and physiological differences among human populations that presumably reflect genetic adaptation to unique ecological conditions, random genetic drift, and sex selection. In contemporary populations, these differences are evident in morphological differences between ethnic groups, as well as in differences in drug responsiveness and in susceptibility and resistance to disease.

On a basic level, human population structure can be represented in terms of BioGeographical Ancestry (BGA), which is the heritable component of “race” or heritage, and which is relevant on any scale of resolution. For example, on a crude level, BGA can be determined for 2 groups (e.g., European vs. others); or on a fine level, e.g., it can refer to “race” in terms of 4 groups such as IndoEuropeans, East Asians, sub-Saharan African and Native American; or on a finer level, e.g., it can refer to ethnicity within the European group (for example, Mediterranean or Scandinavian); or on a still finer level, e.g., it can even refer to groups of families within ethnic groups, such as groups of O'Reilly's descendent from a set of common ancestors within the Irish group. The measurement of BGA is relevant for most any type of genetics or epidemiological study design. For example, BGA is an important component in the variability of drug response (Burroughs et al., J. Natl. Med. Assoc. 94:1-26, 2002). The reason for this relationship is that genetic drift, geographical and/or reproductive isolation, and regional selective pressures have molded the allele frequencies of our ancestors for compatibility with alkaloids, tannins (self-defense chemicals), and other xenobiotics found in indigenous diets. Most drugs are derived from such chemicals and, therefore, it is no coincidence that the family of enzymes that allow humans to detoxify drugs are found at different frequencies in different populations. This scenario is not unique to drug responsiveness, and many other parts of the genome that are unrelated to drug responsiveness are subject to these same types of pressures.

Investigators generally have been concerned with identifying gene variants that cause a disease (the so called “phenotypically active” loci), rather than identifying gene variants that are simply correlated with disease. As such, whatever the trait being examined, and for most study designs involving unrelated individuals, it has been considered important to control for population structure so as to avoid identifying markers of structure that correlate with trait value in a given sample rather than those in linkage disequilibrium (LD) with phenotypically active loci (Risch et al., Genome Biology 3:1-12, 2001 Wang et al., Amer. J. Hum. Genet. 71:1227-1234, 2002; Burroughs et al., supra, 2002; Rao and Chakraborty, Amer. J. Hum. Genet. 26:444-453, 1974). There are two sources of population structure in a sample collection: 1) sampling effects, which can create structure even if sampling is performed from homogeneous populations, and 2) natural human demography. The first source of population structure is a nuisance for genetics studies, and associations found from a study due to this type of structure are generally considered an artifact of the collection process rather than a reflection of human demography. Most geneticists generally consider the second kind of structure to be a nuisance as well. As such, associations identified as being due to population structure have been considered spurious findings or artifacts, and have generally been discarded; only findings due to true linkage or LD have been published, as such markers are considered linked to biologically relevant genes.

Much effort has been directed to quantifying both types of population structure (above) in groups of individuals. Such methods essentially measure the departure from expected levels of heterozygosity within a group of samples as an indication of structure (though none of these methods are capable of reading within-individual structure). Many common diseases exhibit locus and/or allelic heterogeneity as a function of BGA, and many authors have suggested that inappropriate attention to population structure during the study design step has produced at least some of the so-called “false positive” results implicated in the rash of irreproducible Common Disease/Common Variant results obtained to date (Terwilliger et al., Curr. Opin. Genet. Devel. 12:726-734, 2002). In order to control for the influence of population structure, several tests are appropriate (Cockerham, Evolution 23:72-83, 1969; Cockerham, Genetics 74:679-700, 1973; Wier and Cockerham, Evolution 38:1358-1370, 1984; Long, Genetics 112:629-647, 1986, Excoffier et al., Genetics 131:343-359, 1992). These methods can be grouped in two main categories—genomic control methods (Devlin and Roeder, Biometrics 55:997-1004, 1999), and structured association (SA) methods (Pritchard and Donelly, Theor. Popul. Biol. 60:227-237, 2001). Both methods require genotyping of a panel of unlinked markers to estimate and correct for the effect of genetic structure, but they are usually applied for sample collections. However, should a pool of samples fail such a test, it is usually not clear which samples should be eliminated to rectify the problem. An equally vexing problem with this method is that it is often applied for a study sample after the creation of expensive data, thus creating a circular logic problem in addition to an economic problem; these methods are usually employed to extract information on population structure using the characteristics of the data within which associations are sought.

In order to minimize the influence of population structure from the outset of an effort to identify phenotypically active loci, where structure or admixture is not to be used as a statistical fuel, it is generally desirable to qualify samples based on crude population stratifications such as BGA so that cases and controls can be matched and homogenized in composition. For example, it is not uncommon in the execution of case-control studies to ensure equal proportions or “racial homogeneity” within and between cases and controls. However, for most research purposes, the subjective methods used to measure population affiliation are unsatisfactory. As currently measured using biographical questionnaires, little knowledge of population structure other than the obvious is obtained, and only basic connections between population structure and drug response can be apparent and/or controlled. Consistency is a significant problem with the self-reporting of race on questionnaires, and one that the Food and Drug Administration is attempting to address during the clinical trial design process. However, using such subjective and imprecise methods of data collection, consistency can be a difficult end to achieve.

Rather than reformulating how questions are asked on questionnaires, consistency can be better addressed by replacing the subjective nature of the exercise with objective, reproducible scientific methods. Standardization and objectivity is of paramount importance for the collection of race data because its measure can be as subjective as that of any other human attribute. The self-reporting of race is not as trivial an exercise as the self-reporting of gender, and many people do not know their race or are of sufficient admixture that they have trouble classifying themselves into a single group. Such a scenario is particularly common in countries such as the United States, in which numerous cultures have been combined due to immigration. For example, a woman of mainly sub-Saharan African descent, raised in Puerto Rico, may describe herself as Hispanic. Though the socio-culturally identifies with Hispanics, however, her xenobiotic metabolism and drug target polymorphisms may more likely be associated with those shared among other sub-Saharans. By using nonanthropologic designations that describe the socio-cultural construct of society, current guidelines for considering information on race in the study design process can effect poor predictive power and false positive results. Where a person was raised and lives, and the cultural or sociological customs they observe, may have an impact on how that person responds to a drug or proclivity to develop a disease. Thus, non-biological metrics are required, but the evidence suggests that BGA also has an impact and, therefore, needs to be measured in a scientifically accurate and reproducible manner.

Genetic markers present in a person's DNA provide the best opportunity to reliably determine the BGA an individual, and it has long been recognized that such a means is possible. For example, Reed (Science 244:575-576, 1973) and Neel (Mutat. Res. 26:319-328, 1974) referred to such markers as “private”, and used them to estimate mutation rates. Reed (supra, 1973) used the term “ideal” (in reference to the utility of the markers in individual ancestry estimation) to describe hypothetical genetic marker loci at which different alleles are fixed in different populations. Chakraborty et al. (Ethnic. Dis. 1:245-256, 1991) referred to variants that are found in only one population as “unique alleles”, and showed how allele frequencies could be inverted to provide a likelihood estimate of population, or BGA affiliation. The most useful “unique alleles” for the inference of BGA are those that also have large differences in allele frequency among populations (Reed, supra, 1973; Chakraborty et al., Genetics 130:231-243, 1992; Stephens et al., Amer. J. Hum. Genet. 55:809-824, 1994), and that have been referred to as “population-specific alleles” (PSAs, Shriver et al., Amer. J. Hum. Genet. 60:957-964, 1997; Parra et al., Amer. J. Hum. Genet. 63:1839-1851, 1998), but which are now referred to as “Ancestry Informative Markers” (AIMs; Shriver et al., Hum. Genet. 112:387-399, 2003, Frudakis et al., J. Forens. Sci. 48(4) 771-782, 2003).

Within the field of forensics, statistical methods that use short tandem repeats (STRs) to infer the highest level of ancestry in a particular individual (majority BGA using proportional ancestry notation) can be fairly robust, but can not always be reliably used to estimate major majority BGA affiliation. Although STR tests can effectively resolve majority ancestral origin in most cases, an unacceptable number (5-10%) of classifications are ambiguous. Aside from sampling errors caused by rare alleles, and the fact that STRs were not selected from the genome for their ability to resolve population affiliation (i.e., STR allele frequency differentials are not necessarily nor optimally informative for this purpose), the major reason the high level of ambiguity likely is due to admixture, which is clearly a factor of the genetic variation for many human populations (Parra et al., supra, 1998, Cavalli-Sforza and Bodmer, In The genetics of human populations (Dover Publications, NY; see pages 387-507) 1999; Rosenberg et al., supra, 2002). For a given study design, whether using self-reported information or DNA marker testing, and whether attempting to solve a pharmacogenomic or forensic problem, classifying a patient into a single group sacrifices the subtle, but not insignificant, information related to population structure and sub-structure; for example, there is no allowance to assign a person of 50% African and 50% European affiliation into a group. Unfortunately, markers and methods for allowing an accurate inference as to the BGA for more than just two groups at a time for an individual have now been described. Generally, the more markers of an individual that are examiner, the greater the likelihood that an inference as to BGA will be accurate. Thus, a need exists for convenient and cost effective high throughput methods for determining such marker in nucleic acid samples. The present invention satisfies this need, and provides additional advantages.

### SUMMARY OF THE INVENTION

The present invention provides methods and compositions for measuring, with a desired predetermined level of confidence, within individual population structure, which, as disclosed herein, allows inferences to be drawn, for example, as to ancestry, pigmentation traits, drug responsiveness, and disease susceptibility of the individual. By way of example, the present methods and compositions were used in a forensics capacity, wherein DNA samples obtained at the crime scenes of a serial murder/rapist in Louisiana were examined. Based on psychological profiling, police were of the belief that the serial killer was a Caucasian male, and had tested the DNA of over 1,000 Caucasian men without finding a match. The police then turned to the inventors, who, using the compositions and methods of the invention, determined that the individual committing the crimes was African American and, more specifically, had a proportional and confidence qualified ancestry of 85% sub-Saharan African and 15% Native American. Based on this result and additional results as disclosed herein, the police were further advised that the average African American is of 20% IndoEuropean ancestry, that greater levels of IndoEuropean ancestry correlate with lighter skin tone, and, therefore, that the person committing the crimes was likely an African American with average to darker than average skin tone. Within two months of refocusing their efforts based on this information, the police arrested an African American man of average skin tone (for African Americans); DNA testing determined that he was the person whose DNA was found at the crime scenes.

Accordingly, the present invention relates to a method of inferring, with a predetermined level of confidence, a trait of an individual. Such a method can be performed, for example, by contacting a sample, which includes nucleic acid molecules of a test individual, with hybridizing oligonucleotides, wherein the hybridizing nucleotides can detect nucleotide occurrences of single nucleotide polymorphisms (SNPs) of a panel of at least about ten ancestry informative markers (AIMs) indicative of a population structure correlated with the trait, and wherein said contacting is performed under conditions suitable for detecting the nucleotide occurrences of the AIMs of the individual by the hybridizing oligonucleotides; and identifying, with a predetermined level of confidence, a population structure that correlates with the nucleotide occurrences of the AIMs in the individual, wherein the population structure correlates with a trait. As disclosed herein, a panel of at least about ten AIMs (e.g., 8, 9, 10, 11, 12, 13, 14, 15, 20, 25, 30, or more) is examined in practicing a method of the invention. Generally, the greater number of AIMs examined, the greater the confidence level of an inference made using the method.

A trait for which an inference is made according to a method of the invention can be any trait, including a trait for which an ethnic predisposition is known or suspected to occur and a trait for which it known that no ethnic predisposition occurs or for which it is not known or unclear as to whether there is an ethnic predisposition. In one embodiment, the trait is biogeographical ancestry (BGA). In one aspect, the panel of AIMs used to examine BGA includes AIMs as set forth in SEQ ID NOS:1 to 71. In another aspect, the panel includes AIMs as set forth in SEQ ID NOS:7, 21, 23, 27, 45, 54, 59, 63, and 72 to 152; in SEQ ID NOS:3, 8, 9, 11, 12, 33, 40, 59, 63, and 153 to 239; or in SEQ ID NOS:1, 8, 11, 21, 24, 40, 172, and 240 to 331, as well as a panel containing combinations of AIMs as set forth in SEQ ID NOS:1 to 331. As disclosed herein, AIMs useful in practicing a method of the invention can, but need not, be linked to a gene linked to the trait (i.e., a gene known to be involved in the trait phenotype) and generally are not in linkage disequilibrium with the gene (or locus). For example, an AIM useful for inferring drug responsiveness of an individual according to a method of the invention need not be linked to a gene involved in responsiveness to the drug (e.g., a drug metabolism gene or a drug transport gene such as a cytochrome P450 gene or P-glycoprotein gene). Similarly, an AIM useful for inferring a pigmentation trait of an individual according to a method of the invention need not be linked to a gene involved in pigmentation (e.g., a tyrosinase gene or a melanocortin-1 receptor gene). Thus, in one aspect, at least one (e.g., 1, 2, 3, 4, or 5) AIM of a panel is not linked to a gene involved in the trait for which an inference is being made.

Where BGA is the trait for which an inference is being made, an individual being examined can have an ancestry that includes any one or a combination of ancestral groups, including, for example, a proportion of sub-Saharan African ancestry, Native American ancestry, IndoEuropean ancestry, East Asian ancestry, Middle Eastern ancestry, Pacific Islander ancestry, or a combination including one or more of these ancestries. As such, the proportional ancestry of an individual can comprise one ancestry (e.g., 100% IndoEuropean ancestry), or any proportion of two, three, four, or more ancestral groups. As such, a test individual (or individual of known proportional ancestry) can have, for example, a proportion of at least three ancestral groups, which can include proportions of sub-Saharan African ancestry and two other ancestries, or can include proportions of sub-Saharan African and IndoEuropean ancestral groups and a third ancestry; or Native American and IndoEuropean ancestral groups and a third ancestry; or East Asian and Native American ancestral groups and a third ancestry; or IndoEuropean and East Asian ancestral groups and a third ancestry; or can include proportions of Native American, East Asian, and IndoEuropean ancestral groups, or of sub-Saharan African, Native American, and IndoEuropean ancestral groups, and the like.

In another embodiment, a trait of a test individual for which an inference is being made is responsiveness of the individual to a drug, particularly a therapeutic drug. As such, a method of the invention provides a tool for realizing personalized medicine. A drug for which an inference can be made as to whether a test individual will be responsive, in either a positive or negative manner, can be, for example, a cancer chemotherapeutic agent such as paclitaxel, or a drug such as a statin, which can be useful for maintaining or lowering cholesterol levels. In one aspect of this embodiment, AIMs of the panel of AIMs used to practice the method includes AIMs of genes other than genes known to be involved in melanin synthesis or metabolism.

In still another embodiment, a trait of a test individual for which an inference is being made is a susceptibility or predisposition of the individual to a disease. As disclosed herein, various traits are associated with population structure at a continental level, whereas other traits are associated with population structure at finer levels. As such, a method of the invention can provide an means for making an inference with respect to a trait such as disease susceptibility for diseases such as diabetes, hypertension, and cancers that are known to have an ethnic predisposition (i.e., known to occur with higher frequencies in individuals of certain ethnic/ancestral groups), as well as for disease such as such as alcoholism, or schizophrenia, Parkinson's disease, and other neurological disorders, which do not (or at least are not known to) have an ethnic predisposition.

In yet another embodiment, a trait of a test individual for which an inference is being made is a pigmentation trait. The pigmentation trait can be any such trait including, for example, eye color or shade, skin color, hair color, or a combination thereof. In one aspect of this embodiment, AIMs of the panel of AIMs used to practice the method includes AIMs of genes other than genes known to be involved in melanin synthesis or metabolism, or other aspects of pigmentation.

A method of inferring a trait of a test individual by determining a population structure that correlates with nucleotide occurrences of AIMs in the individual can further include identifying, with a predetermined level of confidence, a sub-population structure of the population structure, wherein the sub-population structure correlates with a trait. For example, a population structure of an individual can correlate to an intercontinental group with which, by inference, the individual shares ancestry, for example, IndoEuropean, and a sub-population structure can further correlate with an intracontinental group with which the individual shares IndoEuropean ancestry, for example, Mediterranean ethnicity.

The hybridizing oligonucleotides useful in the methods of the invention can be oligonucleotide probes or oligonucleotide primers. Oligonucleotide probes useful in the present methods can hybridize to a nucleotide sequence that includes the SNP position for an AIM, wherein the nucleotide at the position of the hybridizing oligonucleotide that corresponds to the position of the SNP for the AIM either matches or does not match the nucleotide occurrence at the SNP position. Additional oligonucleotide probes useful in the methods of the invention include oligonucleotide probes that hybridize to a polynucleotide sequence adjacent to and upstream and/or adjacent to and downstream of the SNP position, and that can, but need not, include a nucleotide corresponding to the nucleotide position of the SNP, and wherein such a corresponding nucleotide, when present in the probe, can, but need not match the nucleotide occurrence at the SNP.

Oligonucleotide primers useful in the methods of the invention include oligonucleotide primers useful for a primer extension reaction, as well as oligonucleotide primers that, in combination, allow for amplification of template polynucleotide comprising the AIM. Such amplification primer pairs generally include a forward primer and a reverse primer useful for amplification of a template polynucleotide comprising an AIM of interest. It will be recognized, however, that 2, 3, 4, or more different forward primers can be used with a common reverse primer for amplification of different template polynucleotides comprising the AIM (e.g., in a multiplex reaction) and a common gene sequence (e.g., AIMs of a family of related gene sequences) or for generating amplification products of different sizes from a single template. Similarly, one common forward primer can be used with one or a plurality of different reverse primers.

Accordingly, in one embodiment, a method of the invention is performed using oligonucleotide primers. In one aspect of this embodiment, the method includes contacting the sample with the oligonucleotide primers and with a polymerase, under condition suitable for generation of a primer extension product. In such a method, the nucleotide occurrence of a SNP can be determined by detecting the presence of the primer extension product, or by sequencing the primer extension product (or a product thereof) and identifying the nucleotide at the position corresponding to the position of the SNP. In another aspect of this embodiment, the method includes contacting the sample with oligonucleotide primers that comprise amplification primer pairs and with a polymerase, under condition suitable for generation of an amplification product. In such a method, the nucleotide occurrence of a SNP can be determined by detecting the presence of the amplification product, or by sequencing the amplification product (or a product thereof) and identifying the nucleotide at the position corresponding to the position of the SNP.

The methods of the invention are particularly adaptable to being performed in a high throughput format, including in a multiplex format, thus allowing examination of a large number of AIMs and/or a large number of samples of test individuals, as well as controls, in parallel. As such, the methods can be performed using a format in which the samples being examined are arranged in an array, particularly an addressable array, e.g., on in wells in a tray or on a glass slide or silicon chip, and can be partly or fully automated using robotics. Where a multiplex platform is used, it will be recognized that the AIMs examined need not necessarily be those having the greatest delta values for the particular trait, but also can be selected to balance the delta value with the compatibility of primers in a multiplex set, for example, to select AIMs such that hybridizing oligonucleotides (e.g., amplification primer pairs) can be designed that can be used in a single reaction for examining a panel of AIMs but that do not substantially cross-hybridize with AIMs other than the target AIM for which the hybridizing oligonucleotides are designed.

The present invention also relates to a method of estimating, with a predetermined level of confidence, proportional ancestry of at least two ancestral groups of a test individual. Such a method can be performed, for example, by contacting a sample, which includes nucleic acid molecules of the test individual, with hybridizing oligonucleotides that can detect nucleotide occurrences of SNPs of a panel of at least about ten AIMs that are indicative of BGA for each ancestral group examined, wherein the contacting is under conditions suitable for detecting the nucleotide occurrences of the AIMs of the test individual by the hybridizing oligonucleotides; and identifying, with a predetermined level of confidence, a population structure that correlates with, or is most likely given, the nucleotide occurrences of the AIMs of each of the ancestral groups examined, wherein the population structure is indicative of proportional ancestry.

The proportional ancestry estimated according to a method of the invention can be a proportion of any ancestral group, including, for example, a proportion of sub-Saharan African, Native American, IndoEuropean, East Asian, Middle Eastern, or Pacific Islander ancestral group, and generally is a combination of two or more of such ancestral groups. Thus, the proportional ancestry of a test individual can include proportions of sub-Saharan African and IndoEuropean ancestral groups (e.g., 80% sub-Saharan African and 20% IndoEuropean; or 60% sub-Saharan African, 20% IndoEuropean, and 20% of a third ancestral group); or can include proportions of Native American and IndoEuropean ancestral groups; East Asian and Native American ancestral groups; IndoEuropean and East Asian ancestral groups; and the like. Similarly, the proportional ancestry can include proportions of Native American, East Asian, and IndoEuropean ancestral groups; sub-Saharan African, Native American, and IndoEuropean ancestral groups; sub-Saharan African, Native American, and East Asian ancestral groups; and the like.

A panel of AIMs useful for estimating proportional ancestry of an individual can include AIMs as set forth in SEQ ID NOS:1 to 331, for example, AIMs as set forth in SEQ ID NOS:1 to 71, which can be useful for determining proportional ancestries including IndoEuropean, sub-Saharan African, East Asian, and Native American; or AIMs as set forth in SEQ ID NOS:7, 21, 23, 27, 45, 54, 59, 63, and 72 to 152, which can be useful for determining proportional ancestry of East Asians and sub-Saharan Africans; or in SEQ ID NOS:3, 8, 9, 11, 12, 33, 40, 59, 63, and 153 to 239, which can be useful for determining proportional ancestry of East Asians and IndoEuropeans; or in SEQ ID NOS:1, 8, 11, 21, 24, 40, 172, and 240 to 331, which can be useful for determining proportional ancestry of IndoEuropeans and sub-Saharan Africans.

In one embodiment, an estimate is made wherein the proportional ancestry includes proportions of three ancestral groups. In one aspect of this embodiment, identifying a population structure that correlates with, or is most likely given, the nucleotide occurrences of the AIMs of the test individual is practiced by performing a likelihood determination for affiliation with each of a sub-Saharan African ancestral group, a Native American ancestral group, an IndoEuropean ancestral group, and an East Asian ancestral group; thereafter selecting three ancestral groups having a greatest likelihood value; determining a likelihood of all possible proportional affiliations among the three ancestral groups having the greatest likelihood value, whereby a population structure or proportional affiliation that correlates with the nucleotide occurrences of the AIMs of the test individual is identified; and identifying a single proportional combination of maximum likelihood.

In another aspect of this embodiment, identifying a population structure that correlates with, or is most likely given, the nucleotide occurrences of the AIMs is practiced by performing six two-way comparisons comprising likelihood determinations for affiliation between each group with each other group; thereafter selecting three ancestral groups having a greatest likelihood value; determining a likelihood of all possible proportional affiliations among the three ancestral groups having the greatest likelihood value, whereby a population structure or proportional affiliation that correlates with, or is most likely given, the nucleotide occurrences of the AIMs of the test individual is identified; and identifying a single proportional combination of maximum likelihood.

In still another aspect of the embodiment wherein an estimate is made wherein the proportional ancestry includes proportions of three ancestral groups, the method is practiced by performing three three-way comparisons among the groups; determining a likelihood of all possible proportional affiliations among the three ancestral groups having the greatest likelihood value, whereby a population structure or proportional affiliation that correlates with, or is most likely given, the nucleotide occurrences of the AIMs of the test individual is identified; and identifying a single proportional combination of maximum likelihood. In another aspect of this embodiment, the method can further include generating a graphical representation of the comparison of the three ancestral groups, wherein the graphical representation comprises a triangle with each ancestral group independently represented by a vertex of the triangle, and wherein the maximum likelihood value of proportional affiliation for an individual comprises a point within the triangle. If desired, the graphical representation can further include a confidence contour that indicates a level of confidence associated with estimating the proportional ancestry.

In another embodiment, an estimate is made wherein the proportional ancestry includes proportions of four ancestral groups. In various aspects of this embodiment, identifying a population structure that correlates with, or is most likely given, the nucleotide occurrences of the AIMs of the test individual is practiced by performing six two-way comparisons, or by performing three three-way comparisons, or by performing one four-way comparison among the groups; determining a likelihood of all possible proportional affiliations among the four ancestral groups having the greatest likelihood value, whereby a population structure or proportional affiliation that correlates with, or is most likely given, the nucleotide occurrences of the AIMs of the test individual is identified; and identifying a single proportional combination of maximum likelihood. In one aspect of this embodiment, the method can further include generating a graphical representation of the comparison of the three ancestral groups, wherein the graphical representation comprises a pyramid with each ancestral group independently represented by a vertex of the pyramid, and wherein the maximum likelihood value of proportional affiliation for an individual comprises a point within the pyramid. If desired, the graphical representation can further include a confidence contour comprising a sphere around the point, wherein the sphere indicates a level of confidence associated with estimating the proportional ancestry.

The method of estimating, with a predetermined level of confidence, proportional ancestry of at least two ancestral groups of a test individual by identifying a population structure indicative of the proportional ancestry can further include identifying a sub-population structure indicative of ethnicity associated with one of the ancestral groups for which the test individual has a proportional ancestry. According to this method, a sub-population structure of the population structure that correlates with the nucleotide occurrences of the AIMs in the test individual is identified, wherein the sub-population structure correlates with ethnicity of the test individual. Such a method of identifying a sub-population structure can be performed, for example, by identifying those chromosomes of the test individual that contain the AIMs indicative of affiliation with a BioGeographical ancestral group (where the individual is proportionally affiliated with more than one BioGeographical Ancestry group), contacting a sample including nucleic acid molecules of the test individual with second hybridizing oligonucleotides that can detect nucleotide occurrences of SNPs of a second panel of AIMs, wherein the AIMs of the second panel are informative for ethnicity within one of these groups and are present on the same chromosomes of the test individual that contain the AIMs indicative of the larger (intercontinental) ancestral group within which the ethnicity occurs; and identifying a sub-population structure that correlates with the nucleotide occurrences of the AIMs of the second panel, wherein the sub-population is indicative of ethnicity of the ancestral group of the test individual.

According to such a method, using hybridizing oligonucleotides specific for the first panel of AIMs (e.g., AIMs of the 71 exemplified AIMs; SEQ ID NOS:1 to 71), a test individual can be determined to be 60% IndoEuropean (IE) and 40% East Asian. In such a case, only a fraction of the total possible AIMs that can be indicative of the IE ancestral group will have been positive (if all were positive, the individual would have been 100% IE) and, therefore, only some of the individuals chromosomes or chromosomal regions will be of IndoEuropean origin. The chromosomes of the individual containing the positive AIMs for IE are then identified, and second hybridizing oligonucleotides specific for a second panel of AIMs are selected (e.g., from a group of 1000 or so AIMs that cover all 23 pairs of human chromosomes), wherein the AIMs of the second panel are limited to those that are highly variable in allele frequencies between IE ethnic groups and, therefore, indicative of IE ethnicity, and also are present on the chromosomes for which the first panel AIMs were IE positive. A sub-population structure that correlates with the nucleotide occurrences of the AIMs of the second panel is then identified, thus indicating an ethnicity with respect to the IE ancestral group of the test individual, for example, that the IE ancestral group derives from a Northern European, a Mediterranean, a Middle Eastern, or a South Asian Indian ethnicity. As such, the method provides a means to identify the ethnic origin of particular chromosomes (e.g., a Mediterranean origin of chromosomes previously determined to be of IndoEuropean origin) that contain AIMs that correlate with a population structure indicative of IndoEuropean BioGeographical Ancestry, and further contain AIMs that correlate more specifically with a sub-population structure indicative of Mediterranean ethnicity.

In another embodiment, the method of estimating proportional ancestry of a test individual can include generating an ancestral map of the world, wherein locations of populations having a proportional ancestry corresponding to the proportional ancestry of the test individual are indicated on the ancestral map. As such, the method can supplement genealogical information. For example, the method can further include overlaying the ancestral map with a genealogical map, wherein the genealogical map indicates locations of populations having geopolitical relevance with respect to the test individual, and statistically combining the information of the ancestral map and genealogical map to obtain a most likely estimate of family history of the test individual.

Identifying a population structure that correlates with, or is most likely given, the nucleotide occurrences of the AIMs, according to a method of the invention, can be performed by comparing the nucleotide occurrences of the AIMs of the test individual with known proportional ancestries corresponding to nucleotide occurrences of AIMs indicative of BGA. The known proportional ancestries corresponding to nucleotide occurrences of AIMs indicative of BGA can be contained in a table or other list, and the nucleotide occurrences of the test individual can be compared to the table or list visually, or can be contained in a database, and the comparison can be made electronically, for example, using a computer. Further, each of the known proportional ancestries corresponding to nucleotide occurrences of AIMs indicative of BGA can be associated with a photograph of a person from whom the known proportional ancestry was determined, thus providing a means to further infer physical characteristics of a test individual. In one aspect, the photograph is a digital photograph, which comprises digital information that can be contained in a database that can further contain a plurality of such digital information of digital photographs, each of which is associated with a known proportional ancestry corresponding to nucleotide occurrences of AIMs indicative of BGA of the person in the photographs.

In another aspect, a method of the invention can further include identifying a photograph of a person having a proportional ancestry corresponding to the proportional ancestry of the test individual. Such identifying can be done by manually looking through one or more files of photographs, wherein the photographs are organized, for example, according to the nucleotide occurrences of AIMs of the person in the photograph. Identifying the photograph also can be performed by scanning a database comprising a plurality of files, each file containing digital information corresponding to a digital photograph of a person having a known proportional ancestry, and identifying at least one photograph of a person having nucleotide occurrences of AIMs indicative of BGA that correspond to the nucleotide occurrences of AIMs indicative of BGA of the test individual.

Accordingly, the present invention also relates to an article of manufacture, which is at least one photograph of a person having a known proportional ancestry corresponding to a population structure comprising nucleotide occurrences of AIMs indicative of BGA, as well as to a plurality of such articles, each article of the plurality comprising one (or more) photograph(s) of a person having a known proportional ancestry corresponding to a population structure comprising nucleotide occurrences of AIMs indicative of BGA. The article can be contained in a file, or a plurality of the articles can be contained in a filed, for example, a file containing a plurality of photographs of different persons, wherein the some or all of the persons have the same or different known proportional ancestries that correspond to a population structure comprising nucleotide occurrences of AIMs indicative of BGA.

Accordingly, a plurality of such articles is provided, as is a plurality of files, each file of which can contain one or more articles, i.e., photographs, which can be of one or more persons having the same or different known proportional ancestries that correspond to a population structure comprising nucleotide occurrences of AIMs indicative of BGA. For example, different files of the plurality each can contain one (or more) photograph(s) of one person having a known proportional ancestry corresponding to a population structure comprising nucleotide occurrences of AIMs indicative of BGA. Different files of the plurality also can contain photographs of two or more different persons, each of whom has the same or substantially the same proportional ancestry corresponding to a population structure comprising nucleotide occurrences of AIMs indicative of BGA. As such, a plurality of files can contain files, each of which contains one or more photographs of one or more persons, and when containing one or more photographs of two or more different persons, the different persons can have the same or different known proportional ancestries.

In one embodiment, the article of manufacture, i.e., the photograph of a person having a known proportional ancestry corresponding to a population structure comprising nucleotide occurrences of AIMs indicative of BGA, is a digital photograph, which comprises digital information. As such, the digital information of the digital photograph, or of a plurality of digital photograph articles of manufacture of the invention can be contained in a database. As such, the present invention further provides a plurality of the articles of manufactures, including at least two digital photographs each of which comprises digital information. In one aspect of this embodiment, the digital information for one or a plurality of the articles is contained in a database, which can be contained in any medium suitable for containing such a database, including, for example, computer hardware or software, a magnetic tape, or a computer disc such as floppy disc, CD, or DVD. As such, the database can be accessed through a computer, which can contain the database therein, can accept a medium containing the database, or can access the database through a wired or wireless network, e.g., an intranet or internet.

The present invention also relates a kit, which contains a plurality of hybridizing oligonucleotides, each hybridizing oligonucleotide including at least fifteen contiguous nucleotides of a polynucleotide as set forth in SEQ ID NOS:1 to 331, or a polynucleotide complementary thereto, and the plurality including at least five of such oligonucleotides, each based on different polynucleotides as set forth in SEQ ID NOS:1 to 331. In one embodiment, the hybridizing oligonucleotides that include at least fifteen contiguous nucleotides of at least five polynucleotides as set forth in SEQ ID NOS:1 to 71, or polynucleotides complementary to any of SEQ ID NOS:1 to 71.

The hybridizing polynucleotides of a kit of the invention can include probes, which are useful for detecting a particular AIM, including a particular nucleotide occurrence at the SNP position or DIP (deletion/insertion polymorphism) position of the AIM; can include primers, including primers useful for a primer extension reaction and primer pairs useful for a nucleic acid amplification reaction; or can include combinations of such probes and primers. In one embodiment, a hybridizing oligonucleotide of the plurality includes a nucleotide corresponding to nucleotide position of the AIM (e.g., nucleotide 50 of any of SEQ ID NOS:1 to 34 and most others, nucleotide 56 of SEQ ID NO:35, nucleotide 44 of SEQ ID NO:50, or nucleotide 26 of SEQ ID NO:56), or to a nucleotide sequence complementary thereto, such a hybridizing oligonucleotide being useful as a probe to identify the presence or absence of a particular nucleotide occurrence at the SNP position of the AIM.

In another embodiment, the kit contains at least one pair of hybridizing oligonucleotides useful for detecting the nucleotide occurrence(s) at the SNP (or DIP) position of an AIM. In one aspect of this embodiment, a pair of hybridizing oligonucleotides includes one oligonucleotide that hybridizes upstream and adjacent to the SNP position of an AIM and a second oligonucleotide that hybridizes downstream of and adjacent to the SNP (or DIP) position of the AIM, wherein one or the other of the pair further contains a nucleotide complementary to a nucleotide occurrence suspected of being at the SNP (or DIP) position of the AIM (i.e., one of the polymorphic nucleotides), such a pair of hybridizing oligonucleotides being useful in an oligonucleotide ligation assay. In another aspect of this embodiment, a pair of hybridizing oligonucleotides includes an amplification primer pair, including a forward primer and a reverse primer, such a pair of hybridizing oligonucleotides being useful for amplifying a portion of polynucleotide that includes the SNP (or DIP) position of the AIM.

A kit of the invention can further contain additional reagents useful for practicing a method of the invention. As such, the kit can contain one or more polynucleotides comprising an AIM, including, for example, a polynucleotide containing an AIM for which a hybridizing oligonucleotide or pair of hybridizing oligonucleotides of the kit is designed to detect, such polynucleotide(s) being useful as controls. Further, hybridizing oligonucleotides of the kit can be detectably labeled, or the kit can contain reagents useful for detectably labeling one or more of the hybridizing oligonucleotides of the kit, including different detectable labels that can be used to differentially label the hybridizing oligonucleotides; such a kit can further include reagents for linking the label to hybridizing oligonucleotides, or for detecting the labeled oligonucleotide, or the like. A kit of the invention also can contain, for example, a polymerase, particularly where hybridizing oligonucleotides of the kit include primers or amplification primer pairs; or a ligase, where the kit contains hybridizing oligonucleotides useful for an oligonucleotide ligation assay. In addition, the kit can contain appropriate buffers, deoxyribonucleotide triphosphates, etc., depending, for example, on the particular hybridizing oligonucleotides contained in the kit and the purpose for which the kit is being provided.

The present invention relates to methods for performing multiplex reactions to analyze the nucleotide occurrences of single nucleotide polymorphisms (SNPs) as exemplified by ancestry informative markers (AIMs). Accordingly, the invention also relates to kits useful for performing such multiplex reactions. The compositions and methods of the invention are exemplified by twelve panels of polymerase chain reaction (PCR) primer pairs and a corresponding twelve panels of single base extension (SBE) primers useful for determining the nucleotide occurrences of 174 AIMs in twelve reactions. Also provided are SNPs useful as AIMs, including the SNPs set forth in SEQ ID NOS:371 to 398, 400 to 408, 410 to 413, 415, 418, 420, 422, 423, 425, 431 to 433, 438 to 441, 443, 450 to 452, 455, 456, 461 to 463, 467 to 475, 477 to 485, 487, 495 to 498, 502 to 504, 506, 508 to 512, 514, 516, 519 to 521, 526, 529, and 533 to 537, and to a method of inferring, with a predetermined level of confidence, a trait of an individual by detecting a nucleotide occurrence of a single nucleotide polymorphism as set forth in any of SEQ ID NOS:371 to 398, 400 to 408, 410 to 413, 415, 418, 420, 422, 423, 425, 431 to 433, 438 to 441, 443, 450 to 452, 455, 456, 461 to 463, 467 to 475, 477 to 485, 487, 495 to 498, 502 to 504, 506, 508 to 512, 514, 516, 519 to 521, 526, 529, and 533 to 537.

A panel of 174 AIMs useful for estimating proportional ancestry of an individual is set forth as SEQ ID NOS:364 to 537 (Appendix 3), and primers that selectively hybridize immediately 5′ to the SNP position of SEQ ID NOS:364 to 537, or a complement thereof, are exemplified, respectively, in Appendix 1. The exemplified primers are useful, for example, as templates to perform SBE reactions to determine the nucleotide occurrence at the SNP position (see Example [1]). As shown in Appendix 1, the primers are organized into Panels (e.g., Panel 3, Panel 41, etc) that can be used in a multiplex reaction to sample pluralities of AIMs in a single reaction (e.g., Panel 3 allows the sampling of 7 AIMs; Panel 41 allows the sampling of 10 AIMs, etc.). Primer pairs useful for amplifying gene sequences containing the 174 AIMs (SEQ ID NOS:364 to 537) are exemplified in Appendix 2 and, in correspondence with Appendix 1, are organized into Panels that can be used in multiplex reactions (see Example 1; see, also, Appendix 4, showing results of multiplex reactions).

Corresponding primers (Appendix 1), primer pairs (Appendix 2), and AIMs (Appendix 3) can be identified, for example, by the “SNP name”, which is shown in the second column in each of Appendices 1 and 2, and just above the SEQ ID NO: for each AIM in Appendix 3. It should be recognized that additional primer pairs useful, for example, for amplifying a gene sequence including a SNP position of an AIM as set forth in SEQ ID NOS:364 to 537 can be designed based on the exemplified sequences (Appendix 3), or on further gene sequences upstream or downstream of those shown in Appendix 3, which can be identified from databases using the disclosed sequences to perform a search. In this respect, an “N” or “n” is shown in some of the AIMs of SEQ ID NOS:364 to 537 to illustrate repetitive sequences that are not particularly useful for performing such searches or as targets for primers or primer pairs.

Accordingly, the present invention relates to a kit that contains one or more pluralities of primers useful for performing a multiplex reaction to analyze (e.g., by SBE) the nucleotide occurrences of SNPs in a sample. Generally, but not necessarily, the pluralities of primers are provided in the kit as a mixture. In one embodiment, a kit of the invention contains one or more of a plurality of 7 primers as set forth in Panel 3 of Appendix 1; a plurality of 10 primers as set forth in Panel 41 of Appendix 1; a plurality of 7 primers as set forth in Panel 42 of Appendix 1; a plurality of 9 primers as set forth in Panel 43 of Appendix 1; a plurality of 24 primers as set forth in Panel 4452 of Appendix 1; a plurality of 21 primers as set forth in Panel 4553 of Appendix 1; a plurality of 20 primers as set forth in Panel 4654 of Appendix 1; a plurality of 22 primers as set forth in Panel 4755 of Appendix 1; a plurality of 12 primers as set forth in Panel 48 of Appendix 1; a plurality of 18 primers as set forth in Panel 4957 of Appendix 1; a plurality of 15 primers as set forth in Panel 5051 of Appendix 1; or a plurality of 9 primers as set forth in Panel 56A of Appendix 1. As such, the kit, which contains at least a first plurality of primers, can include one plurality of primers, two separate (discrete) pluralities of primers, three separate pluralities of primers, and so on (i.e., 4, 5, 6, 7, 8, 9, 10, 11) up to all twelve pluralities of primers set forth as Panels 3, 41, 42, 43, 4452, 4553, 4654, 4755, 48, 49567, 5051, and 56A as set forth in Appendix 1

A kit of the invention can further contain one or more reagents for performing a primer extension reaction using a primer of the plurality as a substrate (e.g., an SBE reaction). The reagent(s) can include, for example, a polymerase (e.g., a DNA dependent DNA polymerase), one or more deoxyribonucleotide triphosphate(s) and/or deoxyribonucleotide triphosphate analog(s), which can be incorporated into a primer by a polymerase; and/or one or more polynucleotide(s) that contain an AIM for which a primer of the plurality is designed to detect, such polynucleotide(s) being useful as controls. In addition, the kit can contain reagents useful for detectably labeling one or the plurality of primers of the kit, including different detectable labels that can be used to differentially label the hybridizing oligonucleotides. In one embodiment, the kit contains one or more dideoxyribonucleotide triphosphate(s), including dideoxyadenosine, dideoxycytidine, dideoxyguanidine, and/or dideoxythymidine. In one aspect of this embodiment, the kit contains all four dideoxyribonucleotide triphosphates, wherein each of the dideoxyribonucleotide triphosphates is detectably labeled with a different label, or wherein the kit contains each of the detectable labels, which can be bound to the individual dideoxyribonucleotide triphosphates.

In another embodiment, a kit of the invention contains at least a first plurality of primer pairs, which can be used, for example, to generate amplification products that include SNPs of interest. As such, the kit can further contain one or more of comprising a plurality of 14 primer pairs as set forth in Panel 3 of Appendix 2; a plurality of 20 primer pairs as set forth in Panel 41 of Appendix 2; a plurality of 14 primer pairs as set forth in Panel 42 of Appendix 2; a plurality of 18 primer pairs as set forth in Panel 43 of Appendix 2; a plurality of 48 primer pairs as set forth in Panel 4452 of Appendix 2; a plurality of 42 primer pairs as set forth in Panel 4553 of Appendix 2; a plurality of 40 primer pairs as set forth in Panel 4654 of Appendix 2; a plurality of 44 primer pairs as set forth in Panel 4755 of Appendix 2; a plurality of 24 primer pairs as set forth in Panel 48 of Appendix 2; a plurality of 36 primer pairs as set forth in Panel 4957 of Appendix 2; a plurality of 30 primer pairs as set forth in Panel 5051 of Appendix 2; and/or a plurality of 9 primer pairs as set forth in Panel 56A of Appendix 2. In various aspects, the kit contains the first plurality of primer pairs; the first and a second plurality of primer pairs; the first, as second, and a third plurality of primer pairs; and so on up to all twelve pluralities of primer pairs as set forth in Appendix 3. Where a kit of the invention contains one or more pluralities of primer pairs, it can further contain reagents useful, for example, for generating amplification products using the primer pairs, particularly in a multiplex reaction (e.g., a temperature stable polymerase and/or one or more deoxyribonucleotide triphosphate(s) or analogs thereof, which can, but need not, be labeled).

In still another embodiment, a kit of the invention can contain at least one plurality of primers as set forth in Appendix 1 and at least one plurality of primer pairs as set forth in Appendix 2. In one aspect, the kit contains one or more pluralities of primer pairs that correspond to one or more pluralities of primers contained in the kit (e.g., the plurality of primers in Panel 3 of Appendix 1 and the plurality of primer pairs in Panel 3 of Appendix 2, such primer and primer pairs being useful to sample the AIMs set forth in SEQ ID NOS:364 to 370 of Appendix 3). Such a kit can further contain one or more reagents useful for performing a primer extension reaction (e.g., an SBE reaction) and/or an amplification reaction using primers of the plurality or primer pairs of the plurality, respectively, as a substrate.

The present invention also relates to a method of inferring, with a predetermined level of confidence, a trait of an individual. Such a method can be performed, for example, by contacting at least a first sample (i.e., 1, 2, 3, 4, or more sample(s)) that contain test nucleic acid molecules of the individual with at least a first plurality of primers as set forth in the panels of Appendix 1, under conditions suitable for SBE of the primers; and detecting at least one SBE product of a primer of the first (or other) plurality. As disclosed herein, such SBE products are informative of nucleotide occurrences of a SNPs of AIMs that are indicative of a population structure correlated with a trait and, therefore, allow an inference, with a predetermined level of confidence, of a trait of the individual. In one aspect, the method comprises examining 2 or more samples, which are different, using the same plurality of primers or different pluralities of primers. In another aspect, the method comprises examining 2 or more same samples (e.g., aliquots of a single sample) using the same plurality of primers, thus providing a means to obtain statistically improved results. In still another aspect, the method comprises examining 2 or more same samples (e.g., aliquots of a single sample) using different pluralities of primers. It another aspect, the method comprises examining 12 same samples (i.e., of a single individual), wherein one of each of the 12 pluralities of primers as set forth in Appendix 1 in included in one of each of the sample, thus allowing for a determination to made of 174 AIMs as set forth as SEQ ID NOs:364-537 in Appendix 3.

A method of the invention can be performed by generating SBE products that contain a detectable label, particularly a detectable label that is indicative of the single base (nucleotide) that has been extended onto (ligated to) a primer of a plurality. Detection of the SBE product(s) can be performed using any method that can distinguish the SBE products of a multiplex reaction, including, for example, an electrophoresis, mass spectrometry, or gel chromatography method. In one aspect, an electrophoresis method is utilized. In another aspect, capillary gel electrophoresis is utilized to detect SBE product(s) generated according to a method of the invention.

A method of the invention can utilize a genomic DNA sample of an individual, or can use a product generated from genomic DNA. Accordingly, in one embodiment, the test nucleic acid sample is a PCR product. In one aspect of this embodiment, prior to SBE, multiplex PCR is performed using at least a first plurality of primer pairs as set forth in a Panel of Appendix 2, wherein the plurality of primer pairs is from a Panel corresponding to a Panel containing a plurality of primers that will be used for the SBE multiplex reaction.

A trait for which an inference can be drawn according to the present methods can be any trait for which the exemplified AIMs (SEQ ID NOS:364 to 537) or other AIMs and/or SNPs that are examined are predictive. As such, the trait can be biogeographical ancestry (BGA), including, for example, proportional ancestry such as a proportion of a sub-Saharan African, Native American, IndoEuropean, or East Asian ancestral group, or a combination thereof to which the individual providing the test nucleic acid sample is a member. The trait for which an inference can be drawn also can be, for example, responsiveness of the individual to a drug such as a cancer chemotherapeutic agent, or a statin; or susceptibility to a disease, for example, a disease having an ethnic predisposition; or can be a pigmentation trait (e.g., eye color or natural hair color).

In one aspect, the method allows an inference to be drawn as to the proportional ancestry of at least two ancestral groups of the individual. The proportional ancestry estimated according to a method of the invention can be a proportion of any ancestral group, including, for example, a proportion of sub-Saharan African, Native American, IndoEuropean, East Asian, Middle Eastern, or Pacific Islander ancestral group, and generally is a combination of two or more of such ancestral groups. Thus, the proportional ancestry of an individual being examined can include proportions of sub-Saharan African and IndoEuropean ancestral groups (e.g., 80% sub-Saharan African and 20% IndoEuropean; or 60% sub-Saharan African, 20% IndoEuropean, and 20% of a third ancestral group); or can include proportions of Native American and IndoEuropean ancestral groups; East Asian and Native American ancestral groups; IndoEuropean and East Asian ancestral groups; and the like. Similarly, the proportional ancestry can include proportions of Native American, East Asian, and IndoEuropean ancestral groups; sub-Saharan African, Native American, and IndoEuropean ancestral groups; sub-Saharan African, Native American, and East Asian ancestral groups; and the like. Where the method is utilized to drawn an inference of proportional ancestry with respect to three or more ancestral groups of the individual, the method can further include generating a graphical representation of the comparison of the ancestral groups. For example, where three ancestral groups are determined, the graphical representation comprises a triangle with each ancestral group independently represented by a vertex of the triangle, and wherein the maximum likelihood value of proportional affiliation for an individual comprises a point within the triangle. In one aspect, the graphical representation can further include a confidence contour indicating a level of confidence associated with estimating the proportional ancestry.

In another embodiment, a method of inferring, with a predetermined level of confidence, a trait of an individual is performed by contacting each of 12 aliquots of a nucleic acid sample of test individual with one of each of the plurality of primer pairs as set forth in the Panels of Appendix 2, under conditions suitable for PCR of target nucleotide sequences in the nucleic acid sample, thereby generating 12 PCR amplification samples; depleting each of the 12 PCR amplification samples of single stranded primers of the plurality of primer pairs and deoxyribonucleotide triphosphates; thereafter contacting each of the 12 PCR amplification samples with one of each corresponding plurality of primers as set forth in the Panels of Appendix 1, and with dideoxyadenosine, dideoxycytidine, dideoxyguanidine, and dideoxythymidine, each of which comprises a different detectable label, under conditions suitable for SBE of the primers, thereby generating 12 SBE samples, each of which comprises a plurality of SBE products; and detecting SBE products in each of the 12 SBE samples, wherein the SBE products are informative of nucleotide occurrences of SNPs of AIMs indicative of a population structure correlated with a trait. In one aspect, detection of the SBE products is performed using capillary gel electrophoresis.

Such a method can further include identifying a population structure that correlates with, or is most likely, given the nucleotide occurrences of the AIMs. Such a method can be performed by comparing the nucleotide occurrences of the AIMs of the test individual with known proportional ancestries corresponding to nucleotide occurrences of AIMs indicative of BGA. The known proportional ancestries corresponding to nucleotide occurrences of AIMs indicative of BGA can be contained in a table or other list, and the nucleotide occurrences of the test individual can be compared to the table or list visually, or can be contained in a database, and the comparison can be made electronically, for example, using a computer. Further, each of the known proportional ancestries corresponding to nucleotide occurrences of AIMs indicative of BGA can be associated with a photograph of a person from whom the known proportional ancestry was determined, thus providing a means to further infer physical characteristics of a test individual. In one aspect, the photograph is a digital photograph, which comprises digital information that can be contained in a database that can further contain a plurality of such digital information of digital photographs, each of which is associated with a known proportional ancestry corresponding to nucleotide occurrences of AIMs indicative of BGA of the person in the photographs.

In another aspect, a method of the invention can further include identifying a photograph of a person having a proportional ancestry corresponding to the proportional ancestry of the test individual. Such identifying can be done by manually looking through one or more files of photographs, wherein the photographs are organized, for example, according to the nucleotide occurrences of AIMs of the person in the photograph. Identifying the photograph also can be performed by scanning a database comprising a plurality of files, each file containing digital information corresponding to a digital photograph of a person having a known proportional ancestry, and identifying at least one photograph of a person having nucleotide occurrences of AIMs indicative of BGA that correspond to the nucleotide occurrences of AIMs indicative of BGA of the test individual. Such a database can be contained in any medium suitable for containing a database of digital information, including, for example, computer hardware or software, a magnetic tape, or a computer disc such as floppy disc, CD, or DVD. As such, the database can be accessed through a computer, which can contain the database therein, can accept a medium containing the database, or can access the database through a wired or wireless network, e.g., an intranet or internet.

The present invention also relates to a method of inferring, with a predetermined level of confidence, a trait of an individual. Such a method can be performed, for example, by detecting the nucleotide occurrence of at least one SNP as set forth in any of SEQ ID NOS: 371 to 398, 400 to 408, 410 to 413, 415, 418, 420, 422, 423, 425, 431 to 433, 438 to 441, 443, 450 to 452, 455, 456, 461 to 463, 467 to 475, 477 to 485, 487, 495 to 498, 502 to 504, 506, 508 to 512, 514, 516, 519 to 521, 526, 529, and 533 to 537.

The invention also provides primers for detecting an AIM and pluralities of primers. In on embodiment, a plurality of primer pairs for detecting an AIM, is provide that contains at least 2 different primer pairs including a first primer having 15-50 contiguous nucleotides of a sequence set forth in SEQ ID NOs:364-537 and a second primer having 15-50 contiguous nucleotides of the complement of a sequence set forth in SEQ ID NOs:364-537. These primer pairs can be used to amplify a polynucleotide comprising a nucleotide occurrence of a single nucleotide polymorphism as set forth in any of SEQ ID NOs: 364-537.

The plurality of primer pairs can be sequences from a single panel such as panel 3: SEQ ID NOs:712-725; panel 41: SEQ ID NOs:726-745; panel 42: SEQ ID NOs:746-759; panel 43: SEQ ID NOs:760-777; panel 4452: SEQ ID NOs:777-825; panel 4553: SEQ ID NOs:826-867; panel 4654: SEQ ID NOs:868-907; panel 4755: SEQ ID NOs:908-951; panel 48: SEQ ID NOs:952-975; panel 4957: SEQ ID NOs:976-1011; panel 5051: SEQ ID s: 1012-1041; and panel 56A: SEQ ID NOs:1042-1059.

In another embodiment, the plurality of primer pairs contains all the primers of a panel, such as 7 primer pairs of panel 3; 10 primer pairs of panel 41; 7 primer pairs of panel 42; 9 primer pairs of panel 43; 24 primer pairs of panel 4452; 21 primer pairs of panel 4553; 20 primer pairs of panel 4654; 22 primer pairs of panel 4755; 12 primer pairs of panel 48; 18 primer pairs of panel 4957; 15 primer pairs of panel 5051; or 9 primer pairs of panel 56A.

In other embodiments, the plurality of primer pairs contains sequences from at least 2 panels, and may contain all the primer of the panels disclosed herein.

The invention also provides pluralities containing at least 2 different primers for detecting an AIM, where each primer contains 15-100 contiguous nucleotides of a sequence set forth in SEQ ID NOs:364-537, or 15-100 contiguous nucleotides of the complement of a sequence set forth in SEQ ID NOs:364-537, and the primers can be used to detect a nucleotide occurrence of a single nucleotide polymorphism as set forth in any of SEQ ID NOs: 364-537.

The plurality of primers can be from a single panel or can be all the primer of a single panel. In other embodiments of the invention, the plurality of primers can be from different panels and can be all the primer of all the panels disclosed herein.

Also provide are kits containing the plurality of primer and/or primer pairs of the invention. The kits can contain additional reagents for performing DNA amplification of primer extension reaction. such as polymerase, deoxyribonucleotide triphosphates, deoxyribonucleotide triphosphate analogs, dideoxyadenosine, dideoxycytidine, dideoxyguanidine, dideoxythymidine. In certain embodiments, the deoxyribonucleotide triphosphates or deoxyribonucleotide triphosphate analogs contain a detectable label.

### BRIEF DESCRIPTION OF THE DRAWINGS

FIG. 1 provides a diagram indicating the fashion in which chromosomal segments are shuffled by recombination over time in an admixed population. Initially, the parental populations have chromosomal segments that are continuous with respect to AIMs along the segment. In the first filial (F1) generation all persons have one complete chromosomal segment from each parental population. In the F2 generation, many more combinations are possible. The relative likelihood of the non-recombinant vs. the recombinant genotypes shown in F2 is dependent on the size of the chromosomal segment. Segments of the order of the size of human chromosomes will average several recombination events in a single meiosis (one recombination is equally likely every 50 cM of genetic distance). F3 shows an example of a likely genotype for a person with two parents from the F2 generation. F(N)×F1 diagrams a genotype of a person with one F(N) parent and one F1 parent; and F(N)×F2 diagrams a genotype of a person with one F(N) parent and one F2 parent.

FIGS. 2A and 2B show triangle graphs generated using the algorithm described in Example 6 (see, also, Table 12). NAM, Native American; AFR, sub-Saharan African; EUR, IndoEuropean.

FIG. 2A illustrates extension of a line from the NAM vertex to the opposite leg of the triangle, wherein the opposite leg represents 0% Native American ancestry. A circle is shown at the position of the estimated proportional ancestry (see FIG. 2B), with the hatch mark on the line indicated the percent of Native American ancestry (approximately 15%).

FIG. 2B shows additional lines drawn from the AFR and EUR vertices. The position on each line corresponding to the position of the circle represents the proportion of each respective ancestry; i.e., 15% Native American, 60% IndoEuropean, and 25% African.

FIG. 3 shows a triangle plot depicting one approach to illustrate the value and precision of individual ancestry estimates. Typical distributions of three populations are shown (European Americans: filled squares; African-Americans: open triangles; and an African/Native American population: open circles). Also shown is a single individual with likelihood intervals represented as concentric rings surrounding the point estimate (filled circle). Like a topological map, each concentric ring represents a decrease in the likelihood by 1 log unit (10 times less likely). In this example, the individual has a likelihood interval space that is symmetrical and circular. Interval spaces will take many shapes depending on the admixture proportions of the subject in question and the allele frequencies of the markers that have been typed.

FIG. 4 provides a triangular plot showing average admixture estimates for three African-American samples (filled circles: WASH-Washington D.C., AFCAR-AfroCaribbeans, and BOG-Bogalusa), a European-American sample (open circle: SCO-State College), and a Spanish-American sample (open diamond: SLV-San Luis Valley Colo.). In parenthesis is shown the average African (AFR), IndoEuropean (EUR), and Native American (NAM) genetic contribution to each sample.

FIGS. 5A and 5B show the genetic structure in US resident populations.

FIG. 5A shows the percentage of unlinked AIMs showing significant association. Expected values are based on a 5% significance level. Values for the Washington D.C. sample are based on 33 AIMs, for San Luis Valley Colo. on 19 AIMs, and for State College Pa. on 34 AIMs.

FIG. 5B shows the correlation between individual ancestry estimates based on independent subsets of informative markers. Average correlation is based on 100 replicates. The total number of markers is the same as for FIG. 5A. The corresponding p values are indicated at the bottom of the graph.

FIGS. 6A and 6B show the triangle plots for a father (FIG. 6A) and mother (FIG. 6B).

FIGS. 7A to 7C show the triangle plots for each of three children of the father and mother represented in FIG. 6.

FIG. 8 shows the distribution of AIMs in the genome (chrom. number, chromosome number).

FIGS. 9A and 9B demonstrate the robustness of BGA admixture proportion analysis using AIMs (see Example 2). The confidence (contour lines) of the maximum likelihood estimate (MLE; point) is predictably affected by the elimination of AIMs informative for a particular pair-wise comparison. The first contour line extending from the MLE defines the triangle plot space within which the likelihood is 2 times lower than that of the MLE, and the second contour line defines the space in which the likelihood is 5 times lower than the MLE.

FIG. 9A shows the MLE and confidence contours obtained using 71 AIMs; actual percentages are indicated.

FIG. 9B shows the results obtained after eliminating those AIMs used to obtain the results shown in FIG. 9A from the analysis that are informative for East Asian-Native American distinction. The MLE is relatively unaffected, and the confidence contours along the East Asian-Indo European (European) and Native American-European axes remain undistorted, but the confidence contours are distorted along the East Asian-Native American axis.

FIG. 10 shows the BGA admixture proportions determined for each of eight individuals of a family pedigree. Circles represent females, squares males and the BGA affiliation for each individual is shown as a fraction where the numerator represents IndoEuropean BGA and the denominator represents Native American BGA. None of the individuals harbored sub-Saharan African or East Asian BGA except as indicated by the asterisk (*), which indicates that the individual was determined to be of 4% East Asian BGA.

FIG. 11 shows a family tree demonstrating how a Chinese great grandparent in an otherwise IndoEuropean family tree can produce a grandchild with IndoEuropean/East Asian ancestry. The individuals that are 100% East Asian (Chinese) are shown with shading; the admixture results for the male (square) at the bottom of the pedigree (short arrow) are of interest. The grandparent indicated by the long arrow is about a 50%/50% East Asian/IndoEuropean mix, and her daughter, the subject's mother, is expected to be a 25%/75% East Asian/IndoEuropean mix (see Example 3).

FIG. 12 shows the distribution of all SNPs available for genotyping by chromosomal arm for a group of patients treated for elevated cholesterol levels.

FIG. 13 shows the distribution of SNPs in Caucasian individuals taking Lipitor™ (n=180) for whom response was known in terms of cholesterol (lip TC), low density lipoprotein (lip LDL), liver transaminase AST-SGOT (lip SGOT) and ALT-GPT (lip GPT) measurements. SNPs with delta values of significance (>0.20) among the various trait classes were selected. For example, in about 70% of patients, Lipitor™ causes a decrease in LDL. For any given SNP, the delta value (□) is the difference in minor allele frequency among those individuals for whom LDL decreased by at least 20% versus those for whom LDL did not change.

FIG. 14 shows a similar analysis as for FIG. 13, except that response is measured following treatment with Zocor™ (n=150), and only total cholesterol (zoc TC) and LDL (zoc LDL) were examined.

FIG. 15 shows a distribution of SNPs (□>0.11) among chromosome for 1,000 individuals of known eye color.

FIG. 16 shows a distribution of SNPs (□>0.11) among chromosome for 1,000 individuals of known hair eye color.

FIG. 17 is a diagram illustrating a protocol for AIM detection using a kit.

FIG. 18 shows instruments that can be used to carry out methods of the invention.

FIG. 19 A shows the output for analysis of Panel 3.

FIG. 19 B shows the output for analysis of Panel 41.

FIG. 19 C shows the output for analysis of Panel 42.

FIG. 19 D shows the output for analysis of Panel 43.

FIG. 19 E shows the output for analysis of Panel 4452.

FIG. 19 F shows the output for analysis of Panel 4453.

FIG. 19 G shows the output for analysis of Panel 4454.

FIG. 19 H shows the output for analysis of Panel 48.

FIG. 19 I shows the output for analysis of Panel 4957.

FIG. 19 J shows the output for analysis of Panel 5051.

FIG. 19 K shows the output for analysis of Panel 56a.

### DETAILED DESCRIPTION OF THE INVENTION

The present invention is based on the identification of ancestry informative markers (AIMs) useful for inferring a level of population structure of an individual, which, in turn, allows an inference as to various traits of the individual. Further, the AIMs of the present invention are demonstrated to correlate with a trait, regardless of whether the marker is in linkage disequilibrium with a gene or locus known to be involved in the trait. As such, the AIMs of the present invention are distinguishable from previously described markers, which only were considered useful if they were linked with a trait, i.e., if the marker was physically close to a gene known to be involved in the trait as characterized, for example, in having a low cross-over percentage with respect to gene (or locus) known to be involved in (or associated with) the trait. In contrast, there is no requirement that the markers (AIMs) useful in the present methods be in linkage disequilibrium with a gene/trait and, in fact, AIMs that are disclosed herein as correlating with a trait can be located on different chromosomes from each other and from a gene/locus known to be associated with the trait.

AIMs are genetic loci that show alleles with high frequency differences between populations. AIMs are exemplified herein generally by single nucleotide polymorphisms (SNPs; see, e.g., SEQ ID NO:1), as well as by deletion/insertion polymorphisms (DIPs; see, e.g., SEQ ID NO:363). As disclosed herein, AIMs can be used to estimate BioGeographical Ancestry (BGA) of an individual or collection of individuals at the population level (in terms of races), at the sub-population level (in terms of ethnicities), and at the micro-group level (in terms of familial lines within ethnic groups), as well as at a practical, phenotypically qualified level (e.g., cases and controls). Such ancestry estimates at the subgroup and individual level can be directly instructive regarding the genetics of phenotypes that are different qualitatively or in frequency between populations, including, for example, the likelihood that an individual will respond to a particular medication or the propensity of an individual to develop a disease. Ancestry estimates also can provide a compelling foundation for the use of Admixture Mapping (AM) methods to identify the genes underlying these traits.

As exemplified herein, a panel of 71 AIMs (SEQ ID NOS:1 to 71) was identified from an examination of over 800 candidate AIMs (see, also, SEQ ID NOS:72 to 331), and methods were developed to examine these AIMS as a means to obtain accurate estimates of proportional ancestry. The methods and markers of the invention have been validated in studies using skin pigmentation as a model phenotype (see, also, Intl. Publ. No. WO 02/097047 (PCT/US02/16789), which is incorporated herein by reference). Initial markers were genotyped in two population samples with primarily African ancestry, African Americans from Washington D.C. and an African Caribbean sample from England, and in a sample of European Americans from Pennsylvania (see Example 1). In the two African population samples, very strong correlations were observed between estimates of individual ancestry and skin pigmentation as measured by reflectometry (R2=0.21, p<0.0001 for the African-American sample and R2=0.16, p<0.0001 for the British African-Caribbean sample). These correlations confirmed the validity of the ancestry estimates and also indicated the high level of population structure related to admixture, which characterizes these populations and is detectable using other tests to identify genetic structure. These results demonstrate that an estimate of an individual's ancestry can be made based on a DNA analysis using a relatively small number of well defined genetic markers (AIMs).

The methods and genetic markers disclosed herein provide tools for several distinct purposes, including, for example, 1) for the estimation of ancestry proportions in individuals from their DNA; 2) for the estimation of genetic structure for the control of study designs commonly used for genetic research; 3) for the construction of physical profiles through the inference of characteristics related to ancestry, which may have implications in forensic investigations; 4) for the identification of disease predisposition, referred to as “Mapping by Ancestry Linkage Disequilibrium” (MALD); and 5) for predicting a significant portion of an individual patient's response to prescription and over-the-counter medications. As such, the present invention provides, for example, 1) statistical methods for the determination of ancestral proportions from genetic sequences within individuals and examples of use; 2) several hundred AIMs culled from the publicly available single nucleotide polymorphism (SNP) database and identified using statistical methods as useful for the determination of ancestral proportions within individuals or study groups; 3) several hundred AIMs that are demonstrated as useful for the determination of ancestral proportions within individuals or study groups; and 4) software programs that can be used for the determination of ancestral proportions within individuals or study groups.

Previously, efforts have been made to control the two sources of population structure, including sampling effects and natural human demography, which were believed to confound efforts to identify markers of genes associated with particular traits. However, as disclosed herein, population structure is reflective of human demography, and markers that correlate with a trait value are useful as reporters of structure that correlate with trait value (rather than markers in LD with phenotypically active loci), and, therefore, provide a valuable tool that enables accurate classification in a cost-effective and practical manner. Alleles associated with a trait due to population structure are not linked to phenotypically active loci, but are merely correlated with trait value because they are enriched for in branches of the human family tree for which the trait value is more common. As disclosed herein, the distribution of trait values among the various branches of the human family tree are such that accurate classification can be obtained only through an appreciation of that structure, rather than a full understanding of the biological mechanism of the trait, and, as a result, markers that were considered false positives when considered with respect to their use for identifying phenotypically active loci, in fact, can enable accurate classification analysis; i.e., they are true positives provided the structure from which they were derived is reflective of human demography rather than sampling effects. The present methods are based on correlation between markers and BGA, where BGA is itself on some level of complexity correlated with a trait value, not linkage or linkage disequilibrium.

Accordingly, the present invention provides a method of inferring, with a predetermined level of confidence, a trait of an individual. In one embodiment, a method of the invention is performed by contacting a nucleic acid sample of a test individual with hybridizing oligonucleotides that can detect nucleotide occurrences of single nucleotide polymorphisms (SNPs) of a panel of at least about ten AIMs; and identifying, with a predetermined level of confidence, a population structure that correlates with, or is most likely given, the nucleotide occurrences of the AIMs in the individual, wherein the population structure correlates with a trait. The panel of AIMs are selected on their delta value (see below) and, where relevant, based on the particular platform used to perform the method, and are indicative of a population structure correlated with the trait. AIMs are exemplified herein by the polynucleotides set forth as SEQ ID NOS:1 to 331, wherein the SNP position generally is at nucleotide position 50 (but see, e.g., SEQ ID NO:35, nucleotide 56; SEQ ID NO:51, position 48; SEQ ID NO:56, position 26).

A test individual for whom a trait is to be inferred can be any individual for whom it is desired to infer a trait, and generally is a human. However, the methods of the invention also can be used for inferring traits of other mammals, including, for example, domestic animals such as cats, dogs, or horses; farm animals such as cattle, sheep, pigs, or goats; or other animals. The trait to be examined can be any trait of interest, including, as exemplified herein, proportional ancestry (BGA); hair, skin or iris pigmentation; or drug responsiveness.

The methods of the invention are particularly useful because they allow for an inference to be made of a desired trait with a predetermined level of confidence. As used herein, reference to a “predetermined level of confidence” means that an inference or estimate of the invention is made using statistical methods that provide a confidence interval to be determined about a mean or a maximum likelihood value. In addition to determining the maximum likelihood value of within-individual or within-sample structure, other similarly likely values can also be determined and these can be combined to define the x-fold likelihood confidence intervals, where x is any number such as 2, 5 or 10. For example, all of the structure results corresponding to a likelihood value 10 times lower than the Maximum Likelihood Value can be plotted or listed to define the 10-fold likelihood confidence interval. As for any statistical test, an assay of the invention is designed such that performance of the test results in a value having a desired confidence level. As disclosed herein, a method of the invention can be performed such that the result has a predetermined level of confidence by varying the number of AIMs examined with respect to a trait. For example, use of a certain panel of ten AIMs will allow an inference to be made as to whether an individual has a particular trait, e.g., responsiveness to Lipitor™, with a certain level of confidence, whereas use of a panel of twenty AIMs, which can, but need not be partially overlapping with the panel of ten AIMs, will allow the same inference to be made, but with a higher level of confidence. Similarly, use of two panels of ten AIMs each can allow an inference to be made that an individual has, for example, 80% IndoEuropean ancestry and 20% East Asian ancestry (with an error, e.g., of +10%), whereas the use of two panels of twenty AIMs each can allow the same inference, but with an error, e.g., of +5%.

A sample useful for practicing a method of the invention can be any biological sample of a test individual that contains nucleic acid molecules, including portions of the gene sequences containing AIMs that are to be examined or, wherein the polymorphism of an AIM results in an amino acid change in an encoded polypeptide, any biological sample that contains the encoded polypeptides. As such, the sample can be a cell, tissue or organ sample, or can be a sample of a biological fluid such as semen, saliva, blood, cerebrospinal fluid, and the like.

A nucleic acid sample useful for practicing a method of the invention will depend, in part, on whether the SNPs to be identified are in coding regions or in non-coding regions. Where one or more SNPs is present in a non-coding region of a gene, the nucleic acid sample generally is a deoxyribonucleic acid (DNA) sample, particularly genomic DNA or an amplification product thereof. However, where the AIM is contained within a transcribed sequence, e.g., rDNA, microsatellite DNA, or heteronuclear ribonucleic acid (RNA), which includes unspliced mRNA precursor RNA molecules including non-coding RNA sequence, an RNA sample can be used and examined directly, or a cDNA or amplification product thereof can be examined according to the present methods. Where one or more SNPs is present in a coding region of a gene, the nucleic acid sample can be DNA or RNA, or products derived therefrom, for example, amplification products. Furthermore, while the methods of the invention are exemplified with respect to a nucleic acid sample, it will be recognized that particular SNPs, when present in coding regions of a gene, can result in polypeptides containing different amino acids at the positions corresponding to the SNPs due to non-degenerate codon changes. As such, in one aspect, the methods of the invention are practiced using a sample containing polypeptides of the subject.

A method of the invention is performed by contacting the sample and hybridizing oligonucleotides under conditions suitable for detecting the nucleotide occurrences of the AIMs of the individual by the hybridizing oligonucleotides. Further, in aspects of the methods of the invention, the sample can be contacted with second hybridizing oligonucleotides, for example, to determine a sub-population structure. It should be recognized that the term “second”, when used in reference to hybridizing oligonucleotides (or to a panel of AIMs), is used for convenience of discussion so as to allow a clear distinction, e.g., of steps for performing a method. In this respect, it should be further recognized that one or more hybridizing oligonucleotides used, e.g., to determine a population structure, also can be included among the second hybridizing oligonucleotides.

Conditions suitable for detecting the nucleotide occurrences of AIMs will vary depending on the sequences of the hybridizing oligonucleotides, including their length and complementarity, as well as on the particular assay being used and, for example, whether the assay is being performed as a multiplex assay. The hybridizing oligonucleotides, which are at least 15 nucleotides in length, can contain deoxyribonucleotides or ribonucleotides, which are linked together by a phosphodiester bond, and can be single stranded or double stranded, though they generally are used in a single stranded form. Such hybridizing oligonucleotides can be prepared using methods of chemical synthesis or by enzymatic methods such as by the polymerase chain reaction (PCR).

The hybridizing oligonucleotides, or other polynucleotides useful in a methods or contained in a kit of the invention also can contain nucleoside or nucleotide analogs, and can have a backbone bond other than a phosphodiester bond, such oligonucleotides providing certain advantages such as having increased stability or more desirable hybridization properties. Nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference). The covalent bond also can be any of numerous other bonds, including a thiodiester bond, a phosphorothioate bond, a peptide-like bond or any other bond known to those in the art as useful for linking nucleotides to produce synthetic oligonucleotides (see, for example, Tam et al., Nucl. Acids Res. 22:977-986, 1994; Ecker and Crooke, BioTechnology 13:351360, 1995, each of which is incorporated herein by reference). The incorporation of non-naturally occurring nucleotide analogs or bonds linking the nucleotides or analogs can be particularly useful where the oligonucleotide is to be exposed to an environment that can contain a nucleolytic activity, including, for example, a tissue culture medium or sample comprising a cell extract because the modified oligonucleotides can be less susceptible to degradation.

Generally, the hybridizing oligonucleotides useful for purposes of the present invention are at least about 15 bases in length, which is sufficient to permit the oligonucleotide to selectively hybridize to a target polynucleotide comprising the AIM, and can be at least about 18 nucleotides or 21 nucleotides or 25 nucleotides or more in length. Typically, the oligonucleotides of the invention will be 15-100 nucleotides in length. As used herein, a numerical range such as 15-50, includes the beginning number, the ending number and any number in between. Thus, 15-50 can be 15, 16, 17, 18 . . . 97, 98, 99 or 100.

The term “selective hybridization” or “selectively hybridize” refers to hybridization under moderately stringent or highly stringent physiological conditions, which can distinguish related nucleotide sequences from unrelated nucleotide sequences. In nucleic acid hybridization reactions, the conditions used to achieve a particular level of stringency are known to vary, depending on the nature of the nucleic acids being hybridized, including, for example, the length, degree of complementarity, nucleotide sequence composition (e.g., relative GC:AT content), and nucleic acid type, i.e., whether the oligonucleotide or the target nucleic acid sequence is DNA or RNA. An additional consideration is whether one of the nucleic acids is immobilized, for example, on a filter, bead, chip, or other solid matrix.

Methods for selecting appropriate stringency conditions can be determined empirically or estimated using various formulas, and are well known in the art (see, for example, Sambrook et al., supra, 1989). An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed. As such, final conditions will vary, depending on the particular hybridization reaction involved, and can be determined empirically. It should be recognized that a variety of conditions can be utilized to provide selective hybridization conditions. For example, when a multiplex assay is to be performed using a plurality of different hybridizing oligonucleotides specific for different AIMs of a panel, the conditions (as well as the AIMs/hybridizing oligonucleotides) can be selected such that selective hybridization occurs for all of the hybridizing oligonucleotides in the reaction.

In various embodiments, it can be useful to detectably label a polynucleotide or hybridizing oligonucleotide. Detectable labeling of a polynucleotide is well known in the art and includes, for example, the use of detectable labels such as chemiluminescent labels, radionuclides, enzymes, haptens such as digoxygenin and biotin, fluorophores, and unique oligonucleotide sequences. For example, PCR products can be performed, wherein one primer is biotinylated and the other primer contains digoxygenin. The amplification products can then be bound to a streptavidin plate, washed, reacted with an enzyme-conjugated antibody to digoxygenin, and developed with a chromogenic, fluorogenic, or chemiluminescent substrate for the enzyme. Alternatively, a radioactive method can be used to detect generated amplification products, for example, by including a radiolabeled deoxynucleoside triphosphate into the amplification reaction, then blotting the amplification products onto DEAE paper for detection. In addition, if one primer is biotinylated, then streptavidin-coated scintillation proximity assay plates can be used to measure the PCR products. Additional methods of detection can use a chemiluminescent label, for example, a lanthanide chelate such as used in the DELFIA® assay (Pall Corp.), a fluorescent label, or an electrochemiluminescent label such as ruthenium tris-bipyridyl (ORI-GEN).

Methods for detecting a nucleotide occurrence at a SNP or DIP position of an AIM can utilize one or more oligonucleotide probes or primers, including, for example, an amplification primer pair, that selectively hybridize to a target polynucleotide spanning the AIM. Oligonucleotide probes useful in practicing a method of the invention can include, for example, an oligonucleotide that is complementary to and spans a portion of the target polynucleotide, including the position of the SNP (or DIP), wherein the presence of a specific nucleotide at the position of the SNP is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe. A pair of probes that specifically hybridize upstream and adjacent and downstream and adjacent to the site of the SNP, wherein one of the probes includes a nucleotide complementary to a nucleotide occurrence of the SNP, also can be used in an oligonucleotide ligation assay, wherein the presence or absence of a ligation product is indicative of the nucleotide occurrence at the SNP site. An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP or DIP site can be useful, wherein the amplification product is examined to determine the nucleotide occurrence at the SNP site or to determine whether there is an insertion or a deletion at the DIP site.

Numerous methods are known for determining a nucleotide occurrence at a particular position in a polynucleotide (i.e., of a SNP or DIP). Such methods can utilize one or more oligonucleotide probes or primers, including, for example, an amplification primer pair, that selectively hybridize to a target polynucleotide, which contains one or more SNP positions. Hybridizing oligonucleotide useful in practicing a method of the invention can include, for example, an oligonucleotide that is complementary to and spans a portion of the target polynucleotide, including the position of the SNP or DIP (including whether the DIP has a deletion or insertion), wherein the presence of a specific nucleotide at the SNP site or the presence of a deletion or insertion at the DIP site is detected by the presence or absence of selective hybridization of the oligonucleotide probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe.

An oligonucleotide ligation assay also can be used to identify a nucleotide occurrence at a SNP site, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

A hybridizing oligonucleotide also can be useful as a primer, for example, for a primer extension reaction, wherein the product (or absence of a product) of the extension reaction is indicative of the nucleotide occurrence at a SNP site or an insertion or deletion at a DIP site. In addition, a primer pair useful for amplifying a portion of the target polynucleotide including the SNP or DIP site can be useful, wherein the amplification product is examined to determine the nucleotide occurrence at the SNP site or the presence of a deletion or an insertion at the DIP site. Particularly useful methods include those that are readily adaptable to a high throughput format, to a multiplex format, or to both.

Conditions that allow generation of an amplification product in a sample in which an amplification reaction is being performed are such that the reaction contains the necessary components for the amplification reaction to occur. Such conditions include, for example, appropriate buffer capacity and pH, salt concentration, metal ion concentration if necessary for the particular polymerase, appropriate temperatures that allow for selective hybridization of the primer or primer pair to the template target polynucleotide, as well as appropriate cycling of temperatures that permit polymerase activity and melting of a primer or primer extension or amplification product from the template or, where relevant, from forming a secondary structure such as a stem-loop structure. Such conditions and methods for selecting such conditions are routine and well known in the art (see, for example, Innis et al., “PCR Strategies” (Academic Press 1995); Ausubel et al., “Short Protocols in Molecular Biology” 4th Edition (John Wiley and Sons, 1999), each of which is incorporated herein by reference).

A primer extension or amplification product can be detected directly or indirectly and/or can be sequenced using various methods known in the art. Amplification products that span a SNP site can be sequenced using traditional sequence methodologies, including, for example, the dideoxy-mediated chain termination method (Sanger et al., J. Molec. Biol. 94:441, 1975; Prober et al. Science 238:336-340, 1987) or the chemical degradation method (Maxam et al., Proc. Natl. Acad. Sci. USA 74:560, 1977) to determine the nucleotide occurrence at the SNP loci.

The nucleotide occurrence at a SNP site also can be determined using a microsequencing method, wherein the identity of only a single nucleotide is determined at a predetermined site (U.S. Pat. No. 6,294,336). Microsequencing methods include the Genetic Bit Analysis method (WO 92/15712). Additional, primer-guided, nucleotide incorporation procedures for assaying polymorphic sites in DNA have also been described (Komher et al., Nucl. Acids. Res. 17:7779-7784, 1989; Sokolov, Nucl. Acids Res. 18:3671, 1990; Syvanen et al., Genomics 8:684-692, 1990; Prezan et al, Hum. Mutat. 1:159-164, 1992; Nyren et al., Anal. Biochem. 208:171-175, 1993). These methods differ from Genetic Bit™. Analysis in that they all rely on the incorporation of labeled deoxyribonucleotides to discriminate between bases at a polymorphic site. In such a format, the signal is proportional to the number of deoxyribonucleotides incorporated, and polymorphisms that occur in runs of the same nucleotide generate signals that are proportional to the length of the run (Syvanen et al. Amer. J. Hum. Genet. 52:46-59, 1993).

Another method for determining the nucleotide occurrence at a SNP position is described by Macevicz (U.S. Pat. No. 5,002,867), wherein a nucleic acid sequence is determined via hybridization with multiple mixtures of oligonucleotide probes. In accordance with such a method, the sequence of a target polynucleotide is determined by permitting the target to sequentially hybridize with sets of probes having an invariant nucleotide at one position, and a variant nucleotides at other positions. The nucleotide sequence is determined by hybridizing the target with a set of probes, then determining the number of sites that at least one member of the set is capable of hybridizing to the target (i.e., the number of matches). This procedure is repeated until each member of a sets of probes has been tested. U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

The nucleotide occurrence of a SNP in a sample also can be determined using the SNP-IT™ method (Orchid BioSciences, Inc., Princeton, N.J.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide trisphosphate at the target SNP site, which provides a second level of specificity. In a third step, the extended nucleotide trisphosphate can be detected using a variety of known formats, including: direct fluorescence, indirect fluorescence, an indirect calorimetric assay, mass spectrometry, fluorescence polarization, etc. Reactions can be processed in 384 well format in an automated format using a SNPstream™ instrument (Orchid BioSciences, Inc., Princeton, N.J.). Phase known data can be generated by inputting phase unknown raw data from the SNPstream™ instrument into the Stephens and Donnelly's PHASE program.

Melting curve analysis of SNPs (McSNP® analysis) provides another method for detecting a nucleotide occurrence in an AIM (Akey et al., supra, 2001). McSNP® analysis provides the additional advantages that it does not require a step of gel electrophoresis, thus minimizing the time and cost for detecting a SNP, and that it is readily adaptable to high throughput formats, thus allowing examination of one or more panels of AIMs and/or samples in parallel.

Where the particular nucleotide occurrence of a SNP is such that the nucleotide occurrence results in an amino acid change in an encoded polypeptide, the nucleotide occurrence can be identified indirectly by detecting the particular amino acid in the polypeptide. The method for determining the amino acid will depend, for example, on the structure of the polypeptide or on the position of the amino acid in the polypeptide. Where the polypeptide contains only a single occurrence of an amino acid encoded by the particular SNP, the polypeptide can be examined for the presence or absence of the amino acid. For example, where the amino acid is at or near the amino terminus or the carboxy terminus of the polypeptide, simple sequencing of the terminal amino acids can be performed. Alternatively, the polypeptide can be treated with one or more enzymes and a peptide fragment containing the amino acid position of interest can be examined, for example, by sequencing the peptide, or by detecting a particular migration of the peptide following electrophoresis. Where the particular amino acid comprises an epitope of the polypeptide, the specific binding, or absence thereof, of an antibody specific for the epitope can be detected. Other methods for detecting a particular amino acid in a polypeptide or peptide fragment thereof are well known and can be selected based, for example, on convenience or availability of equipment such as a mass spectrometer, capillary electrophoresis system, magnetic resonance imaging equipment, and the like.

In another embodiment, a method of the invention utilizes an antibody, or antigen binding fragment thereof, that specifically binds, for example, to a polypeptide comprising an amino acid encoded by a nucleotide sequence comprising one nucleotide occurrence of a SNP, but not substantially to a polypeptide comprising an different amino acid encoded by the codon comprising the SNP; or that specifically binds, for example, to a polypeptide comprising an amino acid sequence encoded by one form a DIP (e.g., that having the insertion), but not substantially to that encoded by the alternative form (e.g., that having the deletion). As used herein, the term “specific interaction,” or “specifically binds” means that two molecules form a complex that is relatively stable under physiologic conditions. The term is used herein to refer to various interactions, including, for example, the interaction of an antibody that binds a target polynucleotide including the SNP site only if the SNP has a specified, but not an alternative, nucleotide occurrence (e.g, an A, but not a T); or the interaction of an antibody that binds a polypeptide that includes one amino acid that is encoded by a codon that includes a SNP site, but not a polypeptide having an alternative amino acid encoded by the codon comprising the SNP.

A specific interaction can be characterized by a dissociation constant of at least about 1×10-6 M, generally at least about 1×10-7 M, usually at least about 1×10-8 M, and particularly at least about 1×10-9 M or 1×10-10 M or greater. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

Antibodies useful in a method of the invention include antibodies that specifically bind polynucleotides that encompass an AIM, or that bind polypeptides that include an amino acid encoded by a codon that includes a SNP or that include amino acids due to an insertion at a DIP site. Such antibodies are selected such that they specifically bind a polypeptide that includes a first amino acid encoded by a codon that includes the SNP loci, but do not bind, or bind measurably more weakly to a polypeptide that includes a second amino acid encoded by a codon that includes a different nucleotide occurrence at the SNP.

The term “antibody” is used broadly herein to refer to immunoglobulin molecules and antigen binding portions of immunoglobulin molecules that specifically bind an antigen. As such, antibodies useful in a method of the invention can be polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies, and the like, as well as antigen/epitope binding fragments of such antibodies. Antigen binding fragments of antibodies include, but are not limited to, Fab, Fab′ and F(ab′)2, Fd, single-chain Fv's (scFv), single-chain antibodies, disulfide-linked Fv fragments (sdFv) and fragments comprising either a VL or VH domain. Thus, antigen-binding antibody fragments, including single-chain antibodies, can comprise the variable region(s) alone or in combination with the entirety or a portion of the hinge region, CH1, CH2, and/or CH3 domains. The antibodies can be from any animal origin including birds and mammals, or can be expressed recombinantly, for example, in insect or mammalian host cells or in plants.

There is much that can be learned today through the use of genetic markers in numerous scientific fields. The use of genetic sequences has become routine for forensics and disease research, but the majority of the benefits from the recently completed human genome project still await discovery. Within the genome exist sequences and patterns of sequences that will prove useful for a variety of purposes including increasing crop yields, extending human life spans, minimizing the suffering caused by drugs and enhancing the quality of our lives through better, more effective and specific treatments. Until now, biomedical research has been conducted on relatively simple terms. Nevertheless, more one thousand simple Mendelian traits have been mapped by following the transmission of genetic markers in families.

Many statistical methods are available for studying genetic traits, including traditional family-based linkage analysis, variance component methods, sib-pair linkage, measured genotype, transmission disequilibrium, genomic control, and structure analysis. Some of the genes underlying variation in susceptibility to common diseases (e.g., heart disease, obesity, type 2 diabetes, hypertension, and cancer) eventually will be identified using genetic approaches. However, there are a number of complexities in genetic research on common diseases because many of these conditions are multifactorial (i.e., have several sources of variability in risk) and polygenic (i.e., result due to the actions and interactions among several genes). Additional difficulties in the study of common diseases can derive from the late onset of symptoms and heterogeneity in etiology. Thus, identifying the genes involved in complex diseases remains one of the greatest challenges in the field of human genetics.

There has been increased interest in association studies as a useful approach to map common disease and drug response genes (Risch and Merikangas, Science 273:1516-1517, 1996; Jorde, Genome Res. 10:1435-1444, 2000; Nordborg and Tavaré, Trends Genet. 18:83-90, 2002). Until the present disclosure, however, the implication of ancestry for identifying these genes has not been fully appreciated. As such, the methods of the invention provide a previously undescribed platform for the identification of genes associated with disease susceptibility and drug responsiveness, as well as for the development of advanced forensic methods. As such, compositions and methods are provided for inferring an individual's response to commonly used medications, which, remarkably, is a function of individual ancestry; the disclosed markers and methods are, to a differing extent for each drug, useful for the inference of such response. In addition, compositions and methods are provided for inferring individual and/or group ancestral proportions from knowledge of the individual's or group's DNA sequences. Further, compositions and methods are provided for using knowledge of ancestry relevant DNA sequences to identify disease susceptibility and drug response genes through the MALD process. Also, compositions and methods are provided for qualifying and normalizing study groups for more traditional methods of mapping disease genes. Each of these processes requires an accurate knowledge of ancestry, which can be determined using the methods and compositions disclosed herein.

The populations that will be best suited for linkage disequilibrium (LD) mapping has prompted much discussion and debate (see Wright et al., Nat. Genet. 23:397-404, 1999; Eaves et al., Nat. Genet 25:320-323, 2000; Nordborg and Tavaré, supra, 2002; Kaessmann et al., Amer. J. Hum. Genet. 70:673-685, 2002). The extent of LD is a complex function of a number of genetic and evolutionary factors such as mutation, recombination and gene conversion rates, demographic and selective events, and the age of the mutation itself. Some of these factors affect the whole genome, while others only affect particular genome regions. Additionally, variation in mutation, recombination, and gene conversion rates throughout the genome are expected to create LD differences between genomic regions (see, for example, Taillon-Miller et al., Nat. Genet. 25:324-328, 2000).

It has been proposed that small, isolated and inbred populations will have advantages over other populations, due to the lower heterogeneity and the larger extent of linkage disequilibrium (Wright et al., supra, 1999; Nordborg and Tavaré, supra, 2002; Kaessmann et al., supra, 2002). Other populations well suited for mapping are recently admixed populations (e.g., Hispanics and African Americans), which offer the advantage that LD has been created recently due to the admixture process. Because this LD is recent, it can extend over large chromosomal regions. However, it is also extremely important to control for the genetic structure (inter-individual variation in admixture proportions) present in these populations in order to avoid false positives (Parra et al., supra, 1998; Lautenberger et al., Amer. J. Hum. Genet. 66:969-978, 2000; Pfaff et al., Amer. J. Hum. Genet. 68:198-207, 2001; Nordborg and Tavaré, supra, 2002, each of which is incorporated herein by reference). Interest in admixture mapping has increased in recent years (McKeigue et al., Ann. Hum. Genet. 64:171-186, 2000; Smith et al., J. Invest. Dermatol. 111: 119-122, 2001; Collins-Schramm et al., Amer. J. Hum. Genet. 70:737-750, 2002, each of which is incorporated herein by reference). A general description of admixture mapping is provided below, as are some details about a statistical approach developed for admixture mapping and its application to skin pigmentation as a model phenotype.

Admixture generates allelic associations between all marker loci where allele frequencies are different between the parental populations (Chakraborty and Weiss, Proc. Natl. Acad. Sci., USA 85:9119-9123, 1988). These associations decay with time in a way that is dependent on the genetic distance between them. Thus, disease (or trait) risk alleles that are different between the parental populations can be mapped in admixed populations using special panels of genetic markers showing high frequency differences between the parental populations. These markers, termed AIMs, are characterized by having particular alleles that are more common in one group of populations than in other populations. One measure of the informativeness of such markers is the allele frequency differential, delta (δ), which is simply the absolute value of the difference of a particular allele between populations (Chakraborty and Weiss, supra, 1988; Dean et al., supra, 1994).

In admixed populations, allelic associations were generated recently and, therefore, are more easily detected for a given sample size because they extend over longer distances than in non-admixed populations (up to 10-20 centiMorgans (cM) or more). The statistical basis of this approach was first explored by Chakraborty and Weiss (supra, 1988) and subsequently by Stephens, Briscoe and O'Brien, who named the method “mapping by admixture linkage disequilibrium” (MALD; Stephens et al., Amer. J. Hum. Genet. 55:809-824, 1994; Briscoe et al., J. Hered. 85:59-63, 1994). Further, whether one is using a MALD approach or a more traditional LD approach for genetic research, to eliminate associations of the trait with alleles at unlinked loci, it is necessary to control in the analysis for individual ancestry estimated from the marker data. The SNP sequences (markers; AIMs) and methods disclosed herein (BGA test) are a particularly efficient means by which to accomplish this task. An Analysis of Covariance (ANCOVA) test has been employed using the estimate of individual admixture as a conditioning variable to control for the effect of individual ancestry in two ways: 1) leaving out the locus under consideration (ANCOVA/IAE minus marker); and 2) using the complete individual ancestry estimate for the conditioning (ANCOVA/IAE). This method is described in detail herein.

An alternative approach to exploiting admixture has been developed that, while based on earlier work, has little in common with classical LD mapping, and is more analogous to linkage analysis of an experimental cross (McKeigue, Amer. J. Hum. Genet. 63:241-251, 1998, which is incorporated herein by reference; McKeigue et al., supra, 2000). For this reason, the term “admixture mapping” has been proposed as more appropriate than “mapping by admixture linkage disequilibrium”. Instead of testing for allelic associations, according to the present methods, the underlying variation in ancestry is modeled on chromosomes of mixed descent to extract all the information about linkage that is generated by admixture. The methods and markers disclosed are necessary and sufficient to accomplish this process. Advanced statistical methods are utilized to apply this approach in practice, though the underlying principle on which it relies to detect linkage is straightforward. Suppose, for instance, that a locus accounts for some of the variation in pigmentation between West Africans and Europeans. If individuals of mixed descent are classified according to whether they have 0, 1 or 2 alleles of African ancestry at this locus, then in a comparison of these three groups with other factors held constant, the mean pigmentation level will vary with the proportion of alleles at the locus that are of African ancestry. Controlling the analysis for parental admixture eliminates association of the trait with ancestry at unlinked loci and ensures that the comparison is made with other factors held constant.

To infer the ancestry of the alleles at the locus from the marker genotype, the conditional probability of each allelic state is required given the ancestry of the allele (ancestry specific allele frequencies), e.g., West African or European. There is growing evidence that admixture mapping will be an effective means of gene identification, and it has been reported that, in admixed populations, strong allelic association is observed between linked markers spaced at substantial distances (Parra et al., supra, 1998; Parra et al., Amer. J. Phys. Anthropol. 114:18-29, 2001; McKeigue et al., supra, 2000; Lautenberger et al., supra, 2000; Smith et al., supra, 2001; Wilson and Goldstein, Amer. J. Hum. Genet. 67:926-935, 2000; Pfaff et al., supra, 2001). Given the very high levels of association observed over long genetic distances, it is expected that phenotypes different between parental populations because of some genetic factor will also show associations with linked. AIMs. A phenotype that is well suited to apply admixture mapping is skin pigmentation.

Notwithstanding the power of AIMs for disease gene and forensics analysis, no studies have been conducted to elucidate this power. As disclosed herein, 1) SNPs or deletion/insertion polymorphisms (collectively referred to as AIMs) in the human genome that are of potential use for drug response, disease gene or forensics research were identified; 2) biochemical and genetic test results are provided that demonstrate these AIMs can be useful for disease gene and forensics research; 3) the usefulness of AIMs derived from systematic screens of the human genome in actual drug response, disease gene or forensics research is demonstrated; 4) the usefulness of AIMs derived from systematic screens of the human genome to make an inference as to whether an individual is susceptible to acquire a disease, or to not respond to a drug, is demonstrated; 5) the usefulness of AIMs derived from systematic screens of the human genome to make an inference as to whether a crime scene DNA specimen was derived from an individual of, for example, an 80% European, 10% African and 10% Asian heritage or some other ratio/mix is demonstrated; 6) the usefulness of AIMs derived from systematic screens of the human genome to infer the ancestral proportions of an individual from their DNA (e.g., whether the individual is of 80% European, 10% African and 10% Asian heritage, or some other ratio/mix) is demonstrated; and 7) the usefulness of AIMs derived from systematic screens of the human genome to infer the ancestral proportions of a group of individuals from their DNA (for example, whether the group, which can be a population sample, a family, or a clinically defined group of persons, is of 80% European, 10% African and 10% Asian heritage, or some other ratio/mix) is demonstrated.

The present results demonstrate that AIMs are useful for the applications described above, and the sequences exemplified herein, as well as additional AIMs identified using the methods disclosed herein, enable these applications. The AIMs and methods of the invention are useful for the study of human diseases, drug response, and physical traits and, therefore, provide exceptional commercial potential. For example, in this dawning era of personalized drug prescription and disease risk assessment, the markers and methods of the invention provide the tools needed to proceed in this fledgling industry. As exemplified herein, an individual's response to a particular medication was dependent on the degree to which that individual exhibited a certain population structure (i.e., was of certain ancestral heritage) in addition to, but irrespective of, the person's genotype for drug target or xenobiotic metabolism gene sequences. As such, the compositions and methods of the invention provide a means to predict an individual's likelihood to respond to a particular drug.

For example, in screen of genetic markers associated with patient response to the cholesterol lowering drug, Lipitor™, in terms of low-density lipoprotein (LDL) response, which is an indicator of favorable response, some of the most powerful markers identified for LDL response to Lipitor™ were gene types that are not immediately recognized as relevant for drug response, including, for example, TYR, OCA2, TYRP, FDPS, and HMGCR (see, also, Intl. Publ. No. WO 03/002721 (PCT/US02/20847), and Intl. Publ. No. WO 03/045227 (PCT/US02/38345), each of which is incorporated herein by reference). When combined with markers from genes that are biologically relevant for response, they augment the ability to make accurate inferences of response from the DNA. Each of these markers also is an excellent AIM, indicating that the linkage of the AIMs to drug response is likely a function of ancestral differences in response proclivity (see Example 5). As such, ancestral heritage can be predictive of favorable response to Lipitor™. This association has been observed for almost every type of response (n=54) to almost every type of drug (n=23) examined, thus confirming that the inference of drug response can be accomplished, at least in part, through the inference of ancestral proportions. As such, it appears that the genes truly relevant for drug response are a function, at least in part, of individual ancestry, and that the gene sequences relevant for drug response are statistically linked with markers that are informative as to ancestry (i.e., AIMs).

Screening genomes for the true identity of genes associated with a particular trait such as drug responsiveness is extraordinarily expensive and time consuming. As such, the use of AIMs for making inferences about individual proclivity to drugs provides a significant short-cut for the rapid development of tests that can be used to match patients with those drugs most appropriate for their genetic constitution. Thus, in addition to being useful for the admixture mapping of disease genes, the disclosed methods and exemplified markers provide tools that can direct treatment protocols by clinicians. The identification of AIMs from publicly available human genome data, and the ability to effectively use the AIMs for the development of patient-drug classification sets, admixture screening panels and forensics tools, was accomplished using the disclosed method, including screening the SNP database (see, for example, world wide web (“www”) at URL “nih.ncbi.nlm.gov”) for AIMs; screening the AIMs against a multi-ancestral panel of DNA samples to verify those that, indeed, are good AIMs; using the disclosed statistical and software methods for using the AIM sequences to make biologically relevant inferences; and recognizing that an individual's likelihood to respond to a drug or develop a disease can be predicted through a knowledge of their ancestry, which, in turn, is indicated through the individual's AIM sequences.

Prior to the present disclosure, individual ancestry could be estimated using two independent methods: a Maximum Likelihood approach (Hanis et al, Amer. J. Phys. Anthropol. 70(4):433-441, 1986, which is incorporated herein by reference), and a Bayesian method implemented in the STRUCTURE program (Pritchard et al., Genetics 155:945-959, 2000, which is incorporated herein by reference). While the Maximum Likelihood method and the Bayesian method provide point estimates of proportional ancestry or admixture, there are several deficits in these methods that are addressed by the disclosed methods. For example, using the disclosed algorithm (see Example 6; see, also, Table 12, containing flow chart exemplifying algorithm), 1) the most likely group(s) from which the individual is derived were estimated simultaneously with the estimate of proportional ancestry; 2) multidimensional confidence intervals were computed and projected, thus reducing the complexity for presentation; 3) an approach to estimate the number of ancestors and their admixture proportions at each level in the past (parental, grandparental, great-grandparental, etc.) was developed; and 4) proportional BGA affiliation within individuals for more than two BGA groups at a time was derived, thus providing, for example, improved and more accurate forensics applications, as well as allowing for the development of classifiers for quantitative or continuously distributed traits (i.e., not dichotomous), the trait values of which are at least in part a function of BGA.

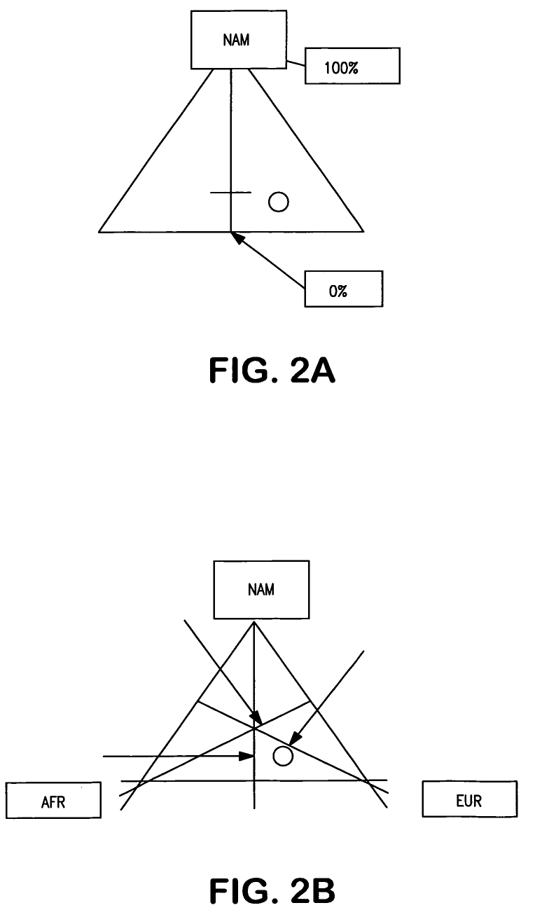

Independent methods for classification of individuals into population groups have been developed (Shriver et al., supra, 1997, Frudakis et al., supra, 2002, each of which is incorporated herein by reference). The present methods differ from previous classification methods in that they allow the simultaneous estimation of the best group within which a particular individual would fall, as well as the proportional assignment of the individual to multiple parental groups (Example 6; see, also, Table 12). Thus, where previous methods allowed one to make the statement that a person is much more likely to be African-American than European-American, the present approach allow the same statement, and also provides the proportional ancestry of the individual with confidence intervals (CI); e.g., 25% (95% CI 15-35%); European ancestry; 75% (95% CI 60-80%) African ancestry; and 0% (95% CI 0-6%) Native American ancestry. Further, the confidence intervals can be expressed in multidimensional space to provide a clearer representation of the ancestry measured for the person in question (see below; see, also, FIG. 2). Though methods for constructing such a representation were known, the present disclosure is the first to provide for the representations to be presented with quantifiable confidence.

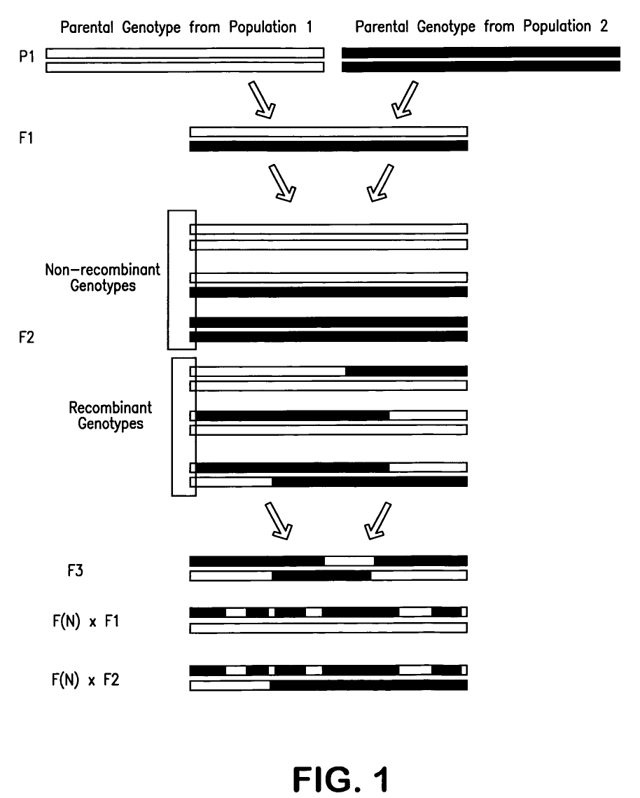

There are clear differences in the patterns of chromosomal segment ancestry (PCSA) among persons with different ancestral histories (see FIG. 1). A series of AIMs across the chromosomes can facilitate the estimation of the most likely parental combinations that lead to the profile of sequences observed in a given person. One example of where estimates of PCSA is important is in the discrimination of persons of Hispanic ancestry from those having primarily European ancestry with some proportion of recent Native American ancestry. Indeed, this is an important determination as the political and legal rights claimed by and provided to these two groups can depend on their ancestry. Hispanic populations such as Mexican-Americans (MA) have approximately 30-40% Native American ancestry, while the balance is European with a minor portion (5% or so) African ancestry. A person who is one quarter Native American will have 25% Native American ancestry and, therefore, will overlap with many MA persons in his level of estimated ancestry. It is expected that PCSA patterns will be significantly different for these two cases and may provide some of the only genetic evidence that would facilitate an accurate definition of the ancestry in such a case. As disclosed herein, PCSA can be used in ancestry studies.

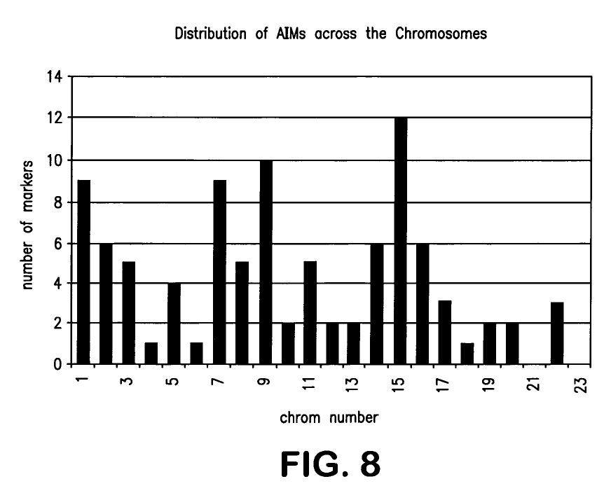

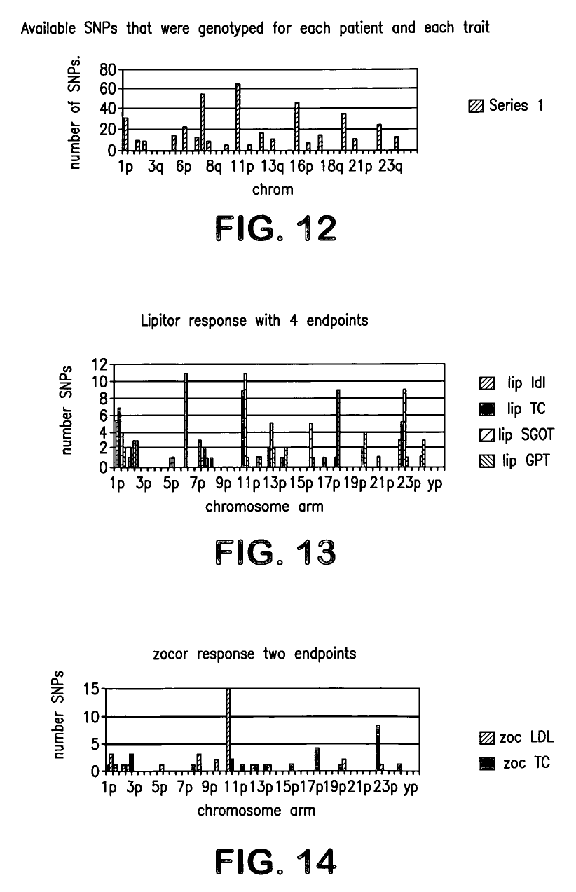

An important step in these determinations is the phasing of the AIMs along chromosomal segments (see Example 2, FIG. 8; see, also Example 5, FIGS. 12 to 16). Phasing AIMs along the chromosome can be accomplished by several methods, including 1) estimation from the genotypes of the individual, 2) molecular haplotyping (e.g., allele specific PCR combined with genotyping), and 3) single sperm analysis (for female subjects the sperm of a male full sibling would provide the same profile). In addition, the disclosed methods allow simultaneous consideration of the two sex chromosomes (X and Y) and the mtDNA for ancestral inferences. AIMs are found on each of these sources, and can be informative for many of the questions regarding the ancestral proportions of a person and the population(s) from which a particular person is derived. For example, Hispanic/Latino populations have very high (65-100%) frequencies of Native American mtDNA haplogroups, while showing only a minority contribution from Native American populations in autosomal markers. Thus, for example, a person with reputed Native American ancestry on her father's side, with a non-Native American mtDNA haplogroup, is more likely not Hispanic than partially Native American as she may suspect, than were she to have a Native American mtDNA haplogroup.

Linkage disequilibrium (LD) is increasingly being used as a mapping tool for both fine-scale determination of gene position and for the initial localization of disease genes in special populations. Allelic associations are significantly non-random and correlated with physical distance within small (<60 kb) genomic regions (see Jorde, Amer. J. Hum. Genet. 66:979-988, 1995; Jorde, Genome Res. 10:1435-1444, 2000, for review), possibly reflecting an underlying “block structure” that characterizes many genomic regions (Reich et al., 2001; Daly et al., 2001). Thus, if disease alleles in a population share a recent common origin, nearby genetic markers with the strongest associations will be closest to the disease-causing locus. This approach has been important in the positional cloning of several simple Mendelian diseases, including the cystic fibrosis gene, the Huntington's disease gene, and the diastrophic dysplasia gene.

In addition to applications in fine-mapping or positional cloning, LD can be used for initial disease gene mapping in homogeneous populations that have undergone recent increases in size or are genetically inbred. In such populations, disease alleles were probably present in a small number of founders, and recombination has had limited opportunity to randomize associations between these alleles and alleles at linked marker loci. An analysis of allelic associations between affected and unaffected individuals from these populations can thus facilitate the localization of the disease locus. A number of Mendelian diseases have been mapped using this approach: several diseases in the Finish population, Hischsprung's disease in Mennonites, benign recurrent intrahepatic cholestasis in an isolated Dutch fishing community, familial persistent hyperinsulinemic hypoglycemia of infancy in a consanguineous group of Saudi Arabian families, and Bardet-Biedl syndrome in Bedouins.

There has been much debate as to which populations will be best suited for LD mapping of complex polygenic diseases (see, e.g., Wright et al., supra, 1999; Eaves et al., supra, 2000; Nordborg and Tavaré, supra, 2002; Kaessmann et al., supra, 2002). The extent of LD is a complex function of a number of genetic and evolutionary factors such as mutation, recombination and gene conversion rates, demographic and selective events, and the age of the mutation itself. Some of these factors affect the whole genome while others only affect particular genome regions. Additionally, variation of mutation, recombination and gene conversion rates throughout the genome is expected to create LD differences between genome regions.

With regard to the populations to use in disease discovery efforts driven by LD-based methods, it has been proposed that small, isolated and inbred populations will have advantages over other populations, due to the lower heterogeneity and the larger extent of linkage disequilibrium (see, e.g., Wright et al., supra, 1999; Nordborg and Tavaré, supra, 2002; Kaessmann et al., supra, 2002). On the other side, admixed populations such as Hispanics and African Americans offer the advantage that linkage disequilibrium has been created recently due to the admixture process, and it can extend over large chromosomal regions, although it is extremely important to control for the genetic structure present in these populations in order to avoid false positives (Parra et al., supra, 1998; Lautenberger et al., supra, 2000; Pfaff et al., supra, 2001; Nordborg and Tavaré, supra, 2002). Despite the increased research focus in LD based methods, however, many issues regarding LD in human populations remain largely unexplored. Currently the NHGRI is organizing a systematic project to help develop informational tools for gene identification studies by identifying the common haplotypes in several populations. This “Haplotype Map Project” (HMP) will likely be a large-scale multicenter effort focused on finding common haplotypes in general population samples. The HMP will likely prove to be an important data resource for identifying AIMs as disclosed herein because several populations will be investigated for haplotype block structure, thus providing additional candidate AIMs and a basic plan for the fine scale LD structure in some of the parental populations.

Admixture mapping as disclosed herein is complementary to, but distinct from, the HMP. First, the primary focus of the HMP is to understand the fine scale structure of individual genomic regions throughout the genome, whereas the present methods allows an understanding of the LD that results specifically from admixture. The level of LD from admixture is on the order of millions of bases (Mb; megabases) and tens of Mb, while the HMP is focused on the level of 10's to 100's of kilobases (kb), and genomic and population features that affect the results from one project may not be noted in the other. Second, admixture mapping require accurate parental allele frequency estimates. As such, a large number of different African, Native American, European, and Asian populations have been typed (see Table 6, below), while the HMP will likely focus on one or two samples of the major population groups.

Third, large samples (n=500) of African-Americans and Hispanics have been typed, thus providing sufficient statistical power to test the coverage of the admixture map and to compare analytical methods. In addition, several representative populations from different regions of the country were typed so that geographical variation in ancestral proportions and admixture dynamics can be examined. Although some admixed populations will likely be included in the HMP, the numbers of individuals and numbers of different population samples being discussed are fewer than those as disclosed herein and, therefore, will not allow the same comparisons. For example, having a sample of 10 for each of 4 ancestral groups is not adequate for the identification of sequences present preferentially in one or some of those groups; as disclosed herein, at least 50 individuals were tested for each of several tens of ancestral groups (not just four) in order to comprehensively identify these markers.

Fourth, the focus of current population variation efforts (e.g., the SNP Consortium allele frequency project) and, very likely, the HMP has been on East Asian, African, and European samples to the exclusion of Native American populations for a number of complex reasons. The exclusion of these populations, however, results in a deficit in an understanding of the genetics of the fastest growing group of US resident populations, i.e., Hispanics, who have a significant level of Native American ancestry (20% to 40%). With the markers and methods disclosed herein, the disease genetics of Hispanic populations can be examined. Similarly, several diverse Native American populations may represent important parental populations for the numerous distinct groups often grouped together as Hispanic.

The population-based association methods disclosed herein provide several advantages over traditional linkage studies. Localizing disease genes by traditional genetic linkage methods relies on the use of related persons, either extended multigenerational families or pairs of related individuals. These approaches are effective and very powerful when investigating diseases caused by single genes. However, polygenic and multifactorial diseases like Type 2 diabetes, hypertension, and prostate cancer result from the interaction of several genes and multiple environmental influences, and are more difficult to study using traditional methods. The identification of genes contributing to susceptibility to common disease is complicated by heterogeneity. The source of the genetic heterogeneity determines which mapping methods are most likely to work for gene identification. Two primary types of genetic heterogeneity are locus heterogeneity, wherein more than one locus is affecting a genetic trait, and allelic heterogeneity, wherein within a particular causative locus there are multiple alleles that are important in altering the phenotype. Traditional linkage analysis using extended families is generally insensitive to allelic heterogeneity, but can be adversely affected by locus heterogeneity. Alternatively, LD based methods are generally adversely affected by allele heterogeneity, but less affected by locus heterogeneity.

Provided there is little allelic heterogeneity, association-based approaches like measured genotype and transmission disequilibrium test (TDT) may be more sensitive than family-based LOD score or sib-pair methods. Risch and Merikangas (supra, 1996) compared the number of individuals needed for sib-pair studies and TDT studies, and showed that the number of individuals needed to detect linkage is much smaller for TDT than for sib-pair studies. This is especially true when the disease locus has a small effect. For example, for a locus with risk ratio of 2.0 and a gene frequency of 50%, 2500 sib-pairs or 340 case/parents for TDT would be required. There are some examples in which the demonstration of association using Haplotype Relative Risk (HRR) or case/control design, or linkage with TDT, has preceded the demonstration of linkage with sib-pairs. A classic example is the association between the insulin gene and IDDM, which was demonstrated in cases and controls, then confirmed using TDT, but often not observed in sib-pair linkage studies (reviewed in Spielman et al., Amer. J. Hum. Genet. 28:317-331, 1993). Yaouanq et al. (Science, 1997) reported very significant (p<10-9) evidence for linkage between the HLA and multiple sclerosis using TDT in a series of 157 French families (99 simplex and 58 multiplex). When the 58 multiplex families were analyzed alone, p values of 0.0001 and 0.03 resulted for TDT and sib-pair methods, respectively.

Although association studies based on candidate genes have relatively higher power to detect disease genes than linkage analysis in families (Risch and Merikangas, supra, 1996), thoroughly testing all the genes in a genome with over 40,000 genes is currently not practical. The Haplotype Map Project may succeed in creating the informational resources necessary to perform gene identification based on linkage disequilibrium. However, even if the block structure models of the human genome can be explained by four haplotypes in each gene, the minimum number of SNPs and DIPs would then be 80,000 and the actual number likely higher. Although genotyping technologies are advancing rapidly, typing this number of markers in a large number of research subjects is not yet practical. Additionally, there are some important assumptions that are implicit in plans to identify genes using LD in large populations. One important difficulty using linkage disequilibrium in genome-wide screening is that LD decays exponentially with the recombination fraction between the marker and the disease locus and with the age of the disease-causing mutations. For older mutations that predispose to diseases, LD becomes very weak even between the disease allele and alleles at relatively closely spaced marker loci.

LD mapping has been useful in mapping of rare genetic diseases such as cystic fibrosis and diseases in special populations like the Finns and Bedouins, populations that have been subject to significant population bottlenecks, inbreeding, or founder effects. In these situations, LD exists because the variant allele is relatively young, as in the case of cystic fibrosis, or the population has reduced genetic variability, which elevates the LD throughout the genome. A leading model for the genetics of common disease stipulates predisposing alleles at a number of loci which, when present in particular combinations, increase an individuals risk (Greenberg, Amer. J. Hum. Genet. 52:135-143, 1993; Lander and Schork, Science 265:2037-2048, 1994; Risch and Merikangas, supra, 1996). If the disease is common, then for this model, the predisposing alleles also are expected to be at relatively high frequencies. However, assuming the neutral model, the frequency of an allele in a population is on average related to the age of the allele such that more frequent alleles are older than rare alleles. This fact poses a problem for the application of LD-based methods to identify common disease genes in populations that are not isolated or inbred since, in homogeneous populations, the LD is inversely related to the age of the allele and risk alleles for common disease are expected on average to be relatively old.

The application of the compositions and methods of the present invention for admixture mapping allows for a more precise and reliable mapping of complex traits. Admixture mapping takes advantage of the LD created when previously isolated populations admix, and can circumvent these problems in mapping complex traits. It was first recognized that admixed populations could be useful in determining genetic linkage by Chakraborty and Weiss (supra, 1988). When genetically divergent populations hybridize, non-random allelic associations result among loci that have significant allele frequency differentials, even among unlinked loci. This LD quickly decays when the genetic loci in question are not located close together on the same chromosome.

LD decays as a function of the recombination rate (O) between the two markers and the number of generations (n) since their hybridization, and can be represented as Dn=(1−θ)n D0, where Dn is the linkage disequilibrium n generations after hybridization and D0 is the initial linkage disequilibrium (Chakraborty and Weiss, supra, 1988). Given this exponential relationship between the decrease in LD and genetic distance, it is possible to discriminate between LD in an admixed population (if the time since admixture is short) that remains high because markers are close together, genetically linked, and background linkage disequilibrium among unlinked loci. For example, after 10 generations, the linkage disequilibrium at unlinked loci is reduced to 0.1% of the initial level, while at loci 10 cM and 1 cM apart, the disequilibrium due to true linkage will still be 34.9% and 90.4%, respectively, of the initial level. The critical parameters for effective detection of linkage in an admixed population identified are the frequency differential (δ) between the parental populations and the number of generations since hybridization. Linkage by association analysis in admixed populations worked efficiently if δ was large (not less than 0.2) and the number of generations since admixture small (on the order of 10 generations; Chakraborty and Weiss, supra, 1988).

Stephens et al. (supra, 1994) and Briscoe et al. (supra, 1994) studied this approach using computer simulations (MALD) and detailed practical considerations for study design. Using simple models of the type of admixture that has occurred in the Americas, they suggested that using sample sizes of 200-300 patients, typed for 200-300 evenly spaced markers, each having δ>0.3, one would have >95% chance of locating the causative gene. A consistent result from the several models studied was a primary dependence of the power of MALD on the allele frequency differentials of the markers used. If δ is small, the initial LD will be small and difficult to distinguish from the background noise.

Stephens et al. (supra, 1994) suggested using loci where δ>0.4 between the admixed parental populations for effective admixture mapping. They also demonstrate that admixture mapping is most effective in populations that hybridized between 4 and 20 generations ago, and that incremental admixture (the slow introgression of one population into another; also known as the continuous gene flow model) affects the power of admixture mapping, but not critically, provided there has been no new introgression from the parental populations in the past three generations. The disclosed admixture mapping technique can identify the location of disease susceptibility genes by the analysis of admixed populations composed of parental populations where there is a large difference in the frequency of the susceptible genotype. As such, applications of admixture mapping include the study of Type 2 diabetes susceptibility in Pacific Island populations, hypertension obesity, and prostate cancer in African Americans, and Type 2 diabetes, obesity and gallbladder disease in Hispanic populations.

McKeigue developed an approach to exploit admixture in mapping genes that builds on earlier work (McKeigue, supra, 1997; McKeigue, supra, 1998, McKeigue et al., supra, 2000). Although the approach is powered by the LD that is generated by admixture, it is more analogous to linkage analysis of an experimental cross. For this reason, the term “admixture mapping” was proposed. Instead of testing for allelic associations, one can model the underlying variation in ancestry on chromosomes of mixed descent to extract all the information about linkage that is generated by admixture.

As discussed above, advanced statistical methods are required to apply this approach in practice. Conditioning on parental admixture eliminates association of the trait with ancestry at unlinked loci and ensures that the comparison is made with other factors held constant. In non-statistical language, a comparison is made in each individual of the proportion of alleles at the marker locus that are of a particular descent with the expected proportion given the admixture of that individual's parents. One simple way to do this is to use the Analysis of Covariance (ANCOVA) test (see Tables 2 and 3), though this simpler approach does not use all of the information available. As such, Bayesian methods also have been used (see Tables 2 and 3).

To infer the ancestry of the alleles at the locus from the marker genotype, the ancestry-specific allele frequencies are required; i.e., the conditional probability of each allelic state given the ancestry of the allele (West African or European, in this example). The total population of alleles at any locus in the admixed population can be considered to be made up of two subpopulations—alleles of African ancestry and alleles of European ancestry. As long as the ancestry-specific allele frequencies are correctly specified for the admixed population under study, Bayes' theorem can be applied to invert these conditional probabilities and calculate the posterior distribution of ancestry at the locus (0, 1 or 2 alleles of African ancestry) for each individual under study. If the information conveyed by typing a single marker is not sufficient to assign the ancestry of each allele at the marker locus to one of the two founding populations, markers can be combined in a multipoint analysis to estimate ancestry at adjacent loci.

Simulation studies showed that, with enough markers, a high proportion of information about ancestry at each locus can be extracted even though no single marker is fully informative for ancestry (McKeigue, supra, 1998). Based on these simulations, panels of markers with Fst>0.4 at an average spacing of 2-3 cM for a total of 1,000 AIMs can be constructed as disclosed herein. It should be recognized that the panel of 1,000 AIMs for a particular population (e.g., an African-American group that is primarily West African and European) will often overlap with panels for other groups. In other words, it is often the case that AIMs selected for one level of distinction (e.g., African/European) are also informative for other distinctions (e.g., Native American/European). Table 1 lists an initially identified panel that includes of 32 AIMs (SEQ ID NOS:332 to 363; see, also, Example 1). Using a cutoff of d>0.3, only four of these markers are restricted in informativeness to one of the three comparisons (African/European; African/Native American; Native American/European); the rest are informative for two of the comparisons, and one marker is informative for all three comparisons. In a further study, a panel of 71 AIMs was identified (SEQ ID NOS:1 to 71; Table 6) that are informative as to IndoEuropean, sub-Saharan African, Native American, and East Indian (see Example 2).

There is growing evidence that admixture mapping will be an effective means of gene identification. At least three independent groups have reported strong admixture linkage disequilibrium (ALD) between linked markers spaced at substantial distances (see, e.g., Parra et al., supra, 1998 and 2001; Pfaff et al., supra, 2001; McKeigue et al., supra, 2000). Given the very high levels of association that have been observed over long genetic distances, it is expected that phenotypes dramatically different between parental populations because of some genetic difference will also show associations with linked AIMs. However, as promising as that MALD approach appears, until the present disclosure, no systematic screen has been reported identifying SNP based versions of the AIMs required. McKeigue and others have identified panels of STR AIMs for use with this approach, but the use of STRs for this purpose is problematic because of the allelic complexity of STRs and the massive databases required in order to accurately estimate allele frequencies. Even small errors or faulty assumptions on the frequencies of unobserved alleles can amplify to cripple the statistical power of a study.

Heterogeneity within the parental populations can have a confounding effect on admixture mapping studies. In the case of African-American populations, the process of admixture that took place in the New World involved a heterogeneous group of populations mainly from West-Central Africa and Europe, as well as some Native American populations. Regarding the European genetic contribution, the most important source populations came from Great Britain, Ireland, Germany and Italy. In spite of the diverse geographical areas of origin of the parental European populations, it is important to indicate the relative homogeneity of European populations from the genetic point of view (see for example Cavalli-Sforza et al., supra, 1994).

With respect to the African contribution, it is well known that the African continent contains a tremendous amount of genetic diversity. However, only a subset of the African genetic diversity contributed to the formation of African-American populations. The majority of enslaved Africans came from West-Central Africa, approximately from Senegal in the North, to Angola in the South (Curtin, In The Atlantic Slave Trade; Madison, University of Wisconsin Press 1969); other areas of Africa were not affected by the slave trade. Of the four main linguistic families present in Africa, Niger-Congo Kordofanian, Nilo-Saharan, Afro-Asiatic and Khoisan (Greenberg, supra, 1963), the majority of enslaved Africans forcefully brought to the New World were members of the Niger-Congo family. This widespread family encompasses West African languages (spoken by peoples from Senegal to Nigeria) and Bantu languages (dominant in Central and Southern Africa). The Bantu languages were dispersed throughout Africa by a “recent” expansion that took place about 3,000 years ago, and probably originated in West Africa (Nigeria and Cameroon; see Excoffier et al., Yearbook Phys. Anthropol. 30:151-194, 1987 and Cavalli-Sforza et al., supra, 1994). This recent origin is reflected in the linguistic and genetic homogeneity of the Bantu (Excoffier et al., supra, 1987, Weber et al., supra, 2000). Thus, the available historical, linguistic and genetic evidence indicate that only a subset of the diversity found in sub-Saharan Africa has contributed to the African-American gene pool, and that potential problems of heterogeneity are much less than if the diversity of the whole continent of Africa were represented in contemporary African-American populations. Unfortunately, the extent of the heterogeneity present in West and Central Africa remains largely unknown due to the lack of available information for the populations of this area.

Since the extent of heterogeneity within European populations and within West and Central Africa remains largely unknown, potential effects of heterogeneity need to be addressed, particularly when considering an admixture mapping approach. There are two levels at which heterogeneity can affect an admixture mapping effort. First, heterogeneity can lead to erroneous estimates of the parental frequencies for the markers used in the map, thus biasing the estimate of admixture. Given that the goal of admixture mapping is to infer linkage conditioning on parental admixture, it is important to avoid misspecification of the ancestry-specific allele frequencies, because this could affect the final outcome of the analysis. Second, heterogeneity can affect the number of loci for the phenotype being studied.

The effect of heterogeneity in biasing the estimates of the genetic contributions to an admixed population can be reduced by selecting markers showing homogeneity within the main parental populations (Europeans and Africans). In this way, the problem of contribution of different geographical areas to the parental populations is minimized, reducing the bias in admixture estimates. This strategy was implemented in previous admixture studies (Parra et al., supra, 1998, 2001; Pfaff et al., supra, 2001), wherein potentially informative markers in different European and African populations were systematically analyzed. As an example, currently, to test for heterogeneity within Africa, each potentially informative marker was genotyped in samples from five African populations, two from Nigeria, two from Sierra Leone and one from Central African republic, and the markers showing significant heterogeneity were excluded from the analysis (see below). All of these samples came from areas that were affected by the slave trade. If desired, a sample from Angola, which is a region that was the source of around 40% of enslaved Africans, can be incorporated, thus providing another sample of African parental populations. In addition to this strategy, it is important to note that there are statistical methods to test for misspecification of parental frequencies (see, e.g., McKeigue et al., supra, 2000).

With respect to the potential problem of heterogeneity in the phenotypes being studied, it is expected that heterogeneity due to the presence of multiple genes (locus heterogeneity) affecting a phenotype will reduce the power of admixture mapping to detect significant genotypic effects, as it does with any other mapping method. Heterogeneity also can be due to multiple functional alleles within a particular gene (allelic heterogeneity). One example is MC1R, where approximately six relatively common variants lead to red hair, freckles, and pale skin among Native Europeans and their descendant populations. Within Europeans these variants are on different haplotype backgrounds, thus decreasing the power to detect an effect of the MC1R gene in association studies relative to the case where a single mutation had occurred and risen to high frequencies. However, in an admixed population (e.g., European/African), these variants will all be in allelic association with markers informative for ancestry (e.g., the MC1R marker, see Table 1) and, since they all have the effect of lightening the skin, their information will be compounded making the identification of MC1R by admixture mapping no different with six functional variants than were there only one functional variant unique to Europeans. So long as the effects of functional variants within a particular parental population are in the same direction (for example, in lowering the risk of disease), allelic heterogeneity will not be a serious problem in admixture mapping.

The majority (80-90%) of genetic variation among human individuals is inter-individual; only 10-20% of the variation is due to population differences (e.g., Nei, supra, 1987; Cavalli-Sforza et al., supra, 1994, Deka et al., supra, 1995). Most populations share alleles and those alleles that are most frequent in one population generally are also frequent in others. There are very few classical (blood group, serum protein, and immunological) or DNA genetic markers which are either population-specific or have large frequency differentials among geographically and ethnically defined populations (Roychodhury and Nei, supra, 1988; Cavalli-Sforza et al., supra, 1994). Despite this apparent lack of unique genetic markers, there are marked physical and physiological differences among human populations that presumably reflect long-term adaptation to unique ecological conditions, random genetic drift, and sex selection. In contemporary populations, these differences are evident both in morphological differences between ethnic groups and in differences in susceptibility and resistance to disease.

The most useful unique alleles for admixture and mapping studies are those that also have large differences in allele frequency among populations (Reed, supra, 1973; Chakraborty et al., supra, 1992; Stephens et al., supra, 1994). The fact that they are totally absent from all other populations does simplify some of the statistical computations, and can facilitate more confident parental allele frequency estimates, but is not the primary reason for their utility. The designation population-specific alleles (PSAs) was initially used to describe genetic markers with large allele frequency differentials between populations (Shriver et al., supra, 1997; Parra et al., supra, 1998), but these markers are now referred to by the more correct and descriptive term, Ancestry Informative Markers (AIMs). For a biallelic marker, the frequency differential (δ) is equal to px−py, which is equal to qy−qx, where px and py are the frequencies of one allele in populations X and Y and qx and qy are the frequencies of the other. Median δ levels among major ethnic groups range between 15% and 20%, and the vast majority (>95%) of arbitrarily identified biallelic genetic markers have 6<50% (Dean et al., supra, 1994, which is incorporated herein by reference). Statistical estimates of power in an admixture mapping study based on using markers with an Fst>0.4 were previously presented (McKeigue et al., supra, 2000). With 1,000 such markers evenly spaced across the genome, it was demonstrated that it was possible to have a statistical power of 80% to identify a disease gene that explains a 2 fold relative risk between the parental populations.

AIMs, and their use according to a method of the invention are demonstrated in Examples 1 to 6 (below). In addition, allele frequency data for markers informative for admixture mapping in African-American and Hispanic populations have been reported (Smith et al., supra, 2001; Collins-Schramm et al., supra, 2002). In order to apply the methods of the invention to an analysis of disease predisposition or drug responsiveness, estimation of admixture proportions and admixture dynamic is important. Controlling for genetic structure in admixed populations requires knowledge of the ancestral proportions and the genetic structure of these populations. Reliable estimates of admixture proportions can allow the informed identification of populations to consider. Since the admixture LD that is created during hybridization is dependent on the level of admixture, sampling should focus generally on those areas of the country where there has been more admixture.

In addition to knowing the ancestral proportions, it is important to understand the level of population structure present in the populations under consideration. A homogeneous population is one in which there is no assertive mating, a panmictic population in which families are formed more or less by random combination and without regard to DNA genotypes. In most large cosmopolitan populations, homogeneity is expected and found. If, however, there exists stratification within the population such that individuals do not mate at random, the population will not be homogeneous. Admixture is one of the possible mechanisms introducing genetic structure in a population, and taking into account this genetic structure facilitates admixture mapping.

The effect of genetic structure is considered at two levels. First, parental populations are evaluated to determine whether they show heterogeneity in the allele frequencies of the selected AIMs; heterogeneity can affect the estimate of admixture proportions, as discussed above. Several methods that can detect the presence of genetic structure. These methods can be grouped in two main categories, termed genomic control (GC) methods (Devlin and Roeder, supra, 1999), and structured association (SA) methods (Pritchard and Donelly, supra, 2001). Both methods require genotyping of a panel of unlinked markers to estimate and correct for the effect of genetic structure, which, as discussed above, may have been due to sampling effects, or due to real demographic strata in the sampled population. The SA method (Pritchard et al., supra, 2000; Pritchard and Donelly, supra, 2001) was used to test for genetic structure in the parental populations. This method is based on using the genotypic information provided by the unlinked markers to infer population structure, and has been implemented in a software program available from Jonathan Pritchard. In addition, to test for the presence of structure, the program estimates individual ancestry proportions, and, for the present studies, this Bayesian method was used to complement the Maximum Likelihood Estimate method. These two methods produce estimates of individual ancestry that are highly correlated.

The second source of genetic structure in admixed populations is due to the admixture process itself, in which newly created linkage disequilibrium is introduced in the admixed population. AIMS, such as those exemplified herein, are particularly sensitive indicators of population structure that is related to ancestral proportions. To evaluate the presence of population structure, samples are tested for the non-random association of alleles both within a locus (Hardy-Weinberg disequilibrium) and among loci (gametic disequilibrium), and the distribution of individual ancestry estimates also is examined (see, Pfaff et al., supra, 2001; Parra et al., supra, 2001).

The history of African Americans can be traced back to 1619, when the first Africans arrived at the British colonies (Jamestown), although as early as 1526 the presence of African slaves was reported in Spanish expeditions to what would become the United States (South Carolina, Georgia, Florida and New Mexico). Institutional slavery began very soon after, but it was not until the beginning of the 18th century that the importation of slaves reached increased rates, in parallel with the demand for workers to cultivate the tobacco, indigo, and rice plantations in the southern colonies; peaks occurred in the decade from 1790-1800 and the first years of the 19th century. In 1808, slave trade became illegal but continued at a low rate for several more decades. Different estimates have been offered on the total number of slaves brought into the United States with generally accepted numbers ranging between 380,000 and 570,000.

Although it is difficult to precisely determine the ethnic origins of the African slaves, information from shipping lists has provided an approximate picture of their geographic provenance. The slave trade affected a very wide area of Western and Western-Central Africa, mainly the coastline between the present day countries of Senegal in the North and Angola in the South. The most important regions were Senegambia (Gambia and Senegal), Sierra Leone (Guinea and Sierra Leone), Windward Coast (Ivory Coast and Liberia), Gold Coast (Ghana), Bight of Benin (From the Volta river to the Benin river), Bight of Biafra (East of Benin river to Gabon), and Angola (Southwest Africa, including part of Gabon, Congo and Angola). Curtin (supra, 1969) has offered, based on data on the English trade of the 18th century (the peak of the Atlantic slave trade), estimates of the proportional contribution by areas, showing that Angola and Bight of Biafra were the regions contributing the highest numbers of slaves imported into the North American mainland (around 25% each). However, there were significant differences in ethnic origin depending on the port of entry in the United States, and the figures for the Colonies of Virginia and South Carolina differed considerably.

The history of African Americans has been marked not only by the forced migration from Africa, but also by the admixture with the other ethnic groups they met when they arrived in North America, including with Europeans and Native Americans. However, few historical records address the issue of admixture. Additionally, there have been important factors that, in the time since the abolition of slavery until the present, have configured the present African-American population. Of special interest is the pattern of migration of African Americans within the United States over the past 150 years. In this sense, the redistribution of African Americans in the Southern States during the 19th century, and the Great Migration from the rural South to the urban areas in the North beginning after World War I are of particular relevance, and have had an enormous impact in defining the present distribution of the African-American population in the US (Johnson and Campbell, In Black Migration in American: A Social Demographic History; Duke University Press, Durham N.C. 1981).

With respect to Hispanics, the term “Hispanic” was coined mainly for governmental demographic purposes, and is generally employed to identify persons of Latin American origin or descent, living in the United States. Although this definition lumps together people with very different historical, cultural and linguistic backgrounds, this classification has been widely used. Even though Central America, the Caribbean, and South America have been for centuries under the domination of the Iberian imperial powers (Spain and Portugal), they have had quite different regional histories, both before and after the Colonial period. Populations from four continents, North and South America, Europe, and Africa, have contributed to the formation of contemporary Hispanic populations. The anthropological background of the main three Hispanic groups currently living in the United States—Mexican Americans, Puerto Ricans and Cuban Americans, which together makeup more than 80% of the total US Hispanic population—is considered here.

Mexican Americans show the highest Amerindian contribution of the three aforementioned groups. Soon after the Spanish conquest of Mexico, at the beginning of the 16th century; intermixture of the Spanish men with Amerindian women resulted in an increasingly important mixed population (Mestizos), and this racial mixing continued through the three centuries of Spanish domination in “New Spain”, configuring both biologically and culturally the Mexican population. The majority of estimates have indicated an Amerindian component in Mexican Americans ranging between 30% and 40% (Hanis et al., supra, 1986; Long et al., 1991; Hanis et al., Diabetes Care 14:618-627, 1991; Merriwether et al., Amer. J. Phys. Anthrop. 102:153-159, 1997). It is interesting to point out, as well, that some studies have shown differences in the amount of Amerindian ancestry depending on socioeconomic status (Chakraborty et al., Genet. Epidemiol. 3:435-454, 1986; Mitchell et al., Ethnicity and Disease 3:22-31, 1992). There was also a substantial African presence in the Mexico territory during the Spanish rule. Curtin (supra, 1969) has estimated the total number of Africans imported into Mexico during the entire period of Slave trade to be around 200,000. Their contribution to the Mexican gene pool, however, has been estimated to be much lower than the European and Amerindian contribution, ranging from zero to 10% (see, e.g., Hanis et al., supra, 1991).

In the Caribbean Colonies (Cuba and Puerto Rico), the situation was very different from the Mainland. The Native American population was far smaller, and was decimated by slavery and disease very soon after the first contact with the Europeans. Nevertheless, the rate of admixture during the initial phases of the colonization was high enough to result in an appreciable genetic contribution (about 18%) from the Arawaks and Caribs, the original inhabitants of the Hispanic Caribbean (Hanis et al., supra, 1991). Another distinctive feature of this region is a significant African influence, which is also reflected in many aspects of the present societies of countries like Cuba, Puerto Rico, and the Dominican Republic. African slaves were imported to work in the sugar plantations in large numbers, even outnumbering the population of European origin (Kanellos and Perez, In Chronology of Hispanic-American history: from pre-Columbian times to the present; New York, Gale Research 1995). Accordingly, the percentage of African genetic contribution in contemporary Cubans (20%) and Puerto Ricans (37%) is significantly higher than in other Hispanic populations (Hanis et al., supra, 1991).

It is clear that race is a complex concept and, in general usage, reflects both a cultural and biological feature of a person or group of people. Given the fact that physical differences between populations are often accompanied by cultural differences, it has been difficult to separate these two elements. There has been a movement in several fields of science to oversimplify the issue declaring that race is merely a social construct. While this often can be true, depending on what aspect of variation between people is being considered, it can be false for many particular instances of differences between the populations of the world. One clear example of a biological difference is skin color. Culture or environment has almost no effect on the level of pigmentation in a person's skin. Yet there are dramatic differences across populations. Pigmentation is, of course, only skin deep and is quite simple in light of the complex environments in which we live and how these affect individual and group quality of life.

The human species is relatively young and, as a species, most likely originated in east Africa 100,000 years ago, and diverged as groups to settle the globe (Cavalli-Sforza and Cavalli-Sforza, In The Great Human Diasporas. The History of Diversity and Evolution (Perseus Books, Cambridge Mass. 1995). During these migrations, and in the time since, there has been some degree of independent evolution of the populations that settled the various continents of the world. The simplest evidence of this evolution is seen in the differences in allele frequencies at genetic markers. Generally, alleles that are found in one population are also found in all populations, and the alleles that are the most common in one population also are common in others. These similarities between populations highlight the recent common origin of all populations. However, there are examples of genetic markers that are different between populations and, as disclosed herein, these markers, AIMs, can be used to estimate the Ancestral origins of a person or population.

The present invention provides methods of estimating proportional ancestry of at least two ancestral groups of a test individual and, in particular, provides a confidence level with respect to the proportional ancestry. A method of the invention can be performed by contacting a sample, which includes nucleic acid molecules of the test individual, with hybridizing oligonucleotides that can detect nucleotide occurrences of SNPs of a panel of at least about ten AIMs that are indicative of BGA for each ancestral group examined, wherein the contacting is under conditions suitable for detecting the nucleotide occurrences of the AIMs of the test individual by the hybridizing oligonucleotides; and identifying, with a predetermined level of confidence, a population structure that correlates with the nucleotide occurrences of the AIMs of each of the ancestral groups examined, wherein the population structure is indicative of proportional ancestry.

The term “biogeographical ancestry” or “BGA” is used herein to describe the biological or genetic component of race. BGA is a simple and objective description of the ancestral origins of a person, in terms of the major population groups (e.g., Native American, East Asian, Indo-European, and sub-Saharan African). BGA estimates can represent the mixed nature of many people and populations today. In many countries, including the United States, there has been extensive mixing among populations that initially had been separate. The term “admixture” is used herein to refer to such population mixing. In this respect, BGA estimates can be understood as individual admixture proportions, which take the form of a series of percentages that add to 100%. For example, a person can have 75% Indo-European, 15% African, and 10% Native American ancestry, or can have 100% Indo-European ancestry, or the like.

The proportional ancestry estimated according to a method of the invention can be a proportion of any ancestral group, including, for example, a proportion of sub-Saharan African, Native American, IndoEuropean, East Asian, Middle Eastern, or Pacific Islander ancestral group, and generally is a combination of two or more of such ancestral groups. Thus, the proportional ancestry of a test individual can include proportional affiliation among the sub-Saharan African and IndoEuropean ancestral groups (e.g., 80% sub-Saharan African and 20% IndoEuropean; or 60% sub-Saharan African, 20% IndoEuropean, and 20% of a third ancestral group); or can include proportional affiliation among the Native American and IndoEuropean ancestral groups; East Asian and Native American ancestral groups; IndoEuropean and East Asian ancestral groups; and the like.

A panel of AIMs useful for estimating proportional ancestry of an individual can include AIMs as set forth in SEQ ID NOS:1 to 331, for example, AIMs as set forth in SEQ ID NOS:1 to 71, which can be useful for determining proportional ancestries including IndoEuropean, sub-Saharan African, East Asian, and Native American; or AIMs as set forth in SEQ ID NOS:7, 21, 23, 27, 45, 54, 59, 63, and 72 to 152, which can be useful for determining proportional ancestry of East Asians and sub-Saharan Africans; or in SEQ ID NOS:3, 8, 9, 11, 12, 33, 40, 59, 63, and 153 to 239, which can be useful for determining proportional ancestry of East Asians and IndoEuropeans; or in SEQ ID NOS:1, 8, 11, 21, 24, 40, 172, and 240 to 331, which can be useful for determining proportional ancestry of IndoEuropeans and sub-Saharan Africans.

An estimate can be made, for example, of an individual's proportional ancestry with respect to three ancestral groups. In this method, identifying a population structure within an individual that correlates with the nucleotide occurrences of the AIMs of the test individual can be practiced by performing a likelihood determination for affiliation with each of a sub-Saharan African ancestral group, a Native American ancestral group, an IndoEuropean ancestral group, and an East Asian ancestral group; thereafter selecting three ancestral groups having a greatest likelihood value for the individual; determining a likelihood of all possible proportional affiliations among the three ancestral groups having the greatest likelihood value, whereby a population structure or proportional affiliation that correlates with the nucleotide occurrences of the AIMs of the test individual is identified; and identifying a single proportional combination of maximum likelihood. Alternatively, identifying a population structure that correlates with the nucleotide occurrences of the AIMs can be practiced by performing six two-way (binary) comparisons comprising likelihood determinations for affiliation of each group compared to each other group; thereafter selecting three ancestral groups having a greatest likelihood value across all comparisons; determining a likelihood of all possible proportional affiliations among the three ancestral groups having the greatest likelihood value, whereby a population structure or proportional affiliation that correlates with the nucleotide occurrences of the AIMs of the test individual is identified; and identifying a single proportional combination of maximum likelihood. Such a methodology works as well for individuals of three-way admixture as individuals that are 100% affiliated with a single group.

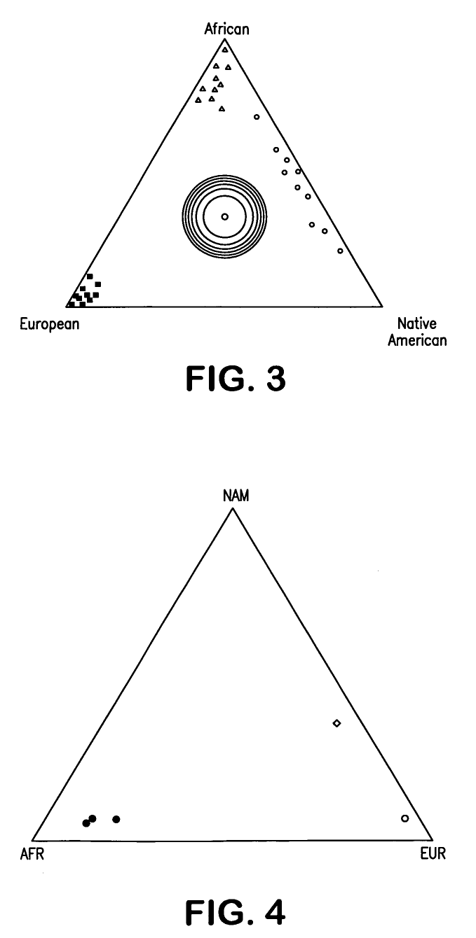

An estimate of an individual's proportional ancestry that includes proportions of three ancestral groups also can be made by performing three three-way comparisons among the groups; determining a likelihood of all possible proportional affiliations among the three ancestral groups having the greatest likelihood value, whereby a population structure or proportional affiliation that correlates with the nucleotide occurrences of the AIMs of the test individual is identified; and identifying a single proportional combination of maximum likelihood. An advantage of the present methods is that a graphical representation of the comparison of the three ancestral groups can be generated, wherein the graphical representation comprises a triangle with each ancestral group independently represented by a vertex of the triangle, and wherein the maximum likelihood value of proportional affiliation for an individual comprises a point within the triangle (see FIGS. 2 and 3). If desired, the graphical representation can further include a confidence contour that indicates a level of confidence associated with estimating the proportional ancestry.

An estimate of an individual's proportional ancestry also can be made where the proportional ancestry includes proportions of four ancestral groups. In various aspects of this method, identifying a population structure that correlates with the nucleotide occurrences of the AIMs of the test individual is practiced by performing six two-way comparisons, or by performing three three-way comparisons, or by performing one four-way comparison among the groups; determining a likelihood of all possible proportional affiliations among the four ancestral groups having the greatest likelihood value, whereby a population structure or proportional affiliation that correlates with the nucleotide occurrences of the AIMs of the test individual is identified; and identifying a single proportional combination of maximum likelihood. If desired, the method can further include generating a graphical representation of the comparison of the three ancestral groups, wherein the graphical representation comprises a pyramid with each ancestral group independently represented by a vertex of the pyramid, and wherein the maximum likelihood value of proportional affiliation for an individual comprises a point within the pyramid. If desired, the graphical representation can further include a confidence contour comprising a sphere around the point, wherein the sphere indicates a level of confidence associated with estimating the proportional ancestry.

As disclosed herein, such methods are useful, for example, as a forensic tool. The present methods provide substantially greater information for forensics because, using a DNA sample obtained at a crime scene, the methods can provide an investigator with prospective information as to the likelihood of an individuals ancestry, as well as hair, skin and eye pigmentation. In comparison, present DNA methods only allow provide retrospective information because they require that a DNA sample from a crime scene be compared with DNA samples contained in a database or taken from specific individuals. Thus, while the latter methods can provide confirmation that a suspect is likely the perpetrator of a crime, they provide no useful information until the suspect is apprehended, except in cases where the suspect's DNA sample already has been entered into a database.

The methods of estimating proportional ancestry of a test individual as disclosed herein also provide a tool that can supplement genealogical information, which generally is based on relationships established using geopolitical information (see Example 3). For example, the present methods provide information that can be used to generate an ancestral map of the world, wherein locations of populations having a proportional ancestry corresponding to the proportional ancestry of the test individual are indicated on the ancestral map. As such, the method can further include overlaying the ancestral map with a genealogical map, wherein the genealogical map indicates locations of populations having geopolitical relevance with respect to the test individual, and statistically combining the information of the ancestral map and genealogical map to obtain a most likely estimate of family history of the test individual.

Identifying a population structure that correlates with the nucleotide occurrences of the AIMs, according to a method of the invention, can be performed by comparing the nucleotide occurrences of the AIMs of the test individual with known proportional ancestries corresponding to nucleotide occurrences of AIMs indicative of BGA. The known proportional ancestries corresponding to nucleotide occurrences of AIMs indicative of BGA can be contained in a table or other list, and the nucleotide occurrences of the test individual can be compared to the table or list visually, or can be contained database, and the comparison can be made electronically, for example, using a computer. A particularly useful application of a method of the invention involves associating known proportional ancestries corresponding to nucleotide occurrences of AIMs indicative of BGA of individuals, with a photograph of a person from whom the known proportional ancestry was determined, thus providing a means to further infer physical characteristics of a test individual. In one aspect, the photograph is a digital photograph, which comprises digital information that can be contained in a database that can further contain a plurality of such digital information of digital photographs, each of which is associated with a known proportional ancestry corresponding to nucleotide occurrences of AIMs indicative of BGA of the person in the photographs.

A method of the invention can further include identifying a photograph of a person having a proportional ancestry corresponding to the proportional ancestry of the test individual. Such identifying can be done by manually looking through one or more files of photographs, wherein the photographs are organized, for example, according to the nucleotide occurrences of AIMs of the person in the photograph. Identifying the photograph also can be performed by scanning a database comprising a plurality of files, each file containing digital information corresponding to a digital photograph of a person having a known proportional ancestry, and identifying at least one photograph of a person having nucleotide occurrences of AIMs indicative of BGA that correspond to the nucleotide occurrences of AIMs indicative of BGA of the test individual.

According to the present invention, BGA can be determined using any of several variations of the disclosed BGA test, including three BGA tests referred to as the ANCESTRYbyDNA™ 1.0 test, the ANCESTRYbyDNA™ 2.0 test, and the ANCESTRYbyDNA™ 3.0 test (DNAPrint genomics, Inc.; Sarasota Fla.), which utilize selected panel of Ancestry Informative Markers (AIMs) that have been characterized in a large number of well-defined population samples. The AIMs are selected on the basis of a showing of substantial differences in frequency between population groups and, as such, provide information as to the origin of a particular person whose ancestry is otherwise unknown. For example, the Duffy Null allele (FY*0) is very common (approaching fixation or an allele frequency of 100%) in all sub-Saharan African populations, but is not found outside of Africa. Thus, a person with this allele is very likely to have some level of African ancestry. Upon analysis of AIMs in a DNA sample from a person of unknown origin, a likelihood (or probability) can be determined that the person is derived from particular parental populations by calculating all of the possible mixes of parental populations. The population (or combination of populations) where the likelihood is the highest is taken as the best estimate of the ancestral proportions of the person; confidence intervals on these point estimates of ancestral proportions are also calculated.

An objective assessment of the biological component of human ancestry provides important knowledge about the person whose DNA is examined. For example, an analysis of the biological component of ancestry can elucidate health disparities by identifying, for example, genetic contributions to the higher rates of hypertension and diabetes in African Americans, or the higher rates of dementia in European Americans. Estimates of BGA also can help connect individuals separated by adoption or some other event with their ancestral populations. And even if a person is not particularly motivated to reconnect with ancestors, he or she can uncover the past of their family, for example, to verify family legends or identify forgotten roots. Because the disclosed method is based on an analysis of DNA, it provides a personal demographics tool, which, unlike a census, can provide highly accurate demographics data.

There are several commercially available tests that analyze mitochondrial DNA (mtDNA) or Y chromosome markers, and have been promoted as a means of learning one's ancestral origins. Although these tests can provide information regarding the provenance of some of a person's ancestors, the tests are very limited. For example, one generation ago a person has two ancestors, one mother and one father; five generations ago, a person has 32 ancestors; while 10 generations ago, a person has 1024 ancestors. Ten generations is roughly 250 years and well within the time frame of genealogical interest, especially when considering, for example, the settlement of North America. Because the mtDNA and Y chromosome tests only look at a small portion of the genome (the matrilineal and patrilineal lineages, respectively), they can only provide information relating to a very small proportion of a person's ancestors. The BGA test of the invention utilizes sequences throughout a person's genome and, therefore, can provide information about a greater number of ancestors.

Accordingly, the present invention provides a method of estimating, with a predetermined level of confidence, proportional ancestry of at least two ancestral groups of a test individual. Such a method, referred to as a “biogeographical ancestry test” or “BGA test”, can be performed, for example, by contacting a sample, which includes nucleic acid molecules of the test individual, with hybridizing oligonucleotides that can detect nucleotide occurrences of SNPs of a panel of at least about ten AIMs that are indicative of BGA for each ancestral group examined, wherein the contacting is under conditions suitable for detecting the nucleotide occurrences of the AIMs of the test individual by the hybridizing oligonucleotides; and identifying, with a predetermined level of confidence, a population structure that correlates with the nucleotide occurrences of the AIMs of each of the ancestral groups examined, wherein the population structure is indicative of proportional ancestry.

As used herein, the term “proportional ancestry” refers to the percent contribution of each (if more than one) ancestral group to which an individual belongs. The proportional ancestry estimated according to a method of the invention can be a proportion of any ancestral group, including, for example, a proportion of sub-Saharan African, Native American, IndoEuropean, East Asian, Middle Eastern, or Pacific Islander ancestral group, and generally is a combination of two or more of such ancestral groups. Thus, the proportional ancestry of a test individual can include proportions of sub-Saharan African and IndoEuropean ancestral groups (e.g., 80% sub-Saharan African and 20% IndoEuropean; or 60% sub-Saharan African, 20% IndoEuropean, and 20% of a third ancestral group); or can include proportions of Native American and IndoEuropean ancestral groups; East Asian and Native American ancestral groups; IndoEuropean and East Asian ancestral groups; and the like. Similarly, the proportional ancestry can include proportions of Native American, East Asian, and IndoEuropean ancestral groups; sub-Saharan African, Native American, and IndoEuropean ancestral groups; sub-Saharan African, Native American, and East Asian ancestral groups; and the like.

A panel of AIMs useful for estimating proportional ancestry of an individual can include AIMs as set forth in SEQ ID NOS:1 to 331, for example, AIMs as set forth in SEQ ID NOS:1 to 71, which can be useful for determining proportional ancestries including IndoEuropean, sub-Saharan African, East Asian, and Native American. For example, the AIMs as set forth in SEQ ID NOS:7, 21, 23, 27, 45, 54, 59, 63, and 72 to 152 can be useful for determining proportional ancestry of East Asians and sub-Saharan Africans; the AIMs as set forth in SEQ ID NOS:3, 8, 9, 11, 12, 33, 40, 59, 63, and 153 to 239 can be useful for determining proportional ancestry of East Asians and IndoEuropeans; and the AIMs as set forth in SEQ ID NOS:1, 8, 11, 21, 24, 40, 172, and 240 to 331 can be useful for determining proportional ancestry of IndoEuropeans and sub-Saharan Africans.

The ANCESTRYbyDNA™ 1.0 test (DNAPrint genomics, Inc.) is a first version of the BGA test that was specifically designed to provide information on the proportions of ancestry at the continental level. As such, the ANCESTRYbyDNA™ 1.0 test allowed information to be obtained as to levels of Native American, European, and African ancestry, as three component groups. The ANCESTRYbyDNA™ 2.0 test, in comparison, provides information on the proportions of ancestry at the continental level for most continents, including Native American, Indo-European (includes European, Middle Eastern and South Asian groups such as Indians), African, and East Asian (which includes Pacific Islanders, and can distinguish ancestries within Asia and the Pacific Rim. The ANCESTRYbyDNA™ 3.0 test can further define the levels of ancestry within continents, for example, by distinguishing Japanese from Chinese, or Northern European from Middle Eastern, thus providing greater insight into where within a particular continent a person's ancestors were derived.

For the ANCESTRYbyDNA™ 2.0 test, a logical grouping into four BGA delineations was made, wherein South Asian, Middle Eastern and European are grouped into a single group called IndoEuropean (see Example 2). This grouping was based on anthropological evidence and cultural connections between these groups (e.g., their languages are derived from a common base). The results disclosed herein demonstrate that these groups are far more similar to one another in genetic sequence content than to other groups. The ANCESTRYbyDNA™ 2.0 test also performs more accurately when Pacific Islanders are grouped with East Asians. As such, the four groupings used in the ANCESTRYbyDNA™ 2.0 test include 1) Native American (i.e., those who migrated to inhabit South and North America); 2) IndoEuropean (Europeans, Middle Easterners and South Asians such as Indians; 3) East Asians (Japanese, Chinese, Koreans, Pacific Islanders); and 4) Africans (sub-Saharan). The ANCESTRYbyDNA™ 3.0 test can further distinguish between South Asian and European, and between Pacific Islander and East Asian, thus providing 6 proportions (Native American, European, African, South Asian, East Asian and Pacific Islander), although the confidence intervals are larger than those obtained with the ANCESTRYbyDNA™ 2.0 test. Further improvement to the tests are provided, wherein the confidence intervals are reduced. Confidence intervals around a point estimate can be reduced, thus increasing the accuracy of the test, by analyzing a complementary panel, thereby improving the confidence intervals by about 50%.

The algorithm used to determine the ancestral proportions was developed based on the idea that it is possible to use certain statistical methods to make an inference of the proportionality of ancestry in an individual sample based on their sequence (see Example 6; see, also, Table 12). The method of making this inference using the present algorithm is similar to those of others, wherein, if the frequency of an allele in a population is known, and this frequency is significantly different from population to population, a “Maximum Likelihood Estimation” (MLE) can be used to determine the probability that a person with the allele belongs to one of the groups. Expanded to include multiple alleles from multiple genetic loci and multiple populations, the process is the same. By way of simplification, Bayes' theorem states that the probability of an event given a circumstance (called a posterior probability) is a function of the frequency of the circumstance given the event (a conditional probability) and the frequency of the event itself (the prior probability). By determining the probability of the event given the circumstance for a wide range of possible events, that with the highest probability can be selected, thus obtaining the MLE for the probability.

In the present algorithm, the event is a proportionality of ancestry, and the circumstance is the genotype of the individual. If the minor allele frequency for 10 SNPs in 2 populations of human beings is known, and the sequence of a person at each of the 10 SNPs is known, a simple binary classification into one of the two groups can be made by choosing that for which the conditional probability is highest. This would offer little improvement over current methods for determining the BGA from a DNA sample. What is provided by the present invention is the ability to obtain the proportionality of ancestry for more complex and realistic scenarios of ancestry. There are many possible combinations such as 99%, African, 1% European, 0 Native American, 0% East Asian; or 98% African, 1% European, 1% Native American, 0% East Asian; and the like. The posterior probabilities for each of the thousands of possibilities are not the same for any particular individual, given his or her multilocus genotype (i.e., genotypes of many AIMs), and, in fact, there is one that has the highest posterior probability or likelihood for each genotype. It is this combination that the present algorithm selects (i.e., the MLE).

Previous methods have been limited in that the confidence of the estimate was not known. The present algorithm addresses this limitation by plotting the MLE graphically, including plotting the confidence regions around the MLE such that a level of confidence can be ascertained (see FIGS. 2 and 3). Further, the algorithm (i.e., the software code) that performs the MLE calculation operates in an unusually efficient manner. The triangle plot provided by the algorithm is an original method to graphically represent the MLE calculations and their confidence intervals. To read a triangle plot (see below), a perpendicular line is dropped from each vertex (triangle point) of the triangle to the opposite edge (base) of the triangle (see FIG. 2A). In this figure, the circle represents the MLE, and a line has been dropped from the Native American (NAM) vertex to the line below; the line serves as a scale for the percentage of Native American ancestry, from 0% at the base to 100% at the vertex (or tip). Projecting the circle on this line can be analogized to holding a flashlight to the right of the triangle at the same level as the circle and observing the shadow the circle makes on the line. Where this shadow falls on the line indicates the percentage of Native American ancestry. In this example, the individual is about 15% Native American, as indicated by the hash mark on the line.

The results provided using the disclosed method provide a statistical estimate of BGA admixture for an individual (the Maximum Likelihood Estimate (MLE)), which is indicated as a point on a triangle plot to represent the proportions of the most relevant three groups for the individual. While the MLE is the most likely estimate, the true value for the individual can be a different set of proportions. A triangle plot with calculated and plotted estimates that are 2 times, 5 times and 10 times less likely than the MLE is exemplified. The first contour around the MLE delimits the space within which the estimates are up to 2 times less likely, with those positions near the line reflecting values close to 2 and those near the MLE closer to 1; the second contour around the MLE delimits the space within which the estimates are 5 times less likely in the same graded fashion proceeding from the first contour line to the second contour line. The third contour delimits the space within which the estimates are from 5 fold (near the second contour line) up to 10 times less likely (near the third contour line). The greater the number of DNA positions read, the closer these contour lines approach the MLE point. On the triangle plot, the likelihood (probability) that the true value is represented by a different point than the MLE increases until the MLE is met, where the probability is maximum (i.e., the Maximum Likelihood Estimate; MLE). The test can be performed so that the contour lines are very close to the MLE by sequencing a very large collection of markers. However, to keep the test affordable and efficient, the survey can be limited to a desired number of markers (e.g., 10, 15, 20, 25, 30, 40, 50, 60, 70, 80, 90, 100, or more) that is sufficient to determine the most likely proportions with good confidence. In this respect, a variety of different panels of 100 SNP markers have been examined, a panel of 71 AIMs has been used in a number of studies, and a panel of 175 AIMs is being examined such that very confidence is achieved.

The BGA test of the invention has been validated by determining the frequency of DNA sequence variants in various human populations. In addition, the test has been evaluated using a large number of people from a wide range of ancestral groups, and the estimates have corresponded well to what is known from anthropological and historical data. For example, Hispanics are known to have arisen as an ethnic group from the blending of colonial Europeans with Native Americans, and the hundreds of Hispanics examined using the BGA test aligned with these two groups almost exclusively. As another example, though Nigerians plot as of almost pure African BGA, African Americans plot more as a mixture between this group and Europeans, which is what would be expected from knowledge about the admixture between Africans and Europeans in the United States.

The method also was validated through pedigree challenge (see Example 1); i.e., when the BGA is determined from a mother and father, that of their children should plot somewhere between the two. Numerous family pedigrees have been examined using the test, and the ancestral proportions of offspring have always plotted between those of the child's parents. When the MLE estimates are tested objectively (blindly), they prove to be excellent estimates of ancestral proportions. For example, the data for a European American man, whose mother is European mix and father is mostly Greek, showed the man to be of 85% European ancestry, but also of 15% Native American ancestry (Example 1). In fact, his paternal great-grandmother was full-blood Cherokee, thus confirming the result of the test (based on the laws of genetics, the man would be predicted to have a 12% Native American Ancestry if his great-grandmother was 100% Native American and none of his other relatives were of Native American ancestry). Further, the man's wife is Mexican, and she was determined to be of mostly Native American, but with some Native American and African heritage. This was also expected based on what is known from anthropological origin of Hispanics, who derived from the union of Spanish explorers with Native Americans in Colonial Caribbean and Latin America. Each of the three children of the man and woman plotted roughly half way between both parents, as expected. None of the children showed any Asian or Pacific Islander ancestry, which would have been impossible (assuming an accurate test) because none of the parents showed any significant Asian or Pacific Islander heritage, and none of the children were found to have more African ancestry than their mother, which would also be impossible given the fact that the father has virtually none. Thus, the results of the children were consistent with those of the parents, and the MLE values were accurate estimates when tested against what was known from biographical data.

The genotypes (nucleotide letters) determined to date are quite accurate. Because the latest genetic reading equipment available is used, an accuracy greater than 99% accuracy is routinely achieved for each site. If an accurate value was not obtained for a particular site in a particular sample, an “FL” is indicated, instead of the genotype letters for that site. Having a few FL's generally does not prevent a good ancestry estimate. A sample can produce an FL for a site because, for example, a small region of the chromosome around this site is missing or is of different sequence character than for most (this result is not uncommon given the highly variable nature of the chromosomal positions we measure); or because not enough DNA was obtained from the buccal swab used to collect a DNA sample.

The genome was scanned for a useful panel of BGA AIMs and the best 71 AIMs were identified using the maximum likelihood algorithm to measure BGA admixture proportions were selected (Table 6). Using these AIMS, majority BGA affiliations were measurable in a manner consistent with self-held notions on BGA, and BGA admixture proportions were measurable with significantly improved precision, accuracy and reliability compared to previously described methods for the inference of race (see Example 2; see, also, Example 1, using 32 marker test). This test can be used during study design to help reduce or eliminate the insidious effects that cryptic or micro population structure imposes. The test also can be useful for forensic scientists who currently use imprecise and sometimes inaccurate means by which to infer race from crime scene DNA.

The present invention also provides articles of manufacture, including one or a plurality of photographs, each photograph being of a person having a known proportional ancestry corresponding to a population structure comprising nucleotide occurrences of AIMs indicative of BGA, the known proportional ancestry being associated with the photograph in the article. An article of manufacture of the invention (i.e., a photograph and the proportional ancestry information) can be contained in one or more files (e.g., the photograph and information in one file, or the photograph in one file and the information in a second file, which is or can be linked to the photograph). If desired, more than one photograph of an individual having a known proportional ancestry can be contained in the same or a linked file, for example, photographs containing different profiles of the individual or photographs of the individual at various ages.

Similarly, a plurality of the articles (i.e., photographs and proportional ancestry information) can be contained in a file, for example, a file containing a plurality of photographs of different persons, wherein the some or all of the persons have the same or different known proportional ancestries that correspond to a population structure comprising nucleotide occurrences of AIMs indicative of BGA. Such a plurality of articles also can be contained in different files, including, for example, a plurality of files, each containing one photograph and information regarding the known proportional ancestry of the individual in the photograph, or each containing two or more photographs of different individuals, each of which contains the same known proportional ancestry, or each containing two or more photographs of different individuals, some or all of which have a different proportional ancestry as compared to another individual whose photograph is contained in the file. Accordingly, a plurality of such articles is provided, as is a plurality of files, each file of which can contain one or more articles, i.e., photographs, which can be of one or more persons having the same or different known proportional ancestries that correspond to a population structure comprising nucleotide occurrences of AIMs indicative of BGA; and the plurality of files can contain files, each of which contains one or more photographs of one or more persons, and when containing one or more photographs of two or more different persons, the different persons can have the same or different known proportional ancestries.

The article of manufacture, i.e., the photograph of a person having a known proportional ancestry corresponding to a population structure comprising nucleotide occurrences of AIMs indicative of BGA can be a digital photograph, which comprises digital information, including for the photographic image and any other information that may be relevant or desired (e.g., the age, name, or contact information of the subject in the photograph, or the subject's answer on a questionnaire as to what the subject believes his or her ancestry to be). Such digital information of one or more digital photographs can be contained in a database thus facilitating searching of the photographs and/or known proportional ancestry information using electronic means. As such, the present invention further provides a plurality of the articles of manufactures, including at least two digital photographs, each of which comprises digital information. Where the digital information for one or a plurality of the articles is contained in a database, it can comprise any medium suitable for containing such a database, including, for example, computer hardware or software, a magnetic tape, or a computer disc such as floppy disc, CD, or DVD. As such, the database can be accessed through a computer, which can contain the database therein, can accept a medium containing the database, or can access the database through a wired or wireless network, e.g., an intranet or internet.

The present invention also provides kits useful for practicing a method of the invention. Such kits can contain, for example, a plurality of hybridizing oligonucleotides, each of which has a length of at least fifteen contiguous nucleotides of a polynucleotide as set forth in SEQ ID NOS:1 to 331 (or a polynucleotide complementary thereto), the plurality including at least five (e.g., 5, 6, 7, 8, 9, 10, 15, 20, 25, 30, etc.) of such oligonucleotides, each based on different polynucleotides as set forth in SEQ ID NOS:1 to 331. In one embodiment, the hybridizing oligonucleotides that include at least fifteen contiguous nucleotides of at least five polynucleotides as set forth in SEQ ID NOS:1 to 71, or polynucleotides complementary to any of SEQ ID NOS:1 to 71. In another embodiment, the hybridizing oligonucleotides are specific for at least ten AIMs as set forth in SEQ ID NOS:1 to 71. A kit of the invention also can contain at least two panels of such hybridizing oligonucleotide, including, for example, a panel of at least five (e.g., 5, 6, 7, 8, 9, 10, 11, 12, 13, 14, 15, etc.) hybridizing oligonucleotides specific for AIMs as set forth in SEQ ID NOS:7, 21, 23, 27, 45, 54, 59, 63, and 72 to 152; or a panel of at least five hybridizing oligonucleotides specific for an AIM as set forth in SEQ ID NOS:3, 8, 9, 11, 12, 33, 40, 59, 63, and 153 to 239; or a panel of at least five hybridizing oligonucleotides specific for AIMs as set forth in SEQ ID NOS:1, 8, 11, 21, 24, 40, 172, and 240 to 331; or two or more of such panels and/or a panel of at least five hybridizing oligonucleotides specific for AIMs as set forth in SEQ ID NOS:1 to 71.

The hybridizing polynucleotides of a kit of the invention can include probes, which are useful for detecting a particular AIM, including a particular nucleotide occurrence at the SNP position of the AIM; can include primers, including primers useful for a primer extension reaction and primer pairs useful for a nucleic acid amplification reaction; or can include combinations of such probes and primers. A hybridizing oligonucleotide of the plurality can, but need not, include a nucleotide corresponding to nucleotide position of the SNP or DIP of an AIM, e.g., nucleotide 50 of an AIM as set forth in any of SEQ ID NOS:1 to 55 and 57 to 331 or nucleotide 26 of SEQ ID NO:56, or to a nucleotide sequence complementary thereto, such a hybridizing oligonucleotide being useful as a probe to identify the presence or absence of a particular nucleotide occurrence at the SNP position of the AIM.

A kit of the invention also can contain at least one pair of hybridizing oligonucleotides useful for detecting the nucleotide occurrence at the SNP position or the presence or absence of a nucleotide sequence the DIP position of an AIM. For example, a pair of hybridizing oligonucleotides can include one oligonucleotide that hybridizes upstream and adjacent to the SNP position of an AIM and a second oligonucleotide that hybridizes downstream of and adjacent to the SNP position of the AIM, wherein one or the other of the pair further contains a nucleotide complementary to a nucleotide occurrence suspected of being at the SNP position of the AIM (i.e., one of the polymorphic nucleotides), such a pair of hybridizing oligonucleotides being useful in an oligonucleotide ligation assay. In another example, a pair of hybridizing oligonucleotides can include an amplification primer pair, including a forward primer and a reverse primer, such a pair of hybridizing oligonucleotides being useful for amplifying a portion of polynucleotide that includes the SNP or DIP position of the AIM.

A kit of the invention can further contain additional reagents useful for practicing a method of the invention. As such, the kit can contain one or more polynucleotides comprising an AIM, including, for example, a polynucleotide containing an AIM for which a hybridizing oligonucleotide or pair of hybridizing oligonucleotides of the kit is designed to detect, such polynucleotide(s) being useful as controls. Further, hybridizing oligonucleotides of the kit can be detectably labeled, or the kit can contain reagents useful for detectably labeling one or more of the hybridizing oligonucleotides of the kit, including different detectable labels that can be used to differentially label the hybridizing oligonucleotides; such a kit can further include reagents for linking the label to hybridizing oligonucleotides, or for detecting the labeled oligonucleotide, or the like. A kit of the invention also can contain, for example, a polymerase, particularly where hybridizing oligonucleotides of the kit include primers or amplification primer pairs; or a ligase, where the kit contains hybridizing oligonucleotides useful for an oligonucleotide ligation assay. In addition, the kit can contain appropriate buffers, deoxyribonucleotide triphosphates, etc., depending, for example, on the particular hybridizing oligonucleotides contained in the kit and the purpose for which the kit is being provided.

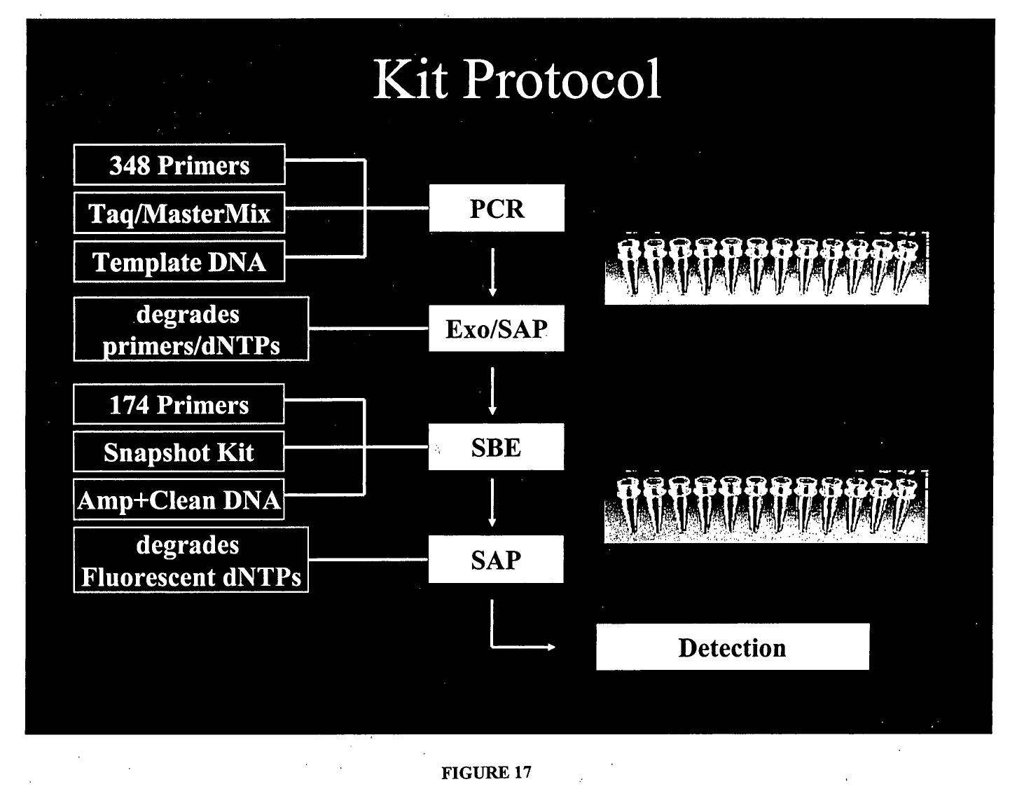

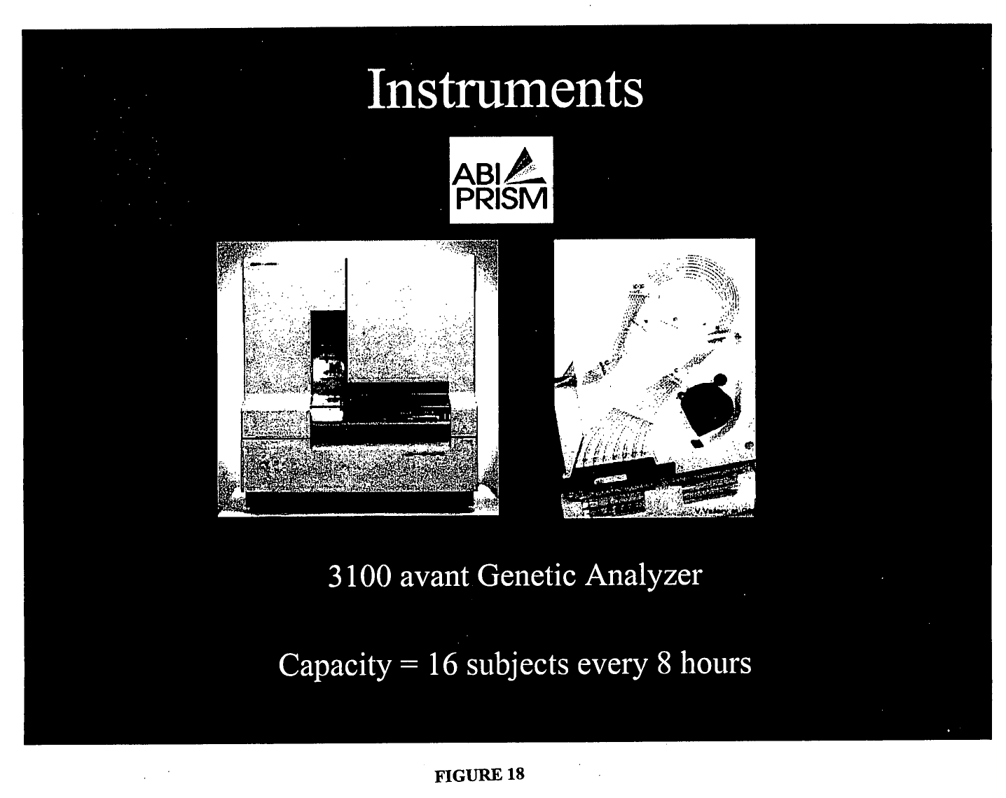

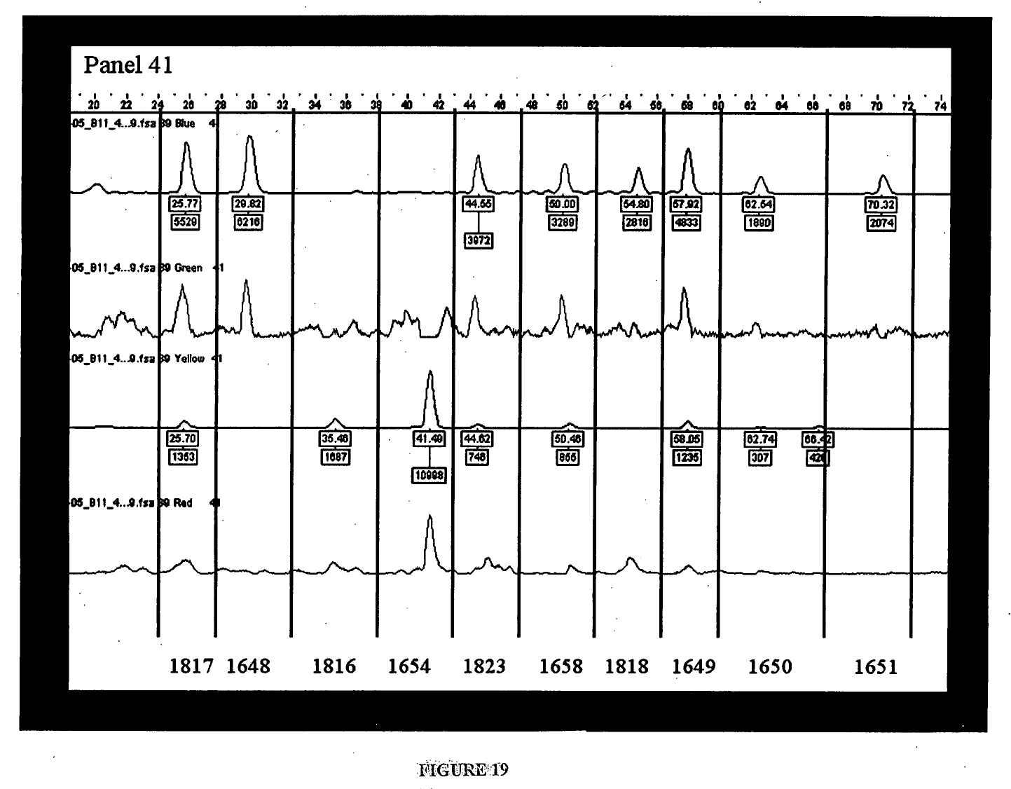

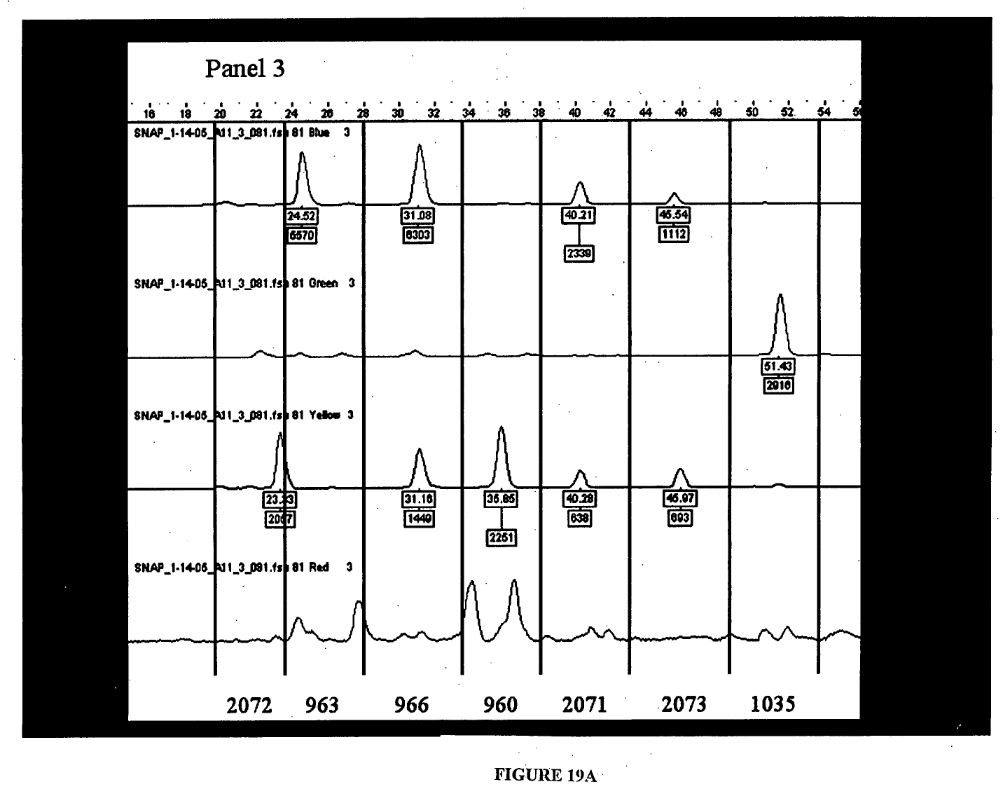

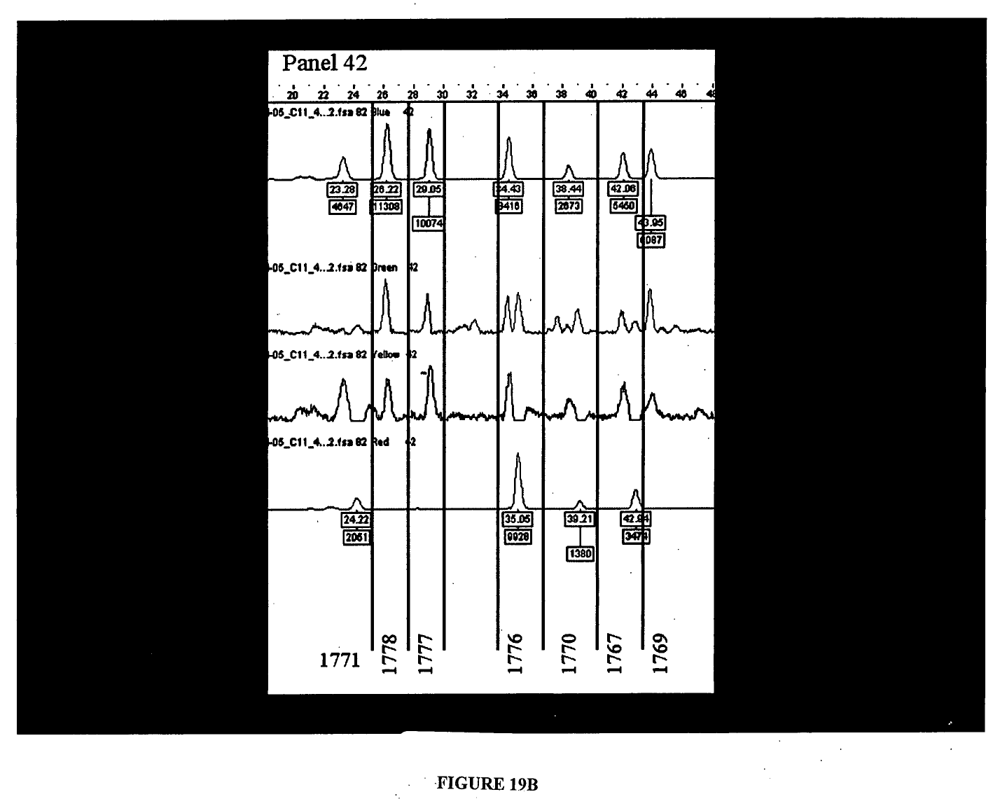

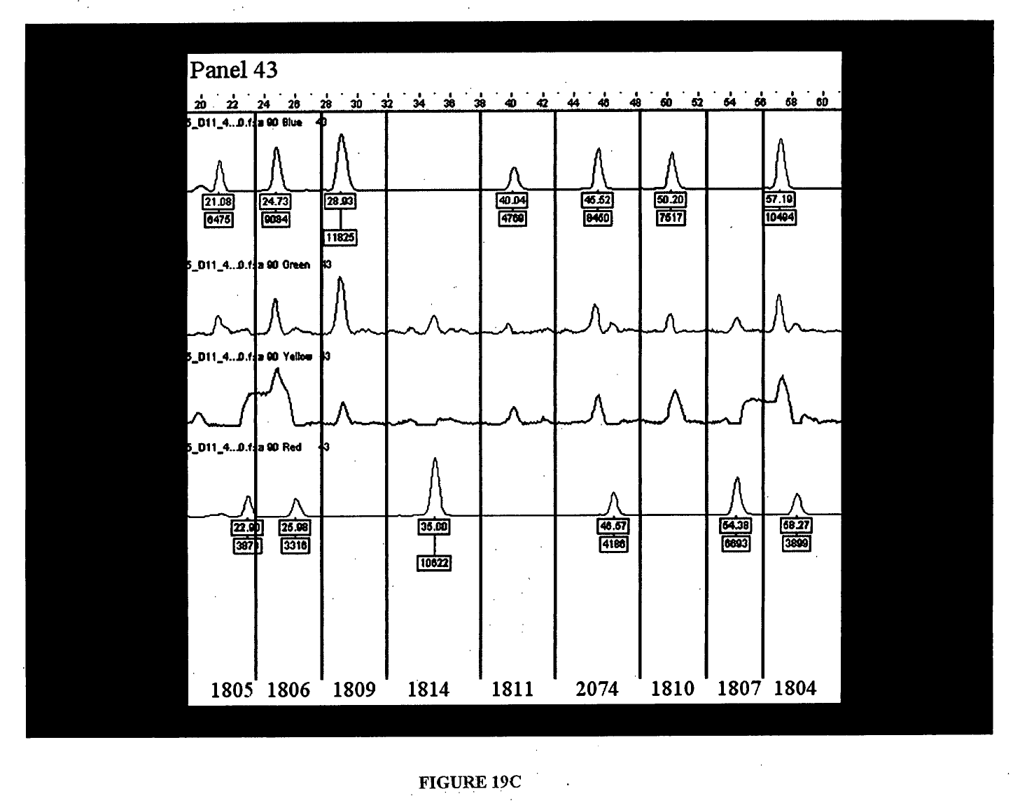

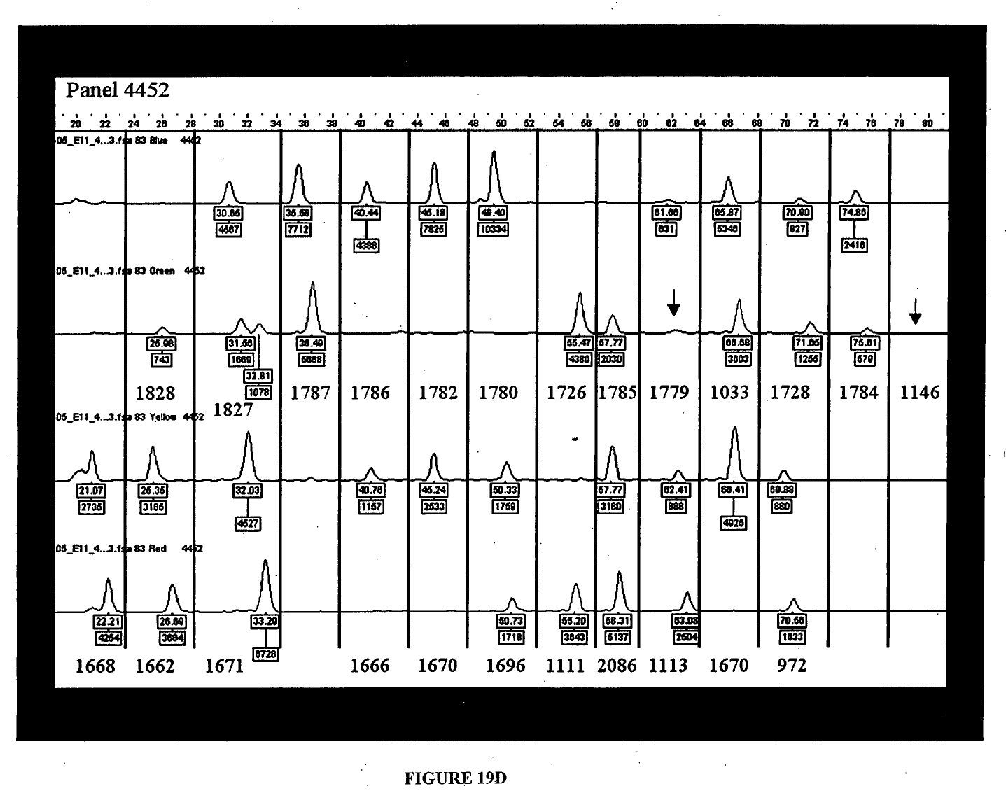

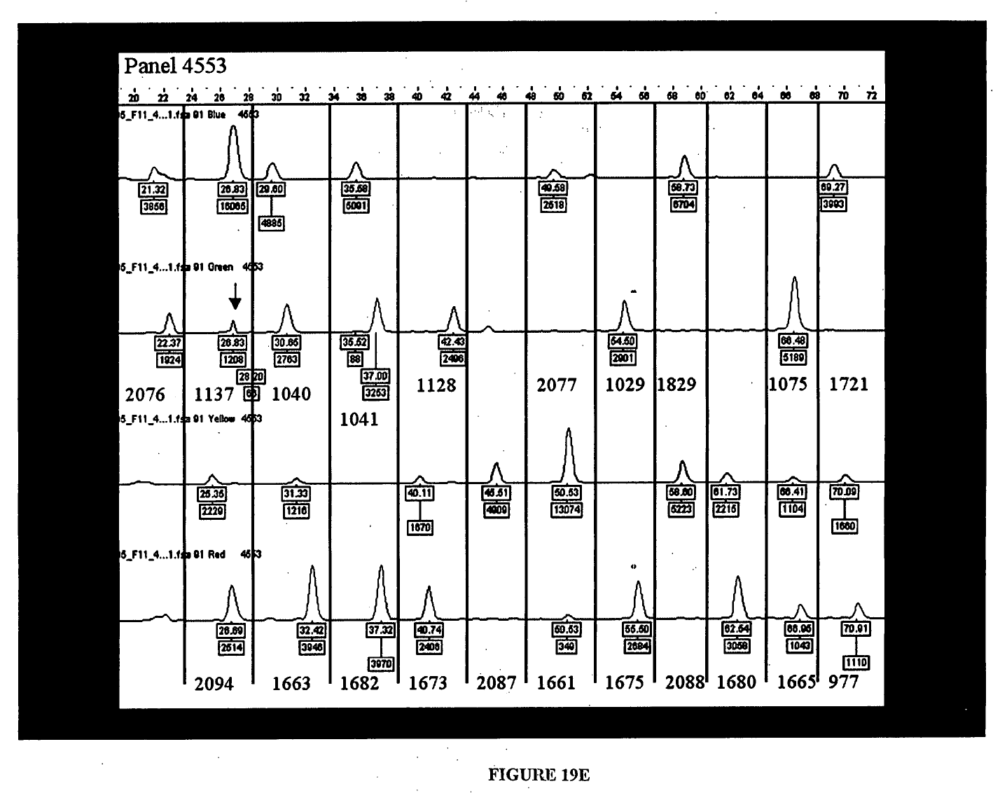

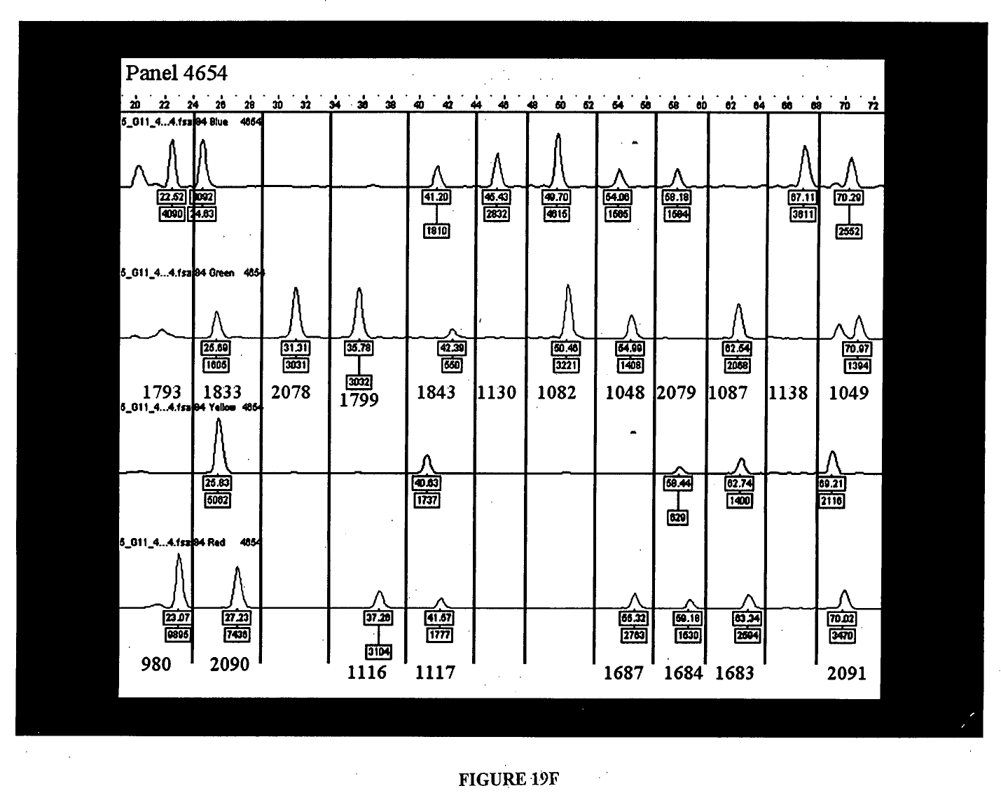

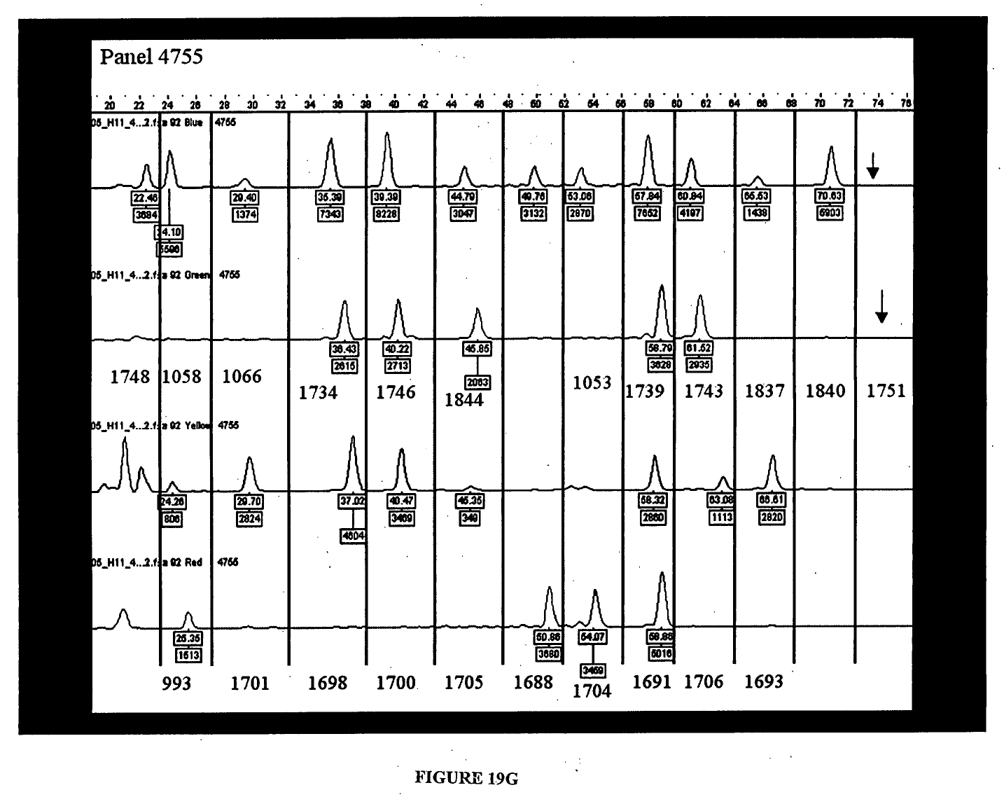

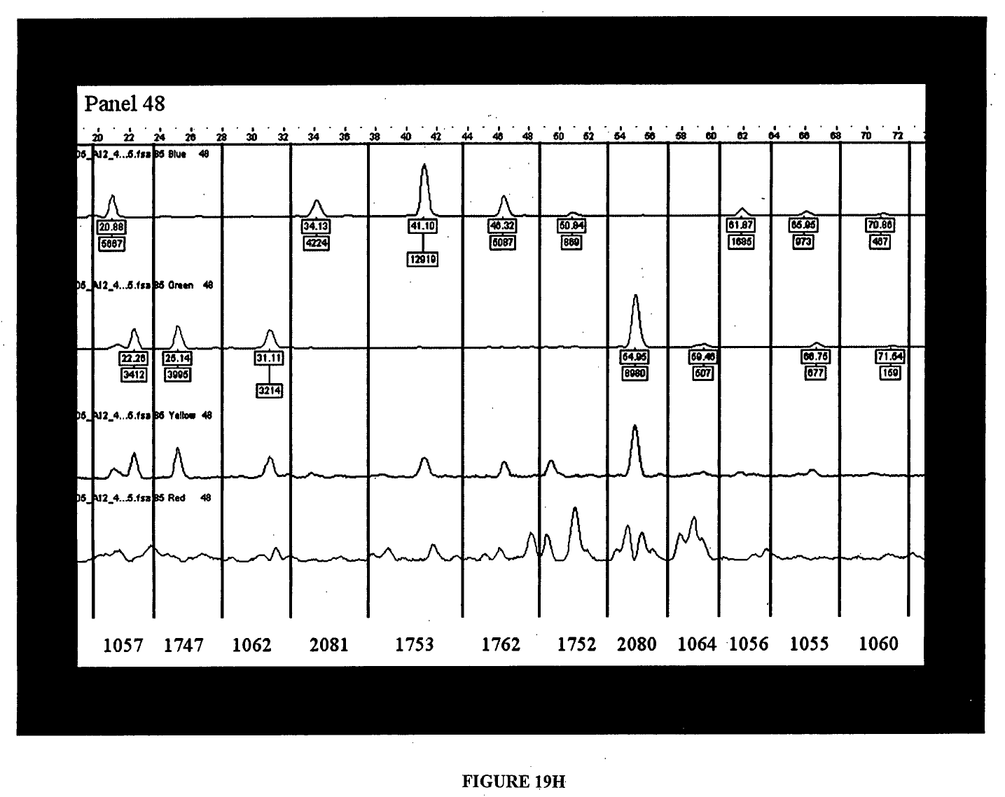

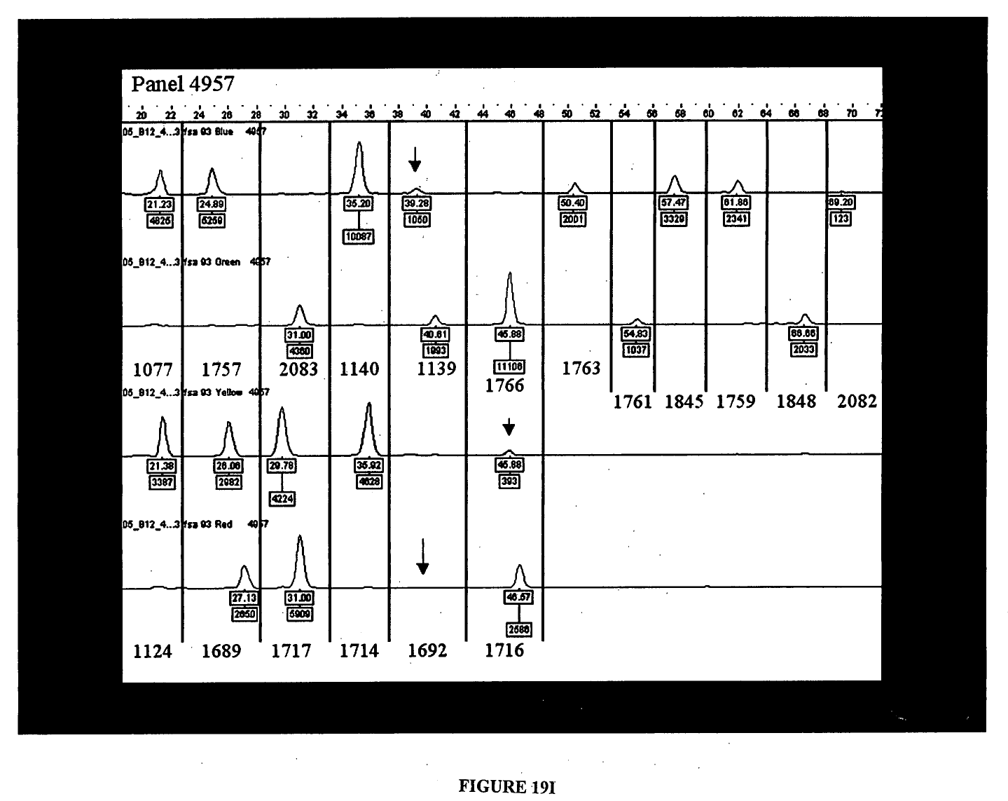

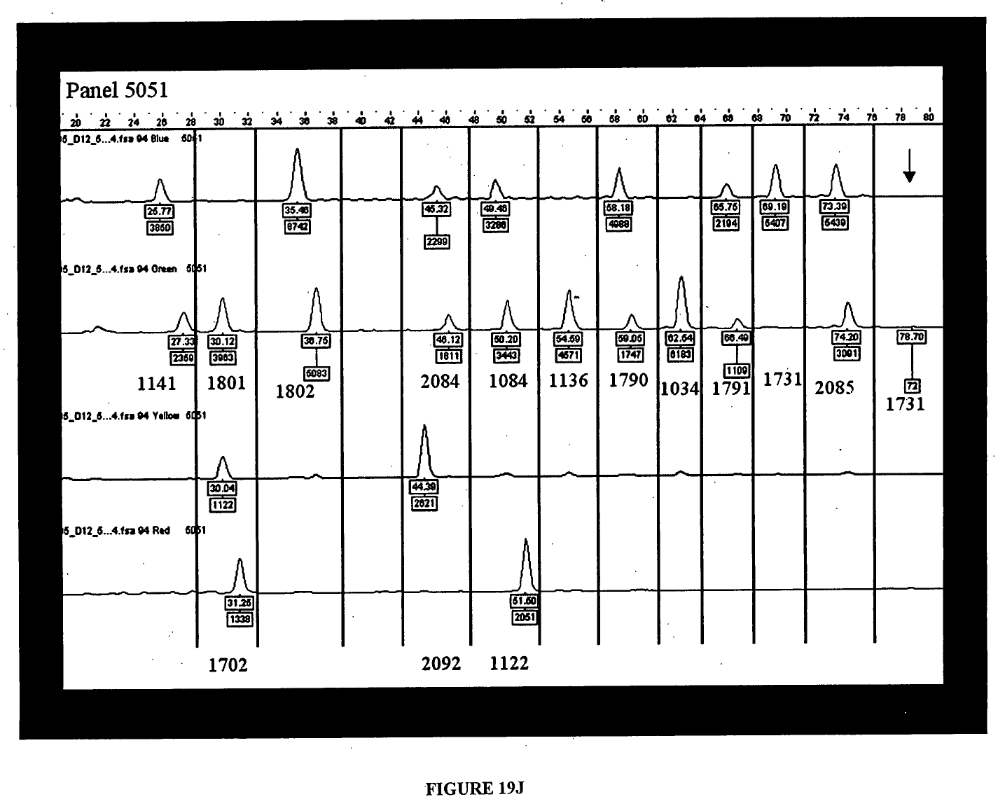

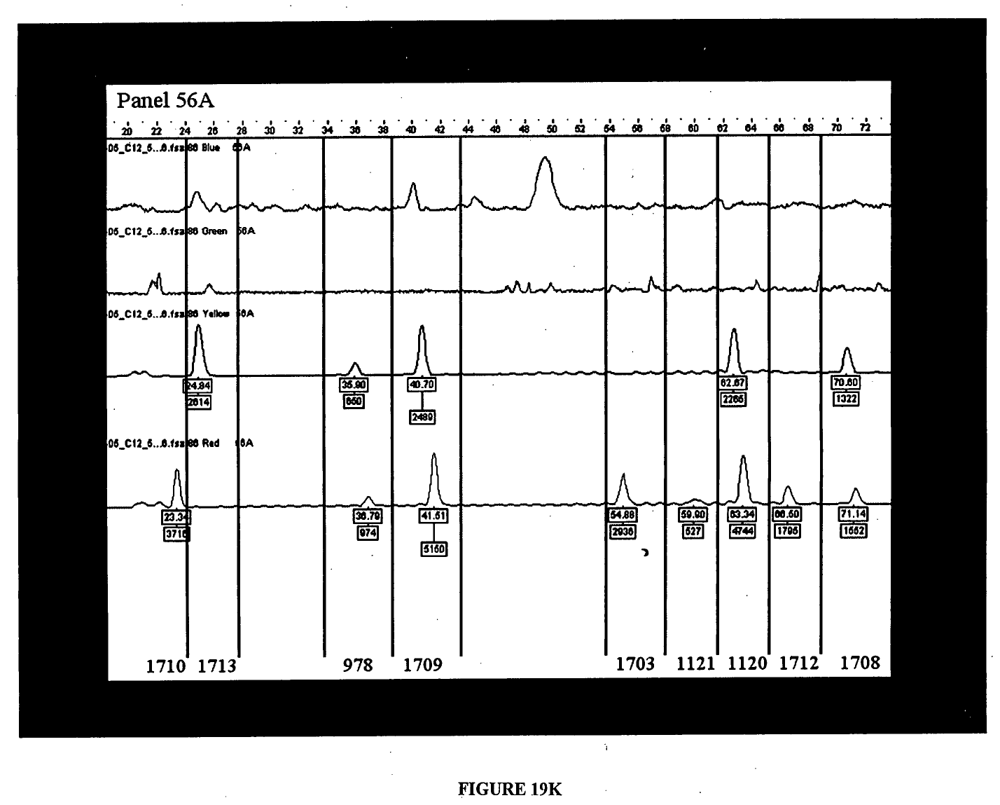

The present invention is based on the development of reagents, including oligonucleotide primers and primer pairs that allow the performing of multiplex reactions for determining the nucleotide occurrences of single nucleotide polymorphisms (SNPs) of ancestry informative markers (AIMs), which are useful for inferring a level of population structure of an individual and, in turn, an inference as to various traits of the individual. A correlation of AIMs used in the exemplified methods with a trait was demonstrated in International Publication No. WO 2004/016768 (PCT/US2003/026229), which is incorporated herein by reference. The AIMs used to exemplify the present methods are set forth as SEQ ID NOS: 364 to 537 in Appendix 3 (42 pages), which is incorporated herein by reference, and include the AIMs (SNPs) set forth in SEQ ID NOS: 371 to 398, 400 to 408, 410 to 413, 415, 418, 420, 422, 423, 425, 431 to 433, 438 to 441, 443, 450 to 452, 455, 456, 461 to 463, 467 to 475, 477 to 485, 487, 495 to 498, 502 to 504, 506, 508 to 512, 514, 516, 519 to 521, 526, 529, and 533 to 537, which, as disclosed herein, are useful for inferring a trait of an individual (e.g., biogeographical ancestry). Panels of single base extension (SBE) primers useful for multiplex analysis of the exemplified AIMs are shown, respectively, in Appendix 1 (11 pages), which is incorporated herein by reference, and polymerase chain reaction (PCR) primer pairs useful for amplifying gene regions containing the exemplified AIMs are shown, respectively, in Appendix 2 (15 pages), which is incorporated herein by reference. Corresponding panels of SBE primers and PCR primer pairs (i.e., panels of primers and primer pairs that can be used together in a single multiplex reaction to sample a plurality of 7 to 24 different AIMs) are indicated in Appendices 1 and 2 (e.g., Panel 3 in Appendices 1 and 2 contain 7 SBE primers and 14 PCR primer pairs, respectively, that can be used in a single multiplex reactions to sample (i.e., determine the nucleotide occurrence) the AIMs set forth as SEQ ID NOS:1 to 7, respectively. The exemplified multiplex reactions and results obtained using the panels of PCR primer pairs (Appendix 2) and SBE primers (Appendix 1) are shown, which is incorporated herein by reference, and in FIGS. 17-19A-K.

The compositions exemplified herein provide a DNA test adapted for use with capillary electrophoresis equipment. The exemplified assay, which examines the Biogeographical ancestry (BGA) proportions of individuals, analyzes 174 SNPs that are informative for continental ancestry (see, International Publ. No. WO 04/016768). The exemplified compositions provide a test kit that allows multiplex analysis of the 174 SNPs in 12 reactions. SBE primers were designed such that the reaction products could be separated and easily read using standard capillary electrophoresis software and equipment (see Example 1; see, also, Appendix 1). As such, the exemplified assay allows the BGA informative SNPs to be examined on a platform that is commonly used in the field, and reduces the cost per test, thus allows tests to be performed economically by a large number of laboratories using readily available reagents.

A multiplex assay is exemplified herein by performing multiplex PCR reactions, then examining the SNP in the PCR products using multiplex SBE reactions. Following the PCR reaction, which amplified the region surrounding each SNP, the unreacted reagents were removed, and a removed by treatment with shrimp alkaline phosphatase (see Example 1), and the primers were detected using a capillary electrophoresis and detection system (Applied Biosystems, Inc. (ABI), Foster City Calif.). An ABI 3700 Genetic Analyzer instrument was used for the exemplified studies, although any similar platform can be used, including, for example, an ABI 310 Genetic Analyzer, 3100 Genetic Analyzer, 3130 Genetic Analyzer, 3700 Genetic Analyzer, or 3730 Genetic Analyzer instrument, or the exemplified system can be modified as disclosed herein and reaction products (SNPs) can be detected, for example, using a mass spectroscopy system (e.g., Sequenom; San Diego Calif.), a BeadArray® system (Illumina; San Diego Calif.); a LightTyper™ system (Roche), a NanoChip® system (Nanogen; San Diego Calif.); a fluorescence polarization/template directed dye termination (FP-TDI) system (Perkin-Elmer; Boston Mass.); or any other system using, for example, slab gel electrophoresis, printed microarrays, colorimetric detection platforms (e.g., flourimetry), or high performance liquid chromatography systems.

In addition to the exemplified combinations of primers used in a multiplex reaction, alternative groups of the primers can be used such that the final SBE products can be resolved by the detecting system (e.g., capillary electrophoresis). The SBE primers used in alternative groupings further can be modified such that products of resolvably different sizes are generated. By employing such modification, the number of reactions that need to be run for each sample (person) can be further reduced, for example, to 10, 8, 6 or fewer multiplex reaction, including as few as a single tube reaction. Such methods would allow examination of nucleic acid molecule samples that are available only in a very low quantity, or are of a low quality. Of course, the number of SNPs examined can be reduced from 174 (e.g., to 150, 100, 70, 50, 25, or fewer), for example, by performing fewer than all 12 of the exemplified multiplex reactions (e.g., 1, 2, 3, 4, 5, 6, 7, 8, 9, 10, or 11 reactions), although the confidence level of the inference will be reduced accordingly.

In addition to altering the exemplified primers and/or combinations of primers, the methods used for removing unreacted products from the reaction(s) can be varied (e.g., using spin columns) or modified, preferably while minimizing sample manipulation. Similarly, labels other than those used in the exemplified methods also can be used, for example, where capillary electrophoresis instruments of other vendors are used (e.g., Beckman-Coulter, or Hitachi), or where different detection systems are used (e.g., mass spectrometry). Additional modifications to the exemplified method can include, for example, performing the reaction in a single tube. For example, the reaction can be performed using PCR primers that include a biotinylated PCR primer, wherein the PCR amplification product is bound to streptavidin, which, in turn, is bound to a solid substrate (e.g., a bead, or a surface of a tube or well in which the reaction is performed); following PCR and washing of the solid substrate, the SBE reaction can be performed in the same tube (or well).

The disclosed compositions and multiplex methods provide a tool for human forensics, as well as for pharmaceutical testing, demographic testing, ancestry testing, identity testing, and remains identification of human. Further, the methods and compositions can be applied across an array of species such as dogs, cats, horses, pigs, cattle, or any domesticated or wild species of mammalian origin. Further, the compositions and methods can be used with instruments other than capillary electrophoresis, and, as mentioned above, can be performed by binding a component of the reaction (e.g., a PCR primer, an SBE primer, or a nucleic acid sample to be tested) to plastic, glass, metal or any other suitable surface, and reaction products comprising the nucleotide occurrences of the SNPs can be measured, for example, by detecting emitted light of either UV or other radiation reaction or change in frequency or other detectible linker using other laboratory instruments. The exemplified methods are performed using liquid-liquid interactions. However, as discussed above, the assays can be modified to be performed using dry-liquid interactions or dry-dry interactions. Additionally, one or more components of a multiplex reaction can be applied to a silicon wafer or other conducting material used in microelectronic applications, and reaction products can be measured, for example, by ion detection or electron capture. Such modifications to the exemplified system can allow the methods to performed using microelectronic or nanotechnology methods. In one aspect, the modifications allow the construction of field service kits. Further, the exemplified compositions can be used in other formats in multiplex reactions, including, for example, allele specific hybridization, allele specific oligonucleotide ligation, sequencing, single stranded conformational polymorphism, heteroduplex analysis, allele specific PCR, and allele specific PCR with locked nucleic acid molecules.

As disclosed herein, multiplex reactions have been designed such that the nucleotide occurrences of SNPs of 174 AIMs can be examined in 12 reactions. AIMs are genetic loci that show alleles with high frequency differences between populations. AIMs are exemplified herein generally by SNPs (see, e.g., SEQ ID NO:1, Appendix 3, wherein the alternative alleles indicated in brackets), although AIMs also occur as deletion/insertion polymorphisms (see WO 2004/016768; e.g., SEQ ID NO:363). AIMs can be used to estimate BGA of an individual or collection of individuals at the population level (in terms of races), at the sub-population level (in terms of ethnicities), and at the micro-group level (in terms of familial lines within ethnic groups), as well as at a practical, phenotypically qualified level (e.g., cases and controls). Such ancestry estimates at the subgroup and individual level can be directly instructive regarding the genetics of phenotypes that are different qualitatively or in frequency between populations, including, for example, the likelihood that an individual will respond to a particular medication or the propensity of an individual to develop a disease. Ancestry estimates also can provide a compelling foundation for the use of Admixture Mapping (AM) methods to identify the genes underlying these traits.

The multiplex methods disclosed herein provide a tool that conveniently allows, for example, 1) for the estimation of ancestry proportions in individuals from their DNA; 2) for the estimation of genetic structure for the control of study designs commonly used for genetic research; 3) for the construction of physical profiles through the inference of characteristics related to ancestry, which may have implications in forensic investigations; 4) for the identification of disease predisposition, referred to as “Mapping by Ancestry Linkage Disequilibrium” (MALD); and 5) for predicting a significant portion of an individual patient's response to prescription and over-the-counter medications, in only a few reactions that, along with the exemplified primers and primer pairs, use readily available reagents and equipment. Accordingly, the present invention provides multiplex methods for inferring, with a predetermined level of confidence, a trait of an individual, and further provides kits for practicing such methods. In one embodiment, a method of the invention is performed by contacting at least a first sample (i.e., 1, 2, 3, 4, or more sample(s)) that contain test nucleic acid molecules of the individual with at least a first plurality of primers, including at least one plurality of SBE primers as set forth in the panels of Appendix 1 (e.g., Panel 3, Panel 41, etc.), under conditions suitable for SBE of the primers; and detecting at least one SBE product of a primer of the first (or other) plurality.

A test individual (i.e., an individual from which test nucleic acid molecules are obtained) for whom a trait is to be inferred can be any individual for whom it is desired to infer a trait, and for whom a nucleic acid sample is available. As disclosed herein, a test nucleic acid sample can be nucleic acid molecules as obtained from an individual (e.g., nucleic acid molecules present in a blood, semen, oral swab, or tissue sample), or can be product produced from such nucleic acid molecules (e.g., PCR amplification products). Generally, an individual providing test nucleic acid molecules is a human, though the methods of the invention also can be used for inferring traits of other mammals, including, for example, domestic animals such as cats, dogs, or horses; farm animals such as cattle, sheep, pigs, or goats; or other animals. The trait to be examined can be any trait of interest, including, as exemplified herein, proportional ancestry (BGA); hair, skin or iris pigmentation; or drug responsiveness.

The methods of the invention are particularly useful because they allow for an inference to be made of a desired trait with a predetermined level of confidence. As used herein, reference to a “predetermined level of confidence” means that an inference or estimate of the invention is made using statistical methods that provide a confidence interval to be determined about a mean or a maximum likelihood value. In addition to determining the maximum likelihood value of within-individual or within-sample structure, other similarly likely values can also be determined and these can be combined to define the x-fold likelihood confidence intervals, where x is any number such as 2, 5 or 10. For example, all of the structure results corresponding to a likelihood value 10 times lower than the Maximum Likelihood Value can be plotted or listed to define the 10-fold likelihood confidence interval. As for any statistical test, an assay of the invention is designed such that performance of the test results in a value having a desired confidence level. A method of the invention can be performed such that the result has a predetermined level of confidence by varying the number of AIMs examined with respect to a trait. For example, use of one exemplified panel to sample a plurality of AIMs (see Example 1 and Appendices 1 and 2) can allow an inference to be made as to whether an individual has a particular trait, e.g., responsiveness to Lipitor™ medication, with a certain level of confidence, whereas the use of two or more of the exemplified panels can allow the same inference to be made, but with a higher level of confidence. Similarly, use of one of the exemplified panels can allow an inference to be made that an individual has, for example, 80% IndoEuropean ancestry and 20% East Asian ancestry (with an error, e.g., of +10%), whereas the use of two or more panels can allow the same inference, but with an error, e.g., of +5%.

A sample useful for practicing a method of the invention can be any biological sample of a test individual that contains nucleic acid molecules, including portions of the gene sequences containing AIMs that are to be examined or, wherein the polymorphism of an AIM results in an amino acid change in an encoded polypeptide, any biological sample that contains the encoded polypeptides. As such, the sample can be a cell, tissue or organ sample, or can be a sample of a biological fluid such as semen, saliva, blood, cerebrospinal fluid, and the like.

A nucleic acid sample useful for practicing a method of the invention will depend, in part, on whether the SNPs to be identified are in coding regions or in non-coding regions. Where one or more SNPs is present in a non-coding region of a gene, the nucleic acid sample generally is a deoxyribonucleic acid (DNA) sample, particularly genomic DNA or an amplification product thereof. The primer pairs exemplified in Appendix 2 can be used to obtain such amplification products, particularly a plurality of amplification products comprising different AIMs, which can be prepared in a multiplex reaction using the exemplified panels of primer pairs (e.g., Panel 3, Panel 41, etc.). Where the AIM is contained within a transcribed sequence, e.g., rDNA, microsatellite DNA, or heteronuclear ribonucleic acid (RNA), which includes unspliced mRNA precursor RNA molecules including non-coding RNA sequence, an RNA sample can be used and examined directly, or a cDNA or amplification product thereof can be examined according to the present methods. Where one or more SNPs is present in a coding region of a gene, the nucleic acid sample can be DNA or RNA, or products derived therefrom, for example, amplification products. Furthermore, while the methods of the invention are exemplified with respect to a nucleic acid sample, it will be recognized that particular SNPs, when present in coding regions of a gene, can result in polypeptides containing different amino acids at the positions corresponding to the SNPs due to non-degenerate codon changes. As such, methods using a sample containing polypeptides of the subject also are envisaged.

Conditions suitable for detecting the nucleotide occurrences of AIMs are exemplified herein (see Example 1), and can otherwise be varied depending, for example, on the sequences of the hybridizing oligonucleotides, including their length and complementarity, as well as on the particular reaction being performed (e.g., an amplification reaction, or a single base extension reaction). The hybridizing oligonucleotides (i.e., SBE primers and PCR primer pairs) can be as exemplified and contain deoxyribonucleotides, or can contain one or more ribonucleotides that do not interfere with the reaction for which the oligonucleotide is to be used. Further, the nucleotides can be linked together by a phosphodiester bond, and can be single stranded or double stranded, though they generally are used in a single stranded form, and can be prepared using methods of chemical synthesis or by enzymatic methods.

The hybridizing oligonucleotides, or other polynucleotides useful in a methods or contained in a kit of the invention also can contain nucleoside or nucleotide analogs, and can have a backbone bond other than a phosphodiester bond, such oligonucleotides providing certain advantages such as having increased stability or more desirable hybridization properties. Nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference). The covalent bond also can be any of numerous other bonds, including a thiodiester bond, a phosphorothioate bond, a peptide-like bond or any other bond known to those in the art as useful for linking nucleotides to produce synthetic oligonucleotides (see, for example, Tam et al., Nucl. Acids Res. 22:977-986, 1994; Ecker and Crooke, BioTechnology 13:351360, 1995, each of which is incorporated herein by reference). The incorporation of non-naturally occurring nucleotide analogs or bonds linking the nucleotides or analogs can be particularly useful where the oligonucleotide is to be exposed to an environment that can contain a nucleolytic activity, including, for example, a tissue culture medium or sample comprising a cell extract because the modified oligonucleotides can be less susceptible to degradation.

Generally, the hybridizing oligonucleotides (i.e., primers and primer pairs) useful for purposes of the present invention are at least about 15 bases in length, which is sufficient to permit the oligonucleotide to selectively hybridize to a target polynucleotide comprising the AIM, and can be at least about 18 nucleotides or 21 nucleotides or 25 nucleotides or more in length. The term “selective hybridization” or “selectively hybridize” refers to hybridization under moderately stringent or highly stringent physiological conditions, which can distinguish related nucleotide sequences from unrelated nucleotide sequences. In nucleic acid hybridization reactions, the conditions used to achieve a particular level of stringency are known to vary, depending on the nature of the nucleic acids being hybridized, including, for example, the length, degree of complementarity, nucleotide sequence composition (e.g., relative GC:AT content), and nucleic acid type, i.e., whether the oligonucleotide or the target nucleic acid sequence is DNA or RNA. An additional consideration is whether one of the nucleic acids is immobilized, for example, on a filter, bead, chip, or other solid matrix. Examples of conditions that allow for selective hybridization of panels of primers and primer pairs useful in multiplex reactions performed in solution are provided in Example 1.

For other reactions conditions or when other primers and/or primer pairs than those exemplified are used, appropriate stringency conditions can be determined empirically or estimated using various formulas, as is well known in the art (see, for example, Sambrook et al., Molecular Cloning: A laboratory manual (Cold Spring Harbor Laboratory Press, 1989). An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed. As such, final conditions will vary, depending on the particular hybridization reaction involved, and can be determined empirically. It should be recognized that a variety of conditions can be utilized to provide selective hybridization conditions. For example, when a multiplex assay is to be performed using a plurality of different hybridizing oligonucleotides specific for different AIMs of a panel, the conditions (as well as the AIMs/hybridizing oligonucleotides) can be selected such that selective hybridization occurs for all of the hybridizing oligonucleotides in the reaction.

In various embodiments, it can be useful to detectably label a polynucleotide or hybridizing oligonucleotide. In addition, when a multiplex reaction is performed as exemplified herein, labeling of an SBE primer using differentially labeled dideoxynucleotides can provide reaction products that conveniently can be detected. Detectable labels useful for the present methods include, for example, chemiluminescent labels, radionuclides, enzymes, haptens such as digoxygenin and biotin, fluorophores, and unique oligonucleotide sequences. In addition to the exemplified labeling by SBE, additional sequences can be labeled depending, for example, on the product to be detected. For example, labeling of PCR products can be performed, wherein one primer is biotinylated and the other primer contains digoxygenin. The amplification products can then be bound to a streptavidin plate, washed, reacted with an enzyme-conjugated antibody to digoxygenin, and developed with a chromogenic, fluorogenic, or chemiluminescent substrate for the enzyme. Such a method can be useful, for example, where a multiplex reaction is to be performed using a solid state protocol. A radioactive method also can be used to detect generated amplification or SBE products, for example, by including a radiolabeled deoxynucleoside triphosphate or dideoxynucleoside triphosphate, respectively, into the reaction. In addition, if one primer is biotinylated, then streptavidin-coated scintillation proximity assay plates can be used to measure the PCR products. Additional methods of detection can use a chemiluminescent label, for example, a lanthanide chelate such as used in the DELFIA® assay (Pall Corp.), a fluorescent label, or an electrochemiluminescent label such as ruthenium tris-bipyridyl (ORI-GEN).

In addition to SBE (see Example 1), methods for detecting a nucleotide occurrence at a SNP position of an AIM can utilize one or more oligonucleotide probes or primers, including, for example, an amplification primer pair, that selectively hybridize to a target polynucleotide spanning the AIM. Oligonucleotide probes useful in practicing a method of the invention can include, for example, an oligonucleotide that is complementary to and spans a portion of the target polynucleotide, including the position of the SNP, wherein the presence of a specific nucleotide at the position of the SNP is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe. A pair of probes that specifically hybridize upstream and adjacent and downstream and adjacent to the site of the SNP, wherein one of the probes includes a nucleotide complementary to a nucleotide occurrence of the SNP, also can be used in an oligonucleotide ligation assay, wherein the presence or absence of a ligation product is indicative of the nucleotide occurrence at the SNP site. An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site or to determine whether there is an insertion or a deletion at the DIP site.

Conditions that allow generation of amplification products and SBE products in a sample using a multiplex method are exemplified in Example 1. In addition, conditions in which an amplification reaction and/or SBE reaction is being performed can be modified from those exemplified, or selected de novo, such that the reaction contains the necessary components for the amplification reaction and/or SBE reaction to occur. Such conditions include, for example, appropriate buffer capacity and pH, salt concentration, metal ion concentration if necessary for the particular polymerase, appropriate temperatures that allow for selective hybridization of the primers or primer pairs of a panel to the template target polynucleotides, as well as appropriate cycling of temperatures that permit polymerase activity and melting of a primer or primer extension or amplification product from the template or, where relevant, from forming a secondary structure such as a stem-loop structure. Such conditions and methods for selecting such conditions are routine and well known in the art (see, for example, Innis et al., “PCR Strategies” (Academic Press 1995); Ausubel et al., “Short Protocols in Molecular Biology” 4th Edition (John Wiley and Sons, 1999), each of which is incorporated herein by reference).

An SBE product can be detected as exemplified in Example 1. Further, a primer extension (e.g., SBE product) or amplification product can be detected directly or indirectly and/or can be sequenced using various methods known in the art. Amplification products that span a SNP site can be sequenced using traditional sequence methodologies, including, for example, the dideoxy-mediated chain termination method (Sanger et al., J. Molec. Biol. 94:441, 1975; Prober et al. Science 238:336-340, 1987) or the chemical degradation method (Maxam et al., Proc. Natl. Acad. Sci. USA 74:560, 1977) to determine the nucleotide occurrence at the SNP loci.

[The nucleotide occurrence at a SNP site also can be determined using a microsequencing method, wherein the identity of only a single nucleotide is determined at a predetermined site (U.S. Pat. No. 6,294,336). Microsequencing methods include the Genetic Bit Analysis method (WO 92/15712). Additional, primer-guided, nucleotide incorporation procedures for assaying polymorphic sites in DNA have also been described (Komher et al., Nucl. Acids. Res. 17:7779-7784, 1989; Sokolov, Nucl. Acids Res. 18:3671, 1990; Syvanen et al., Genomics 8:684-692, 1990; Prezan et al, Hum. Mutat. 1:159-164, 1992; Nyren et al., Anal. Biochem. 208:171-175, 1993). These methods differ from Genetic Bit™. Analysis in that they all rely on the incorporation of labeled deoxyribonucleotides to discriminate between bases at a polymorphic site. In such a format, the signal is proportional to the number of deoxyribonucleotides incorporated, and polymorphisms that occur in runs of the same nucleotide generate signals that are proportional to the length of the run (Syvanen et al. Amer. J. Hum. Genet. 52:46-59, 1993).]

Another method for determining the nucleotide occurrence at a SNP position is described by Macevicz (U.S. Pat. No. 5,002,867), wherein a nucleic acid sequence is determined via hybridization with multiple mixtures of oligonucleotide probes. In accordance with such a method, the sequence of a target polynucleotide is determined by permitting the target to sequentially hybridize with sets of probes having an invariant nucleotide at one position, and a variant nucleotides at other positions. The nucleotide sequence is determined by hybridizing the target with a set of probes, then determining the number of sites that at least one member of the set is capable of hybridizing to the target (i.e., the number of matches). This procedure is repeated until each member of a sets of probes has been tested. U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

Melting curve analysis of SNPs (McSNP® analysis) provides another method that can be adapted to a multiplex procedure and used for detecting a nucleotide occurrence in an AIM (Akey et al., supra, 2001). McSNP® analysis provides the additional advantages that it does not require a step of gel electrophoresis, thus minimizing the time and cost for detecting a SNP.

Many statistical methods are available for studying genetic traits, including traditional family-based linkage analysis, variance component methods, sib-pair linkage, measured genotype, transmission disequilibrium, genomic control, and structure analysis. Some of the genes underlying variation in susceptibility to common diseases (e.g., heart disease, obesity, type 2 diabetes, hypertension, and cancer) eventually will be identified using genetic approaches. However, there are a number of complexities in genetic research on common diseases because many of these conditions are multifactorial (i.e., have several sources of variability in risk) and polygenic (i.e., result due to the actions and interactions among several genes). Additional difficulties in the study of common diseases can derive from the late onset of symptoms and heterogeneity in etiology. Thus, identifying the genes involved in complex diseases remains one of the greatest challenges in the field of human genetics.

There has been increased interest in association studies as a useful approach to map common disease and drug response genes (Risch and Merikangas, Science 273:1516-1517, 1996; Jorde, Genome Res. 10:1435-1444, 2000; Nordborg and Tavaré, Trends Genet. 18:83-90, 2002). Until the present disclosure, however, the implication of ancestry for identifying these genes has not been fully appreciated. The multiplex methods of the invention provide a particularly useful platform for determining the nucleotide occurrences of SNPs of genes associated with disease susceptibility and drug responsiveness, as well as for the development of advanced forensic methods. As such, compositions and methods are provided for inferring an individual's response to commonly used medications, which is a function of individual ancestry (see WO 2004/016768). In addition, the exemplified compositions and methods provide tools for inferring individual and/or group ancestral proportions from knowledge of the individual's or group's DNA sequences, as well as for qualifying and normalizing study groups for more traditional methods of mapping disease genes. Each of these processes requires an accurate knowledge of ancestry, which can be determined using the methods of the invention and exemplified compositions.

Screening genomes for the true identity of genes associated with a particular trait such as drug responsiveness is extraordinarily expensive and time consuming. As such, the use of AIMs for making inferences, for example, about individual proclivity to drugs provides a significant short-cut for the rapid development of tests that can be used to match patients with those drugs most appropriate for their genetic constitution. Thus, in addition to being useful for the admixture mapping of disease genes, the disclosed methods and exemplified markers provide tools for personalized medicine, which can direct treatment protocols by clinicians.

In addition, the exemplified primers and primer pairs, and multiplex reactions, allow for the determination of proportional ancestry, which can be a proportion of any ancestral group, including, for example, a proportion of sub-Saharan African, Native American, IndoEuropean, East Asian, Middle Eastern, or Pacific Islander ancestral group, and generally is a combination of two or more of such ancestral groups. Thus, the proportional ancestry of a test individual can include proportional affiliation among the sub-Saharan African and IndoEuropean ancestral groups (e.g., 80% sub-Saharan African and 20% IndoEuropean; or 60% sub-Saharan African, 20% IndoEuropean, and 20% of a third ancestral group); or can include proportional affiliation among the Native American and IndoEuropean ancestral groups; East Asian and Native American ancestral groups; IndoEuropean and East Asian ancestral groups; and the like. In this method, identifying a population structure within an individual that correlates with the nucleotide occurrences of the AIMs of the test individual can be practiced by performing a likelihood determination for affiliation with each of a sub-Saharan African ancestral group, a Native American ancestral group, an IndoEuropean ancestral group, and an East Asian ancestral group; thereafter selecting three ancestral groups having a greatest likelihood value for the individual; determining a likelihood of all possible proportional affiliations among the three ancestral groups having the greatest likelihood value, whereby a population structure or proportional affiliation that correlates with the nucleotide occurrences of the AIMs of the test individual is identified; and identifying a single proportional combination of maximum likelihood (see, also, WO 2004/016768). Alternatively, identifying a population structure that correlates with the nucleotide occurrences of the AIMs can be practiced by performing six two-way (binary) comparisons comprising likelihood determinations for affiliation of each group compared to each other group; thereafter selecting three ancestral groups having a greatest likelihood value across all comparisons; determining a likelihood of all possible proportional affiliations among the three ancestral groups having the greatest likelihood value, whereby a population structure or proportional affiliation that correlates with the nucleotide occurrences of the AIMs of the test individual is identified; and identifying a single proportional combination of maximum likelihood. Such a methodology works as well for individuals of three-way admixture as individuals that are 100% affiliated with a single group.

An estimate of an individual's proportional ancestry that includes proportions of three ancestral groups also can be made by performing three three-way comparisons among the groups; determining a likelihood of all possible proportional affiliations among the three ancestral groups having the greatest likelihood value, whereby a population structure or proportional affiliation that correlates with the nucleotide occurrences of the AIMs of the test individual is identified; and identifying a single proportional combination of maximum likelihood. An advantage of the present methods is that a graphical representation of the comparison of the three ancestral groups can be generated, wherein the graphical representation comprises a triangle with each ancestral group independently represented by a vertex of the triangle, and wherein the maximum likelihood value of proportional affiliation for an individual comprises a point within the triangle (see WO 2004/016768). If desired, the graphical representation can further include a confidence contour that indicates a level of confidence associated with estimating the proportional ancestry.

An estimate of an individual's proportional ancestry also can be made where the proportional ancestry includes proportions of four ancestral groups. In various aspects of this method, identifying a population structure that correlates with the nucleotide occurrences of the AIMs of the test individual is practiced by performing six two-way comparisons, or by performing three three-way comparisons, or by performing one four-way comparison among the groups; determining a likelihood of all possible proportional affiliations among the four ancestral groups having the greatest likelihood value, whereby a population structure or proportional affiliation that correlates with the nucleotide occurrences of the AIMs of the test individual is identified; and identifying a single proportional combination of maximum likelihood. If desired, the method can further include generating a graphical representation of the comparison of the three ancestral groups, wherein the graphical representation comprises a pyramid with each ancestral group independently represented by a vertex of the pyramid, and wherein the maximum likelihood value of proportional affiliation for an individual comprises a point within the pyramid. If desired, the graphical representation can further include a confidence contour comprising a sphere around the point, wherein the sphere indicates a level of confidence associated with estimating the proportional ancestry.

Such methods are useful, for example, as a forensic tool and, when performed as multiplex assays, allow for a rapid and cost effective way to screen samples. Using a DNA sample obtained at a crime scene, for example, the methods can provide an investigator with prospective information as to the likelihood of an individuals ancestry, as well as hair, skin and eye pigmentation. The methods of estimating proportional ancestry of a test individual also provide a tool that can supplement genealogical information, which generally is based on relationships established using geopolitical information. For example, the present methods provide information that can be used to generate an ancestral map of the world, wherein locations of populations having a proportional ancestry corresponding to the proportional ancestry of the test individual are indicated on the ancestral map (see WO 2004/016768). As such, the method can further include overlaying the ancestral map with a genealogical map, wherein the genealogical map indicates locations of populations having geopolitical relevance with respect to the test individual, and statistically combining the information of the ancestral map and genealogical map to obtain a most likely estimate of family history of the test individual.

Identifying a population structure that correlates with the nucleotide occurrences of the AIMs, according to a method of the invention, can be performed by comparing the nucleotide occurrences of the AIMs of the test individual with known proportional ancestries corresponding to nucleotide occurrences of AIMs indicative of BGA. The known proportional ancestries corresponding to nucleotide occurrences of AIMs indicative of BGA can be contained in a table or other list, and the nucleotide occurrences of the test individual can be compared to the table or list visually, or can be contained database, and the comparison can be made electronically, for example, using a computer. A particularly useful application of a method of the invention involves associating known proportional ancestries corresponding to nucleotide occurrences of AIMs indicative of BGA of individuals, with a photograph of a person from whom the known proportional ancestry was determined, thus providing a means to further infer physical characteristics of a test individual. Where the photograph is a digital photograph, which comprises digital information, the information can be contained in a database, which can further contain a plurality of such digital information of digital photographs, each of which is associated with a known proportional ancestry corresponding to nucleotide occurrences of AIMs indicative of BGA of the person in the photographs.

As such, a method of the invention can further include identifying a photograph of a person having a proportional ancestry corresponding to the proportional ancestry of the test individual. Such identifying can be done by manually looking through one or more files of photographs, wherein the photographs are organized, for example, according to the nucleotide occurrences of AIMs of the person in the photograph. Identifying the photograph also can be performed by scanning a database comprising a plurality of files, each file containing digital information corresponding to a digital photograph of a person having a known proportional ancestry, and identifying at least one photograph of a person having nucleotide occurrences of AIMs indicative of BGA that correspond to the nucleotide occurrences of AIMs indicative of BGA of the test individual.

The term “proportional ancestry” is used herein to refer to the percent contribution of each (if more than one) ancestral group to which an individual belongs. The proportional ancestry estimated according to a method of the invention can be a proportion of any ancestral group, including, for example, a proportion of sub-Saharan African, Native American, IndoEuropean, East Asian, Middle Eastern, or Pacific Islander ancestral group, and generally is a combination of two or more of such ancestral groups. Thus, the proportional ancestry of a test individual can include proportions of sub-Saharan African and IndoEuropean ancestral groups (e.g., 80% sub-Saharan African and 20% IndoEuropean; or 60% sub-Saharan African, 20% IndoEuropean, and 20% of a third ancestral group); or can include proportions of Native American and IndoEuropean ancestral groups; East Asian and Native American ancestral groups; IndoEuropean and East Asian ancestral groups; and the like. Similarly, the proportional ancestry can include proportions of Native American, East Asian, and IndoEuropean ancestral groups; sub-Saharan African, Native American, and IndoEuropean ancestral groups; sub-Saharan African, Native American, and East Asian ancestral groups; and the like. The exemplified panels of primers (Appendix 1) and primer pairs (Appendix 2) allow for the sampling, in multiplex reactions, of AIMs as set forth in SEQ ID NOS:364 to 537 (Appendix 3), which are useful for estimating proportional ancestry of an individual.

The following examples are intended to illustrate but not limit the invention.

### EXAMPLE 1

### Determination of Biogeographical Ancestry Using Ancestry Informative Markers

This Example demonstrates that a panel of 32 Ancestry Informative Markers (AIMs) allows an estimate of the genetic contribution from populations of African, European and Native American ancestry.

The AIMs used in the exemplified study include single nucleotide polymorphisms (SNPs), deletion/insertion polymorphisms (DIPs) and Alu sequences (see Example 2 for identification of AIMs). Markers showing differences between the parental populations greater than 30% were selected (Table 1; see, also, SEQ ID NOS:332-363). Informative genetic markers were identified by testing each candidate marker in a panel of European (Spanish, and German), African (from Nigeria, Sierra Leone, and Central African Republic), and Native American populations (Mayan and South Western Native Americans) to confirm the usefulness of the marker for admixture estimation.

TABLE 1
Ancestry Informative Marker Panel

| MARKER | LOCATION | Mb | AF/EU | AF/NA | EU/NA |
|---|---|---|---|---|---|
| MID 575 (356*) | 1p34.3 | ˜42 | 0.130 | 0.417 | 0.546 |
| MID 187 (357) | 1p32 | ˜50.2 | 0.370 | 0.440 | 0.070 |
| FY-NULL (339) | 1q23.2 | ˜181 | 0.999 | 0.999 | 0.000 |
| AT3 (359) | 1q25.1 | ˜196 | 0.575 | 0.777 | 0.202 |
| F13B (338) | 1q31.3 | ˜220 | 0.641 | 0.674 | 0.033 |
| TSC1102055 | 1q32.1 | ˜234.5 | 0.441 | 0.303 | 0.744 |
| (343) | | | | | |
| WI-11392 (NS**) | 1q42.2 | ˜269.5 | 0.444 | 0.256 | 0.188 |
| WI-16857 (345) | 2p16.1 | ˜56.2 | 0.536 | 0.548 | 0.012 |
| WI-11153 (346) | 3p12.1 | ˜95.0 | 0.652 | 0.022 | 0.629 |
| GC*1F (NS) | 4q13.3 | ˜75.7 | 0.697 | 0.530 | 0.166 |
| GC*1S (NS) | 4q13.3 | ˜75.7 | 0.538 | 0.478 | 0.060 |
| MID-52 (NS) | 4q24 | ˜110.7 | 0.186 | 0.500 | 0.687 |
| SGC30610 (354) | 5q11.2 | ˜61.5 | 0.146 | 0.281 | 0.427 |
| SGC30055 (355) | 5q22.1 | ˜124.7 | 0.457 | 0.675 | 0.218 |
| WI-17163 (347) | 5q33.1 | ˜173.9 | 0.120 | 0.641 | 0.521 |
| WI-9231 (348) | 7p22.3 | ˜1.2 | 0.017 | 0.387 | 0.370 |
| WI-4019 (349) | 7q21.3 | ˜100 | 0.124 | 0.173 | 0.296 |
| CYP3A4 (NS) | 7q22.1 | ˜101.9 | 0.761 | 0.755 | 0.006 |
| LPL (340) | 8p21.3 | ˜22.3 | 0.479 | 0.521 | 0.042 |
| CRH (NS) | 8q13.2 | ˜73.2 | 0.609 | 0.655 | 0.046 |
| WI-11909 (350) | 9q21.31 | ˜81.0 | 0.075 | 0.587 | 0.663 |
| D11S429 (337) | 11q13.3 | ˜70.4 | 0.429 | 0.054 | 0.376 |
| TYR (344) | 11q14.3 | ˜95.4 | 0.444 | 0.055 | 0.389 |
| DRD2-Taq I “D” | 11q23.2 | ˜125.0 | 0.535 | 0.046 | 0.582 |
| (336) | | | | | |
| DRD2-Bcl I (335) | 11q23.2 | ˜125.0 | 0.080 | 0.565 | 0.485 |
| APOA1 (360) | 11q23.3 | ˜128.9 | 0.505 | 0.555 | 0.050 |
| GNB3 (332) | 12p13.31 | ˜7.2 | 0.463 | 0.430 | 0.033 |
| RB1 (361) | 13q14.2 | ˜47.4 | 0.611 | 0.711 | 0.100 |
| OCA2 (342) | 15q12 | ˜24.0 | 0.631 | 0.369 | 0.263 |
| WI-14319 (351) | 15q14 | ˜30.0 | 0.185 | 0.310 | 0.494 |
| CYP19 (334) | 15q21.2 | ˜47.6 | 0.045 | 0.379 | 0.423 |
| PV92 (362) | 16q23.3 | ˜96.5 | 0.073 | 0.551 | 0.624 |
| MC1R314 (341) | 16q24.3 | ˜103.8 | 0.350 | 0.441 | 0.090 |
| WI-14867 (352) | 17p13.2 | ˜3.5 | 0.448 | 0.404 | 0.045 |
| WI-7423 (353) | 17p13.1 | ˜8.2 | 0.476 | 0.074 | 0.402 |
| Sb19.3 (363) | 19p13.11 | ˜27.0 | 0.488 | 0.236 | 0.253 |
| CKM (333) | 19q13.2 | ˜55.8 | 0.150 | 0.694 | 0.545 |
| MID 154 (358) | 20q11.23 | ˜34 | 0.444 | 0.368 | 0.076 |
| MID 93 (NS) | 22q13.2 | ˜38.6 | 0.554 | 0.179 | 0.733 |

TABLE 1 cont'd Shown are the marker name and chromosomal band, approximate location of the marker on the chromosome in megabases (Mb), and difference in frequency between African and European populations (AF/EU), African and Native Americans (AF/NA) and European and Native Americans (EU/NA). Differences greater than 30% are marked in bold letters (see, also, Shriver et al., supra, 2003, which is incorporated herein by reference)
*Numbers in parentheses are SEQ ID NO: for AIMs;
NS - sequences not shown.

The publicly available human genome sequence database and polymorphism database were screened in order to identify SNPs that met the criteria for being a good AIM. Allele frequencies are available for many of the SNPs in the public databases for three populations—Africans, Europeans and Asians. Since these frequencies are obtained from small samples they are not always accurate. The main criteria for selection herein was the delta value that derived from using these frequencies, which is a statistical measure of the difference in minor allele frequency between various populations of human beings. For example, a C or a G polymorphism at a particular place in the human genome, where the C is present mainly in individuals of European descent and the G present mainly in individuals of Native American descent, would have a high delta value and, therefore, qualify as a good AIM. Similarly, an A or a C polymorphism at a particular place in the human genome, where the A is present mainly in individuals of African descent and the C present mainly in individuals of Asian descent would have a large frequency differential between these groups and, therefore, a high delta value, thus qualifying as a good AIM. A list of such “candidate AIMs was compiled, ranked from largest delta value to smallest delta value for each of the possible pair-wise population comparisons, and screened, one at a time, against a panel of “parental” samples. Parental samples are samples from regions of the world that are relatively homogeneous, for example, Niger or Congo for sub-Saharan Africans, Southern Mexico for Native Americans, China for East Asians, and Europe for Europeans.

About half of the candidate AIMs proved to be not very useful because their actual delta values were not as high as expected from the public database allele frequencies (some were not even SNPs, or could not be assayed using the present platform). Sequences that were validated as true AIMs, such as those exemplified herein, were useful for admixture mapping, making inferences of individual ancestry proportions, and making inferences of population group admixture proportions, as well as for screening genomes in order to identify markers with alleles that correlated with certain human traits through their ancestry informativeness. Even though each candidate AIM was initially selected from the public database based on crude population structure differences (i.e., continental populations), many of them were found to carry information on finer levels of structure because the separation of subgroups of humans from larger groups throughout human evolution has provided a fertile opportunity for genetic drift, founder effects, and natural selection to operate in either fixing or eliminating their sequences.

Sequences are shown in the Sequence Listing from 5′ to 3′ (left to right), and, for SEQ ID NOS:1 to 331, with the SNP generally, but not always, at nucleotide position 50 from the 5′ terminus (except for SEQ ID NO:56, position 26). The polymorphism is indicated with an IUB symbol, wherein S=C/G, Y=C/T, R=A/G, K=G/T, W=A/T, etc. As such, the disclosed sequences (SEQ ID NOS:1 to 331) provide information as to the target being examined (i.e., the polymorphism) as well as information for preparing primers and amplification primer pairs, and hybridization probes, for sampling the SNP (i.e., determining the genotype of a sample). Further, the disclosed sequences can be used, if desired, to scan public databases to identify additional upstream and downstream nucleotide sequences.

This panel of markers was extremely powerful for estimating with precision admixture proportions in population samples (standard error typically between 1% and 5%). In addition, the AIMs provided reasonable estimates of individual ancestry, and suggested that an equivalent precision can be obtained using more markers (confirmed in Example 2). Two independent methods, a Maximum Likelihood Estimate (MLE) method (Chakraborty et al., supra, 1986) and a Bayesian method using the program STRUCTURE (Pritchard et al., supra, 2000) estimating individual ancestry, were used; the values obtained by both methods were highly correlated (R2=0.9836) when estimates of individual ancestry were compared in terms of percent African genetic contribution in a sample of African Americans from Washington D.C. These markers are excellent for determining whether there is population structure in a sample from an admixed population. This ability is important in terms of admixture mapping applications because, as discussed below, the process of admixture can produce significant structure in a population, and consequently a high number of false positive results (positive associations caused by genetic structure, not by physical linkage of a marker with a disease causative gene), increasing significantly the risk of misinterpreting mapping results.

The present study confirms that AIMs can be identified using the disclosed methods, and provides a panel of 32 AIMs that can be applied towards an ultimate goal of compiling a panel of approximately 1,000 AIMs spanning the entire human genome. Candidate AIMs were obtained by screening SNP allele frequency data generated through The SNP Consortium (TSC). Six sites, including the Sanger Centre, Celera Genomics, Washington University, Orchid Biosciences, Motorola, and Whitehead Institute, have generated, as of 2003, allele frequencies on 60,000 SNPs located throughout the genome using a central collection of 42 individuals from each of 3 populations (African-American, European-American, and Asian-American). This database, which is freely available to researchers (see, e.g., using hypertext transfer protocol (“http”), at URL “snp.cshl.org”), has been used to provide the present results, thus demonstrating the usefulness of the resource to compile a genome-wide panel of AIMs.

The present study focused on the accuracy of the SNP database and the number of candidate SNPs present therein. With respect to the accuracy of the database, each group involved in the SNP consortium has taken a different approach to generating data. As such, initial concerns regarding how the data can be combined was addressed. Because the genotyping approaches were different for each group, it was necessary to address the question of ascertainment biases that might differentially affect the data of particular groups. For example, most of the groups produced their allele frequencies after sequencing a subset of the TSC diversity panels, then scoring these markers in the larger groups of 42 individuals from the 3 populations. The Washington University group has taken an approach whereby pooled sequencing throughout regions was performed, and the allele frequencies calculated for variable positions discovered during this effort. The Orchid group has not used sequencing but, instead, started with loci from the TSC SNP database that are known to be polymorphic. Given such differences, a systematic characterization was made as the extent, if any, that different biases may have affected the results.

One approach for systematically characterizing such potential bias was to compare the allele frequencies for loci that were genotyped by more than one group. Although there was dispersion around the 45° line, as expected, there was general agreement in the frequency data obtained by the different groups (R2=0.8762), indicating that the extent of the allele frequency bias introduced by the differing genotyping and ascertainment strategies was limited. The next step in testing the accuracy of these data was to separate the data by site and perform pair-wise comparisons, which would allow the identification of particular sites that have allele frequency estimates that deviate more when compared to other sites.

With respect to the number of candidate AIMs, it also was important to determine how many of the 60,000 SNPs characterized by the TSC would be useful for admixture mapping. Since it can be useful ultimately to compile a panel of about 1,000 markers showing large frequency differences between the relevant population groups (Fst>0.4), it was important to evaluate which percentage of the available markers have the desired characteristics. Candidate AIMs were based on the recommendation of McKeigue et al. (supra, 2000). The markers with information available for African, Asian and European populations, the cumulative proportion of markers in each Fst category (0-1, in 0.05 intervals) and the total number of candidate AIMs for each possible comparison. The distribution of pairwise Fst from the TSC allele frequency project was as follows: Asian-European (556 candidate AIMs/25,110 total SNPs; average Fst=0.0720); Asian-African (1026 candidate AIMs/25,578 total SNPs; average Fst=0.0886); and European-African (1306 candidate AIMs/30,103 total SNPs; average Fst=0.0861). As such, the screen revealed that about 2-5% of the markers can be useful for admixture mapping.

The geographical pattern of admixture in the US admixed populations, particularly African Americans and Hispanics, was the subject of an initial examination. The admixture proportions of more than 18 African-American populations were characterized, and a map was generated showing an estimate of the European genetic contribution to African Americans from several different geographic areas in the United States. The European admixture ranged between 3.5% in the Gullah of South Carolina to 22.5% in New Orleans (e.g., 18.8% in Chicago; and 16.4% in Houston). Most of these estimates were obtained using an initial panel of 10 informative AIMs. The observed distribution was interpreted in terms of well known historical and demographic events that have played an important role in African American history (see Parra et al., supra, 1998, Parra et al., supra, 2001). These data allow the application of admixture mapping to identify genes involved in complex diseases. It is expected that admixture mapping will be more suitable in populations showing a high degree of admixture and, therefore, populations such as the Gullah (3.5%) and Jamaicans (6.6%), in which European genetic contribution has been very limited, may not be suitable for this kind of analysis.

In preliminary studies using mitochondrial DNA (mtDNA), it was observed that African Americans have a detectable, although low, Native American genetic contribution, in accordance with the self-reported Native American ancestry often mentioned by African-American individuals. Having identified 30 AIMs informative for African/Native American and 19 AIMs for European/Native American contrasts (see Table 1; see, also, Shriver et al., supra, 2003), the presence of Native American admixture in three African-American populations was examined using nuclear DNA markers. In accordance with the mtDNA estimates (which only provide “maternal” contribution information), evidence of a low Native American genetic contribution was detected in each of the African-American samples (Washington D.C., 6%; Afro-Caribbeans from London, 5%; and Bogalusa, La., 6%).

Regarding admixture in Hispanics, the relative European, Native American and African contribution in a sample of Spanish-Americans from San Luis Valley Colo. was estimated. A 59% European admixture, 35% Native American admixture, and 6% African admixture was observed in this sample, in good agreement with estimates previously described for populations of Mexican ancestry (Chakraborty et al., supra, 1986; Hanis et al., supra, 1991; Tseng et al., Amer. J. Phys. Anthropol. 106:361-71. 1998; Collins-Schram et al., supra, 2002). As shown in Example 2, further characterization of admixture in additional samples from Mexico and in two samples from Hispanics of Puerto Rican ancestry (New York and Puerto Rico) has been performed.

A sample of individuals of European ancestry (N=199) currently living in State College Pa. also were analyzed. The genetic contribution in this sample was predominantly of European origin (91%), with evidence of some African (3%) and Native American (6%) influence. These results are summarized in FIG. 4, using a triangular plot, which clearly reveals the differences in average admixture levels between European Americans, Spanish Americans, and African Americans. The triangular plot shown in FIG. 4 represents the average admixture estimate in a particular sample; the underlying distributions of individual ancestry are complex, with different individuals showing widely dispersed values of African, European and Native American ancestry (not shown). In African Americans, most individuals showed predominantly African genetic contribution, but some persons showed relatively high European contribution and also, to a lesser extent, Native American ancestry. European Americans clustered more tightly near the pole corresponding to high European contribution, with few persons showing evidence of Native American and African ancestry. Spanish Americans showed the highest dispersion of individual ancestry, as expected given the high admixture level observed in this sample.

Notably, individuals showed the whole range of European and Native American ancestry (from 100% European to 100% Native American), and a relatively lower African genetic contribution also was evident in some individuals. Some of the variance observed in individual ancestry was likely due to the stochastic error due to the limited number of markers used to infer ancestry. Thus, while the 20-32 markers used in the exemplified test detected individual ancestry, the standard error of the estimates was fairly high; increasing the number of AIMs is expected to increase the precision of the individual ancestry estimates (see Example 2). The other component of the variation in individual ancestry was due to true differences in ancestry between individuals. The remarkable correlation in individual ancestry values obtained by two totally independent methods, ML and STRUCTURE (discussed above), indicates that this panel of markers can capture the underlying individual ancestry patterns characteristic of these populations. As disclosed below, controlling for variations in individual ancestry allows the avoidance of false positive results.

The effect of admixture dynamics in population structure and the extent of linkage disequilibrium (LD) was examined. The importance of the admixture model (hybrid isolation model vs. continuous gene flow model) in terms of the population structure and LD created in the admixture process was previously described (Pfaff et al., supra, 2001), and two methods to quantify the level of population structure in admixed populations were presented. Population structure is a key aspect of admixture mapping, as well as of any genetic association study in an admixed population. This issue has been explored in an African-American, a Spanish-American and a European-American sample using more informative markers than previously available.

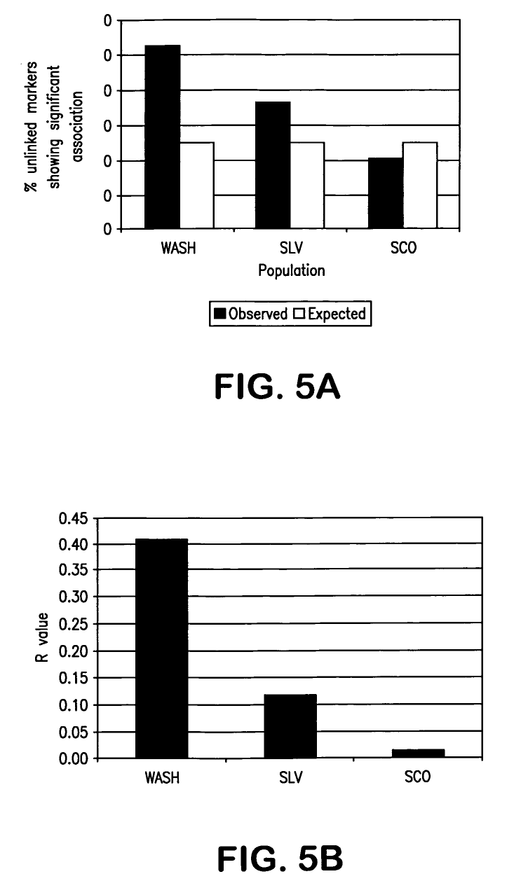

The presence of structure was evaluated in two different ways. First, the observed number of significant associations was compared between unlinked markers with the number expected at the 5% significance level, and second, the average correlation of individual ancestry was estimated using two subsets of genetic markers. In agreement with previously reported data, the African-American population from Washington D.C. showed a significant genetic structure, as reflected by a much higher number of significant associations between unlinked markers than expected by chance (10.5% vs. 5%, FIG. 5A). Very strong associations were observed between markers located as far apart as 24 Mb (AT3-F13B, G=15.21, p<0.0001), providing clear evidence that these significant associations are caused by the admixture process. The alleles that were associated were always combinations of the alleles that are frequent in African populations, with the higher the difference in frequency, the more frequently associations were observed between markers: FY, showing the highest difference in frequency between African and Europeans, was significantly associated with 9 unlinked markers. Thus, the admixture process in this African-American population, showing 17% European ancestry, created a strong association between markers showing high frequency differences between African and European populations. Although these associations are significant both when the markers are linked and when they are unlinked, linked markers tended to show higher G values than unlinked markers, indicating that the association due to true linkage can be distinguished from the association due to genetic structure, as previously demonstrated (McKeigue et al., supra, 2000). Interestingly, significant associations were observed between markers showing high frequency differences between European and African populations, but not between markers showing high frequency differences between African and Native American, or European and Native American populations. This result was likely due to the low Native American ancestry observed in this sample (6%), which was insufficient to create detectable associations due to the admixture process in such a small sample.

Another line of evidence demonstrating the high level of genetic structure present in this African-American sample was the significant correlation between independent estimates of individual ancestry using different subsets of the genetic markers. The average correlation over 100 random selections of independent subsets of markers was r=0.40, p<0.0001 (FIG. 5B). The pattern of genetic structure and LD observed in Washington D.C. African Americans and in other African-American samples analyzed with a more limited set of markers (Jackson Miss., and the Low Country counties, South Carolina, Pfaff et al., supra, 2001) indicated that the best model to describe the admixture process in these populations was the continuous gene flow model. Additional support for this model came from the strong correlation between D0 (the initial expected association between markers originated by the admixture process) and Dt (the current association between markers). As shown using computer simulations (Pfaff et al., supra, 2001), in populations following the continuous gene flow model, positive correlations between D0 and Dt are expected and, in fact, this result was observed in African-American populations. In populations following the hybrid isolation model, significant correlations between D0 and Dt are not expected.

The Spanish-American sample from San Luis Valley that was analyzed showed less genetic structure than any of the African-American populations. The number of observed significant associations between unlinked markers was only slightly higher than expected at a 5% significance level (7.3% vs. 5%, FIG. 5A). This result was interesting considering that the Spanish-American population was considerably more admixed than the African Americans, and under the same model of admixture dynamics, would be expected to show considerably more structure. The correlation of individual ancestry estimates based on independent markers, although significant, was much lower than the values observed in the African-American populations (r=0.11, p<0.0001, FIG. 5B). Also, there was no correlation of D0 and Dt in the San Luis Valley sample, in contrast to the results observed in African Americans. These results demonstrate that admixture dynamics (the way in which the population was formed and has evolved) have been different in the African-American populations and the San Luis Valley population, with the former more closely resembling the continuous gene flow model than the latter. Of course, other Hispanic populations can show different patterns of admixture dynamics than that observed in San Luis Valley.

As expected from the lower admixture levels observed in European Americans, there was no evidence of genetic structure due to admixture in this sample from State College Pa. (FIGS. 5A and 5B). The number of significant associations between unlinked markers was similar to the value expected by chance, and there was no correlation between individual ancestry estimates of independent subsets of markers (p=0.149, NS).

These results demonstrate that the use of selected genetic markers (AIMs) allows an analysis of the dynamics of the admixture process and the effect of this admixture process on the pattern of LD in admixed populations. In admixed populations that have had an admixture process similar to the hybrid isolation model (initial admixture followed by independent evolution of the admixed population without further genetic contribution of the parental populations), few false positive results are expected (recalling, that the falseness is relevant for a “gene hunter” searching for genes that cause traits through LD or linkage, not for those seeking to develop classification tools). In admixed populations that more closely resemble the continuous gene flow model (continuous genetic contribution each generation from one of the parental populations to the admixed population), the LD is expected to extend much longer distances and problems with false positive results will arise. Fortunately for the gene hunter, the information conveyed by the AIMs can control for genetic structure and minimize false positives. An example is provided below demonstrating how such control can be achieved using appropriate statistical methods and skin pigmentation as a model phenotype.

Skin pigmentation and individual ancestry was examined in an African-American sample and a Spanish-American sample. As previously demonstrated, the genetic structure created by admixture can be effectively controlled, and association due to linkage can be distinguished from spurious association due to genetic structure using appropriate statistical tests (McKeigue et al., supra, 2000). In the present study, the same methods were applied in a study of skin pigmentation in two admixed samples (African Americans from Washington D.C. and Spanish Americans from San Luis Valley). Information on skin pigmentation was collected for each individual in both studies, and the subjects were genotyped for a panel of AIMs and individual ancestry proportions were calculated using the maximum likelihood method (Chakraborty et al., supra, 1986). Individual ancestry (% African or % Native American) was plotted against melanin index (African) or skin reflectance (Native American) for each individual. Several of the AIMs showing high differences in frequency between the parental populations were also candidate genes for pigmentation.

In the African-American sample, a strong and highly significant correlation (R2=0.1879, p<0.0001) was observed between individual ancestry and the melanin index, which measures the melanin content of the skin. Individuals with darker skin had, on average, higher levels of African ancestry. The individual ancestry estimates were based on 21 markers, and, therefore, subject to a relatively high variance, thus explaining at least some of the dispersion observed in the graph. An interesting feature of these results was the evident decrease in variance observed in moving from the right (more African ancestry) to the left (more European ancestry). This result is consistent with the higher level of variability in skin color that is found in African populations as compared to Europeans. The high correlation observed between individual ancestry and skin pigmentation can be due to the population structure typical of African-American populations (as discussed above), and related to the limited number of genes that were used for determining the parental population differences contained within this relationship.

A similar plot was prepared for the San Luis Valley sample. Individual ancestry estimates using 15 Native American/European AIMs were plotted against pigmentation level as measured by the percent of light reflected through the PHOTOVOLT 670 Green filter. Because skin pigmentation was measured in different ways (absorbance vs. reflectance) in these two studies, the trends observed when graphed are reversed. In the Spanish-American sample, the correlation between individual ancestry and skin color also was significant (R2=0.0481, p<0.001), but lower than in the African-American sample, possibly due to the reduced genetic structure present in this sample.

Tests for differences in the average pigment levels by genotype for the AIMs typed in the African-American population sample discussed above were performed. The panel of AIMs included three candidate gene markers, OCA2, TYR, and MC1R. The analysis was performed in three alternative ways: first with no consideration of the individual ancestry estimates (ANOVA); second after conditioning to control for the effect of individual ancestry leaving out the locus under consideration (ANCOVA/IAE minus marker); and third using the complete individual ancestry estimate for the conditioning (ANCOVA/IAE). As shown in Table 2, eight of twenty-one (38%) of the markers showed significant differences (p<0.05) among the three genotypes, including two of the four candidate gene markers (OCA2 and TYR). When using an alpha level of 0.05, only 5% of the markers tested were expected to yield significant results. As such, the finding of 38% significant difference indicates that population structure is related to both ancestry and pigmentation (Pfaff et al., supra, 2001, Parra et al., supra, 2001).

One way to remove the effects of population structure is to test for differences conditioning on the individual ancestry estimates (IAE). When the complete IAE was used to condition (ANCOVA/IAE), only one locus showed significant average differences among genotypes, OCA2, the human P gene. When a less conservative conditioning approach was taken, in which the locus under consideration was left out of the individual ancestry estimate (ANCOVA/IAE minus marker), there were four significant results: OCA2, TYR, FY, and SGC30055.

A Bayesian full probability model for admixture and marker genotypes was also set up (McKeigue et al., supra, 2000). Score tests for linkage were based on testing for an independent association of pigmentation with number of alleles of European ancestry at each locus, one at a time, in a regression model that includes individual ancestry (estimated from marker data). The 1-sided probabilities for the score tests are shown in Table 2, where three loci showed evidence of linkage to skin pigmentation at an alpha level of 0.05 {OCA2 (p=0.005), AT3 (p 0.027), and TYR (p=0.033)}. To confirm these results, other markers informative for ancestry in OCA2 were identified and will be analyzed by the score test method. The concordance between these ANOVA results and the Bayesian admixture mapping results was encouraging, and both methods will benefit from the addition of new unlinked AIMs, which will increase the precision of the individual ancestry estimates.

The Spanish-American sample from the San Luis Valley Colo., also was analyzed for linkage and association using the Bayesian and ANOVA methods (Table 3). This analysis included 442 individuals who were typed for 15 marker loci informative for ancestry (2 SNPs in the DRD2 gene treated as one locus). The CYP19E2 marker (located near MYO5A, a pigmentation candidate gene) showed strong evidence for linkage with the ethnic difference in skin pigmentation. However, this result should be interpreted with caution because, unless several closely linked markers informative for ancestry are used, the test for linkage is not robust to misspecification of ancestry-specific allele frequencies. SNPs around the MYO5A gene can be analyzed to confirm these preliminary results.

TABLE 2
Testing for an effect of single-locus genotypes on pigmentation
in an African-American sample

| | DELTA² | | ANCOVA⁴ | | |
|---|---|---|---|---|---|
| | AF vs | | IAE minus | ANCOVA⁵ | Bayesian score |
| Marker¹ | EU | ANOVA³ | marker | IAE | test probability⁶ |
| FY-null | 0.997 | 0.000* | 0.004* | 0.396 | 0.106 |
| LPL | 0.479 | 0.000* | 0.130 | 0.501 | 0.167 |
| WI-14867 | 0.448 | 0.061 | 0.561 | 0.253 | 0.100 |
| AT3 | 0.575 | 0.001* | 0.090 | 0.479 | 0.027* |
| Sb19.3 | 0.488 | 0.497 | 0.784 | 0.993 | 0.402 |
| APOA1 | 0.505 | 0.507 | 0.718 | 0.317 | 0.488 |
| D11S429 | 0.429 | 0.578 | 0.833 | 0.442 | 0.139 |
| RB1 | 0.611 | 0.743 | 0.535 | 0.128 | 0.102 |
| [symbol] | 0.631 | 0.000* | 0.001* | 0.019* | 0.005* |
| [symbol] | 0.428 | 0.601 | 0.482 | 0.590 | 0.486 |
| WI-16857 | 0.536 | 0.745 | 0.789 | 0.311 | 0.137 |
| WI-11153 | 0.652 | 0.026* | 0.225 | 0.698 | 0.223 |
| SGC30055 | 0.457 | 0.001* | 0.048* | 0.291 | 0.061 |
| WI-7423 | 0.476 | 0.193 | 0.544 | 0.169 | 0.058 |
| WI-11392 | 0.444 | 0.032* | 0.200 | 0.507 | 0.257 |
| MID 154 | 0.444 | 0.260 | 0.439 | 0.728 | 0.250 |
| MID 187 | 0.370 | 0.121 | 0.302 | 0.296 | 0.450 |
| DRD2 TAQD | 0.553 | 0.226 | 0.273 | 0.247 | 0.471 |
| GNB3 | 0.463 | 0.984 | 0.530 | 0.166 | 0.065 |
| [symbol] | 0.449 | 0.003* | 0.014* | 0.117 | 0.033* |
| GC | 0.697 | 0.213 | 0.750 | 0.604 | 0.190 |

¹Marker indicates the Ancestry Informative marker used in the test. Markers shown in bold and italics are candidate genes for pigmentation (viz. OCA2, MC1R, TYR).
²“DELTA” (δ) is the allele frequency difference between African and European populations.
³Analysis of variance significance level where sex is the only covariate.
⁴Significance level for a one-way ANCOVA analysis using individual ancestry estimates (M) where the tested locus was excluded as the covariate.
⁵Same as three except the M is based on all 21 markers.
⁶Bayesian Admixture Mapping 1-sided probability.

TABLE 3
Testing for an effect of single-locus genotypes on pigmentation
in a Spanish-American sample

| | | | ANCOVA⁴ | | |
|---|---|---|---|---|---|
| | DELTA² | | IAE | ANCOVA⁵ | Bayesian |
| Marker¹ | NA vs EU | ANOVA³ | minus marker | IAE | score test p-value⁶ |
| MID-575 | 0.546 | 0.240 | 0.383 | 0.248 | 0.93 |
| TSC1102055 | 0.744 | 0.027* | 0.023* | 0.366 | 0.39 |
| WI-11153 | 0.628 | 0.012* | 0.036* | 0.406 | 0.12 |
| SGC-30610 | 0.427 | 0.093 | 0.133 | 0.565 | 0.13 |
| WI-17163 | 0.521 | 0.192 | 0.268 | 0.967 | 0.17 |
| WI-4019 | 0.296 | 0.875 | 0.762 | 0.319 | 0.93 |
| WI-11909 | 0.663 | 0.026* | 0.020* | 0.146 | 0.13 |
| [symbol] | 0.417 | 0.006* | 0.012* | 0.129 | 0.07 |
| DRD2Bcl | 0.485 | 0.254 | 0.517 | 0.827 | 0.35 |
| DRD2TaqD | 0.582 | 0.418 | 0.461 | 0.550 | 0.35 |
| D11S429 | 0.376 | 0.235 | 0.511 | 0.545 | 0.65 |
| WI-14319 | 0.494 | 0.036* | 0.054 | 0.061 | 0.30 |
| [symbol] | 0.423 | 0.000* | 0.000* | 0.002* | 0.001* |
| PV92 | 0.624 | 0.183 | 0.276 | 0.591 | 0.50 |
| WI-7423 | 0.402 | 0.426 | 0.318 | 0.309 | 0.64 |
| CKMM | 0.545 | 0.257 | 0.569 | 0.579 | 0.55 |

¹Marker indicates the Ancestry Informative marker used in the test. Markers shown in bold and italics are in or near candidate genes for pigmentation (viz. TYR-192 and CYP19E2 near MYO5A).
²“DELTA” (δ) is the allele frequency difference between Native American and European parental populations.
³Analysis of variance significance level, where sex is the only covariate.
⁴Significance level for a one-way ANCOVA analysis using individual ancestry estimates (M) where the tested locus was excluded as the covariate.
⁵Same as three except the M is based on all 15 markers.
⁶Bayesian Admixture Mapping 1-sided probability.

Pairwise Population Comparison of SNP Distinction

Table 4 shows the results of genotyping and statistical analysis that demonstrate several different but important points (the sequences for each of the AIMs in Table 4 can be found by reference to Table 6, using the marker number.

TABLE 4
Pair-wise population comparisons of SNP distinction

| | | | | | | | | | | | | | | | | | | | | | | mark- |
|---|---|---|---|---|---|---|---|---|---|---|---|---|---|---|---|---|---|---|---|---|---|---|
| AF-CT | AF-EA | AF-SA | AF-ME | AF-PI | AF-AI | CT-EA | CT-SA | CT-ME | CT-PI | CT-AI | EA-SA | EA-ME | EA-PI | EA-AI | SA-ME | SA-PI | SA-AI | ME-PI | ME-AI | PI-AI | er | |
| 0.4 | 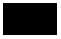 | 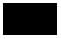 | 0.4 | 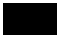 | 0.3 | 0.3 | 0.2 | 0.1 | 0.3 | 0.1 | 0.1 | 0.2 | 0 | 0.4 | 0.1 | 0.2 | 0.3 | 0.3 | 0.2 | 0.4 | 959 | |
| 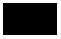 | 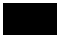 | 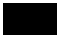 | 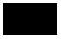 | 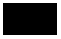 | 0.4 | 0 | 0 | 0 | 0.3 | 0.2 | 0 | 0 | 0.3 | 0.2 | 0 | 0.3 | 0.1 | 0.3 | 0.1 | 0.4 | 961 | |
| 0.1 | 0 | 0.1 | 0.1 | 0.1 | 0.1 | 0.2 | 0 | 0 | 0.2 | 0 | 0.1 | 0.2 | 0.1 | 0.2 | 0 | 0.2 | 0 | 0.2 | 0 | 0.2 | 962 | |
| 0.3 | 0.4 | 0.2 | 0.4 | 0.3 | 0.1 | 0.1 | 0 | 0.1 | 0 | 0.4 | 0.1 | 0 | 0 | 0.5 | 0.2 | 0.1 | 0.3 | 0.1 | 0.5 | 0.4 | 963 | |
| 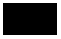 | 0.3 | 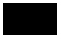 | 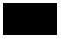 | 0.1 | 0.4 | 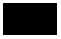 | 0.1 | 0.2 | 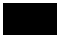 | 0.5 | 0.5 | 0.4 | 0.2 | 0.1 | 0.1 | 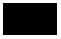 | 0.4 | 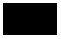 | 0.3 | 0.3 | 964 | |
| 0.3 | 0 | 0.2 | 0.2 | 0 | 0.5 | 0.4 | 0.1 | 0.1 | 0.3 | 0.2 | 0.3 | 0.2 | 0.1 | 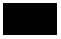 | 0 | 0.2 | 0.3 | 0.2 | 0.3 | 0.5 | 969 | |
| 0.2 | 0.2 | 0.2 | 0.2 | 0.1 | 0.1 | 0 | 0 | 0 | 0 | 0.1 | 0 | 0 | 0.01 | 0 | 0 | 0.1 | 0 | 0.1 | 0.1 | 970 | | |
| 0.3 | 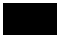 | 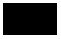 | 0.4 | 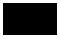 | 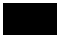 | 0.4 | 0.2 | 0 | 0.2 | 0.2 | 0.2 | 0.3 | 0.1 | 0.1 | 0.2 | 0 | 0 | 0.2 | 0.2 | 0 | 971 | |
| 0.5 | 0.2 | 0.3 | 0.5 | 0.1 | 0.2 | 0.3 | 0.2 | 0 | 0.4 | 0.3 | 0.1 | 0.3 | 0.1 | 0 | 0.2 | 0.2 | 0.1 | 0.3 | 0.3 | 0 | 972 | |
| 0.3 | 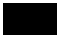 | 0.4 | 0.3 | 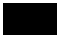 | 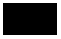 | 0.4 | 0.1 | 0.1 | 0.3 | 0.3 | 0.3 | 0.5 | 0.1 | 0.2 | 0.2 | 0.2 | 0.2 | 0.4 | 0.3 | 0 | 973 | |
| 0.1 | 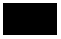 | 0.4 | 0 | 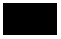 | 0.4 | 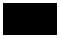 | 0.4 | 0 | 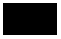 | 0.5 | 0.2 | 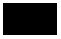 | 0 | 0.1 | 0.4 | 0.2 | 0.1 | 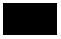 | 0.5 | 0.1 | 975 | |
| 0.4 | 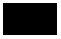 | 0.4 | 0.5 | 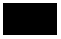 | 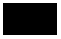 | 0.4 | 0 | 0.1 | 0.5 | 0.5 | 0.3 | 0.3 | 0.1 | 0.1 | 0.1 | 0.4 | 0.4 | 0.4 | 0.4 | 0 | 977 | |
| 0 | 0.1 | 0.1 | 0 | 0.3 | 0.2 | 0.1 | 0 | 0 | 0.4 | 0.1 | 0.1 | 0.1 | 0.4 | 0.1 | 0 | 0.4 | 0.1 | 0.3 | 0.2 | 0.5 | 978 | |
| 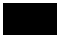 | 0.3 | 0.2 | 0.4 | 0.1 | 0.2 | 0.3 | 0.3 | 0.2 | 0.5 | 0.4 | 0 | 0.1 | 0.2 | 0.1 | 0.1 | 0.2 | 0 | 0.3 | 0.2 | 0.1 | 979 | |
| 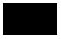 | 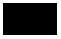 | 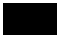 | 0.5 | 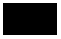 | 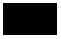 | 0.1 | 0.1 | 0.1 | 0 | 0.1 | 0.2 | 0.2 | 0.1 | 0.2 | 0 | 0.1 | 0 | 0.2 | 0.1 | 0.1 | 980 | |
| 0.2 | 0.1 | 0.5 | 0.5 | 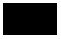 | 0.5 | 0.1 | 0.3 | 0.3 | 0.4 | 0.3 | 0.4 | 0.4 | 0.4 | 0.4 | 0 | 0 | 0 | 0 | 0 | 0.1 | 986 | |
| 0.1 | 0.1 | 0 | 0 | 0.2 | 0 | 0.2 | 0.1 | 0.1 | 0.3 | 0.1 | 0.1 | 0.1 | 0.1 | 0.1 | 0 | 0.2 | 0 | 0.2 | 0 | 0.2 | 993 | |
| 0.1 | 0 | 0.1 | 0.1 | 0.1 | 0.1 | 0.1 | 0.2 | 0.1 | 0.2 | 0 | 0.1 | 0.1 | 0.1 | 0.1 | 0 | 0 | 0.2 | 0.1 | 0.1 | 0.2 | 1000 | |
| 0 | 0 | 0.1 | 0 | 0.2 | 0 | 0 | 0.1 | 0 | 0.2 | 0 | 0.1 | 0 | 0.2 | 0 | 0.1 | 0.2 | 0.1 | 0.2 | 0 | 0.2 | 1001 | |
| 0 | 0.1 | 0.1 | 0.1 | 0.1 | 0.1 | 0.1 | 0.1 | 0.1 | 0 | 0.1 | 0 | 0 | 0.1 | 0 | 0.1 | 0.1 | 0 | 0.2 | 0 | 0.1 | 1003 | |
| 0 | 0 | 0 | 0 | 0.1 | 0 | 0 | 0 | 0 | 0.1 | 0 | 0 | 0 | 0 | 0 | 0 | 0.1 | 0 | 0.1 | 0 | 0 | 1013 | |
| 0 | 0 | 0 | 0 | 0 | 0.1 | 0 | 0 | 0 | 0.1 | 0 | 0 | 0 | 0.1 | 0 | 0 | 0.1 | 0 | 0.1 | 0 | 0.1 | 1015 | |
| 0 | 0 | 0 | 0 | 0 | 0 | 0 | 0 | 0 | 0.1 | 0 | 0 | 0 | 0 | 0 | 0 | 0 | 0 | 0 | 0 | 0 | 1022 | |
| 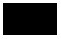 | 0 | 0.3 | 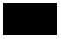 | 0.5 | 0.1 | 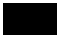 | 0.4 | 0 | 0.2 | 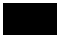 | 0.3 | 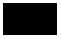 | 0.5 | 0.2 | 0.4 | 0.2 | 0.4 | 0.2 | 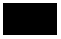 | 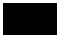 | 1029 | |
| 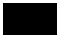 | 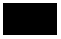 | 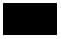 | 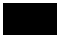 | 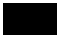 | 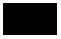 | 0.2 | 0.1 | 0 | 0.3 | 0.3 | 0.1 | 0.2 | 0.1 | 0.2 | 0.1 | 0.2 | 0.3 | 0.3 | 0.3 | 0.1 | 1033 | |
| 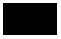 | 0.3 | 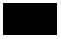 | 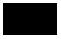 | 0.1 | 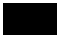 | 0.3 | 0.1 | 0.1 | 0.5 | 0 | 0.4 | 0.5 | 0.2 | 0.3 | 0.1 | 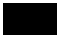 | 0.1 | 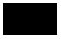 | 0.2 | 0.5 | 1034 | |
| 0.2 | 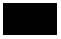 | 0.2 | 0.2 | 0.4 | 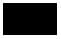 | 0.4 | 0 | 0.1 | 0.2 | 0.5 | 0.4 | 0.3 | 0.2 | 0.1 | 0.1 | 0.2 | 0.5 | 0.1 | 0.4 | 0.3 | 1035 | |
| 0.1 | 0.2 | 0 | 0 | 0.1 | 0.1 | 0.1 | 0.1 | 0.1 | 0 | 0 | 0.2 | 0.2 | 0.1 | 0.1 | 0.1 | 0.1 | 0.1 | 0.2 | 0.1 | 0 | 1037 | |
| 0.2 | 0 | 0.1 | 0 | 0 | 0 | 0.2 | 0.1 | 0.2 | 0.2 | 0.2 | 0.1 | 0 | 0 | 0 | 0.1 | 0.1 | 0.1 | 0 | 0 | 0 | 1038 | |
| 0.1 | 0.1 | 0.1 | 0 | 0.1 | 0.1 | 0.2 | 0.2 | 0 | 0 | 0 | 0 | 0.2 | 0.2 | 0.3 | 0.2 | 0.3 | 0.3 | 0.1 | 0.1 | 0 | 1039 | |
| 0 | 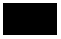 | 0.3 | 0.2 | 0.5 | 0.2 | 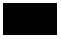 | 0.3 | 0.2 | 0.5 | 0.2 | 0.4 | 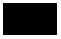 | 0.2 | 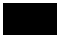 | 0.1 | 0.2 | 0.1 | 0.3 | 0 | 0.3 | 1040 | |
| 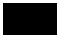 | 0 | 0.1 | 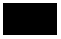 | 0.2 | 0.3 | 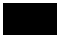 | 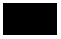 | 0.1 | 0.5 | 0.4 | 0.1 | 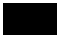 | 0.2 | 0.3 | 0.5 | 0.1 | 0.2 | 0.4 | 0.3 | 0.1 | 1041 | |
| 0.5 | 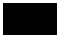 | 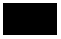 | 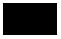 | 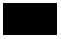 | 0.2 | 0.3 | 0.1 | 0.2 | 0.1 | 0.2 | 0.1 | 0.1 | 0.2 | 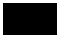 | 0 | 0 | 0.4 | 0 | 0.4 | 0.4 | 1042 | |
| 0.3 | 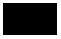 | 0.3 | 0.4 | 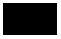 | 0.4 | 0.2 | 0 | 0 | 0.3 | 0.1 | 0.3 | 0.2 | 0 | 0.2 | 0.1 | 0.3 | 0.1 | 0.2 | 0 | 0.2 | 1043 | |
| 0.3 | 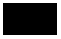 | 0.3 | 0.3 | 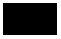 | 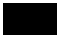 | 0.5 | 0 | 0 | 0.4 | 0.2 | 0.5 | 0.5 | 0.1 | 0.3 | 0 | 0.4 | 0.2 | 0.4 | 0.2 | 0.2 | 1044 | |
| 0.3 | 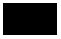 | 0.1 | 0.3 | 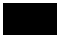 | 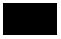 | 0.3 | 0.3 | 0.1 | 0.3 | 0.2 | 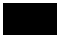 | 0.4 | 0 | 0.1 | 0.2 | 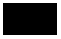 | 0.5 | 0.4 | 0.3 | 0.1 | 1047 | |
| 0.2 | 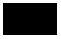 | 0.2 | 0.1 | 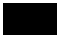 | 0.1 | 0.4 | 0 | 0 | 0.4 | 0.1 | 0.3 | 0.4 | 0 | 0.5 | 0.1 | 0.3 | 0.1 | 0.4 | 0 | 0.5 | 1048 | |
| 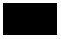 | 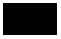 | 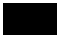 | 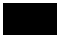 | 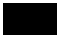 | 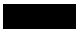 | 0.1 | 0.2 | 0.2 | 0.1 | 0.3 | 0.1 | 0.1 | 0 | 0.2 | 0.1 | 0.1 | 0 | 0.1 | 0.1 | 0.2 | 1049 | |
| 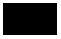 | 0.1 | 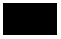 | 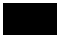 | 0 | 0.3 | 0.5 | 0.1 | 0.1 | 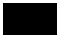 | 0.4 | 0.4 | 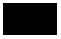 | 0.1 | 0.1 | 0.2 | 0.5 | 0.3 | 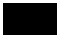 | 0.4 | 0.2 | 1050 | |
| 0.3 | 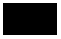 | 0.4 | 0.4 | 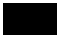 | 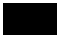 | 0.2 | 0 | 0.1 | 0.3 | 0.2 | 0.2 | 0.2 | 0 | 0 | 0 | 0.2 | 0.2 | 0.2 | 0.2 | 0 | 1051 | |
| 0.1 | 0.3 | 0.3 | 0.3 | 0 | 0.2 | 0.2 | 0.2 | 0.3 | 0 | 0.1 | 0 | 0.1 | 0.2 | 0.1 | 0.1 | 0.2 | 0.1 | 0.3 | 0.1 | 0.2 | 1052 | |
| 0.3 | 0.2 | 0.2 | 0.3 | 0.1 | 0.3 | 0.1 | 0 | 0 | 0.2 | 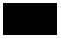 | 0.1 | 0.1 | 0.1 | 0.4 | 0 | 0.2 | 0.5 | 0.2 | 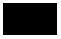 | 0.3 | 1053 | |
| 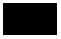 | ### | 0.3 | 0.4 | 0.3 | 0.3 | 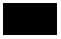 | 0.2 | 0.2 | 0.3 | 0.3 | 0.3 | 0.4 | 0.3 | 0.3 | 0.1 | 0.1 | 0.1 | 0.1 | 0.1 | 0 | 1055 | |
| 0.4 | 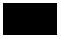 | 0.4 | 0.3 | 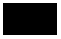 | 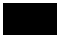 | 0.4 | 0 | 0.1 | 0.4 | 0.4 | 0.4 | 0.4 | 0 | 0 | 0.1 | 0.4 | 0.4 | 0.5 | 0.5 | 0 | 1056 | |
| 0.2 | 0 | 0.1 | 0.1 | 0 | 0 | 0.2 | 0.1 | 0 | 0.2 | 0.2 | 0.1 | 0.2 | 0 | 0 | 0 | 0.1 | 0.1 | 0.2 | 0.2 | 0 | 1057 | |
| 0 | 0.1 | 0 | 0.1 | 0.1 | 0.1 | 0.1 | 0 | 0 | 0.1 | 0.1 | 0.1 | 0.1 | 0 | 0 | 0 | 0.1 | 0.1 | 0.1 | 0.1 | 0 | 1059 | |
| 0.4 | 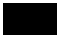 | 0.3 | 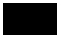 | 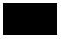 | 0.4 | 0.3 | 0.1 | 0.1 | 0.3 | 0 | 0.4 | 0.2 | 0 | 0.3 | 0.2 | 0.4 | 0.1 | 0.2 | 0.1 | 0.3 | 1060 | |
| 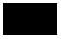 | 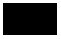 | 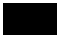 | 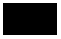 | 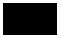 | 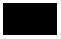 | 0.1 | 0 | 0 | 0.1 | 0.1 | 0.1 | 0.1 | 0.2 | 0.2 | 0 | 0.1 | 0.2 | 0 | 0.1 | 0.1 | 1062 | |
| 0.5 | 0.2 | 0.3 | 0.4 | 0.1 | 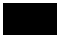 | 0.3 | 0.2 | 0.1 | 0.4 | 0.3 | 0.1 | 0.2 | 0.1 | 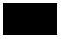 | 0.1 | 0.2 | 0.5 | 0.3 | 0.4 | 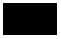 | 1064 | |
| 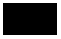 | 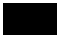 | 0.4 | 0.4 | 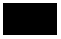 | 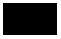 | 0.1 | 0.2 | 0.2 | 0.2 | 0.2 | 0.3 | 0.3 | 0.1 | 0.1 | 0 | 0.4 | 0.4 | 0.4 | 0.3 | 0 | 1065 | |
| 0.2 | 0.2 | 0.2 | 0.2 | 0.2 | 0.2 | 0 | 0 | 0 | 0 | 0 | 0 | 0 | 0 | 0 | 0 | 0 | 0 | 0 | 0 | 0 | 1066 | |
| 0 | 0 | 0 | 0 | 0 | 0 | 0 | 0 | 0 | 0 | 0 | 0 | 0 | 0 | 0 | 0 | 0 | 0 | 0 | 0 | 0 | 1068 | |
| 0.1 | 0.3 | 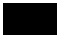 | 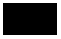 | 0.2 | 0 | 0.1 | 0.5 | 0.4 | 0.1 | 0.1 | 0.3 | 0.3 | 0.1 | 0.3 | 0 | 0.4 | 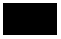 | 0.4 | 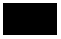 | 0.2 | 1070 | |
| 0.3 | 0 | 0.1 | 0.2 | 0 | 0.1 | 0.3 | 0.2 | 0.1 | 0.3 | 0.4 | 0.1 | 0.2 | 0 | 0.1 | 0.1 | 0.1 | 0.2 | 0.2 | 0.3 | 0.1 | 1071 | |
| 0 | 0 | 0.4 | 0.5 | 0 | 0 | 0 | 0.4 | 0.5 | 0 | 0 | 0.4 | 0.5 | 0 | 0 | 0.1 | 0.4 | 0.4 | 0.5 | 0.5 | 0 | 1072 | |
| 0.1 | 0.1 | 0.1 | 0.1 | 0.1 | 0.1 | 0 | 0 | 0 | 0 | 0 | 0 | 0 | 0 | 0 | 0 | 0 | 0 | 0 | 0 | 0 | 1073 | |
| 0.4 | 0.1 | 0.4 | 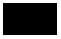 | 0.5 | 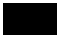 | 0.3 | 0 | 0.2 | 0.1 | 0.2 | 0.3 | 0.4 | 0.4 | 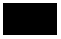 | 0.2 | 0.1 | 0.2 | 0.1 | 0.1 | 0.2 | 1074 | |
| 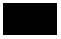 | 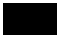 | 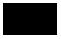 | 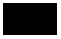 | 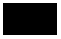 | 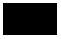 | 0.1 | 0 | 0 | 0.1 | 0.1 | 0.1 | 0.1 | 0 | 0 | 0 | 0.1 | 0.1 | 0.1 | 0.1 | 0 | 1075 | |
| 0.5 | 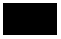 | 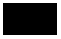 | 0.4 | 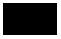 | 0.3 | 0.1 | 0.1 | 0.1 | 0 | 0.1 | 0.1 | 0.1 | 0 | 0.2 | 0.2 | 0.1 | 0.3 | 0.1 | 0.1 | 0.2 | 1076 | |
| 0.3 | 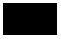 | 0.4 | 0.3 | 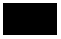 | 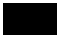 | 0.4 | 0 | 0 | 0.4 | 0.4 | 0.4 | 0.4 | 0 | 0 | 0.1 | 0.4 | 0.4 | 0.5 | 0.4 | 0 | 1077 | |
| | | | ## | | | | | | | | | | | | | | | | | | | |
| 0.3 | 0.1 | 0.1 | # | 0.3 | 0.1 | 0.2 | 0.3 | 0.3 | 0.1 | 0.2 | 0.1 | 0.1 | 0.1 | 0 | 0.1 | 0.2 | 0 | 0.3 | 0.1 | 0.2 | 1078 | |
| 0.3 | 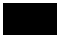 | 0.2 | 0.2 | 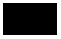 | 0.4 | 0.3 | 0.1 | 0.1 | 0.2 | 0.1 | 0.4 | 0.4 | 0.1 | 0.2 | 0 | 0.3 | 0.2 | 0.3 | 0.2 | 0.1 | 1080 | |
| 0.3 | 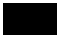 | 0.4 | 0.3 | 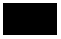 | 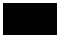 | 0.5 | 0.2 | 0.1 | 0.5 | 0.3 | 0.3 | 0.4 | 0 | 0.1 | 0.1 | 0.3 | 0.2 | 0.4 | 0.3 | 0.1 | 1081 | |
| 0.3 | 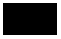 | 0.3 | 0.2 | 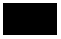 | 0.4 | 0.2 | 0.1 | 0.1 | 0.2 | 0.1 | 0.3 | 0.3 | 0 | 0.1 | 0.1 | 0.3 | 0.1 | 0.3 | 0.2 | 0.1 | 1082 | |
| 0.5 | 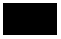 | 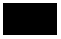 | 0.3 | 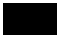 | 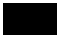 | 0.4 | 0.2 | 0.2 | 0.2 | 0.4 | 0.2 | 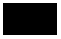 | 0.1 | 0.1 | 0.3 | 0.1 | 0.3 | 0.4 | 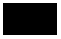 | 0.2 | 1083 | |
| 0 | 0.5 | 0.1 | 0.1 | 0.3 | 0.2 | 0.5 | 0.1 | 0 | 0.4 | 0.2 | 0.4 | 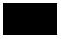 | 0.1 | 0.3 | 0.2 | 0.3 | 0.1 | 0.4 | 0.3 | 0.2 | 1084 | |
| 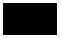 | 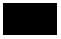 | 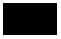 | 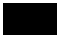 | 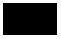 | 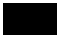 | 0.2 | 0.2 | 0.2 | 0.2 | 0.2 | 0.1 | 0 | 0 | 0 | 0.1 | 0 | 0.1 | 0 | 0 | 0 | 1085 | |
| 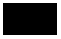 | 0.1 | 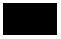 | 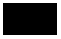 | 0.1 | 0.4 | 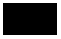 | 0.2 | 0 | 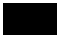 | 0.4 | 0.4 | 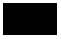 | 0 | 0.2 | 0.1 | 0.4 | 0.2 | 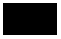 | 0.4 | 0.2 | 1087 | |
| 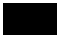 | 0.1 | 0.5 | 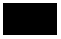 | 0.2 | 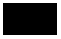 | 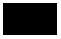 | 0.2 | 0.2 | 0.5 | 0.1 | 0.4 | 0.4 | 0.1 | 0.5 | 0 | 0.3 | 0.1 | 0.3 | 0.1 | 0.4 | 1088 | |
| 43 | 36 | 45 | 45 | 38 | 39 | 39 | 16 | 7 | 41 | 32 | 30 | 36 | 10 | 21 | 5 | 27 | 24 | 37 | 27 | 23 | | |

“DELTA” (δ) values for select AIMs from the list claimed are shown.
The AIM unique identifier is shown in the last column.
Cells having numbers in bold (not shaded) indicate good δ values; cells that are shaded with numbers in bold represent extremely high δ values.
AF-African, CT-European, EA-East Asian, SA-South Asian, ME-middle eastern, PI-Pacific Islander, AI-Native American.

First, Table 4 was derived from screening several hundred candidate AIMs electronically selected from the public databases, thus demonstrating that only a minority of the candidate AIMs from the public databases are real AIMs. As discussed above, the public SNP databases were electronically screened to find good candidate AIMs (since frequency data is provided for three “racial” groups, though the level of admixture for these groups is not known). Second, 384 individuals of various continental and BGA origins were genotyped at each of these sites: 70 African samples collected from Nigeria and Congo, 65 European samples collected from Northern Europe, 70 East Asian samples collected from recent immigrants to San Francisco, Calif.; 35 Middle Eastern samples collected from Turkey, 35 South Asian samples collected from India and 25 Pacific Islander samples collected from the Philippines and US Samoa).

A sampling of the data for about 70 AIMs that passed the screening process from 175 candidate AIMs screened is shown in Table 4. The delta (δ) value is a measure of how well the sequence for a polymorphism enables one to predict membership to one or the other group; i.e., how distinct the two populations are with regard to the sequence at this polymorphism. δ values are shown for 69 of the 175 AIMs; the other 105 had δ values of 0 for each pair-wise population comparison and, therefore, were not true AIMs. AIM 1068 in Table 4 is representative of types of failure (zeros across all pair wise comparisons—some AIMs with zeros across the population pairs are present in Table 4 because they are informative for populations not shown in this particular table). This result confirms that most of the candidate AIMs culled from the public database are not true AIMs and highlights the value of the present invention.

A relatively large investment in genotyping and analysis required to identify which candidate AIMs are true AIMs. While this process can be bypassed, e.g., one could simply genotype samples at 100 candidate AIMs and extract the data from those that prove to really be AIMs, genotyping is an expensive procedure and, as such, the waste incurred would make the test economically impractical. To develop an economical and practical test for ancestral proportions, the test must query large numbers of true AIMs. Many publicly available candidate AIMs are not true AIMs due, for example, to low sample sizes, such that allele frequencies in the public databases are simply not very reliable, though they do offer some information. While it is expected that the frequency of true (i.e., validated) and well-characterized (i.e., population specific frequencies known with certainty) AIMs in a random collection of SNPs would be about 5%, the frequency of true AIMs in a culled set of candidate AIMs is about 50% and, after proceeding as disclosed herein, the frequency of true AIMs in a collection of SNPs is 100%.

Second, the results of Table 4 demonstrate that some of the AIMs are good for the resolution of Africans vs. Europeans, other AIMs are good for the resolution of Native Americans vs. Africans, etc. Though selected based on European/African/Asian allele frequency differentials, some AIMs provide good distinction of other groups such as Pacific Islanders, South Asians and Middle Easterners. This type of information can only be learned by genotyping in larger samples, and a test for ancestral proportions must go through this step in order to be accurate (for example, if the test only worked in the 3-dimensions allowed by data that is publicly available—European, African and Asian—the results obtained, for example, for a Hispanic would be ambiguous). The panel of SNPs in Table 4 provides a well balanced mix of AIMs with resolution power for each of the possible pair-wise comparisons for 7 population groups, and this panel would constitute a good test for ancestry proportions. Data for south Asians, Middle Easterners and Pacific Islanders do not exist in the public databases and, therefore, was generated for these studies. In comparison, one attempting to develop a test for ancestral proportions in 7-dimensions by simply sequencing at candidate AIMs haphazardly selected from the public SNP databases (i.e., without selection through data production) would need to compile a battery of thousands of SNPs to obtain a panel such as that in Table 4 because certain pairs of populations are difficult to resolve (e.g., South Asians and Europeans, which constitute a larger IndoEuropean group united by a common language base).

The results obtained using the panel of AIMs shown in Table 4 is shown in Table 5. The presently disclosed algorithm (see Example 6, Table 12) was used to calculate the proportions for a group of 96 individuals who reside in the southeastern United States, and who describe themselves to be Caucasian.

TABLE 5
Ancestral proportions for a large number of self-reported Caucasians

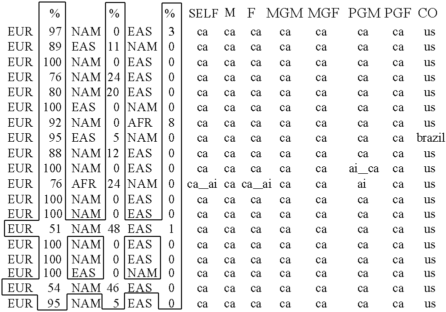

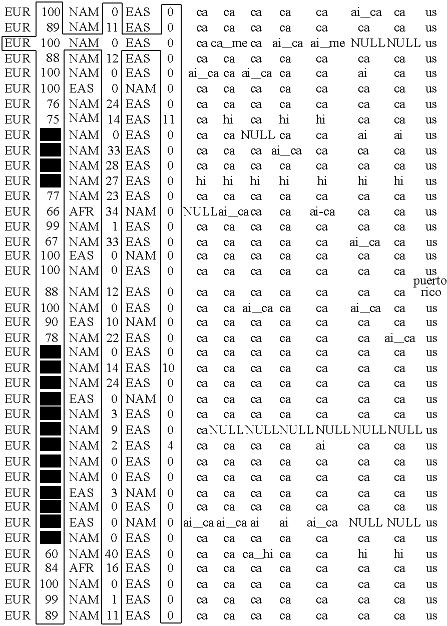

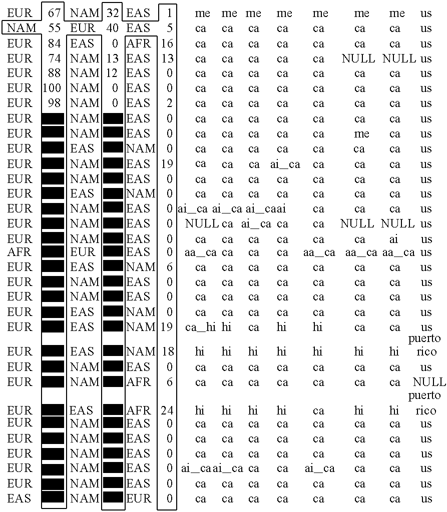

The percentage of ancestry is given after each group:
EUR - IndoEuropean, NAM - Native American, EAS - East Asian/Pacific Islander, AFR - African.
The self-reported race for each individual (SELF), their mother (M), father (F), maternal grandmother (MGM), maternal grandfather (MGF), paternal grandmother (PGM), paternal grandfather (PGF) and the country of their birth is shown.

The results of Table 5 demonstrate that most people who describe themselves to be Caucasian are, indeed, of majority IndoEuropean ancestry as determined using the BGA test, i.e., using the panel of markers in Table 4 and the algorithm (Example 6, Table 12). About 40% of these Caucasians were measured as 100% European ancestry, with no admixture, whereas 60% showed detectable admixture. By way of critical evaluation, the proportions obtained can be compared with self-reported admixture. For individuals of Table 5 that claim all of their parents and grandparents are unmixed Caucasians (“ca” across all columns), the rate at which 90% or greater European ancestry is found is higher (55%) for persons who report no mixture in their pedigree than for persons who report mixture in their pedigree (35%). The fact that half of those reported no mixture in their pedigree illustrates how little most people know about their BGA, at least in anthropological as compared to geopolitical terms.

The public databases employed a small number of samples for each of the three groups (African-Americans, Europeans and Asians). Thus, from the public databases, the actual allele frequencies for the claimed SNPs is uncertain and was only determined with accuracy from the present work. Further, the use of African-Americans as a parental group is faulty since, as disclosed herein, they are an admixed population (between Africans and Europeans). The best way to find SNP markers that are useful for the present methods is to genotype a number of samples from the major BGA groups of the world for all of those of apparently different minor allele frequency between at least two of the groups, calculate the δ values, and rank them.

Family Pedigree

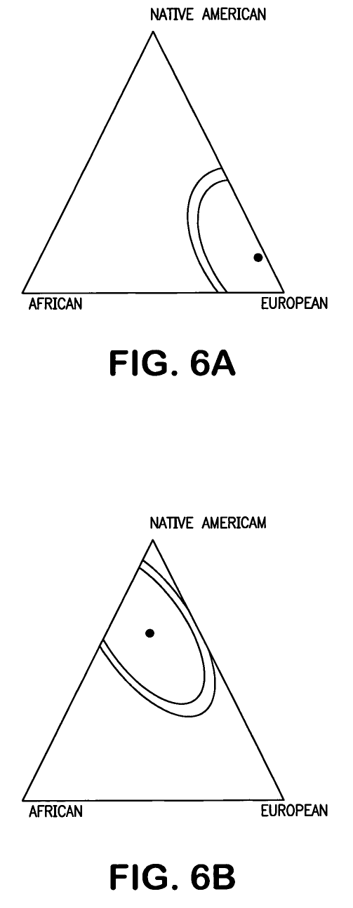

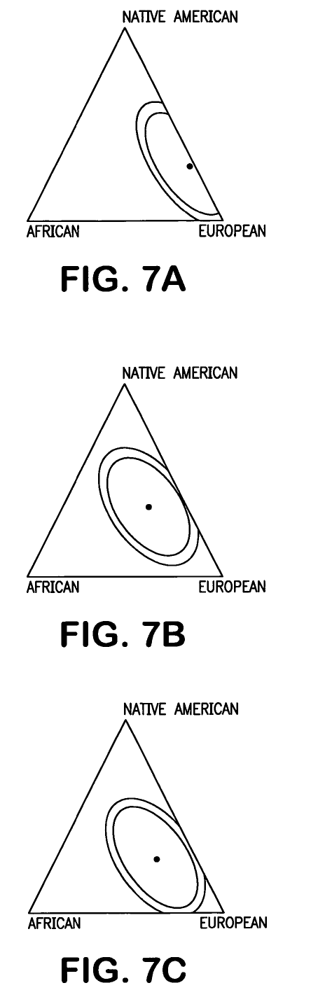

The BGA method for the use of SNPs (AIMs) to determine ancestral proportions was applied to the examination of a family pedigree of a father, mother and their 3 children (see FIGS. 6 and 7). The man was mostly European and his wife is Mexican, so, if the test is accurate, their three children should plot somewhere between the man and his wife and this is exactly what was observed. Three of the father's grandparents on the father's side of the family are relatively pure Greek/European, but one was almost pure Cherokee. All four of his grandparents on his mother's side were European mix. The plots for the mother (FIG. 6B) and father (FIG. 6A) are shown. By drawing lines from the vertices that bisect the triangle edge opposite that vertex, where the vertex represents 100% and the triangle edge 0%, it can be seen that the father is about 85% European, 11% Native American, 4% African (he was of no detectable East Asian, South Asian or Pacific Islander ancestry). The 11% Native American seems to have come from his paternal grandparent. The percent that would be predicted, knowing that ⅞ of his great-grandparents were European mix and ⅛ were Native American, is 12% (⅛), which is in good agreement with the data produced from the test. The mother is from Mexico and is of Hispanic descent. As discussed previously, Hispanics are an admixed population with European and Native American contribution. It can be seen here that the mother is of 11% European, 76% Native American and 13% African descent (there was no detectable East Asian, South Asian or Pacific Islander ancestry).

Because the three children each received one chromosome from each of their mother and father, they should each plot somewhere in between him and his wife. Using the method the children plot as expected (FIG. 7). It is clear from these results that the point estimate of the children is between that of the parents, as would be expected. From these results it seems clear that Child #1 (FIG. 7A; 80% European, 18% Native American, 2% African) has ancestral proportions more similar to his father than his mother, whereas Child #2 (FIG. 7B; 61% European, 31% Native American, 8% African) and Child #3 (FIG. 7C; 54% European, 37% Native American, 9% African) are of ancestral proportions about half way between their two parents.

Although each child receives a chromosome from the mother and one from the father, the children are different because the mother has chromosomes of mostly Native American descent, though some have European and African flavor as well, and the father has chromosomes of mostly European flavor though some are of Native American flavor as well. When a child is conceived, he or she receive a chromosome from each parent, but which of the two the child receives from the mother is random (i.e., “independent assortment”). Since some of the mother's chromosome pairs have a member of the pair with European flavor, some of the children will receive the “European flavored chromosome” and other children will not. From the present study, it is clear that Child #1 (FIG. 7A) received more of his mother's European flavored chromosomes than did the other two children. Thus, while each child is a 50/50 blend of maternal and paternal chromosomes, their ancestral proportions are unique and random functions of those of their parents.

### EXAMPLE 2

### Four Way Admixture Estimate of Ancestry

This example demonstrates that a four way admixture BGA test provides the same results obtained using three 3 way BGA tests.

As indicated above, BioGeographical Ancestry (BGA) is the heritable component of race. Because socio-cultural and geo-political metrics for measuring human race are human, not natural, constructs, their use in genetics research makes it difficult to control for population genetic structure, and may obscure important correlations between BGA and human biology. This example provides methods and compositions to accurately measure genetic structure within individuals. The human genome was mined for candidate Ancestry Informative Markers (AIMs), which were validated on an ultra-high throughput genotyping platform and used to establish parental population allele frequencies. Using 71 of the most informative AIMs (Table 6), which cover most of the chromosomes, and coalescing the human population to four main continental population groups (sub-Saharan African, East Asian, IndoEuropean and Native American), the MLE method was used to determine individual BGA admixture proportions and their associated confidence intervals. As disclosed herein, self-reported population affiliations correlated almost perfectly with the majority BGA population affiliation determined for a sample of 2,024 international samples. BGA admixture results were surprisingly frequent, and when observed, were generally not inconsistent with anthropological and geopolitical history. The admixture proportions produced tracked in family pedigrees in a manner consistent with the law of independent assortment, and simulation revealed that the markers relevant for resolving the group affiliations functioned independently within the confines of our algorithm. Because a large number of high 6 value markers were used, the test was surprisingly robust; reasonable levels of simulated allele frequency errors that could be caused by biased parental sampling had no significant impact on the BGA proportions determined. These results demonstrate that BGA admixture can be reliably determined from a DNA sample.

Sample Collection:

Parental Samples—For establishing the parental group allele frequencies, 100 relatively homogeneous descendents of four crudely defined human population groups were genotyped. These four groups corresponded to a coalescence of a simplified human pedigree back in time to a point where populations were relatively isolated to the sub-Saharan African (sub-Saharan Africans), Europe and the Middle Eastern (IndoEuropeans), North/South American (Native Americans) and East Asian (East Asians) continental regions. In essence, the extremes of the existing populations has been taken in terms of physical characteristics and known human migration patterns, and the human pedigree simplified by assuming that, because the rest of the population exhibits physical features along a continuum defined by these extremes, all of humanity arose from radiation within and admixture between these four main continental groups.

Collection efforts were focused on individuals residing in each region and of relatively homogeneous affiliation with descendents of each group in terms of self-described “race”; each subject exhibited a strong physical appearance associated with descendents of each group and reported homogeneous affiliation with that group. There exists no de-facto final arbiter of BGA affiliation, or race, but collecting without regard to ethnicity might introduce systematic errors in frequency estimation for a given group if systematic admixture is a function of ethnicity. Where possible, an attempt was made to collect from as wide a variety of ethnicities as possible within each parental group, expecting admixture within each parental sample to balance out. Though it would be better to collect from individuals of known homogeneous affiliation, if sampling was not biased in terms of admixture or other population structure, the admixture extant to samples that are practical to collect from would tend to reduce the power of the test rather than introduce systematic bias. The existence of Hardy-Weinberg Equilibrium for each marker within each BGA group was relied on as an indication that a reasonably good sample was obtained. The sub-Saharan African samples were collected in Nigeria and Congo, Africa; the European samples were collected from various locales in the United States; the East Asian samples were collected from Japan and China; and the Native American samples were collected from “Nativos” inhabiting a remote region of southern Mexico. All samples were collected under IRB guidelines for the purposes of genetic studies of human population variation.

Experimental Samples—After reading and signing an approved IRB consent form, subjects completed a biographical questionnaire and provided either a buccal swab or 4 ml of blood. On the questionnaire, the subjects described themselves, their mother, father and maternal and paternal grandparents as belonging to the “African”, “American Indian”, “Asian”, “Caucasian”, “Hispanic” or “Other” group, with the option of reporting “Don't Know” for each family member. For some of the subjects, digital photographs were taken; explicit permission was received from those subjects whose photographs were presented. DNA was extracted from circulating lymphocytes or buccal swabs using commercial kits (Qiagen), and a primer extension protocol employing a 25K SNPstream ultra-high throughput (UHT) genotyping system was used (Orchid Biosciences).

Estimating BioGeographical Ancestry (BGA)

A software program was written based on the algorithm of Hanis et al. (supra, 1986) for using multilocus AIM genotypes to determine the Maximum Likelihood Estimate (MLE) of individual BGA admixture (Example 6; see, also, Table 12). The delta (δ) value is an expression of the ancestry informativeness of the marker (Dean et al., supra, 1994). For a biallelic marker, the frequency differential (δ) is equal to px−py, which is equal to qy−qx, where px and py are the frequencies of one allele in populations X and Y and qx and qy are the frequencies of the other. To test the departures from independence in allelic state within and between loci, the MLD exact test was used (Zaykin et al., Genetica 96:169-78, 1995).

The collection of 71 AIMs used for in this Example was selected to maximize the cumulative δ value within, and minimize differences in the cumulative δ value between each of the six possible pairs of the four dimensional (sub-Saharan African, Native American, IndoEuropean and East Asian) problem. The algorithm inverts the population specific allele frequencies to obtain a likelihood estimate of proportional affiliation corresponding to a multilocus genotype using three groups at a time (mainly for computational convenience and because a 4-dimensional admixture is likely to be relatively rare). For example, the likelihood of 100% IndoEuropean, 0% Native American, 0% East Asian is calculated, then the likelihood of 99% IndoEuropean, 1% Native American, 0% East Asian is calculated next, and so on until all possible IndoEuropean, Native American and East Asian proportions are considered, then the process is repeated for all possible IndoEuropean, Native American and African proportions, and all possible Native American, African and East Asian proportions. The likelihood of maximum value is selected as the Maximum Likelihood Estimate (MLE).

When plotting a single MLE on a triangle plot, the space within which the likelihood is within 2-fold, 5-fold and 10-fold of the MLE is delimited (these intervals are not plotted when multiple MLEs are shown in a single triangle plot). For calculating the MLE using all four of the BGA groups together, the procedure was executed in the same exact manner; instead of 3 possible 3-way BGA combinations, only one 4-way BGA combination is possible. All of the MLEs described in this Example were calculated using the 3-way calculation scheme. Alternative versions of this type of test are possible, for example, using different AIMs and different parental groups corresponding to different coalescences of the human pedigree, which would be expected to provide results meaningful with respect to a different anthropological time scale than those provided here.

At the time the database was screened, the SNP Consortium (TSC) had contributed data on approximately 27,000 SNPs where frequencies were available on three populations (African American, European American, and East Asian). This database was screened for candidate AIMs, i.e., SNPs of δ>0.40 between any two of the four continental population groups (see Example 1; see, also, Shriver et al., supra, 1997). Parental samples of sub-Saharan Africans (AA), IndoEuropeans (IE), East Asians (EA) and Native Americans (NA) were screened for each of the 200 candidate AIMs with the largest δ values and, of these, 71 were validated as true AIMs (i.e., true SNPs), with minor allele frequency greater than 1% and of δ>0.40 for at least one of the group pairs. The 71 AIMS are shown as SEQ ID NOS:1 to 71; the top 100 candidate AIMs for the group pairs were as follows: EA by AA (SEQ ID NOS: SEQ ID NOS:7, 21, 23, 27, 45, 54, 59, 63, and 72 to 152); EA by IE (SEQ ID NOS:3, 8, 9, 11, 12, 33, 40, 59, 63, and 153 to 239); and IE by AA (SEQ ID NOS:1, 8, 11, 21, 24, 40, 172, and 240 to 331). It should be noted that some AIMs identified by one pairwise comparison also can be AIMs for a second pairwise comparison (e.g., SEQ ID NO:59 was identified as an AIM for EA by AA and EA by IU comparisons), although such AIMs are an exception. In addition, many of the 71 AIMs are not in the list of the top 100 candidate AIMs shown for any of the pairs (but were in the top 200 candidate AIMs); candidate AIMs were not used, for example, because they did not genotype well due to the SNP type of amplification parameters used for the exemplified platform, or for other reasons as disclosed herein.

TABLE 6
Pair-wise δ values for the 71 AIMs used in the BGA test

| | | | | | | | SEQ |
|---|---|---|---|---|---|---|---|
| AF-CT | AF-EA | AF-AI | CT-EA | CT-AI | EA-AI | AIM | ID NO: |
| 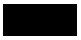 | 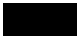 | 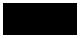 | 0.12 | 0.09 | 0.03 | 958 | 31 |
| 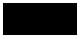 | 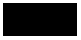 | 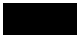 | 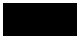 | 0.11 | 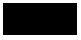 | 960 | 65 |
| 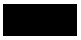 | 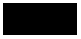 | 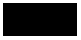 | 0.01 | 0.04 | 0.02 | 961 | 52 |
| 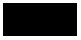 | 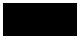 | 0.08 | 0.08 | 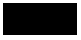 | 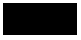 | 963 | 39 |
| 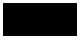 | 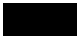 | 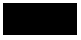 | 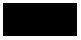 | 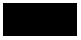 | 0.08 | 964 | 70 |
| 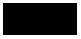 | 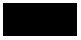 | 0.01 | 0.14 | 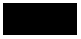 | 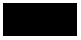 | 966 | 37 |
| 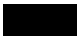 | 0.097 | 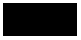 | 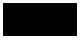 | 0.17 | 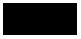 | 969 | 63 |
| 0.163 | 0.171 | 0.06 | 0.01 | 0.11 | 0.11 | 970 | 40 |
| 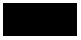 | 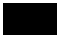 | 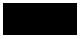 | 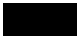 | 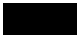 | 0.13 | 971 | 64 |
| 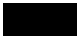 | 0.186 | 0.16 | 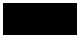 | 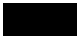 | 0.03 | 972 | 1 |
| 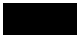 | 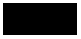 | 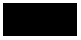 | 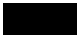 | 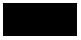 | 0.16 | 973 | 22 |
| 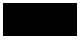 | 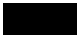 | 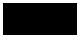 | 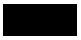 | 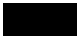 | 0.13 | 977 | 57 |
| 0.041 | 0.122 | 0.18 | 0.08 | 0.14 | 0.06 | 978 | 3 |
| 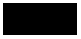 | 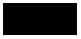 | 0.19 | 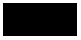 | 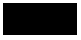 | 0.07 | 979 | 34 |
| 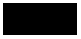 | 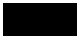 | 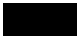 | 0.07 | 0.08 | 0.15 | 980 | 21 |
| 0.102 | 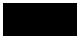 | 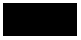 | 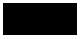 | 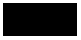 | 0.16 | 993 | 54 |
| 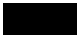 | 0.071 | 0.09 | 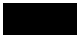 | 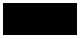 | 0.02 | 1000 | 18 |
| 0.038 | 0.1 | 0.14 | 0.06 | 0.1 | 0.04 | 1015 | 24 |
| 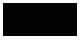 | 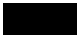 | 0.06 | 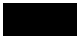 | 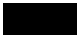 | 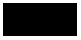 | 1022 | 20 |
| 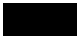 | 0.025 | 0.14 | 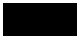 | 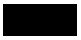 | 0.17 | 1029 | 13 |
| 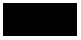 | 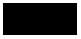 | 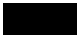 | 0.19 | 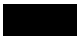 | 0.16 | 1033 | 41 |
| 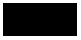 | 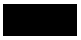 | 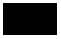 | 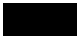 | 0.03 | 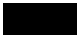 | 1034 | 46 |
| 0.172 | 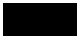 | 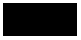 | 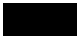 | 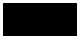 | 0.09 | 1035 | 25 |
| 4E-04 | 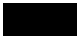 | 0.17 | 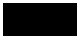 | 0.17 | 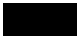 | 1040 | 29 |
| 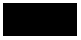 | 0.031 | 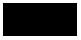 | 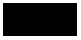 | 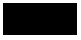 | 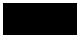 | 1041 | 5 |
| 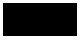 | 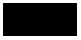 | 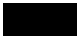 | 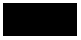 | 0.06 | 0.16 | 1043 | 35 |
| 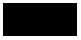 | 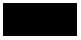 | 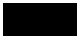 | 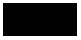 | 0.19 | 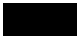 | 1044 | 16 |
| 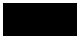 | 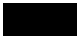 | 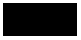 | 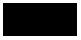 | 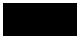 | 0.13 | 1047 | 61 |
| 0.168 | 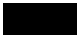 | 0.08 | 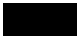 | 0.08 | 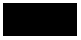 | 1048 | 19 |
| 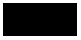 | 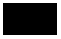 | 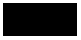 | 0.11 | 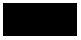 | 0.16 | 1049 | 26 |
| 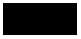 | 0.114 | 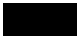 | 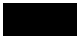 | 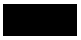 | 0.14 | 1050 | 48 |
| 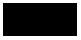 | 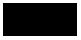 | 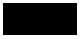 | 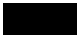 | 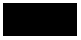 | 0.01 | 1051 | 42 |
| 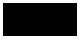 | 0.171 | 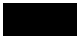 | 0.09 | 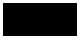 | 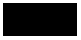 | 1053 | 62 |
| 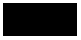 | 0 | 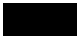 | 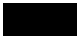 | 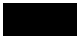 | 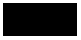 | 1055 | 67 |
| 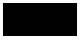 | 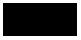 | 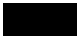 | 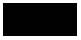 | 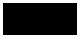 | 0.01 | 1056 | 44 |
| 0.153 | 0.022 | 0.03 | 0.18 | 0.18 | 0.01 | 1057 | 67 |
| 0.043 | 0.043 | 0.04 | 0 | 0 | 0 | 1058 | 59 |
| 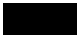 | 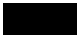 | 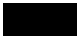 | 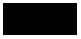 | 0 | 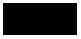 | 1060 | 27 |
| 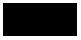 | 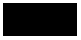 | 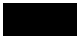 | 0.1 | 0.14 | 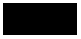 | 1062 | 23 |
| 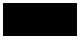 | 0.193 | 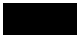 | 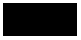 | 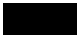 | 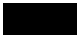 | 1064 | 10 |
| 0.157 | 0.157 | 0.16 | 0 | 0 | 0 | 1066 | 7 |
| 0.043 | 0.043 | 0.04 | 0 | 0 | 0 | 1068 | 11 |
| 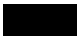 | 0.013 | 0.11 | 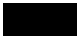 | 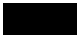 | 0.1 | 1071 | 53 |
| 0.071 | 0.071 | 0.07 | 0 | 0 | 0 | 1073 | 8 |
| 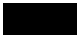 | 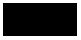 | 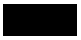 | 0.1 | 0.1 | 0 | 1075 | 69 |
| 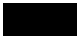 | 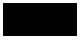 | 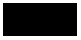 | 0.07 | 0.13 | 0.2 | 1076 | 71 |
| 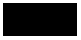 | 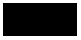 | 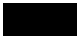 | 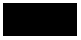 | 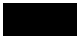 | 0.01 | 1077 | 45 |
| 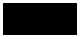 | 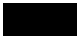 | 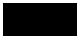 | 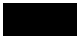 | 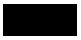 | 0.13 | 1081 | 43 |
| 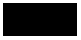 | 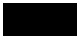 | 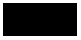 | 0.19 | 0.06 | 0.13 | 1082 | 36 |
| 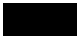 | 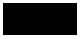 | 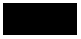 | 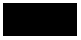 | 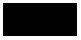 | 0.05 | 1083 | 6 |
| 0.036 | 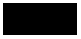 | 0.17 | 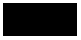 | 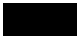 | 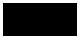 | 1084 | 51 |
| 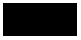 | 0.117 | 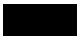 | 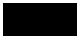 | 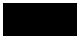 | 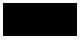 | 1087 | 28 |
| 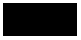 | 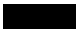 | 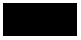 | 0.01 | 0.01 | 0.01 | 1111 | 30 |
| 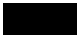 | 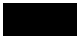 | 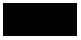 | 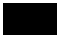 | 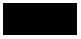 | 0.12 | 976 | 68 |
| 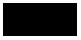 | 0.093 | 0.15 | 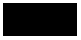 | 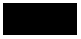 | 0.06 | 1113 | 4 |
| 0.133 | 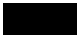 | 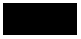 | 0.07 | 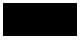 | 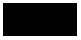 | 1116 | 50 |
| 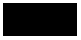 | 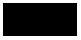 | 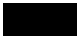 | 0.14 | 0.05 | 0.09 | 1117 | 47 |
| 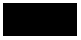 | 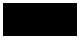 | 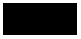 | 0.08 | 0.07 | 0.01 | 1120 | 60 |
| 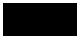 | 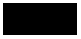 | 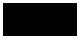 | 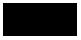 | 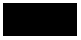 | 0.09 | 1121 | 33 |
| 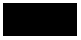 | 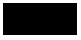 | 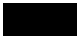 | 0.09 | 0.02 | 0.07 | 1122 | 58 |
| 0.177 | 0.086 | 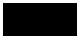 | 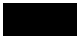 | 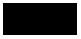 | 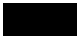 | 1124 | 17 |
| 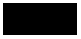 | 0.089 | 0.02 | 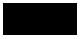 | 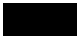 | 0.07 | 1128 | 2 |
| 0.108 | 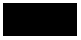 | 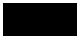 | 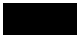 | 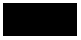 | 0.07 | 1036 | 9 |
| 0.149 | 0.1 | 0.1 | 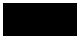 | 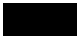 | 0 | 1130 | 55 |
| 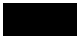 | 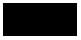 | 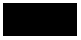 | 0.16 | 0.14 | 0.02 | 1136 | 38 |
| 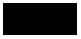 | 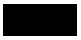 | 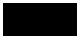 | 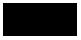 | 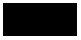 | 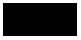 | 1137 | 56 |
| 0.076 | 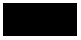 | 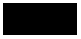 | 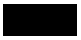 | 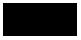 | 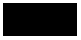 | 1138 | 66 |
| 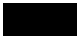 | 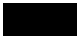 | 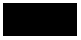 | 0.05 | 0.01 | 0.04 | 1139 | 32 |
| 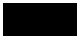 | 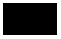 | 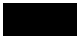 | 0.17 | 0.14 | 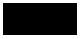 | 1140 | |
| 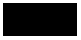 | 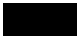 | 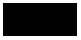 | 0.08 | 0.12 | 0.04 | 1141 | 49 |
| 0.155 | 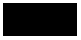 | 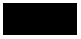 | 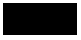 | 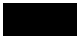 | 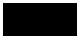 | 1146 | 15 |
| 54 | 50 | 50 | 45 | 41 | 24 | | |

AF - sub-Saharan African, CT - Indo European, EA - East Asian, AI - Native American.
The AIM unique identifier is shown (AIM) as well as the GENBANK accession number provided by the authors upon submission of the SNP sequence to the NCBI:dbSNP database.

The number of AIMs with Λ>0.40 for each pair-wise comparison is shown at the bottom of the list.

The 71 AIMs used in the exemplified panel were spread throughout 21 of the 23 autosomal chromosomes (FIG. 8), with the average chromosome containing 3 AIMs (see Table 6). Each had alleles in Hardy-Weinberg equilibrium, both overall with all four BGA groups considered together, and within each BGA group, and none were found to be in linkage disequilibrium with one another. The software program used individual genotypes for these AIMs with a maximum likelihood algorithm (see Example 6, Table 12; see, also, Example 1). The use of the 71 markers with this algorithm provides another example of the “BGA test”.

The BGA test was used to calculate BGA admixture proportions for the parental Native American, African and Indo European samples used in the construction of the test. After calculating the admixture proportions for each sample, they were plotted in a triangle plot to allow for the relative proportions of a 3-way mixture to be represented in two dimensions. Because these were the same samples that comprised the parental groups and from which the population allele frequencies were derived, they were expected to exhibit relatively homogeneous BGA (i.e., of low admixture) and, in fact, the sub-Saharan Africans, Native Americans and European parental samples all registered with relatively homogeneous BGA (i.e., they plotted towards the appropriate vertices of a BGA triangle).

The BGA test was next used to determine BGA proportions for 1,186 individuals of self-reported race (43 African Americans, 1,120 Caucasians, and 23 Hispanics). 306 of the individuals (26%) showed homogeneous BGA (100% for any one group). 101 of the 1,186 (8.5%) harbored >5% BGA affiliation for three groups, indicating that, for these individuals, a modification of the software to perform 4-group calculations might be more appropriate, and the vast majority of the samples were characterized by 2-way admixture. Significantly greater European admixture was identified in African Americans relative to that of Nigerians (visualized as a dispersal of points away from the sub-Saharan African vertex). In contrast, the IndoEuropean samples plotted as neatly in the IndoEuropean vertex as had the parental samples, though low levels of Native American or East Asian admixture were not uncommon—roughly two-thirds of subjects harbored detectible, though generally low levels of such admixture. The Hispanic subjects plotted in an even distribution along the Native American/IndoEuropean axis, consistent with the knowledge Hispanics arose from the blending of colonial Europeans and resident Native Americans about 500 years ago.

In order to determine whether, and to what extent, the major proportions obtained with the BGA test corroborated with self reported race, BGA admixture proportions were calculated for 2,048 individuals of self-reported race, and blindly (in a computational sense) compared the majority BGA determined from the test against each individual's self-reported majority race. A very strong concordance was observed between the major BGA group determined with the test and the self-reported majority race (Table 7). Using the test, 1252/1252 self-described European-Americans (U.S. born Caucasians) registered with majority IndoEuropean BGA. 191 of 201 self-described African Americans showed majority sub-Saharan BGA, with the remaining 11 showing sub-Saharan BGA as the minor affiliation with IndoEuropean as the majority affiliation. Hispanics showed roughly equal distribution in majority BGA between IndoEuropean and Native American, consistent with the results observed in the triangle plot and the anthropological history of this group.

TABLE 7
Comparison of Majority BioGeographical Ancestry with Self-Reported
Race

| | | | Native | East |
|---|---|---|---|---|
| Self-Reported Race | European | African | American | Asian |
| European-American (US | 1252 | 0 | 0 | 0 |
| white) | | | | |
| Africans from Nigeria | 0 | 217 | 0 | 0 |
| African-American (US black) | 11 | 190 | 0 | 0 |
| Natives (“Nativos”) from | 1* | 0 | 72 | 0 |
| Guerrero, Mexico | | | | |
| Hispanics born in US, of | 101** | 0 | 90 | 0 |
| Mexican heritage | | | | |
| Chinese | 0 | 0 | 0 | 10 |
| Japanese | 0 | 0 | 0 | 10 |
| Other general Asian from US | 1*** | 0 | 0 | 33 |
| South Asians from India | 36 | 0 | 0 | 0 |

*Minor proportion was Native American
**Significant Native American as second type.
***Minor proportion was East Asian

Even when unexpected results were obtained, such as the finding that one individual from Southern Mexico was majority European, the results were more or less concordant in that the expected affiliation was the minor affiliation, rather than being absent altogether, and the major affiliation (Indo European) made sense in light of the history of the region (colonized by the Spanish hundreds of years ago). In this particular case, the level of Native American ancestry was only slightly less than 50%. Not one gross error was observed where, for example, a self-reported European-American was classified as majority East Asian. Though the 11 self described African-Americans of majority IndoEuropean BGA would appear to be a gross error, the results were similarly concordant—each of these African American samples exhibited roughly equal IndoEuropean/African proportions, suggesting admixture, consistent with the historical tapestry of the region from which the samples were taken, rather than test error.

To conduct a truly blind test (as opposed to a test that is blind in a computational sense), the San Diego Police Department Crime Lab (SDPD) and the National Center for Forensic Science at the University of Central Florida (UCF) each submitted ten buccal swabs of numerically encoded identity for BGA tests. The BGA test was performed and the results returned to SDPD and UCF, each of which independently evaluated their results and revealed the self-reported population affiliation of the sample. The major percentages determined from the BGA admixture proportions test were not inconsistent with the self-reported population affiliations (see Table 8). Several of the samples were from individuals affiliated with groups that are logically considered to be admixed—e.g., Filipinos (SDPD2, SDPD3, Table 8), African American or Carribean (SDPD5, SDPD6, UCF7, UCF8, Table 8), Mexican Americans (Hispanic, SDPD8, SDPD10, Table 8) and Puerto Rican (UCF6, Table 8); significant admixture was detected for each of these samples. Moreover, the type of admixture detected was reasonable with respect to the anthropological history of the affiliated population. For example, people of sub-Saharan African, Native American and IndoEuropean descent live in Puerto Rico, whereas East Asians are relatively rare, and the test results for the Puerto Rican individual tested showed IndoEuropean and Native American, not East Asian admixture.

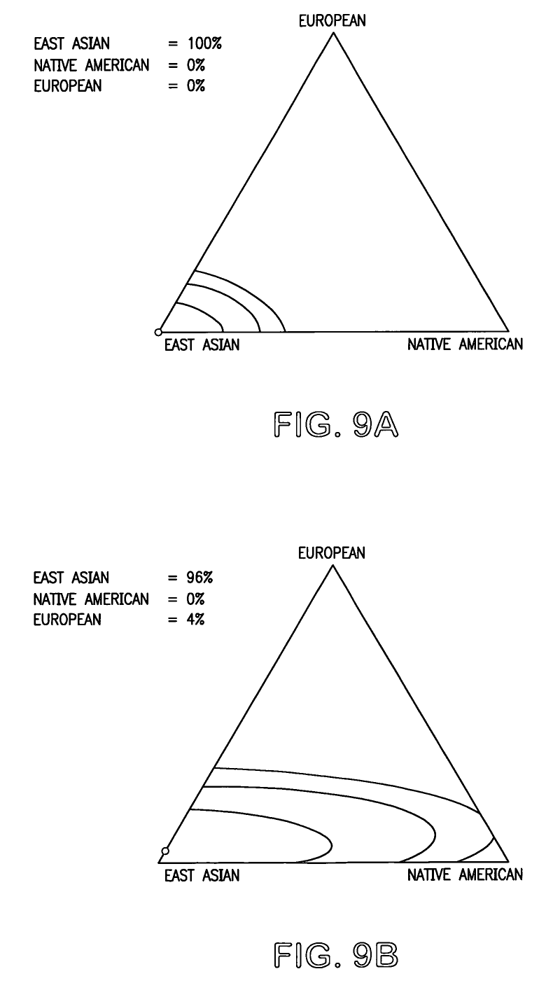

In addition to determining the MLE of admixture, the software program was designed to survey the probability space, and define that space within which the likelihood of proportional affiliation was 2-fold, 5-fold, and 10-fold less likely to be the correct answer than the MLE (confidence contours, which are plotted on the triangle plot as rings around the MLE). One way to test the accuracy of the maximum likelihood algorithm for determining MLE and confidence contours was to observe whether and how these values change when certain of the AIM markers are eliminated from the analysis by replacing the genotypes for each with “failure” readings. For example, if for a given sample genotype, all of the genotypes for markers of high δ value for the African/East Asian distinction were replaced with “failure” or “no data”, an accurate test would be expected to show warped confidence contours only in this dimension of the triangle plot. Accordingly, the BGA test was used to plot one sample of majority East Asian BGA with its associated confidence intervals (FIG. 9A), then all 24 of the markers in the test were eliminated with informative δ for Native American vs. East Asian BGA, and the MLE and confidence estimates were recalculated with the remaining AIMs (FIG. 9B). Upon recalculation with the missing AIMs, the confidence rings were dramatically skewed from the East Asian towards the direction of the Native American BGA vertex (FIG. 9B), indicating, as expected, that the lack of AIMS with good δ values for East Asian/Native American distinction produced an estimate for which the confidence along the East Asian/Native American axis was not high. Presumably because the sample typed as of majority East Asian, and AIMs for the distinction of East Asian/IndoEuropean and East Asian/African affiliation were left unmolested, the MLE shift itself was minimal; most of the uncertainty along the East Asian/Native American axis was apparent in the shift of the contours. Similar experiments with other samples and AIMs produced similar results.

TABLE 8
Blind Challenge of BGA Test by the San Diego Police Department and
the Center for Forensic Science at the University of Central Florida

| ID# | | % | Admix Ratio | % | | % | Self Reported Race |
|---|---|---|---|---|---|---|---|
| SDPD1 | EUROPEAN | 96 | EAST-ASIAN | 0 | NATIVE- | 4 | European-American |
| | | | | | AMERICAN | | |
| SDPD2 | EAST-ASIAN | 53 | EUROPEAN | 47 | NATIVE- | 0 | Filipino |
| | | | | | AMERICAN | | |
| SDPD3 | EAST-ASIAN | 61 | NATIVE- | 28 | EUROPEAN | 11 | Filipino |
| | | | AMERICAN | | | | |
| SDPD4 | EUROPEAN | 100 | EAST-ASIAN | 0 | NATIVE- | 0 | European-American |
| | | | | | AMERICAN | | |
| SDPD5 | AFRICAN | 69 | EUROPEAN | 31 | NATIVE- | 0 | Caribbean |
| | | | | | AMERICAN | | |
| SDPD6 | AFRICAN | 67 | EUROPEAN | 33 | EAST-ASIAN | 0 | African American |
| SDPD7 | EUROPEAN | 99 | EAST-ASIAN | 1 | NATIVE- | 0 | European-American |
| | | | | | AMERICAN | | |
| SDPD8 | NATIVE- | 57 | EUROPEAN | 43 | EAST-ASIAN | 0 | Mexican American |
| | AMERICAN | | | | | | |
| SDPD9 | EAST-ASIAN | 86 | NATIVE- | 0 | EUROPEAN | 14 | Filipino |
| | | | AMERICAN | | | | |
| SDPD10 | NATIVE- | 36 | EAST-ASIAN | 28 | EUROPEAN | 36 | Mexican American |
| | AMERICAN | | | | | | |
| UCF1 | EUROPEAN | 90 | EAST-ASIAN | 0 | NATIVE- | 10 | Ukraine/Italy |
| | | | | | AMERICAN | | |
| UCF2 | EAST-ASIAN | 98 | NATIVE- | 2 | EUROPEAN | 0 | Chinese |
| | | | AMERICAN | | | | |
| UCF3 | EUROPEAN | 88 | EAST-ASIAN | 3 | NATIVE- | 9 | Ukraine |
| | | | | | AMERICAN | | |
| UCF4 | EUROPEAN | 100 | EAST-ASIAN | 0 | NATIVE- | 0 | European-American |
| | | | | | AMERICAN | | |
| UCF5 | EUROPEAN | 87 | EAST-ASIAN | 0 | NATIVE- | 13 | Greek |
| | | | | | AMERICAN | | |
| UCF6 | EUROPEAN | 62 | NATIVE- | 38 | EAST-ASIAN | 0 | Puerto Rican |
| | | | AMERICAN | | | | |
| UCF7 | AFRICAN | 83 | EUROPEAN | 17 | EAST-ASIAN | 0 | African American |
| UCF8 | EUROPEAN | 69 | AFRICAN | 31 | NATIVE- | 0 | Jamaican |
| | | | | | AMERICAN | | |
| UCF9 | EUROPEAN | 84 | EAST-ASIAN | 12 | NATIVE- | 4 | Finland |
| | | | | | AMERICAN | | |
| UCF10 | EUROPEAN | 98 | EAST-ASIAN | 2 | NATIVE- | 0 | Scotland |
| | | | | | AMERICAN | | |

In order to determine the reproducibility and consistency of the BGA admixture proportion determinations, five samples were genotyped and analyzed on separate occasions. One sample was chosen of self-reported majority affiliation with the European American, African American, Hispanic and Asian groups, and a fifth sample was chosen from the parental Native American group. With the exception of failed loci, the genotypes at each marker in each individual were 100% consistent between runs, indicating that the AIMs genotype reliably. A 1-3% variation in BGA admixture proportions was observed from run to run; simulation studies showed that the variation was attributable to these genotyping failures. This result indicates that the failed loci did not pose a significant barrier to reproducibility of the BGA admixture determination for an individual, either in terms of majority BGA or admixture levels. From these simulations, it was determined that the BGA test tolerates about ten locus failures if the samples are of least binary admixture, and for samples of no admixture, larger numbers of failed loci are tolerable (i.e., change in admixture percentages were less than 5%).

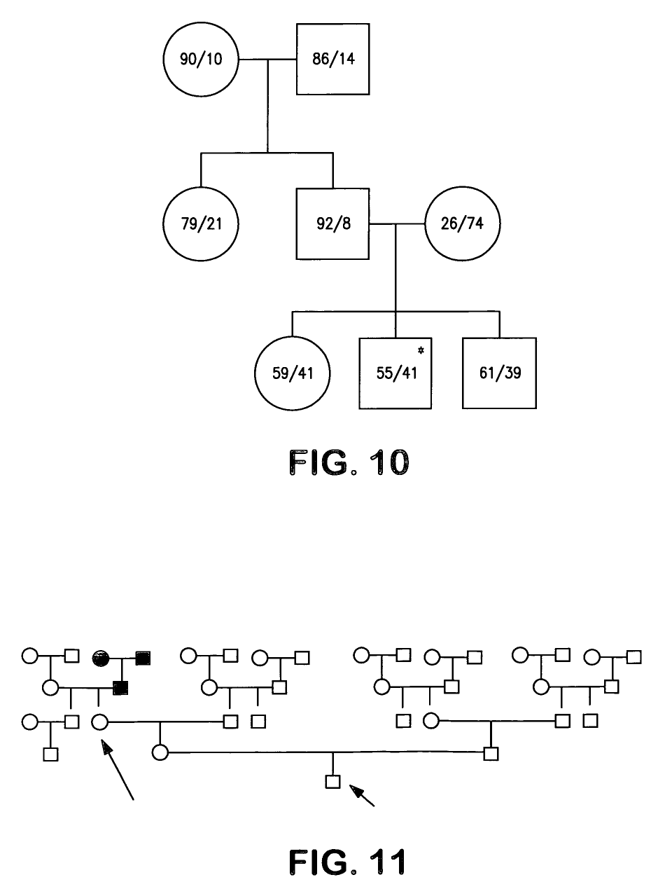

In order to determine if the BGA admixture proportions determined with the test were sensible given the rules of family inheritance, proportions for three generations from several family pedigrees were calculated. A typical result is shown in FIG. 10, which depicts the ratios obtained for a family of confirmed paternity (using STR tests) with substantial European/Native American admixture. The first generation individuals were self-reported European-Americans that were determined with the BGA test to harbor significant Native American admixture, which was passed to their son and daughter in different proportions that were not inconsistent with the law of independent assortment. The son's spouse was a Hispanic native of Mexico, and was determined to be of 26% European/74% Native American admixture. Each of their offspring harbor roughly intermediate levels of Native American and IndoEuropean admixture between their parents, again not inconsistent with the law of independent assortment. One of the sons typed as of a small percentage of East Asian ancestry, but the level (4%) was close to the reliable limit established as discussed above (about 3%). Other pedigrees tested (n=8) showed similarly concordant results, indicating that, in terms of majority BGA and admixture levels, the BGA test results were sensible within the context of family pedigrees.

Because the BGA test relies on parental population allele frequencies, the extent to which the admixture proportions produced by the test were influenced by parental allele sampling bias was investigated. A pair of populations was selected, as were the AIMs relevant for resolving affiliation between these two populations (Table 6—selected those with the highest 8 values for this pair of groups), then the allele frequency was adjusted for each of these AIMs in one of the groups such that the 6 value for the AIM was reduced by 20% (with respect to these two groups). In effect, the power of the test was intentionally degraded by 20% for the resolution of affiliation between a specific pair of groups; this degraded test is referred to as the Ancestry 2.1EA/EU BGA test, where the EA/EU refers to degradation in the distinction between the East Asian (EA) and IndoEuropean (EU) groups. 31 samples were randomly selected, the genotypes were run against the Ancestry 2.1EA/EU BGA test in exactly the same manner as for the original (ANCESTRYbyDNA™ 2.0) test, and the results were compared with those obtained with the ANCESTRYbyDNA™ 2.0 test.

If the results from the ANCESTRYbyDNA™ 2.0 test were highly sensitive to parental allele frequency error caused by sampling bias, which might be expected to be on the order of a few percent at the most, the 20% change introduced for the Ancestry 2.1EA/EU AIMs should result in admixture proportions substantially different from those of ANCESTRYbyDNA™ 2.0. Since the number of IndoEuropean/East Asian mixes observed was significantly greater than other types of mixes, such as African/Asian mixes or Native American/African mixes, the European/East Asian pair was selected for the first test—Ancestry 2.1EA/EU—and the BGA group pair for which the number of AIMs and cumulative δ value is the lowest is Native American/East Asian, the δ values for this pair was altered in the second test—Ancestry 2.1NA/EA. The average change observed in admixture proportions between ANCESTRYbyDNA™ 2.0 and Ancestry 2.1EA/EU was 1.4% (Standard Deviation 2.44%). For the Native American/East Asian pair, the average change between ANCESTRYbyDNA™ 2.0 and Ancestry 2.1NA/EA was 1% (Standard Deviation 2.3%).

Socio-cultural or self-held notions of race are not likely to be as tightly linked to human biology as BGA, which is the heritable component of race. Because it is subjective, imprecise and sometimes inaccurate, the use of self-identified race for the inference of BGA, as is currently practiced, obscures how and why human biology is related to human anthropology. Furthermore, the rigid binning of patients into prefabricated racial groups is a practice of generalization that is wholly unsatisfying because many individuals can trace their origins to multiple populations through the process of admixture. A repeatable, testable anthropological approach to define BGA can provide a means to draw connections between BGA and heritable diseases, whether through straight correlation and/or better study designs, or more subtle means such as through gene mapping methods that rely on the admixture process, such as MALD.

As disclosed herein, a 71 marker test allowed a determination of BGA proportions and their confidence intervals. The test enabled a determination of the relative proportionality of BGA within individuals, thus distinguishing the BGA test from other tests previously used for inferring ancestry from DNA. In terms of the majority BGA affiliation, more than 2200 tests were performed and no result was obtained that was inconsistent with self-held notions of race. Previous tests of ancestry have been accurate only to the upper 90% range (Shriver et al., supra, 1997; see, also, Frudakis et al., supra, 2003, which is incorporated herein by reference). The enhanced performance observed with the BGA test can be because CODIS and other STRs that have been commonly used for inferring ancestry from DNA were not selected for their δ values, but were selected for their polymorphic complexity in the world population. For the BGA test disclosed herein, the entire genome was systematically scanned and the best AIMs for this purpose were selected. In addition, most efforts to infer ancestry from DNA using STRs or Alu sequences have attempted to categorize or bin samples into single “racial” groups. For individuals of extensive admixture, such as a 50/50 mix, such a method would seem to produce a “wrong” answer as many times as a “right” answer. In contrast, with the BGA test, ancestry is determined in terms of proportional affiliation, thus ameliorating this problem.

The BGA test is distinguishable from other tests in that it employs SNPs that cover most of the chromosomes. Pan-chromosomal coverage using the BGA test provides a substantial advantage over tests using CODIS STRs, which only cover a fraction of the chromosomes. In addition, the BGA method appears to be the first that quantifies the confidence limits for its answers. The BGA test as exemplified is largely heuristic, and divides the world into four main anthropological groups that fall largely along continental lines. Though the geographical divisions are respectful of the anthropological history of human migrations, the use of four groups is indeed a simplification of a very complex situation and can be considered to be arbitrary. Further, the problem of determining proportional affiliation has been simplified by calculating the most likely 3-way (rather than 4-way) combination, because individuals of 4-dimensional BGA are thought to be rare, and because it is more convenient in a computational sense. However, while more complex tests may be able to capture more of the extant detail of anthropological history, even a crude 4 population test provides data of meaningful and historical content, provided the results are interpreted strictly with respect to these divisions and to the parental samples used in the construction of the test.

The choice of specific parental groups and of dividing the world into certain groups simply provides a point in coalescent time by which to scale the inferences provided by the test. In fact, the difference between the answer provided by a test based on 4 world population groups and a more complex test based on 25 would be one of anthropological time scale, not “accuracy”. For example, most Hispanics born in the United States that have been tested and most individuals claiming American Indian heritage type that were tested revealed minor Native American admixture on an IndoEuropean background. However, unlike the case for Hispanics, some of the individuals claiming American Indian heritage were typed as of minor East Asian admixture instead of Native American admixture. Because the founders for Native Americans migrated from East Asia, possibly in different waves at different times in history, the genetic distance between Native Americans and East Asians is lower than between Native Americans and sub-Saharan Africans or IndoEuropeans (Cavalli-Sforza and Cavalli-Sforza, supra, 1995). Among pre-colonial North Americans, proportional affiliation to East Asian or Native Americans would be expected to be different for individuals whose ancestors were part of the first waves than from those whose ancestors were part of later waves.

The parental samples for Native Americans used in the present study were derived from southern Mexico, and the AIM allele frequencies established for Native Americans obtained from this sample might be expected to be more representative of the ancestors from earlier waves of migration across the Strait. Native Americans from Latin America and South America would likely be more closely affiliated with early wave ancestors than, e.g., those from North America such as the Aleut Indians (and others), which would probably be more closely affiliated with ancestors from later waves across the Strait. The East Asian affiliation for those individuals claiming affiliation with American Indians may be a by-product of the choice of Southern Mexico Nativos as the parental source, and the use of only a 4 group anthropological scheme, but nonetheless, the answer is not a “wrong” answer in a scientific sense. Rather, it reports affiliations with respect to a coalescent time scale defined by the source of the parental samples and the anthropologically meaningful way in which the world was divided for this study.

A different test, with more markers to resolve affiliation within the North American groups from each other and from East Asians, would operate on a different coalescent time scale and likely classify these individuals as of minor “American Indian” or “North American Native American” admixture. Nonetheless, the fact that the exemplified BGA test revealed that a significant number of individuals of “Native American” ancestry show more affiliation with East Asians than Native Americans, as defined by the metrics built into the test, may be yet another example that social or human history based notions of population affiliation are semantic, subjective and not always accurate in a biologically meaningful way. Even though Aleuts may appear to resemble East Asians as much as, or even more than, most Native Americans in terms of physical features, and even though they are indigenous to a geographical locale as proximal to East Asia as to temperate North America, they are considered by most to be North American Indians and by extension, Native Americans because their home lies east of the Bering Strait. Similar examples have been observed for certain other population groups, as discussed below, illustrating the disconnect between the measurement of population affiliation using genetic markers and that from geographical and social borders man has devised to ascribe racial identity.

With deference to the qualifications of the BGA test, it is interesting to compile the results in order to extract some meaningful anthropological and/or sociological knowledge. Using the BGA test, 11 of 201 African Americans tested showed major IndoEuropean and minor African BGA, and Puerto Ricans of majority African descent almost always describe themselves as Hispanic, again pointing towards the deficiency with current notions of race as a dichotomous entity based on man-made constructs. As was observed in the present study, Risch et al. (supra, 2002) and Rosenberg et al. (supra, 2002) have shown that, when tested against methods of reporting BGA that rely on genome markers, majority population affiliation is quite accurately reported on questionnaires. The present testing of over 2,000-individuals indicates that the majority BGA affiliation can be accurately predicted from the self-reported race, that discordance between the two is not a very significant event, and that determination of majority ancestry affiliation is not the main problem with current self-reporting methods. However, perhaps the most surprising result was the extent of admixture for each population tested. When individuals claimed admixture, it was almost always confirmed with the BGA test; each case of expected sub-Saharan African and East Asian admixture was confirmed with the BGA test, and every Hispanic of Mexican descent registered with either major or minor Native American admixture, as expected.

Slightly more than two-thirds of all “Caucasians” tested exhibited minor East Asian or Native American admixture, and virtually none of these individuals reported any significant pedigree admixture on his or her questionnaire. Some of this admixture appears to be a function of ethnicity. Not only did individuals of relatively homogeneous self-reported Northern and Eastern European heritage more commonly show East Asian BGA, but Rosenberg et al. (supra, 2002) showed there to be significant structure within the “European-American” or “European” population that support this observation; specifically they showed that Russians commonly exhibit minor East Asian heritage. There are multiple times in history where such East-Asian/IndoEuropean admixture may have taken root, including, for example, the Mongolian invasions of Europe, and the Caucasoid expansion to Scandinavia, whose Lapp inhabitants were derived from Northern Asians, exhibit Mongolian features, and share a common history and culture with East Asians (Cavalli-Sforza and Cavalli-Sforza, supra, 1995). Some of the Native American admixture observed is concordant with history; that Filipinos exhibit extensive Native American admixture, for example, is not entirely surprising in view of the Spanish having conquered most of Latin America and exported Native American slaves to these islands, which were a Spanish territory until recently. The extent of Native American admixture observed for some Filipinos was quite high, perhaps reflecting that Native American admixture is relatively common in this part of the world, and that the pedigree for many Filipinos is dominated by large numbers of individuals of relatively low Native American admixture, rather than recent admixture with individuals of highly polarized BGA proportions. The Native American admixture commonly observed in “Caucasians” likely came from a blending of European and Native American peoples in North America.

In most cases of systematic admixture, such as between Scandinavian/Russian IndoEuropeans and East Asians, sub-Saharan African/IndoEuropean in the U.S., Native American/East Asian mixture in the Philippines or IndoEuropean/Native American in the U.S., the geographical proximity and/or historical mixture of the relevant groups is well established by history. For instance, an African/East Asian mixture was rarely observed in these studies, and history knows few examples of times when these two populations lived in close proximity to or mixed with one another. The type of admixture observed was also interesting when compared to self-held notions of “race”. For example, African Americans were more admixed with IndoEuropean Ancestry than were Caucasians with African ancestry, highlighting a difference in how Caucasians and Africans view their heritage, and invoking recollections of the “one drop rule”. Given the extent of admixture observed, it is likely that hidden or cryptic BGA structure arising from the process of admixture (a process that is not completely documented and quantifiable from our anthropological literature, as it is based on human constructions) is of potential concern for creating gross (or finer) structure differences between groups of study samples. Such a difference in structure would be expected to reduce the efficacy and power of large population based study designs;

Given that the majority ancestry was not inconsistent with self-held notions of affiliation for over 2,200 blind test subjects of polarized (low admixture) and self-reported race, the question arises as to how to assure the accuracy of minor admixture proportions such as these, and how such accuracy can be measured given that there is no ultimate arbiter of BGA in existence (except, possibly, genealogical information, see below). Several experiments were performed that address this question and, when considered together, indicate that the minor proportions are accurately determined. First, the minor admixture percentages transmit along family pedigrees in a manner consistent with the genetic law of independent assortment. By definition, a large, unbiased error would make it such that minor proportions were incoherent in the context of family pedigrees, i.e., the results would not be possible given the law of independent assortment, assuming the proportions of the parents to be correct.

Second, the minor admixture proportions are consistent, on average, with self-held notions of admixture. If there was a large, systematic and unbiased error in the estimation of BGA affiliation, this error would impact the integrity of the minor proportion percentages more than that of the major proportion percentages, since most individuals are of relatively polarized BGA affiliation, but the correlation of minor admixture proportions/presence with self-held notions of admixture would probably be very weak, if not imperceptible altogether. This was not observed to be the case. For example, if the error rate was as high as 20%, as many individuals reporting minority Hispanic ancestry would show minor sub-Saharan African and East Asian ancestry as Native American; the present results clearly demonstrate this is not the case.

Third, of about 2,200 samples blindly tested from North America, an individual with majority East Asian with substantial (>10%) sub-Saharan African admixture, or vice versa, has never been observed. This non-observation is relevant because such individuals are exceedingly rare in North America, from which the validation sample was derived. If there was a large, unbiased and systematic error rate, East Asian/African mixes would be observed as frequently as European-Asian mixes, which were frequently observed.

Fourth, a highly significant correlation was observed between minority IndoEuropean admixture and skin melanin content in African Americans (see Example 1). If there was a large, unbiased error rate, such a correlation would not likely be obtained.

Fifth, when the true allele frequencies for AIMs relevant for the resolution of affiliation between two groups is adjusted, such that the parental δ value was decreased by 20% for each relevant AIM for a given pair of groups, the overall power of the BGA test was degraded with respect to resolving affiliation proportions between these two groups, but essentially the same results were produced, both in terms of major affiliation and, more importantly, of minor admixture estimations. Parental sampling bias can cause inaccuracies in parental allele frequency estimation and δ values in this manner, though certainly less than 20% given that the parental sample for the present studies was comprised of about 100 individuals. Further, the error in allele frequency estimation would have to be obtained in the same direction for most of the AIMs relevant for resolving affiliation between a pair of groups for such an error to exist. Nonetheless, this result indicates that, even if such an error existed, the performance of the BGA test would be relatively unaffected. In other words, this experiment demonstrated that the BGA test is relatively robust in the face of parental sampling bias and allele frequency estimation.

Sixth, the distortion of confidence contours along only the axis corresponding to that for which genotyping failures were relevant shows a healthy disconnect among the interdigitating components (subsets of AIMs) of the test. In other words, if samples are fit to several templates for the determination of proportional fit, the elements of these templates should be independent for the test to be valid, and this was the result obtained.

It is difficult to imagine how significant systematic and unbiased error could exist in light of the above six observations. While each observation, on its own, may not prove the point of accuracy, taken together the results provide ample evidence that there is little or no systematic and unbiased error in the disclosed BGA test. It nevertheless could be argued that the error in the test is not random, but biased in a linear manner. However, the fifth observation (above) argues against this possibility. For example, the finding of minor East Asian admixture on an IndoEuropean background was more frequent than had been expected, and such a result could occur if there was the “right” amount of allele frequency estimation error in the “right” number of markers in the “right” direction (an unlikely, but not impossible, situation). However, because the AIM markers used for these studies are not mutually exclusive to particular group pairs, such an error would manifest itself in the resolution of affiliation for many pairs, not just one, and the first four observations (above) indicate that this is not the case. Also, such an error would seem to require parental sampling from a highly inbred group. While great care was taken to avoid such sampling, every element of population structure was not controlled for because there exists no test to do so a priori. As such, a simulation was performed to estimate the contribution of linear error to the test results (fifth observation, above). The fact that admixture results were relatively impervious to substantial (20%) reductions in δ value demonstrates that the quantity and quality of the AIMs used in the present studies were of adequate power such that reasonable levels of sampling error that may have been expected did not have a significant detrimental impact on the quality of the results. In terms of marker quality, this result is plausible because, given the selection process for AIMs, some of the best markers in the genome were used to determine BGA affiliation. In terms of marker quantity, these results are consistent with the observations that the results from the 71 marker test, as disclosed herein, were very similar to that produced from an earlier 30 marker test (Shriver et al., supra, 2003). Thus, reducing the number of markers, while retaining the average marker quality, did not impair the test. Furthermore, in another study, reducing the quality of the makers, but not the quantity, produced the same results. Overall, these observations indicate that the BGA proportions produced by the test are accurate with respect to the confidence intervals presented, and that the test performs in a robust manner.

The present results demonstrate that BGA admixture is more common than previously believed. If true, then it should be asked whether finer levels of BGA admixture are linked to human biology, e.g., drug responsiveness or disease predisposition. Such “cryptic” structure can only be determined using a molecular test because, unlike crude population structure. a questionnaire cannot be used. Finer levels of population structure beyond the more crude continental levels measured using questionnaires are recognized. For example, significant anecdotal evidence suggests that redheads require a 20% larger dose of many common anesthetics, and exhibit a tendency towards hypertension and bleeding while under anesthesia (Cohen, The Scientist 16:10, 2002). These complex physiological responses would be difficult to explain based on melanocortin-1 (MC1R) variants previously linked to some of the variation in red hair color (Robbins et al., Cell 72:827-834, 1993; Smith et al., J. Invest. Dermatol. 111:119-122, 1998; Flanagan et al., Hum. Molec. Genet. 9:2531-2537, 2000). There are likely specific gene variants responsible for these clinical phenotypes and, if these variants correlate with elements of population substructure or microstructure, as they appear to do, any study attempting to identify linkages or LD between markers and the relevant phenotypically active loci will be challenged from the outset at the study design step. The exemplified BGA test can be extended using AIMs in addition to those disclosed herein to quantify the elements of population structure relevant for this particular problem because a precision and objectivity greater than that provided by self-reporting of socio-cultural race will be needed to identify the elements of structure that can interfere with the design of genetics experiments.

### EXAMPLE 3

### Application of the BGA Test to Genealogy

This Example demonstrates that BGA admixture estimates can be integrated with genealogical information obtained using traditional genealogical research methods.

Genealogists collect data that largely is relevant in a geopolitical context (e.g., data relating to which countries a person's ancestors are from, what their religions were, and their last names) rather than in an anthropological context (e.g., what type of population admixture characterizes the person's family tree). There are two main sources for obtaining minority admixture in one's results: 1) recent exogamous admixture events; and 2) ancient affiliations with ethnic groups that are characterized by systematic admixture.

The results of an exogamous event is determined in a recent genealogical time (e.g., the last 250 years). For example, as shown in FIG. 11, a Chinese great grandparent in an otherwise homogeneous IndoEuropean family tree would produce a grandchild of IndoEuropean/East Asian admixture. The individuals that are 100% East Asian (Chinese) are shown with shading (FIG. 11), and the admixture results for the male (square) at the bottom of the pedigree (short arrow) are of interest. A person with a single 100% East Asian great grandparent and seven 100% IndoEuropean great grandparents would be expected to have 12.5% East Asian admixture. By the law of genetic assortment, the expected level is actually a range around 12.5%, with values several percent above and below possible. The grandparent indicated by the long arrow is about a 50%/50% East Asian/IndoEuropean mix and her daughter, the subject's mother, is expected to be a 25%/75% East Asian/IndoEuropean mix (FIG. 11).

Ancient affiliations (i.e., considered with respect to an anthropological time frame) have been preserved in modern times by endogamous, relatively geographically isolated, close knit community stricture (i.e., ethnicity). For example, modern day demography is shaped by not only the migrations our ancestors made to establish new populations, but also by admixture between these populations throughout the world. The map below shows these migrations as measured from Y chromosome sequences, which occurred over many tens of thousands of years.

Admixture between the groups, after each had developed as distinct groups, has occurred many times throughout ancient and even more recent history, and was represented by arrows on the map representing migration patterns. For example, there was extensive East Asian admixture in Russians and Eastern Europeans (Rosenberg et al., supra, 2003), and the extent to which the Mongolian and Hun invasions may have contributed to this admixture over a long time period remain a mystery. There was an even more pronounced East Asian admixture apparent for Native Americans (Rosenberg et al., supra, 2003); arrows were not included on the map for this admixture because far too many would have been required and most of the admixture events are not know. Nonetheless, a person with a fair number of Native Americans or Russians in their family tree could very well exhibit as much East Asian admixture as an individual with a 100% Chinese grandmother and three other 100% IndoEuropean grandparents.

Although not shown, a time scale was constructed showing the time the most significant migrations occurred, and was correlated to a very, very large family tree. The tree is for a single individual, who resides at the bottom apex of a triangle graph; it is large because it goes back 60,000 years when there are tens of thousands of ancestors for this person. The time scale for the migrations applies to the large family tree as well. The tree was the same as that shown in the pedigree map (FIG. 11), only much larger and without the lines connecting the ancestors (spots represented each ancestor, but there were so many that it was not practical to show all of the lines connecting the spots. A pool of spots represented “Russian”, which for purposes of this example were assumed to be an ethnicity that arose about 18,000 years ago. Additional spots represented East Asian, and was based on the assumption that the average Russian harbors 10% East Asian admixture. A third set of spots represented the precursors to Russians; these precursors are unknown but, for purposes of this example, were assumed to be Eastern Europeans.

In this example, most of this person's Russian ethnicity came from the left side of the family tree, which can be assumed to be that part of the tree representative of the subject's father's side of the family. If, as this example indicated, the average Russian harbors 10% East Asian admixture, and half of the person's family tree is predominantly Russian, the person would be expected to harbor 5% East Asian admixture. East Asian admixture is significant for this person even though neither the person's grandmother, grandfather, or any other relative within the past 18,000 years was homogeneous East Asian. The way to visualize this on the family tree is to count all of the “East Asian” spots and divide them by the total number of spots in the tree, to arrive at about 5%. Thus, relatively homogeneous East Asians represented about 5% of the total number of ancestors for this person. Of course, the family tree for some people involves numerous groups that are characterized by small degrees of this type of admixture. Family trees like that exemplified are polarized with certain ethnicities, and it is uncommon to see a tree with an equal distribution of each of the four BGA groups (sub-Saharan African, Native American, IndoEuropean and East Asian) because, until recently, and even now to a certain extent, people have tended to have children with others like themselves. As such, most family trees are not a “mish-mash” of random affiliations, but are highly polarized as exemplified.

The Pennsylvania Dutch provide another example of admixture due to ancient affiliations, wherein, in this community, it appears that there existed significant East Asian content prior to 1700 in German antecedents. These antecedents established communities that populated the valleys in the upper Rhine River Basin, then, later, more further inland. Since these communities remained relatively isolated, the level of East Asian admixture has remained around the 20% level. Dilution of this level would require external admixture with other IndoEuropean ethnicities such as French or Sardinian, for which East Asian admixture is not detectible. In this case, the German antecedents were of substantial average East Asian admixture, perhaps due to sampling from within a more heterogeneous German population.

Most genealogists are interested in the type of admixture found in source arrived at according to the methods of exogamous admixture, which gives information about geopolitical affiliation of recent ancestors, rather than anthropological information on distant ones. This is because there exists little paper data for more distant ancestors compared to recent ones; the further back in time a person goes, the larger number of ancestors exist, making research difficult if not impossible; and the contribution of distant ancestors towards a modern persons genetic constitution is less on average per relative than that of more recent ancestors. As such, genealogists tend to seek information such as that which might be produced due to recent admixture. For example, if a person is trying to prove or disprove a rumor or legend of American Indian ancestry, a 10% Native American admixture result would be very useful if it could be assured that the mechanism for this admixture was due to recent exogamous admixture events; and not to ancient affiliations with ethnic groups that are characterized by systematic admixture. While the BGA test does not allow a distinction between exogamous v. ancient admixture, the results of the test provide an important piece of the genealogical puzzle.

For some genealogists, depending on the family tree, evidence may strongly suggest that the mechanism of admixture is from recent events. As such, for a person whose family has paper evidence of an American Indian great grandparent, a 10% Native American admixture result according to the BGA test can indicates the event likely arose due to recent admixture. In comparison, for a person of confirmed and homogeneous European ancestry, a 10% Native American admixture indicates the event likely arose due to an ancient admixture.

Negative results carry different meaning than positive ones for genealogists. For example, if there is circumstantial, but low quality, data suggesting a pure blood African great-grandfather, and the BGA test reveals 100% IndoEuropean, then the rumor would be discounted (taking into account the genetic law of independent assortment, which would make such a result possible, but unlikely if the data was in fact correct). However, if a person's family is suspected to have had a Chinese great grandfather, one cannot prove it from a 20% East Asian admixture result, since it is not possible to distinguish exogamous admixture from ancient admixture.

It is important for a genealogist to bring other knowledge to bear in order to reconstruct the most likely source for an admixture result. In fact, the BGA admixture data serves as an independent clue for one attempting to reconstruct a family history, and when it is used with genealogical knowledge, the two combine to form evidence that is more powerful than either on their own. As such, the BGA test provides an ancillary tool that can help fashion a system tailored for the genealogy community by providing BGA admixture results in a manner that places equal emphasis on the anthropological sources, which transmitted ancient or very old (relative to a genealogical time frame, which encompasses the last 250-300 years) admixture to us in modern times, and exogamous admixture due to events in the family tree in the last 200 years.

A database of several thousand BGA profiles is built from people of various locations throughout the world such that one can query the database with a profile, plus or minus a pre-selected error range. A list of places for which this type of profile has been commonly found can be provided, or, for example, a map of the world that is color-coded can be provided, wherein the colors indicate the likely regional affiliations corresponding to the admixture profile/range. In other words, given a BGA admixture profile, a map can be provided showing the places from which the person's recent ancestors could have been derived. A person with 10% East Asian and 90% Indo European would show high probability of ancestral derivation from China (exogamous admixture) or Russia (more ancient admixture coupled with ethnic homogeneity over the family tree).

Similarly, the genealogist can provide a map, similarly color-coded, that is derived from paper research, which is based on geopolitical rather than anthropological information. The two maps can then be overlaid, and Bayesian statistical calculations made that combine the information from the BGA test with that of the paper genealogy to provide a most likely estimate of recent family history.

By way of example, a person with 90% IndoEuropean and 10% East Asian BGA and a paper genealogy of Romanian/British/Spanish ancestors would first query the database with his or her BGA result, and be provided a map where the sources would be shown as possibly from East Asia (due to recent admixture), Russia and Northern/Eastern Europe (both due to a large number of more distant ancestors from relatively isolated and admixed groups). The color-coding would give the probability of derivation from the regions based on the frequency with which the compatible BGA groups are found in each region, and it may be quite complex depending on the mixture type and the character of our database, which is a function of world-wide sampling. Second, the person would provide (or be provided with) a separate map based on the probable Romanian/British/Spanish heritage documented from genealogical research, using map drawing tools we could provide. From this map, it would be apparent that the likelihood of recent, homogeneous East Asian ancestors is not high. Third, a program would determine that the most likely origin of the 10% East Asian admixture is from the Romanian ancestors (not the British or Spanish, and not due to a Chinese grandparent, for example).

This type of presentation allow a person to learn the most likely source of an unexpected admixture result, using prior knowledge obtained through other means such as genealogical research. This is valuable to a genealogist seeking to explain the derivation of a genetic constitution. Without this ancillary tool, a person with 90% IndoEuropean and 10% East Asian admixture would have no ready means to determine whether the test suggested a recent Chinese or Japanese grandparent/great grand parent or a certain ethnic affiliation for which minor East Asian admixture is commonly found.

### EXAMPLE 4

### Association of Iris Pigmentation and Biogeographical Ancestry

This Example demonstrates that cryptic population structure as determined using AIMs allows an inference as to a complex genetic trait such as iris color.

In order to determine whether and how common polymorphisms are associated with natural distributions of iris colors, 851 individuals of mainly European descent were surveyed at 335 SNP loci in 13 pigmentation genes and 419 other SNPs distributed throughout the genome and known or though to be informative for certain elements of population structure. Numerous SNPs, haplotypes and diplotypes (diploid pairs of haplotypes) were identified within the OCA2, MYO5A. TYRP1, AIM, DCT and TYR genes and the CYP1A2-15q22-ter, CYP1B1-2p21, CYP2C8-10q23, CYP2C9-10q24 and MAOA-Xp11.4 regions as significantly associated with iris colors. Half of the associated SNPs were located on chromosome 15, which corresponds with results others have previously obtained from linkage analysis. Five additional genes (ASIP, MC1R, POMC, and SILV) and one additional region (GSTT2-22q11.23) with haplotype and/or diplotypes were identified, but not individual SNP alleles associated with iris colors (see, also, Intl. Publ. No. WO 02/097047). For most of the genes, multilocus gene-wise genotype sequences were more strongly associated with iris colors than haplotypes or SNP alleles. Diplotypes for these genes explain 15% of iris color variation. These results provide a comprehensive candidate gene study for variable iris pigmentation, and constitute a classification model useful for the inference of iris color from DNA. The results further demonstrate that cryptic population structure can serve as a leverage tool for complex trait gene mapping if genomes are screened with the appropriate AIMs.

Iris pigmentation is a complex genetic trait that has long interested geneticists, anthropologists, and public at large, but is not yet completely understood. Eumelanin (brown pigment) is a light-absorbing polymer synthesized in specialized melanocyte lysosomes called melanosomes. Within the melanosomes, the tyrosinase (TYR) gene product catalyzes the rate-limiting hydroxylation of tyrosine to 3,4-dihydroxyphenylanine, or DOPA, and the resulting product is oxidized to DOPA quinone to form the precursor for eumelanin synthesis. Though TYR is centrally important for this process, pigmentation in animals is not simply a Mendelian function of TYR or any other single protein product or gene sequence. In fact, study of the transmission genetics for pigmentation traits in man and various model systems suggests that variable pigmentation is a function of multiple, heritable factors whose interactions appear to be quite complex (see, e.g., Akey et al., supra, 2002; Box et al., J. Invest. Dermatol. 116, 224-229, 2001). For example, unlike human hair color (Sturm et al., Gene 277:49-62, 2001), there appears to be only a minor dominance component for mammalian iris color determination (Brauer and Chopra, Anthropol Anz. 36:109-120, 1978), and there exist minimal correlation between skin, hair and iris color within or between individuals of a given population. In contrast, between-population comparisons show good concordance; populations with darker average iris color also tend to exhibit darker average skin tones and hair colors. These observations suggest that the genetic determinants for pigmentation in the various tissues are distinct, and that these determinants have been subject to a common set of systematic and evolutionary forces that have shaped their distribution in the world populations.

At the cellular level, variable iris color in healthy humans is the result of the differential deposition of melanin pigment granules within a fixed number of stromal melanocytes in the iris. The density of granules appears to reach genetically determined levels by early childhood and usually remains constant throughout later life, though a small minority of individuals exhibit changes in color during later stages of life. Pedigree studies suggested iris color variation is a function of two loci; a single locus responsible for de-pigmentation of the iris, not affecting skin or hair, and another pleiotropic gene for reduction of pigment in all tissues (Brues, Amer. J. Phys. Anthropol. 43:387-91, 1975).

Most of what is now known about pigmentation has been derived from molecular genetics studies of rare pigmentation defects in man and model systems such as mouse and Drosophila. For example, dissection of the oculocutaneous albinism (OCA) trait in humans has shown that many pigmentation defects are due to lesions in the TYR gene, resulting in their designation as tyrosinase (TYR) negative OCAs (see, e.g., Oetting and King, Hum. Mutat. 13: 99-115, 1999; see, also, Albinism database, on world wide web (“www”), at URL “cbc.umn.edu/tad/”). TYR catalyzes the rate-limiting step of melanin biosynthesis and the degree to which human irides are pigmented correlates well with the amplitude of TYR message levels. Nonetheless, the complexity of OCA phenotypes has illustrated that TYR is not the only gene involved in iris pigmentation. Though most TYR− negative OCA patients are completely de-pigmented, dark-iris albino mice (C44H), and their human type IB oculocutaneous counterparts exhibit a lack of pigment in all tissues except for the iris (Schmidt and Beermann, Proc. Natl. Acad. Sci. USA 24; 91:4756-4560, 1994). Study of a number of other TYR-positive OCA phenotypes showed that, in addition to TYR, the oculocutaneous 2 (OCA2; Durham-Pierre et al., Nature Genet. 7:176-179, 1994; Durham-Pierre et al., Hum. Mutat. 7:370-373, 1996; Gardner et al., Hum. Mutat. 7:370-373, 1992), tyrosinase like protein (TYRP1; Boissy et al., Amer. J. Hum. Genet. 58:1145-1156, 1996), melanocortin receptor (MC1R; Robbins et al., supra, 1993; Smith et al., supra, 1998; Flanagan et al., supra, 2000) and adaptin 3B (AP3B; Ooi et al., EMBO J. 16:4508-4518, 1997) loci, as well as other genes (reviewed by Sturm et al., supra, 2001) are necessary for normal human iris pigmentation. Each of these genes is part of the main (TYR) human pigmentation pathway.

In Drosophila, iris pigmentation defects have been ascribed to mutations in over 85 loci contributing to a variety of cellular processes in melanocytes (Ooi et al., supra, 1997), but mouse studies have suggested that about 14 genes preferentially affect pigmentation in vertebrates (reviewed in Strum et al., supra, 2001), and that disparate regions of the TYR and other OCA genes are functionally distinct for determining the pigmentation in different tissues. Human pigmentation genes break out into several biochemical pathways, including those for tyrosinase enzyme complex formation on the inner surface of the melanosome, hormonal and environmental regulation, melanoblast migration and differentiation, the intracellular routing of new proteins into the melanosome and the proper transportation of the melanosomes from the body of the cell into the dendritic arms towards the keratinocytes. Nonetheless, the study of human OCA mutants suggests that the number of phenotypically active pigmentation loci is manageably small for genetic analysis.

Though research on pigment mutants has made clear that a small subset of genes is largely responsible for catastrophic pigmentation defects in mice and man, it remains unclear whether or how common SNPs in these genes contribute towards (or are linked to) natural variation in human iris color. A brown-iris locus was localized to an interval containing the OCA2 and MYO5A genes (Eiberg and Mohr, Eur. J. Hum. Genet. 4:237-241, 1996), and specific polymorphisms in the MC1R gene are associated with red hair and blue iris color in relatively isolated populations (see, e.g., Robbins et al., supra, 1993; Flanagan et al., supra, 2000; Valverde et al., Nature Genet. 11:328-330, 1995; Schioth et al., Biochem. Biophys. Res. Comm. 260:488-491, 1999). An ASIP polymorphism was reported to be associated with both brown iris and hair color (Kanetsky et al., Amer. J. Hum. Genet. 70:770-775, 2002). However, the penetrance of each of these alleles appears to be low and, in general, appears to explain but a very small amount of the overall variation in iris colors within the human population (Spritz et al., Nature Genet. 11:225-226, 1995). However single gene studies have not provided a sound basis for understanding the complex genetics of human iris color.

Because most human traits have complex genetic origins, wherein the whole often times is greater than the sum of the parts, innovative genomics-based study designs and analytical methods for screening genetic data in silico are needed that are respectful of genetic complexity—for example, the multi-factorial and/or phase known components of dominance and epistatic genetic variance. The first step however is to define the complement of loci that on a sequence level explain variance in trait value, and of these, those that do so in a marginal, or penetrant sense will be the easiest to find. It is towards this goal that the present study was performed.

A non-systematic, hypothesis-driven genome screening approach was applied to identify various SNPs, haplotypes and diplotypes marginally (i.e., independently) associated with iris color variation. As disclosed in this Example, a surprisingly large number of polymorphisms in a large number of genes were associated with iris colors, indicating that the genetics of iris color pigmentation are quite complex. The sequences that were identified provide the basis of a classifier model for the inference of iris colors from DNA, and the nature of some of these as markers of BioGeographical Ancestry has implications for the design of other complex trait gene mapping studies.

Methods

Specimen Collection

Specimens for re-sequencing were obtained from the Coriell Institute in Camden, N.J. Specimens for genotyping were of self-reported European descent, of different age, sex, hair, iris and skin shades and they were collected using informed consent guidelines under IRB guidance. Donors checked a box for blue, green, hazel, brown, black or unknown/not clear iris colors, and each had the opportunity to identify whether iris color had changed over the course of their lives or whether the color of each iris was different. Individuals for whom iris color was ambiguous or had changed over the course of life were eliminated from the analysis.

For 103 of the subjects, iris colors were reported using a number from 1-11 as well, where 1 is the darkest brown/black and 11 is the lightest blue identified using a color placard. For these subjects, digital photographs of the right iris were obtained, where subjects peered into a box at one end, at the camera at the other to standardize lighting conditions and distance, and from which a judge assigned the sample to a color group. Comparing the two, 86 of the classifications matched. Of the 17 that did not, 6 were brown/hazel, 7 were green/hazel and 4 were blue/green discrepancies though none were gross discrepancies such as brown/green, brown/blue or hazel/blue. Though such an error is tolerable for identifying sequences marginally associated with iris colors, confidence can be increased for use of the sequences described herein for iris color classification by obtaining digitally quantified iris colors.

SNP Discovery

Candidate SNPs were obtained from the NCBI:dbSNP database, which generally provided more candidate SNPs that were possible to genotype. Human pigmentation and xenobiotic metabolism genes were examined, selected based on their gene identities not their chromosomal position. For some genes, the number of SNPs in the database was low and/or some of the SNPs were strongly associated with iris colors, warranting a deeper investigation. For these genes, re-sequencing was performed; of the genes disclosed herein, 113 SNPs were discovered in the CYP1A2 (7 gene regions, 5 amplicons, 10 SNPs found), CYP2C8 (9 gene regions, 8 amplicons, 15 SNPs found), CYP2C9 (9 gene regions, 8 amplicons, 24 SNPs found), OCA2 (16 gene regions, 15 amplicons, 40 SNPs found), TYR (5 gene regions, 5 amplicons, 10 SNPs found) and TYRP1 (7 gene regions, 6 amplicons, 14 SNPs found; see Tables 9 and 10; see, also, Intl. Publ. No. WO 02/097047).

TABLE 9
Candidate Genes Tested for Sequence Associations with Human
Iris Pigmentation

| Gene | Name | Homology/Model Phenotype |
|---|---|---|
| AP3B1 | adaptor-related protein | mouse “pearl” |
| | complex 3, beta 1 subunit | human HPS2 |
| ASIP | agouti signaling protein | mouse “agouti” |
| DCT | dopachrome tautomerase | TYR-related protein 2 |
| | | mouse “slaty” |
| MC1R | melanocortin 1 receptor | mouse “extension” (e) |
| OCA2 | oculocutaneous albinism II | mouse pink-eyed dilution (p) |
| SILV | silver homologue | mouse “silver” (si) |
| TYR | tyrosinase | mouse “albino” (c), |
| | | Himalayan |
| TYRP1 | tyrosinase related protein 1 | mouse “brown” (b) |
| MYO5A | myosin VA | mouse “dilute” (d) |
| POMC | proopiomelanocortin | mouse Pomc1 |
| AIM | membrane associated | mouse “underwhite” (uw) |
| (MATP or | transporter protein | |
| AIM-1) | | |
| AP3D1 | adaptor-related protein | mouse “mocha” (mh) |
| | complex 3, delta 1 subunit | |
| RAB | RAB27A oncogene | mouse “ashen” (ash) |

Resequencing for these genes was performed by amplifying the proximal promoter (avg. 700 bp upstream of transcription start site), each exon (avg. 1400 bp), the 5′ and 3′ ends of each intron (including the intron-exon junctions, average size about 100 bp) and 3′ UTR (avg. 700 bp) sequences from a multiethnic panel of 672 individuals (450 individuals from the Coriell Institutes DNA Polymorphism Discovery Resource, 96 additional European Americans, 96 African Americans and 10 Pacific Islander, 10 Japanese and 10 Chinese; this 672 represented a separate set of samples than that used for the association study described herein). PCR amplification was accomplished using pfu TURBO polymerase according to the manufacture's guidelines (Stratagene). A program was used to design re-sequencing primers in a manner respectful of homologous sequences in the genome, to insure that pseudogenes were not co-amplified or that sequences from within repeats were amplified. BLAST searches confirmed the specificity of all primers used. Amplification products were subcloned into the pTOPO® sequencing vector (Invitrogen) and 96 insert positive colonies were grown for plasmid DNA isolation (the use of 670 individuals for amplification step reduced the likelihood of an individual contributing more than once to this subset of 96 selected).

Sequencing was perform with an ABI3700 sequencer using PE Applied Biosystems BDT chemistry; sequences were deposited into a commercial relational database system (iFINCH, Geospiza; Seattle Wash.). PHRED qualified sequences were imported into the CLUSTAL X alignment program and the output of this was used with a second program to identify quality-validated discrepancies between sequences. Those sequences for which at least two instances of PHRED score 24 or greater variants were identified were selected, and each of these SNPs discovered through re-sequencing was used for genotyping.

Genotyping:

For most of the SNPs, a first round of PCR was performed on the samples using the high-fidelity DNA polymerase pfu TURBO polymerase and the appropriate resequencing primers. Representatives of the resulting PCR products were checked on an agarose gel, and first round PCR product was diluted, then used as template for a second round of PCR. The two rounds were necessary because many of the genes queried were members of gene families; the SNPs resided in regions of sequence homology and the genotyping platform required short (approximately 100 bp) amplicons. For the remaining samples, only a single round of PCR was performed. Genotyping was performed for individual DNA specimens using a single base primer extension protocol and an SNPstream™ 25K/Ultra High Throughput (UHT) instrument (Orchid Biosystems; Princeton N.J.). Genotypes were subject to several quality controls; two scientists independently pass/fail inspected the calls, requiring an overall UHT signal intensity greater than 1,000 for >95% of genotypes and clear signal differential between the averages for each genotype classes (i.e. clear genotype clustering in 2-D space using the UHT analysis software).

Statistical Methods

To test the departures from independence in allelic state within and between loci, the MLD exact test was used (Zaykin et al., supra, 1995). Haplotypes were inferred using the haplotype reconstruction method (Stephens et al., supra, 2001). To determine the extent to which extant iris color variation could be explained by various models, R2 values were calculated for SNPs, Haplotypes, and Multilocus Genotypes data by first assigning the phenotypic value for blue eye color as 1, green eye color as 2, hazel eye color as 3, and brown eye color as 4. BGA admixture proportions were determined as described (Hanis et al., supra, 1986; Shriver et al., supra, 2003) within the context of a software program developed for this purpose. For R2 computation, the following function was used: Adj-R2=1−{(n/(n−p)}(1−R2), where n is the model degrees of freedom and n−p is the error degrees of freedom. To correct for multiple tests, the empirical Bayes adjustments for multiple results method was used (Steenland et al., Cancer Epidemiol. 9:895-903, 2000, which is incorporated herein by reference).

Results

To identify SNP loci associated with variable human pigmentation, 754 SNPs were genotyped, including 335 SNPs within pigmentation genes (AP3B1, ASIP, DCT, MC1R, OCA2, SILV, TYR, TYRP1, MYO5A, POMC, AIM, AP3D1 and RAB, see Table 9), and 419 other SNPs distributed throughout the genome. Alleles for these latter SNPs were informative for certain elements of population structure; 71 were selected from a screen of the human genome based on their exceptionally high 6 values (i.e., exceptional AIMs) for IndoEuropean, sub-Saharan African, Native American and East Asian BGA (see Example 2; SEQ ID NOS:1 to 71; see, also, Shriver et al., supra, 2003), and the rest were found in or around xenobiotic genes, which tend to exhibit dramatic sequence variation as a function of BGA. Genotypes for these 754 candidate SNPs were scored for 851 European derived individuals of self-reported iris colors (292 blue, 100 green, 186 hazel and 273 brown).

Before screening these genotypes for association with iris colors, the 71 non-xenobiotic metabolism AIMs were used to determine BGA admixture proportions for each sample, and were tested for correlation between BGA admixture and iris colors. This test showed that each of the 851 Caucasian samples was of majority IndoEuropean BGA, and, though 58% of the samples were of significant (>4%) non-IndoEuropean BGA admixture, there was no correlation between low levels (less than 33%) of East Asian, sub-Saharan African or Native American admixture and iris colors, and no correlation between higher levels (greater than 33% but lower than 50%) of Native American admixture and iris colors; there was, however, a weak association between higher levels of East Asian and sub-Saharan African admixture and darker iris colors.

It was unclear from the outset whether better success would be realized by considering iris color in terms of 4 colors (blue, green, hazel and brown) or groups of colors. One method of grouping colors is light=blue+green and dark=hazel+brown, and this grouping seems to more clearly distinguish individuals with respect to the detectible level of eumelanin (brown pigment). Given that iris color data was self-reported, partitioning the sample into brown and not-brown, or blue and not blue could provide greater power to detect significant associations, particularly for alleles associated with only one color. To take advantage of each of these 4 methods, all were considered when screening SNPs for associations; the δ value, chi square and exact test p-values were calculated for a) all 4 colors, b) shades, using light (blue and green) vs. dark (hazel and brown), c) blue vs. brown, and d) brown vs. not-brown (blue, green and hazel) groupings. Significance levels were fixed at 5%, and the alleles of 20 SNPs were associated with specific iris colors, 20 with iris color shades, 19 with blue/brown color comparisons and 18 using the brown/not brown comparison. The overlap among these SNP sets was high but not perfect; a SNP with a significant p-value for association using at least one of the four criteria is indicated as “marginally” associated.

When multiple simultaneous hypotheses are tested at set p-values there is the possibility of enhanced type I error. As such, a correction procedure was used to compensate for this risk (Steenland et al., supra, 2002); most of the associations were significant after this correction. Most of the marginally associated SNPs were within the pigmentation genes—OCA2 (11 SNPs at the level of colors), TYRP1 (3 SNPs at the level of colors), MYO5A (2 SNPs at the level of colors), AIM (3 SNPs at the level of colors) and DCT (2 SNPs at the level of colors)—though some associations were found within non-pigmentation genes such as CYP2C8 at 10q23, CYP2C9 at 10q24, CYP1B1 at 2p21 and MAOA at Xp11.3—also referred to as marginally associated SNPs. No significant SNP associations were found within the pigmentation genes SILV, MC1R, ASIP, POMC, RAB or TYR, though TYR had one SNP with a p=0.06. The most strongly associated of the marginally associated SNPs were from the OCA2, TYRP1 and AIM genes, in order of the strength of association.

Since most of the SNPs identified from this approach localized to discrete genes or chromosomal regions, all of the SNPs from each locus were grouped and inferred haplotypes were tested for association with iris colors using contingency analysis. This higher-order analysis was not confined to those genes with marginal SNP associations, but grouped SNPs for all of the genes tested. For each gene, haplotypes were inferred and contingency analyses was used to determine which haplotypes were statistically associated with iris colors. From the chi-square and adjusted residuals, 43 haplotypes for 16 different loci were either positively (agonist) or negatively (antagonist) associated with iris colors (Table 10). The strongest associations were observed for genes with SNPs that were marginally associated; most of these genes had haplotypes and diplotypes (sometimes referred to as multilocus gene-wise genotypes or diploid pairs of haplotypes) positively (agonist) or negatively (antagonist) associated with at least one iris color (Table 10). A few of the genes/regions not harboring a marginally associated SNP had haplotypes and diplotypes either positively and/or negatively associated with iris colors (ASIP gene-1 haplotype, MC1R gene-2 haplotypes, Table 10). In other words, their SNPs were only associated with iris colors within the context of gene haplotypes or diplotypes. For some, associations with iris colors were only found within the context of diplotypes, but not at the level of the SNPs or haplotype (i.e. SILV and GSTT2-22q11.23).

TABLE 10
The common haplotypes and diplotypes for the 16 iris color genes

| | | | | | | |
|---|---|---|---|---|---|---|
| Gene and | | | | | | |
| Sequence | | Agonist² | Agonist² | Antagonist² | Antagonist² | |
| ID. | Haplotypes-16 Genes¹ | Chi P | Color | Chi P | Color | Count³ |

| | | | | | | |
|---|---|---|---|---|---|---|
| AIM | | | | | | |
| 1 | C A C | 0.0010 | Blue | 0.004 | Brown | (1641) |
| 2 | T G T | 0.048 | Brown | 0.003 | Blue | (33) |
| 3 | T A C | — | — | — | — | (23) |
| ASIP | | | | | | |
| 1 | A T A | — | — | — | — | (979) |
| 2 | A T G | — | — | — | — | (508) |
| 3 | G C G | — | — | — | — | (196) |
| 4 | A C A | 0.017 | Hazel | — | — | (13) |
| DCT | | | | | | |
| 1 | C T G A C A | — | — | — | — | (625) |
| 2 | C T C A C A | 0.014 | Hazel | 0.028 | Blue | (242) |
| | | 0.016 | Blue | 0.021 | Brown | |
| 3 | T C G A C A | 0.048 | Green | 0.003 | Hazel | (281) |
| 4 | C T C G T A | — | — | — | — | (320) |
| 5 | C T G A C G | — | — | — | — | (179) |
| MC1R | | | | | | |
| 1 | T C C | 0.016 | Green | — | — | (152) |
| 2 | C C C | — | — | 0.026 | Green | (1294) |
| 3 | C C T | — | — | — | — | (143) |
| 4 | C T C | — | — | — | — | (113) |
| MYO5A | | | | | | |
| 1 | C G A T C G G C C C | 0.000 | Green | — | — | (51) |
| 2 | C G A T C A G C C C | — | — | — | — | (858) |
| 4 | G T G C T G A T C C | — | — | 0.008 | Blue | (163) |
| 5 | C G A T C A A C C C | 0.017 | Blue | — | — | (40) |
| 6 | G T A C T G A T C C | — | — | — | — | (117) |
| 8 | C G A C T G G T T T | — | — | — | — | (165) |
| 10 | C G A C C A G C C C | 0.003 | Brown | — | — | (40) |
| 13 | C T A C T G G T T T | — | — | — | — | (71) |
| 14 | C G A T T A G C C C | — | — | 0.027 | Blue | (40) |
| 16 | C G G C C A A T C C | — | — | — | — | (19) |
| OCA2 | | | | | | |
| 1 | G G G G A C G G C A | 0.002 | Blue | 0.01 | Brown | (44) |
| | A A G | | | | | |
| 2 | G A G G C C G G C A | 0.018 | Hazel | 0.031 | Blue | (43) |
| | A G A | | | | | |
| 3 | G A G G C C A G C A | 0.022 | Brown | 0.026 | Blue | (21) |
| | A G A | 0.042 | Green | | | |
| 4 | G A G G C C G G C A | 0.024 | Brown | 0.014 | Blue | (89) |
| | A A A | | | | | |
| 7 | G A G G C C A G C A | — | — | — | — | (13) |
| | A A A | | | | | |
| 15 | T G G G A C G C T A | — | — | — | — | (17) |
| | A A G | | | | | |
| 19 | G A G G C C A G C G | 0.019 | Brown | — | — | (23) |
| | A G A | | | | | |
| 22 | G A G G C C G G C G | 0.036 | Green | 0.012 | Blue | (15) |
| | A A A | | | | | |
| 25 | T A A G C C A G C G | — | — | — | — | (13) |
| | A A A | | | | | |
| 37 | G G A A A T A G C A | <0.001 | Blue | <0.001 | Brown | (508) |
| | A A A | | | | | |
| 38 | G G A A A T A G C G | 0.025 | Blue | 0.011 | Brown | (200) |
| | A A A | | | | | |
| 39 | G G A A A T A G C A | — | — | 0.039 | Brown | (71) |
| | A G A | | | | | |
| 41 | G G A A A T A G C A | — | — | — | — | (19) |
| | G A A | | | | | |
| 42 | G G A A A T A G C G | 0.029 | Blue | 0.021 | Brown | (174) |
| | A G A | | | | | |
| 45 | T G A A A T A G C G | <0.001 | Brown | 0.007 | Blue | (65) |
| | A A A | | | | | |
| 47 | T G A A A T AG C G | 0.003 | Brown | 0.006 | Hazel | (22) |
| | A G A | | | | | |
| 48 | T G A A A TA G C A | 0.001 | Brown | 0.036 | Blue | (35) |
| | A A A | — | — | 0.015 | Green | |
| 57 | T G A A A T A G C A | — | — | — | — | (30) |
| | A G A | | | | | |
| POMC | | | | | | |
| 1 | T | — | — | — | — | (1187) |
| 2 | C | — | — | — | — | (515) |
| 2/2 | C/C | 0.043 | Blue | — | — | |
| SILV | | | | | | |
| 1 | T C | — | — | — | — | (913) |
| 2 | C C | — | — | — | — | (296) |
| 3 | T T | — | — | — | — | (490) |
| 1/1 | TC/TC | — | — | 0.035 | Blue | |
| TYR | | | | | | |
| 1 | G C G | — | — | 0.01 | Green | (184) |
| 2 | A A G | — | — | — | — | (257) |
| 3 | G C A | — | — | — | — | (467) |
| 4 | A A A | — | — | — | — | (231) |
| 5 | A C A | — | — | — | — | (254) |
| 6 | A C G | — | — | — | — | (189) |
| 7 | G A G | — | — | — | — | (83) |
| 8 | G A A | — | — | 0.023 | Blue | (37) |
| TYRP | | | | | | |
| 1 | T T T T C G | 0.007 | Blue | <0.001 | Brown | (968) |
| 2 | C T T T T T | — | — | — | — | (86) |
| 3 | C T G A C G | 0.006 | Brown | — | — | (454) |
| 4 | C C G A C G | 0.046 | Brown | 0.029 | Blue | (98) |
| 5 | T T G T C G | — | — | — | — | (23) |
| 7 | C T T A C G | — | — | — | — | (24) |
| CYP1A2- | | | | | | |
| 15q22-ter | | | | | | |
| 1 | G | 0.015 | Brown | 0.023 | Hazel | (769) |
| 2 | C | 0.023 | Hazel | 0.015 | Brown | (933) |
| CYP1B1- | | | | | | |
| 2p21 | | | | | | |
| 1 | C C | — | — | — | — | (957) |
| 2 | T T | 0.027 | Hazel | 0.025 | Blue | (403) |
| 3 | C T | — | — | — | — | (337) |
| CYP2C8- | | | | | | |
| 10q23 | | | | | | |
| 1 | C A A | 0.007 | Brown | — | — | (539) |
| 2 | T A G | — | — | — | — | (201) |
| 3 | T G A | — | — | 0.039 | Brown | (513) |
| 4 | T A A | — | — | — | — | (439) |
| CYP2C9- | | | | | | |
| 10q24 | | | | | | |
| 1 | T | 0.023 | Brown | 0.039 | Green | (1325) |
| 2 | C | 0.039 | Green | 0.023 | Brown | (377) |
| GSTT2- | | | | | | |
| 22q11.23 | | | | | | |
| 1 | A G | — | — | — | — | (821) |
| 2 | G A | — | — | — | — | (778) |
| 3 | A A | — | — | — | — | (99) |
| | AG/AG | 0.040 | Green | — | — | (184) |
| | AG/GA | 0.013 | Hazel | — | — | (401) |
| MAOA- | | | | | | |
| Xp11.4-11 | | | | | | |
| 1 | C G | — | — | 0.027 | Blue | (1133) |
| 2 | T A | 0.006 | Blue | 0.026 | Hazel | (480) |
| 3 | C A | — | — | — | — | (87) |

¹Sequences of the highest order of complexity within a locus found to be associated with iris colors. All of the major sequences (count >13 ) for each locus with at least one significantly associated sequence are shown. If no haplotypes or diplotypes for a locus were found to be associated, only the SNP alleles are shown. If no haplotypes were found to be associated for a locus but diplotypes were found to be associated, both the haplotypes and diplotypes are shown.
²Agonist color refers to the color with which the sequence is positively associated. Antagonist color refers to the color with which the sequence is negatively associated. Chi-square P-value is shown.
³Number of times the haplotype was observed in our sample of 851.

At the level of the haplotype, each gene or region had unique numbers and types of associations. For example, OCA2, AIM, DCT and TYRP1 harbored haplotypes both positively associated with blue irides and negatively associated with brown irides (OCA2 haplotypes 1, 37, 38, 42, AIM haplotype 1, DCT haplotype 2, and TYRP1 haplotype 1 Table 10). Others genes such as AIM, OCA2 and TYRP1 harbored haplotypes that were positively associated with brown but negatively associated with blue color (AIM haplotype 2, OCA2 haplotypes 2, 4, 45, 47, TYRP haplotype 4, Table 10), while others such as MYO5A, OCA2, TYRP1 and CYP2C8-10q23 harbored haplotypes that were positively associated with one color but not negatively associated with any other color (MYO5A haplotype 5, haplotype 10, OCA2 haplotype 19, TYRP1 haplotype 3 and CYP2C8-10q23 haplotype 1, Table 10). The MC1R gene harbored haplotypes only associated with green color in our sample and the POMC gene harbored a single SNP with genotypes weakly associated with iris colors (no significant haplotypes or diplotypes were found).

Overall, the diversity of haplotypes associated with brown irides was similar to that of haplotypes associated with blue irides. Most of the haplotypes were even more dramatically associated with iris colors in a multi-racial sample, because many of the SNPs comprising them are good AIMs, and variants associated with darker iris colors were enriched in those ancestral groups of the world that are of darker average iris color. Most of the SNPs within a gene or region were in LD with others in that gene or region (D′<0.1); only 32 SNP pairs, in the MC1R (1 pair), OCA2 (27 pairs), TYR (2 pairs) and TYRP1 (2 pairs) genes were found to be in LD.

These analyses resulted in the identification of 61 SNPs in 16 genes/chromosomal regions associated with iris colors on one level or another, whether the SNP is marginally associated or associated within the context of the haplotype and/or diplotype. The minor allele frequency for most of these SNPs was relatively high (avg. f minor allele=0.22) and most of them were in Hardy Weinberg Equilibrium (those for which HWE p>0.05, 28/34, Table 10). Nine were not and of these, 2 were of relatively low frequency and the evidence for disequilibrium was marginal (p value close to 0.05). Lack of HWE is usually an indication of a poorly designed genotyping assay, and none of the remaining 7 SNPs exhibited genotyping patterns that we have previously associated with such problems (such as an absence of one genotype class, or a preponderance of heterozygotes). Indeed one of those for which the evidence of lack of HWE was the strongest was validated as a legitimate SNP through direct DNA sequencing. The chromosomal distribution of the SNPs that were significantly associated in a marginal sense was independent of the distribution of SNPs actually surveyed, indicating that the associations were not merely a function of SNP sampling.

Chromosome 15q harbored the majority (18/34) of the SNPs that were marginally associated with iris colors, and 14 of these chromosome 15 SNPs were found in two different genes OCA2 and MYO5A. Chromosome 5p had 3 SNPs marginally associated, all in the AIM gene and chromosome 9p had5 SNPs associated, all in the TYRP1 gene. Multiple SNPs were identified on chromosome 10q; the CYP2C8-10p23.33 region had 2 SNPs, and the neighboring region CYP2C9-10p24 also had one. All 3 markers were in tight LD with one another (p<0.001 for each possible pair). Multiple SNPs were also identified on chromosome 2; the POMC SNP located at 2p23 was marginally associated, and SNPs from the CYP1B1-2p21 region were associated within the context of a 2-SNP haplotype (Table 10), and these SNPs were also in LD (p<0.01). Finally, in addition to the OCA2 (15q11.2-q12) and MYO5A (15q21) sequences, a single SNP (15q22-ter) was also implicated on chromosome 15q, but SNPs between each of these three loci were not in LD. SNPs for the MC1R (16q24), SILV (12q13), TYR (11q), MAOA-Xp11.4-11.3 and GSTT2-22q11.23 regions were also associated at the level of the haplotype (Tables 10 and 11), though these were the only regions of these chromosomes for which associations were found.

The p-values that were obtained indicated that diplotypes explained more iris color variation than haplotypes or individual SNPs. To test this, a corrected ANOVA analysis was performed for the data on each of these three levels. All 61 SNPs were considered, as were their haplotypes (Table 10) and diplotypes (not shown). Diplotypes explained 15% of the variation, whereas haplotypes explained 13% and SNPs explained 11% (Table 4) after correcting for the number of variables. The most strongly associated 68 genotypes of the 543 genotypes observed for the 16 genes/regions, based on chi-square adjusted residuals, explained 13% of the variation (row 4, Table 11).

TABLE 11
ANOVA-SNP and Haplotype data

| | | | | No. | | | |
|---|---|---|---|---|---|---|---|
| Row | Source | Model DF | Error DF | Variables | F Value | R2 Value | Adj.R² |
| 1 | SNP data | 62 | 788 | 62 | 2.63 | 0.17 | 0.11 |
| 2 | Haplotype data | 212 | 638 | 216 | 1.58 | 0.34 | 0.13 |
| 3 | Multilocus Gene-wise | 543 | 307 | 572 | 1.27 | 0.69 | 0.15 |
| | Genotype data | | | | | | |
| 4 | Multilocus Gene-wise | 68 | 782 | 68 | 2.82 | 0.2 | 0.13 |
| | Genotype data | | | | | | |

From a screen of 754 SNP loci, 61 were identified that were statistically associated with variable iris pigmentation at one level of intragenic complexity or another. The remaining SNPs had δ values and chi-square p-values that were not significant on any level of intragenic complexity. Diplotypes for these 61 alleles explained most of the iris color variance in the sample; the lowest amount was explained at the level of the SNP, suggesting an element of intragenic complexity to iris color determination (i.e., dominance).

Only about half of the 61 SNPs identified were associated with iris colors independently; the others were associated only in the context of haplotypes or diplotypes. Even at this level of complexity, the sequences from no single gene could be used to make reliable iris color inferences, indicating an element of intergenic complexity (i.e., epistasis) for iris color determination as well. Aside from the fact that many of the identified SNPs were significant after imposing the correction protocol for multiple testing, five lines of evidence indicated that the identified SNPs are not spuriously associated. First, for all of the genes identified as marginally associated SNPs, multiple such SNPs were identified; i.e., the distribution of SNPs among the various genes tested was not random. Second, some of the non-pigment gene SNPs are located near pigment genes, e.g., CYP2C8 (10q24.1) and CYP2C9 (10q24), which are located proximal to two pigment genes not tested directly—HPS1 (10q23.1-4) and HPS6 (10q24.34) and the chromosome 2p SNP at the CYP1B1 locus (CYP1B1-2p21) located proximal to POMC at 2p23 (and in LD with the POMC SNP). Third, though a roughly equal number of pigmentation and non-pigmentation gene SNPs were tested, of the 34 marginally associated SNPs, 28 of them (82%) were in pigmentation genes. Thus, the distribution of SNPs among the various gene types also was not random. Fourth, the associations were generally stronger for the SNPs in the context of within-gene haplotypes, a result that would not necessarily obtain for SNPs spuriously associated (i.e., the result suggests the gene sequences themselves are associated, not merely a single polymorphism within each gene). Fifth, when applied to a sample including individuals of multiple ancestries, the linear and non-linear variables from these and the other genes combined performed even better than when applied just to individuals of majority European ancestry. Since most individuals of non-European, or minority European descent exhibit low variability in iris colors (on average of darker shade than individuals of European descent) this improvement may not seem surprising. However, this result would not have necessarily been obtained were the SNPs not truly associated with iris colors.

Though corrections for multiple testing left most of the SNP-level associations intact, a number of the associations did not pass the multiple-testing examination, but are presented in order to avoid possible type II error; the sequences may be weakly associated with iris colors and possibly relevant within a multiple-gene model for classification (i.e., epistasis). For these, it would seem more prudent to eliminate false-positives downstream of SNP identification, such as from tests of higher order association, using various other criteria such as those described above, or possibly using the utility of the SNP for the generalization of a complex classification model.

Mutations in the pigmentation genes are the primary cause of oculocutaneous albinism, so it was natural to expect that common variations in their sequence explain some of the variance in natural iris colors and, in fact, this result was observed. However, a number of the associations were for SNPs located in other types of genes (CYP2C8 at 10q23, CYP2C9 at 10q24, CYP1B1 at 2p21 and MAOA at Xp11.3). The inclusion of non-pigmentation genes in this study was intentional; the screen was not restricted to pigmentation gene SNPs, but included two types of AIMs—those that were selected from the genome based on δ values between sub-Saharan, IndoEuropean, Native American and East Asian population allele frequencies, and those selected based on their location within xenobiotic metabolism genes. The latter are included based, in part, on previous evidence indicating that xenobiotic metabolism genes harbor an unusual concentration of AIMs, and that some of these AIMs are relevant for the measurement of “cryptic” population structure, presumably because xenobiotic metabolism gene products are responsible for detoxification of floral alkaloids and tannins present in indigenous diets, and selection and genetic drift have shaped the geographical distribution of their sequences. Such cryptic structure may be correlated with iris colors to a degree that enables accurate classification, even though they may not be helpful for elucidating a biological mechanism.

It was hypothesized that a) some of these SNPs would be indicators of not merely crude or continental population structure, but sub-structure and perhaps even microstructure, b) iris colors were correlated with these elements of structure within the Caucasian group, and c) these markers can serve as proxies for phenotypically active loci for the purpose of classification or trait value inference. The commonly held notion is that genetic screening is only properly conducted towards identifying phenotypically active loci through linkage disequilibrium. However, when classification is the goal, rather than the identification of phenotypically active loci, population structure can be helpful if the trait value correlates with the structure and if markers for the structure can be identified. For example, an iris color classification tool can be useful to a forensic scientist for the objective and science-based construction of a partial physical profile from crime scene DNA. Currently, forensics investigators construct physical profiles using surprisingly unscientific means; only in rare cases are eye-witness accounts available, and often times human accounts are subjective and unreliable in certain circumstances. For a forensics application, an investigator is less interested in the biological mechanism of the phenotype than in an ability to make an accurate inference of trait value. Of course, identifying markers in LD with phenotypically active loci (or the phenotypically active loci themselves) provide for more accurate classification, as well as for a better understanding of biological mechanism, but the hunt for these elusive loci in heterogeneous populations is impractical because LD only extends for a few Kb and expensive genome-wide scans are required.

That a number of the SNPs identified herein as associated with iris colors were located in xenobiotic metabolism genes suggests the identified markers are associated with iris colors through correlation with cryptic population structure. In other words, the non-pigmentation gene markers are probably correlated with, but not necessarily in LD with, phenotypically active loci for iris color. Through such a correlation, both markers and active loci are enriched for in certain branches of the IndoEuropean pedigree, even though they may not be in LD with one another. These results based on such correlations are meaningful in a classification context only with respect to the sample used. For example, AIMs selected based on their inter-continental 6 values were not associated with iris colors in individuals of mainly European descent, but were strongly associated with iris colors in a more international sample because the AIMs, are specifically relevant for the element of structure correlated with iris colors in this sample. In contrast, these same AIMs were not associated with iris colors within the sample of majority European-derived individuals examined because there is little variation in crude structure within this sample. Instead, within any (majority) Caucasians or European American sample, there will exist sub-structure or micro-structure (cryptic structure) due to variation in ethnic or other sub-population level affiliations, and only those SNPs specifically relevant for measuring this cryptic structure would be needed if the structure correlates with the trait. It bears noting that no systematic structure in the mainly European-derived study sample unrelated to the phenotype measured was identified, indicating that the use of qualified AIMs is imperative for reproducing the present results with another trait.

Other interpretations of the present results are possible, for example, that the associations could have been observed through LD with as of yet to be defined pigmentation genes. Indeed, CYP2C8 and CYP2C9 are located on chromosome 10 near the HPS1 and HPS2 pigmentation genes (not tested directly), CYP1A2 is located at 15q22-ter, on the same arm as OCA2 and MYO5A, CYP1B1 is located at 2p21, in the vicinity of the POMC gene at 2p23 and MAOA is located on the same arm of the X chromosome (Xp11.4-11.3) as the OA1 pigmentation gene (not tested directly). The distances between these loci associated with iris colors and “neighboring” pigmentation genes is far greater than the average extent of LD in the genome, and even if these associations are through LD, it would seem that, again, population structure would need to be invoked as an explanation.

LD is known to extend for megabases in recently admixed populations, such that as few as a couple thousand AIMs can be used to gain full genome coverage in these populations, and it is of some interest that two-thirds of the European American samples used in this study were of significant (4%) BGA admixture. Though European Americans are not recognized as a traditionally defined admixed group (like Hispanics or African Americans), the BGA admixture observed may be linked to finer, cryptic levels of population structure. While the relevance of the present results to LD and/or population structure is not clear, if the results are due to LD rather than correlation, they would suggest that just as AIMS can be used to leverage population admixture for trait mapping in recently and extensively admixed populations, they also can be used to leverage cryptic population structure in a similar manner. Thus, regardless of whether the results are due to correlation or LD, the large number of non-candidate gene associations identified indicates that the measurement of population structure has broader implications for the cost-effective development of pharmacogenomics and complex disease gene classifiers.

Linkage studies have implicated certain pigmentation genes as specifically relevant for pigmentation phenotypes, and most of the pigmentation gene SNPs identified herein clustered to certain genes such as OCA2, MYO5A, TYRP1 and AIM. Further, certain aspects of the present support the previous literature. Most of the SNPs identified were on chromosome 15, which linkage analyses identified as the primary chromosome for the determination of “brownness” (Eiberg and Mohr, Eur. J. Hum. Genet. 4:237-41, 1996); it was suggested that the candidate gene within the interval containing this locus (BEY2) was most likely the OCA2 gene, though the MYO5A gene is also present within this interval and, as disclosed herein, was associated with iris colors. OCA2 associations were by far the most significant of any gene or region tested, while MYO5A SNPs were only weakly associated (but haplotypes and diplotypes more strongly). MYO5A alleles were not in LD with those of OCA2, suggesting these results were independently obtained and that results described by Eiberg and Mohr may have been a reflection of the activity of two separate genes (Eiberg and Mohr, supra, 1996).

Two OCA2 coding changes were reported to be associated with darker iris colors (Rebbeck et al., Cancer Epidemiol. Biomarkers Prev. 11(8):782-784, 2003). In addition, the “red hair/blue iris” SNP alleles previously (Valverde et al., supra, 1995; Koppula et al., supra, 1997) were identified, confirming that these sequences are associated with iris pigmentation, though the previously described associations were with blue irides and at the level of the SNP, whereas, in the present study, the associations were with green irides and only apparent at the level of the haplotypes and diplotypes. Associations were also identified in the ASIP gene (see Kanetsky et al., supra, 2002), though, in the present study, this gene association was not at the level of the SNP; one of the ASIP SNPs identified herein (marker 861) is the 8818 G-A SNP transversion described as associated with brown iris colors (Kanetsky et al., supra, 2002) though, in the present study, the association was with hazel color at the level of the haplotype.

The associations between TYR haplotypes and iris colors was relatively weak, which is not inconsistent with results obtained by others in the field of oculocutaneous albinism who have failed to find strong associations in smaller samples. Though the present results independently verified findings for OCA2, ASIP and MC1R, they also show that several other pigmentation genes harbor alleles associated with the natural distribution of iris colors (TYRP1, AIM, MYO5A and DCT). As such, the present results indicate that most of the previous studies associating pigmentation gene alleles with iris colors, taken independently, represent mere strokes of a larger, more complex portrait.

Interestingly, most of the SNPs identified herein are non-coding, either silent polymorphisms or residing in the gene proximal promoter, intron or 3′UTR. This result, while not altogether unusual, can indicate that the SNPs are in LD with other phenotypically active loci, or it may be a reflection that variability in message transcription and/or turnover may explain part of the variability observed in human iris colors. Though a large number of SNPs was screened, some of the genes harbor a large number of candidate SNPs and not all were tested. For example, the OCA2 has about 200 known candidate SNPs in NCBI dbSNP. As such, the OCA2 gene may yet have more information variable human iris pigmentation, such information being accessible using methods as disclosed herein.

### EXAMPLE 5

### Use of AIMS to Predict Drug Responsiveness

This Example demonstrates that AIMs can be used to develop chemopredictive and diagnostic tests because many drug response traits, as well as many human genetic traits, correlate with elements of population structure.

The distribution of SNPs available for genotyping by chromosomal arm is shown in FIG. 12. At each of the approximately 400 SNPs examined, Caucasian individuals taking Lipitor™ (180), for whom response was known in terms of cholesterol (TC), low density lipoprotein (LDL), liver transaminase ASTSGOT and ALTGPT measurements, were genotyped. 150 Zocor™ patients of known response in terms of TC and LDL change, and 1,000 individuals of known hair and eye color, also were genotyped. Those SNPs with delta values of significance (δ>0.20) among the various trait classes were selected. For example, in about 70% of patients, Lipitor™ caused a decrease in LDL, whereas in 30% of patients it had no effect. For any given SNP, the delta value (δ) is the difference in minor allele frequency among those individuals for whom LDL decreased by at least 20% versus those for whom LDL did not so decrease. The δ value was measured for each SNP in FIG. 12 for each test (Zocor™: LDL, TC, ASTSGOT, ALTGPT response; Lipitor™: LDL, TC response). For eye color, the δ value was measured in terms of dark (hazel or brown) versus light (blue or green) eyes. For hair color, the δ value was measured in terms of black or brown color versus blonde.

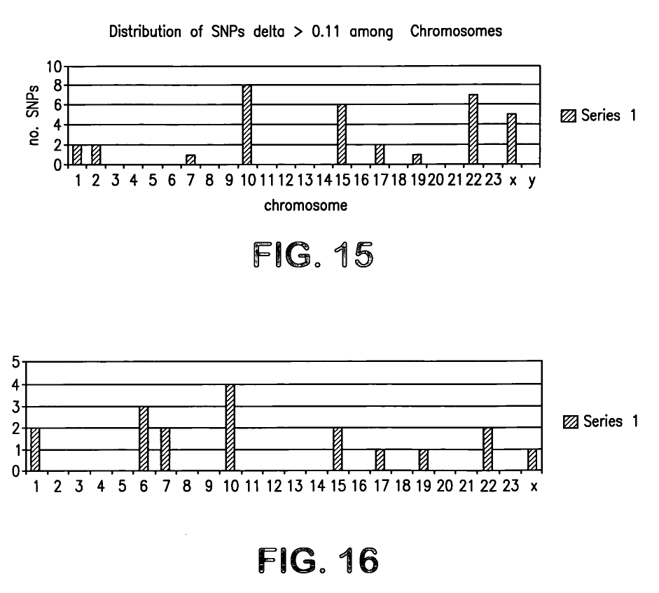

For Lipitor™ response, the number of SNPs of significant (δ>0.20) value for each of the 4 endpoint measures is shown in FIG. 13. Those SNPs with delta values of significance for Zocor™ response using LDL and TC endpoint measures then were selected (FIG. 14). Next, those SNPs with delta values of significance for Iris color were selected (FIG. 15); and, likewise, for hair color (FIG. 16). The distribution of SNPs with good δ values was similar for each trait in Graphs A-E, but with certain elements of specificity. The specificity can be appreciated by focusing on chromosome arm 6p, which has many important SNPs for TC (Total Cholesterol) response to Lipitor™ (FIG. 13), but none for Zocor™ response (FIG. 14). Chromosome 2 harbors SNPs with good δ values for eye color (FIG. 16), but not hair color (FIG. 15). Chromosome 15 harbors many markers predictive of Lipitor™ responsiveness, but not Zocor™ response. This specificity is likely a function of linkage disequilibrium with other loci deterministic for each of these traits, but which has nothing to do with the expression of the other traits; this type of finding is the goal of traditional measures of gene mapping. Alternatively it can be due to correlation with certain elements of population structure

The similarity is apparent in that chromosomes 10 and 22 have a relatively high number of SNPs with good δ values for each of the four mechanistically unrelated traits, as does chromosome 1. Overall, the distribution of important SNPs for one trait is not dissimilar to that for another. It is the similarity between the profiles that is of interest here, and that illustrates the value of the present methods.

The elements that the four graphs share in common correlate with the number of SNPs genotyped (FIG. 12; FIGS. 13 to 16 roughly resemble FIG. 12). At first pass, this result appears to indicate that the “importantness” or significance for these SNP alleles are spurious, and merely a function of the number of SNPs genotyped for each chromosomal arm (i.e., the more SNPs one genotypes from a chromosome, the more SNPs of good delta value one will find on that chromosome). However, the SNPs in FIG. 12 are not just any type of SNP; most of the SNPs available in the FIG. 12 are xenobiotic metabolism and pigmentation gene SNPs, and most all are good AIMs.

For iris color, it was shown that most of the SNP associations remain significant (chi p<0.05 after correction for multiple testing, thus indicating that the SNP associations are not spurious. That the distribution of SNPs associated for each of the 4 traits resemble one another to a large extent, and that this distribution is similar to the distribution of available SNPs, most of which are good AIMs, indicate that most of the SNPs measured in these experiments are reporters of population structure, and that similar elements of population structure are correlated with response to each of the two drugs (however measured) as well as to hair and iris color pigmentation.

It is notable that these four traits appear to be mechanistically unrelated (at least to current knowledge) and it is not intuitively obvious as to how hair or eye color can relate to the response to two randomly selected drugs. However, the similarity in profiles for important SNPs for each of the traits suggests that each can be predicted, to a significant extent, with a knowledge of the sequence for a common set of chromosomal markers. Because these markers are known to be excellent indicators of BGA, the results indicate that each of the four unrelated traits can be predicted to some extent by measuring BGA rather than by measuring the particular SNPs measured in FIGS. 12 to 16.

Simply measuring BGA as disclosed herein does not impart as much predictive power for each of the four traits as measuring the specific AIMs in the plots above. However, using the markers in FIG. 15 enabled a good classification accuracy for iris color. These results indicate that different AIMs in the plots above are informative for different elements of population structure or substructure, and while most of the SNPs in FIG. 12 are good indicators of crude population structure in terms of continental BGA, it has not yet been determined to what extent each is also informative for other finer levels of structure, such as between Scandinavian and Mediterranean ancestry, or even within ethnic groups. Such “cryptic” structure could not previously be defined in a reliable, confidence qualified manner because, prior to the present disclosure, it was not possible to define the subtle elements of structure recognized in large populations of individuals of common race in biogeographically meaningful ways). For example, red haired individuals of majority IndoEuropean descent are known to require 20% more anesthesia than other IndoEuropean (or other) patients (Cohen, supra, 2002). These red haired individuals also exhibit a tendency towards hypertension and bleeding under the influence of certain common anesthetics, thus presenting a serious clinical problem of unknown etiology.

Red haired individuals are common in the United Kingdom (Ireland and Britain), which also contains individuals sharing more ancestors with one another than with individuals of other regions of Europe. As such, they can be considered as a branch of indeterminate structure off the family tree of humanity. Only one gene is linked to red hair color (MCIR), and it is difficult to imagine this gene is of such pleiotropic effect that its sequence contributes to variability in such diverse and complex physiological responses as those to numerous anesthetics. Further, not every person with red hair harbors the known red hair MC1R variants, yet most exhibit the aberrant anesthesia response. Rather, it is more likely that red hair color correlates with an element of population structure that also correlates with anesthesia response; i.e., the gene(s) for red hair color and aberrant anesthesia response are unique to or enriched for in particular branches of a human pedigree, within which these two traits are more common. Thus, the genes for red hair color and aberrant anesthesia response are distributed as a function of population structure, and, similarly, so have many other traits, as disclosed herein.

The AIMs and methods disclosed herein are suitable for the measurement of various levels of structure, including crude continental structure, as well as structure relevant for ethnicity and even cryptic structure (e.g., almost 30 markers have been identified that are capable of resolving Pacific Islanders from other East Asians; i.e., a finer level of structure than continental structure). The informativeness of the AIMs arises over the course of evolutionary human development through founder effects, migration, bottlenecks, genetic drift and/or selection, but these forces need not be focused on hair color or anesthesia response for there to have arisen AIMs correlated with these two types of traits. Much like this case, where there must be AIMs that are informative for disparate phenotypes in North Western Continental Europeans, there also must be AIMs that are informative for disparate phenotypes in IndoEuropeans in general. The results demonstrating the similarity in AIMs with good delta values for Lipitor™ response, Zocor™ response, hair color and eye color (FIGS. 12 to 16) are indicative of a level of population structure that is informative for each of these phenotypes.

It should be noted that the magnitude of association does not match between the traits. For example, the strongest AIM for iris color is not the strongest AIM for Lipitor™ response, etc. Also, the direction of the association is not necessarily the same among the traits; thus, agonist associations (associated positively) for blue iris color can be either agonist or antagonist associations (associated negatively) for a particular Lipitor™ response outcome.

Randomly selected AIMs that distinguish specifically between Africans and East Asians can, but need not, harbor information about a particular set of traits because they can, but need not, be markers of the particular element of human population structure correlated with the trait (i.e., the branch of the human family tree within which these traits are more common). Similarly, AIMs that distinguish between Indo Europeans and Africans would not necessarily carry with them the information necessary to help with the prediction of anesthesia response or red hair. Only those AIMs with alleles that distinguish between individuals of the particular branches of humanity within which the traits values are concentrated or underrepresented carry the information necessary to predict trait value, in this example, anesthesia and red hair, or, as disclosed, response to Lipitor™, Zocor™, hair color and eye color. Certain SNPs are good AIMs for certain gross elements of population structure (Europeans versus sub-Saharan African) or sub-substructure (Northern Europeans versus Mediterranean IndoEuropeans) or micro-structure (Scottish vs. Irish vs. British; or red haired Northern Europeans versus Northern Europeans of other hair colors; or Northern Europeans that respond to a drug versus other Northern Europeans that do not respond to the drug). Some AIMs are informative for some levels of population structure but not others, and most of the SNPs in the human genome do not carry information of any level of population structure at all (i.e. they are not AIMs).

The primary element of the disclosed methods is that most human traits can be predicted through a detailed measurement of AIMs associated with population structure at various levels, provided the trait correlates with that element of structure. A secondary element is that classifiers, or collections of SNP markers, and methods for predicting trait value from DNA can be constructed for most human traits through such an appreciation of population structure. As disclosed herein, such applications can be accomplished through correlation, not just through extended LD found in certain admixed groups such as Hispanics or African Americans, but for any sample of subjects, regardless of race or ethnic background, provided that the AIMs used are appropriate for the element of population structure with which the trait is correlated. These results can be attained because the methods of the invention provide a means to mine the genome for good AIMs, qualify their value as AIMs and accurately measure population structure against the backdrop of human phenotypes.

The trend observed in the studies represented by FIGS. 12 to 16 has been observed for many other traits, where the SNP associations are of such “penetrance” that they withstand corrections for multiple tests (Steenland et al., supra, 2000). As such, by measuring AIMs across the human genome, elements of population structure, sub-structure, or microstructure relevant for predicting or inferring the value of virtually any given human trait can be learned, even if the markers are not in linkage disequilibrium with the phenotypically active loci for the trait. This correlation applies to any human trait, simple or complex, of clinical, recreational, forensics or other value, or not, because every human trait is correlated to greater or lesser degree with certain elements of population structure.

It is not possible to know a priori whether a trait segregates with population structure versus substructure versus sub-substructure versus microstructure unless one measures AIMs across the genome in individuals and identifies which (if any) correlate with trait value. Virtually any trait correlates with at least one element of structure, some with gross structure (as in the case of human skin pigmentation), some with substructure (as in the case of iris, hair or skin pigmentation between South Asian IndoEuropeans (Indians) and Northern European IndoEuropeans (e.g., Irish), and some with microstructure (as in the case of red hair or anesthesia response among Continental Europeans). It is not important to know with which level of population structure a trait correlates in order to measure and find AIMs for the inference of that trait, it is only necessary to measure and test a plurality of general AIMs for statistical association with that trait. As such, methods are provided for linking human gene sequences with traits such that they can be predicted or inferred. Such a method is valuable, for example, in the clinical and forensic fields because it is not important that biologically or mechanistically relevant sequences be measured when predicting a common trait (response to a drug or predisposition to develop disease), as it is to make an accurate inference of that common trait (drug response or disease disposition).

### EXAMPLE 6

### Algorithm for Maximum Likelihood Estimate

An software program was written based on the algorithm of Hanis et al. (supra, 1986) for using multilocus AIM genotypes to determine the Maximum Likelihood Estimate of individual BGA admixture. A flow chart illustrating an algorithm useful for determining proportional ancestry is provided in Table 12. An example as to how the algorithm operates in shown in Table 13, and the results of an ancestry proportion calculation is shown in Table 14.

The □ value is an expression of the ancestry informativeness of the marker (Dean et al., 1994). For a biallelic marker, the frequency differential (6) is equal to px−py which is equal to qy−qx, where px and py are the frequencies of one allele in populations X and Y and qx and qy are the frequencies of the other. To test the departures from independence in allelic state within and between loci, we have used the MLD exact test (Zaykin et al., supra, 1995). The collection of 71 AIMs used in Example 2 was selected to maximize the cumulative □ value within, and minimize differences in the cumulative □ value between each of the six possible pairs of the four dimensional (sub-Saharan African, Native American, IndoEuropean and East Asian) problem.

The algorithm inverts the population specific allele frequencies to obtain a likelihood estimate of proportional affiliation corresponding to a multilocus genotype using three groups at a time; three groups were used mainly for computational convenience, and also because a 4-dimensional admixture is likely to be relatively rare. For example, the likelihood of 100% IndoEuropean, 0% Native American, 0% East Asian is calculated, then the likelihood of 99% IndoEuropean, 1% Native American, 0% East Asian is calculated next, and so on until all possible IndoEuropean, Native American and East Asian proportions are considered, and then the process is repeated for all possible IndoEuropean, Native American and African proportions, and all possible Native American, African and East Asian proportions. The likelihood of maximum value is selected as the Maximum Likelihood Estimate (MLE). When plotting a single MLE on a triangle plot, the space within which the likelihood is within 2-fold, 5-fold and 10-fold of the MLE also are delimited (see FIG. 3); these intervals generally are not plotted when multiple MLEs are shown in a single triangle plot.

TABLE 12
Algorithm for Ancestry Calculation - Flow Chart

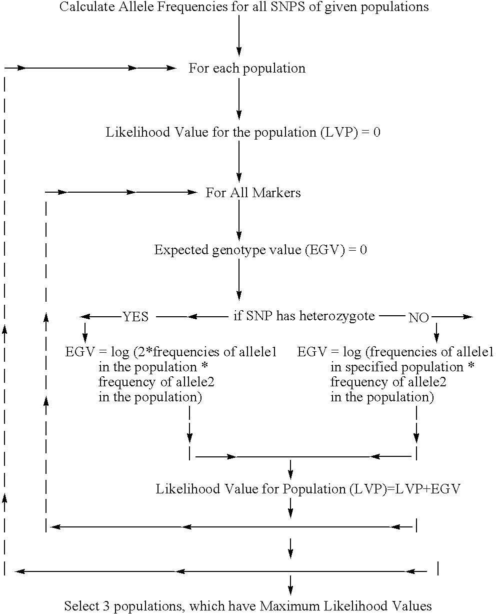

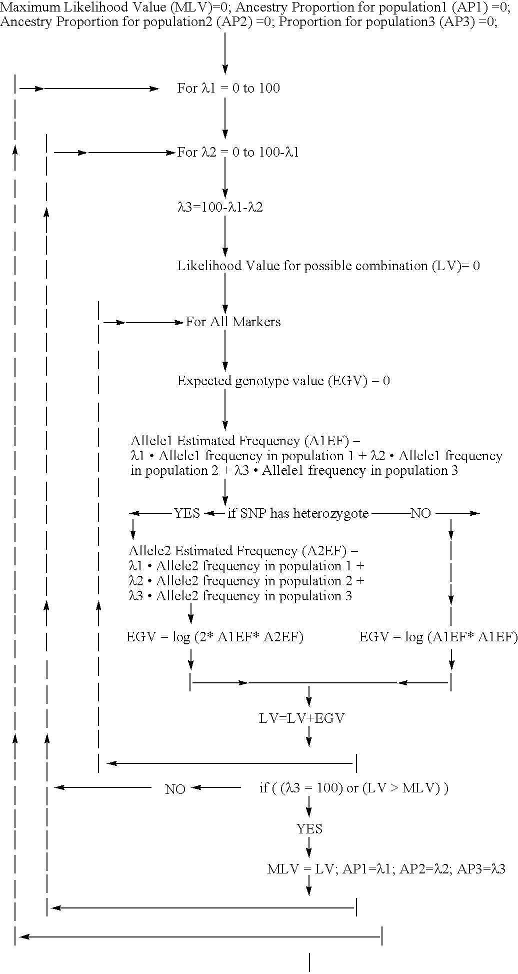

λ1, λ2, λ3 are corresponding Ancestry proportions for Blind Sample.

TABLE 13
Example of Proportional Ancestry Determination Using Algorithm

| SAMPLEID | SNP1 | SNP 2 |
|---|---|---|
| ANC30000 | GT | TT |

I. Pick best three population for blind sample

II. Get proportions which has maximum likelihood value

1. Pick Up Best Three Populations:

Algorithm:

For all populations

```
{
  For all snps
  {
    populationSum <- populationSum + Expected genotype frequency.
  }
}
```

Pick three populations, which have maximum value.

STEP1:

For all population

STEP2:
SNP1 has heterogeneous genotype; Alleles are G and T.
Expected genotype for SNP1−log (2*P(G,1)*P(T,1));
SNP2 has Homogenous genotypes
Expected genotype for SNP2=log (P(T,1)*p(T,1));

Likelihood value population1=Expected GT for SNP1+Expected TT for SNP2

Repeat STEP 2 for all population

Pick best three likelihood values out of those 4 population values.

For those selected three population estimate proportions.

1. Starts with

λ₁₌₀, λ_{2=0 and} λ_{3=1 0+0+1=1}
   2. Compute Likelihood Value:

Estimate Expected genotype for SNP1
SNP1 has heterogeneous genotype; Alleles are G and T.
Estimated Allele Frequency from SAMPLE:
Allele1 Estimated Frequency (A1EF)=λ₁. p(G,1)+λ₂. p(G,2)+λ₃. p(G,3)
Where p(G,1)—G Allele frequency in population 1
p(G,2)—G Allele frequency in population 2
p(G,3)—G Allele frequency in population 3
The mixing proportions λ₁, λ₂ and λ₃ are treated as unknown parameters.
Allele2 Estimated Frequency (A2EF)=λ₁. p(T,1)+λ₂. p(T,2)+λ₃. p(T,3)
Where p(T,1)—T Allele frequency in population 1
p(T,2)—T Allele frequency in population 2
p(T,3)—T Allele frequency in population 3
   The likelihood of the parameters is obtained by multiplying the probabilities for each observed genotype in the new observation under the assumption of Hardy-Weinberg Law.
Since SNP1 has heterogeneous genotype
Expected genotype for SNP1=log(2*A1EF*A2EF);

Estimate Expected Genotype for SNP2
Estimated Allele Frequency from SAMPLE:
Allele1 Expected Frequency (A1EF)=λ₁. p(T,1)+λ₂. p(T,2)+λ₃. p(T,3)
Where p(T,1)—T Allele frequency in population 1
p(T,2)—T Allele frequency in population 2
p(T,3)—T Allele frequency in population 3
The mixing proportions λ₁, λ_{2 and} λ₃ are treated as unknown parameters.
Since SNP2 has Homogenous genotypes
Expected genotype for SNP2=log(A1EF*A1EF);

Likelihood Value
Compute LIKELIHOOD VALUE by adding all Expected genotypes of all SNPS

LIKELIHOOD=Expected genotype for SNP1+Expected genotypes for SNP2;

Compute LIKELIHOOD value by using different unknown parameters. (Repeat step 2)

3. Get Maximum Likelihood value and corresponding unknown parameters.

Those unknown parameters are nothing but proportions

TABLE 14
Ancestry Proportion Calculation:
Ancestry Frequency Table
African frequencies

| | | | | |
|---|---|---|---|---|
| SNP1 | G | 0.8 | T | 0.2 |
| SNP2 | T | 0.7 | A | 0.3 |
| SNP3 | C | 1.0 | | |

European frequencies

| | | | | |
|---|---|---|---|---|
| SNP1 | G | 0.9 | T | 0.1 |
| SNP2 | T | 0.7 | A | 0.3 |
| SNP3 | C | 0.8 | T | 0.2 |

Native American (NA) frequencies

| | | | | |
|---|---|---|---|---|
| SNP1 | G | 0.6 | T | 0.4 |
| SNP2 | T | 0.5 | A | 0.5 |
| SNP3 | C | 0.7 | T | 0.3 |

Middle East (ME) frequencies

| | | | | |
|---|---|---|---|---|
| SNP1 | G | 0.7 | T | 0.3 |
| SNP2 | T | 0.9 | A | 0.1 |
| SNP3 | C | 0.9 | T | 0.1 |

### EXAMPLE

| SAMPLEID | SNP1 | SNP2 | SNP3 |
|---|---|---|---|
| ANC30000 | GT | TT | CT |

III. Pick best three population for blind sample

IV. Get proportions which has maximum likelihood value
a. Pick three best populations
African (Assume blind sample 100% African):
SNP1 Alleles: G, T

$$“ G ” allele frequency in African \qquad P ( G ) = 0.8$$

$$“ T ” allele frequency in African \qquad P ( T ) = 0.2$$

$$\begin{matrix}Expected genotype value for SNP 1 = log ( 2 * P ( G ) * P ( T ) ) \\ = log ( 2 * 0.8 * 0.2 ) \\ = - 0.4948\end{matrix}$$
SNP2 Alleles: T, T

$$“ T ” allele frequency in African \qquad P ( T ) = 0.7$$

$$\begin{matrix}Expected genotype value for SNP 2 = log ( P ( T ) * P ( T ) ) \\ = log ( 0.7 * 0.7 ) \\ = - 0.3098\end{matrix}$$
SNP3 Alleles: C, T

$$“ C ” allele frequency in African \qquad P ( C ) = 0.9999$$

$$“ T ” allele frequency in African \qquad P ( T ) = 0.0001$$

$$\begin{matrix}Expected genotype value for SNP 3 = log ( 2 * P ( C ) * P ( T ) ) \\ = log ( 2 * 0.9999 * 0.0001 ) \\ = - 3.6990\end{matrix}$$

$$Likelihood for African = - 0.4948 - 0.3098 - 3.6990 = - 4.5036$$
European (Assume blind sample 100% European):
SNP1 Alleles: G, T

$$“ G ” allele frequency in European \qquad P ( G ) = 0.9$$

$$“ T ” allele frequency in European \qquad P ( T ) = 0.1$$

$$\begin{matrix}Expected genotype value for SNP 1 = log ( 2 * P ( G ) * P ( T ) ) \\ = log ( 2 * 0.9 * 0.1 ) \\ = - 0.7447\end{matrix}$$
SNP2 Alleles: T, T

$$“ T ” allele frequency in European \qquad P ( T ) = 0.7$$

$$\begin{matrix}Expected genotype value for SNP 2 = log ( P ( T ) * P ( T ) ) \\ = log ( 0.7 * 0.7 ) \\ = - 0.3098\end{matrix}$$
SNP3 Alleles: C, T

$$“ C ” allele frequency in European \qquad P ( C ) = 0.8$$

$$“ T ” allele frequency in European \qquad P ( T ) = 0.2$$

$$\begin{matrix}Expected genotype value for SNP 3 = log ( 2 * P ( C ) * P ( T ) ) \\ = log ( 2 * 0.8 * 0.2 ) \\ = - 0.4948\end{matrix}$$

$$Likelihood for European = - 0.7447 - 0.3098 - 0.4948 = - 1.5493$$
Native American (Assume blind sample 100% NA):
SNP1 Alleles: G, T

$$“ G ” allele frequency in NA \qquad P ( G ) = 0.6$$

$$“ T ” allele frequency in NA \qquad P ( T ) = 0.4$$

$$\begin{matrix}Expected genotype value for SNP 1 = log ( 2 * P ( G ) * P ( T ) ) \\ = log ( 2 * 0.6 * 0.4 ) \\ = - 0.3187\end{matrix}$$
SNP2 Alleles: T, T

$$“ T ” allele frequency in NA \qquad P ( T ) = 0.5$$

$$\begin{matrix}Expected genotype value for SNP 2 = log ( P ( T ) * P ( T ) ) \\ = log ( 0.5 * 0.5 ) \\ = - 0.6020\end{matrix}$$
SNP3 Alleles: C, T

$$“ C ” allele frequency in NA \qquad P ( C ) = 0.7$$

$$“ T ” allele frequency in NA \qquad P ( T ) = 0.3$$

$$\begin{matrix}Expected genotype value for SNP 3 = log ( 2 * P ( C ) * P ( T ) ) \\ = log ( 2 * 0.7 * 0.3 ) \\ = - 0.3767\end{matrix}$$

$$\begin{matrix}Likelihood for Native American = - 0.3187 - 0.6020 - 0.3767 \\ = - 1.2974\end{matrix}$$
Middle East (Assume blind sample 100% ME):
SNP1 Alleles: G, T

$$“ G ” allele frequency in ME \qquad P ( G ) = 0.7$$

$$“ T ” allele frequency in ME \qquad P ( T ) = 0.3$$

$$\begin{matrix}Expected genotype value for SNP 1 = log ( 2 * P ( G ) * P ( T ) ) \\ = log ( 2 * 0.7 * 0.3 ) \\ = - 0.3767\end{matrix}$$
SNP2 Alleles: T, T

$$\begin{matrix}“ T ” allele frequency in ME P ( T ) = 0.9 \\ Expected genotype value for SNP 2 = log ( P ( T ) * P ( T ) ) \\ = log ( 0.9 * 0.9 ) \\ = - 0.0915\end{matrix}$$
SNP3 Alleles: C, T

$$\begin{matrix}“ C ” allele frequency in ME & P ( C ) = 0.9 \\ “ T ” allele frequency in ME & P ( T ) = 0.1\end{matrix}$$

$$\begin{matrix}Expected genotype value for SNP 3 = log ( 2 * P ( C ) * P ( T ) ) \\ = log ( 2 * 0.9 * 0.1 ) \\ = - 0.7447\end{matrix}$$

$$4. \begin{matrix}Likelihood for Middle East = - 0.3767 - 0.0915 - 0.7447 \\ = - 1.2129\end{matrix}$$
Likelihood value for African=−4.5036
5. Likelihood value for European=−1.5493
6. Likelihood value for Native American=−1.2974

7. Likelihood value for Middle East=−1.2129

In this case we drop African and consider other three for proportions.

Maximum Likelihood Value

Now starts giving some values to unknown parameters

I = European J = Native American K = Middle East

| | |
|---|---|
| Always | I + j + k = 1 |

I = 0; j = 0; k=1
{

| SNP1 Alleles: G, T. | |
|---|---|
| “G” allele frequency in European | P(G,1) = 0.9 |
| “G” allele frequency in NA | P(G,2) = 0.6 |
| “G” allele frequency in ME | P(G,3) = 0.7 |
| Allele1 Estimated Frequency(A1EF) | = I * P(G,1) + J*P(G,2) + K* P(G,3) |
| | = 0 * 0.9 + 0*0.6 + 1*0.7 |
| | = 0.7 |
| “T” allele frequency in European | P(T,1) = 0.1 |
| “T” allele frequency in NA | P(T,2) = 0.4 |
| “T” allele frequency in ME | P(T,3) = 0.3 |
| Allele 2 Estimated Frequency(A1EF) | = I * P(T,1) + J*P(T,2) + K* P(T,3) |
| | = 0 * 0.1 + 0*0.4 + 1*0.3 |
| | = 0.3 |
| Expected genotype value for SNP1 | = log (2*A1EF*A2EF) |
| | = log (2*0.7*0.3); |
| | = −0.3767 |
| SNP2 Alleles: T, T. | |
| “T” allele frequency in European | P(T,1) = 0.7 |
| “T” allele frequency in NA | P(T,2) = 0.5 |
| “T” allele frequency in ME | P(T,3) = 0.9 |
| Allele 1 Estimated Frequency(A1EF) | = I * P(T,1) + J*P(T,2) +K* P(T,3) |
| | = 0 * 0.7 + 0*0.5 + 1*0.9 |
| | = 0.9 |
| Expected genotype value for SNP2 (EGV2) | = log(A1EF*A2EF) |
| | = log (0.9*0.9); |
| | = −0.0915 |
| SNP3 Alleles: C, T. | |
| “C” allele frequency in European | P(C, 1) = 0.8 |
| “C” allele frequency in NA | P(C, 2) = 0.7 |
| “C” allele frequency in ME | P(C, 3) = 0.9 |
| Allele 1 Estimated Frequency(A1EF) | = I * P(C,1) + J*P(C,2) + K* P(C,3) |
| | = 0 * 0.8 + 0*0.7 + 1*0.9 |
| | = 0.9 |
| “T” allele frequency in European | P(T,1) = 0.2 |
| “T” allele frequency in NA | P(T,2) = 0.3 |
| “T” allele frequency in ME | P(T,3) = 0.1 |
| Allele 2 Estimated Frequency(A1EF) | = I * P(T,1) + J*P(T,2) + K* P(T,3) |
| | = 0 * 0.2 + 0*0.3 + 1*0.1 |
| | = 0.1 |
| Expected genotype value for SNP3(EGV3) | = log (2*A1EF*A2EF) |
| | = log (2*0.9*0.1); |
| | = −0.7447 |

| | |
|---|---|
| i. | Likelihood value for unknown parameters |

= EGV1+EGV2+EGV3
= −0.3767 − 0.0915 − 0.7447
= −1.2129

for European = 0; NA=0; MiddleEast = 1; likelihood value is −1.2129

}

Repeat above loop for all possible combinations
0.0, 0.0, 1, 0 −1.2129
1.0, 0.0, 0.0,
0.0, 1.0, 0.0,
0.1, 0.0 0.9,
0.1, 0.1, 0.8,
0.1, 0.2, 0.7,
0.1, 0.3, 0.6,
0.1, 0.4, 0.5
etc
Get Maximum likelihood value and corresponding proportions are ancestry proportions.

### EXAMPLE 7

### Multiplex Analysis Using a DNAWitness™ Kit

This Example discloses reagents, including PCR primer pairs and SBE primers, and conditions for performing 12 multiplex reactions that allow for the determination of the nucleotide occurrences of 174 SNPs (AIMs) shown as SEQ ID NOS:364 to 537 in Appendix 3, and demonstrates validation of the multiplex method using capillary electrophoresis. A flowchart illustrating the exemplified protocol and results of multiplex examination of a test nucleic acid sample are shown in FIGS. 17-19A-K.

Multiplex PCR

twelve 0.2 μL tubes were labeled with the Panel Name (see below; P3, P41, etc.) and with a Sample Name. The Panel Name (e.g., P3) corresponds to the Panels as set forth in Appendices 1 to 3, wherein P3 is “Panel 3”, P41 is “Panel 41”, etc.

| Tube # | Panel Name |
|---|---|
| 1 | P3 |
| 2 | P41 |
| 3 | P42 |
| 4 | P43 |
| 5 | P4452 |
| 6 | P4553 |
| 7 | P4654 |
| 8 | P4755 |
| 9 | P48 |
| 10 | P4957 |
| 11 | P5051 |
| 12 | P56A |

Twelve stock PCR Primer Panel Tubes were prepared as shown in Appendix 2. For example, the stock PCR Primer Panel 3 tube was prepared by placing the indicated amount (PCR (μl)) of each stock oligonucleotide (stock (μM)) into a 1.5 mL tube, then the volume was raised to 1.0 mL using deionized (DI) water. Stock PCR Primer Panel Tubes were stored at −20° C. For use, the twelve PCR Primer Panel tubes were thawed and vortexed, and 5 μL PCR primers was dispensed into each of their corresponding labeled PCR Panel tubes. Template DNA in a volume of 2.5 μL was transferred to each labeled PCR tube. A master mix was prepared for the appropriate number of reactions (see below), then 2.5 μL of the Master Mix was added to the PCR Primers/Template DNA mix for each PCR Panel tube.

Master Mix

| | |
|---|---|
| 1.20 μL | ddH₂O |
| 1.00 μL | 10X HOTMASTER Taq Buffer |
| 0.20 μL | dNTP mix (10 mM each) |
| 0.10 μL | HOTMASTER Taq (5 U/μL) |
| 2.50 μL | per reaction |

Touchdown PCR was performed by incubating the samples as indicated below to confer specificity.

| | |
|---|---|
| Step 1 | 95° for 5 minutes |
| Step 2 | 94° for 45 seconds |
| Step 3 | 60° for 1 minute |
| Step 4 | 70° for 1 minute |
| Step 5 | Go To Step 2, 9 times |
| Step 6 | 94° for 45 seconds |
| Step 7 | 55° for 1 minute |
| Step 8 | 70° for 1 minute |
| Step 9 | Go To Step 6, 29 times |
| Step 10 | 72° for 5 minutes |
| Step 11 | store at 4° |

Following PCR, samples were treated with exonuclease I/shrimp alkaline phosphatase to remove unreacted primers and nucleotides. Four μL of exoSAP-IT® reagent (USB, Inc.) was added to each reaction, then incubation was performed in a thermalcycler as indicated below. The cleaned PCR samples then were used for multiplex SBE.

| | |
|---|---|
| Step 1 | 37° for 60 minutes |
| Step 2 | 99° for 15 minutes |
| Step 3 | store at 4° |

Multiplex SBE

For the SBE reaction, twelve 0.2 mL SBE Panel tubes were labeled as above. Twelve stock SBE Primer Panel tubes were prepared as shown in Appendix 1. For example, the stock SBE Primer Panel 3 tube was prepared by placing the indicated amount (SBE (μl)) of each stock oligonucleotide (stock (μM)) into a 1.5 mL tube, then the volume was raised to 1.0 mL using deionized (DI) water. Stock SBE Primer Panel Tubes were stored at −20° C. For use, the twelve stock SBE Primer Panel tubes were thawed and vortexed, and 2 μL was dispensed into each of their corresponding labeled SBE Panel tubes. Three μL of the cleaned PCR reaction was added to each labeled 0.2 mL SBE Panel tube, then 5 μL of ABI Prism® SNaPshot™ Ready Reaction Mix (Applied Biosystems, Inc.) was added to each tube. Multiplex SBE was then performed as indicated below.

| | |
|---|---|
| Step 1 | 96° for 3 minutes |
| Step 2 | 94° for 20 seconds |
| Step 3 | 40° for 11 seconds |
| Step 4 | Go To Step 2, 44 times |
| Step 5 | store at 4° |

Following SBE, the samples were treated with SAP to remove unreacted nucleotides. One 1 μL SAP was combined with 1 μL of SAP dilution buffer for each reaction, then samples were incubated in a thermalcycler as indicated below. The cleaned SBE samples were then analyzed by capillary electrophoresis.

| | |
|---|---|
| Step 1 | 37° for 60 minutes |
| Step 2 | 75° for 15 minutes |
| Step 3 | store at 4° |

Capillary Electrophoresis

Multiplex reactions were analyzed by capillary electrophoresis using an ABI 3700 Genetic Analyzer. For each reaction, 0.5 μL of GS120 Liz size standard (ABI) was diluted into 9 μL of Hi-Di Formamide, then 0.5 μL of the cleaned SBE sample was added. The samples were run, and an Excel™ worksheet was uploaded to map the samples.

Electropherograms generated from the 12 multiplex reactions sampling 174 SNPs are shown in Appendix 4. Each dideoxyribonucleotide triphosphate used in the SBE reaction was labeled with a different dye, and corresponding colors are used in the electropherograms, including blue (ddGTP), green (ddATP), black (ddCTP) and red (ddTTP). Referring to the results obtained using Panel 3, each primer (e.g., 2072, 963, etc.; see Appendix 1) is indicated at the bottom of the electropherogram and the nucleotide number is indicated at the top. As shown for Panel 3, SBE primer 2072 produced a single “black” peak, indicating that the sample was homozygous for “C” at the SNP position. In comparison, SBE primer 966 generated two products shown as a blue peak (G) and black peak (C), indicating that the sample (subject) was heterozygous (G/C) at this SNP position.

It should be noted that, as is common in electrophoresis, the SBE products do not always run exactly at the expected nucleotide number size due, for example, to secondary structure of the oligonucleotide and the presence of the dye. As such, where SBE primer 963 is 25 nucleotides in length (see Appendix 1), and the SBE product would be expected to be 26 nucleotides, the SBE product ran at about 24 to 25 nucleotides (24.52; see first (upper) box below peak). The relative amount of SBE product in each peak, expressed as “relative fluorescent units” (RFU), that was detected for each SBE product also indicated in the electropherograms (e.g., 5570 RFU for SBE primer 963 product; second (lower) box below peak).

Each Panel of electropherograms shows lines corresponding to all four dideoxyribonucleotide triphosphates (dyes). However, in Panel 3, for example, no ddTTP (red) SBE products were detected; all of the “peaks” observed in the electropherogram were below the threshold value of the detection system (approximately 100 RFU).

Referring to the Panel 42 electropherogram, compression of the samples occurred, resulting in a relatively large discrepancy of measured nucleotide size and actual nucleotide size. For example, while SBE products for SBE primer 1767 (50 nucleotides; see Appendix 1) would be expected to be about 51 nucleotides, the SBE products shown in the Panel 42 electropherogram ran at about 42 to 43 nucleotides (42.06, blue; and 42.94, red). Nevertheless, the relative positions of the SBE products were maintained and, therefore, useful results were obtained.

These results demonstrate that 12 multiplex reactions can be used to determine the nucleotide occurrences of 174 SNPs in a test nucleic acid sample.

Although the invention has been described with reference to the above example, it will be understood that modifications and variations are encompassed within the spirit and scope of the invention. Accordingly, the invention is limited only by the following claims.

Although the invention has been described with reference to the above example, it will be understood that modifications and variations are encompassed within the spirit and scope of the invention. Accordingly, the invention is limited only by the following claims.

What is claimed is:

1. A plurality of primer pairs for detecting an AIM, wherein the plurality comprises at least 2 different primer pairs, wherein each pair comprises:
(a) a first primer comprising 15-50 contiguous nucleotides of a sequence set forth in SEQ ID NOs:364-537; and
(b) a second primer comprising 15-50 contiguous nucleotides of the complement of a sequence set forth in SEQ ID NOs:364-537,
wherein each of the at least two different primer pairs can be used to amplify a polynucleotide comprising a nucleotide occurrence of a single nucleotide polymorphism as set forth in any of SEQ ID NOs: 364-537

2. The plurality of primer pairs of claim 1, wherein the at least 2 different primer pairs comprise sequences from a single panel, wherein the panel is selected from:
(a) panel 3: SEQ ID NOs:712-725;
(b) panel 41: SEQ ID NOs:726-745;
(c) panel 42: SEQ ID NOs:746-759;
(d) panel 43: SEQ ID NOs:760-777;
(e) panel 4452: SEQ ID NOs:777-825;
(f) panel 4553: SEQ ID NOs:826-867;
(g) panel 4654: SEQ ID NOs:868-907;
(h) panel 4755: SEQ ID NOs:908-951;
(i) panel 48: SEQ ID NOs:952-975;
(j) panel 4957: SEQ ID NOs:976-1011;
(k) panel 5051: SEQ ID NOs:1012-1041; and
(l) panel 56A: SEQ ID NOs:1042-1059.

3. The plurality of primer pairs of claim 2, comprising:
(a) 7 primer pairs of panel 3;
(b) 10 primer pairs of panel 41;
(c) 7 primer pairs of panel 42;
(d) 9 primer pairs of panel 43;
(e) 24 primer pairs of panel 4452;
(f) 21 primer pairs of panel 4553;
(g) 20 primer pairs of panel 4654;
(h) 22 primer pairs of panel 4755;
(i) 12 primer pairs of panel 48;
(j) 18 primer pairs of panel 4957;
(k) 15 primer pairs of panel 5051; or
(l) 9 primer pairs of panel 56A.

4. The plurality of primer pairs of claim 1, wherein the at least 2 primer pairs comprise sequences from at least 2 panels, wherein the 2 panels are selected from:
(a) panel 3: SEQ ID NOs:712-725;
(b) panel 41: SEQ ID NOs:726-745;
(c) panel 42: SEQ ID NOs:746-759;
(d) panel 43: SEQ ID NOs:760-777;
(e) panel 4452: SEQ ID NOs:777-825;
(f) panel 4553: SEQ ID NOs:826-867;
(g) panel 4654: SEQ ID NOs:868-907;
(h panel 4755: SEQ ID NOs:908-951;
(i) panel 48: SEQ ID NOs:952-975;
(j) panel 4957: SEQ ID NOs:976-1011;
(k) panel 5051: SEQ ID NOs:1012-1041; and
(l) panel 56A: SEQ ID NOs:1042-1059.

5. The plurality of primer pairs of claim 4, comprising:
(a) 7 primer pairs of panel 3;
(b) 10 primer pairs of pane 41;
(c) 7 primer pairs of panel 42;
(d) 9 primer pairs of panel 43;
(e) 24 primer pairs of panel 4452;
(f) 21 primer pairs of panel 4553;
(g) 20 primer pairs of panel 4654;
(h) 22 primer pairs of panel 4755;
(i) 12 primer pairs of panel 48;
(j) 18 primer pairs of panel 4957;
(k) 15 primer pairs of panel 5051; and
(l) 9 primer pairs of panel 56A.

6. A plurality of primers for detecting an AIM, wherein the plurality comprises at least 2 different primers,
wherein each primer comprises 15-100 contiguous nucleotides of a sequence set forth in SEQ ID NOs:364-537,
or 15-100 contiguous nucleotides of the complement of a sequence set forth in SEQ ID NOs:364-537,
wherein each of the at least two different primers can be used to detect a nucleotide occurrence of a single nucleotide polymorphism as set forth in any of SEQ ID NOs: 364-537.

7. The plurality of primers of claim 6, wherein the at least 2 different primers comprise sequences from a single panel, wherein the panel is selected from:
(a) panel 3: SEQ ID NOs:538-544;
(b) panel 41: SEQ ID NOs: 545-554;
(c) panel 42: SEQ ID NOs: 555-561;
(d) panel 43: SEQ ID NOs:562-570;
(e) panel 4452: SEQ ID NOs:571-594;
(f) panel 4553: SEQ ID NOs:595-615;
(g) panel 4654: SEQ ID NOs:616-635;
(h) panel 4755: SEQ ID NOs:636-657;
(i) panel 48: SEQ ID NOs:658-669;
(j) panel 4957: SEQ ID NOs:670-687;
(k) panel 5051: SEQ ID NOs:688-702; and
(l) panel 56A: SEQ ID NOs:703-711.

8. The plurality of primers of claim 7, comprising:
(a) 7 primers of panel 3;
(b) 10 primers of panel 41;
(c) 7 primers of panel 42;
(d) 9 primers of panel 43;
(e) 24 primers of panel 4452;
(f) 21 primers of panel 4553;
(g) 20 primers of panel 4654;
(h) 22 primers of panel 4755;
(i) 12 primers of panel 48;
(j) 18 primers of panel 4957;
(k) 15 primers of panel 5051; or
(l) 9 primers of panel 56A.

9. The plurality of primers of claim 6, wherein the at least 2 different primers comprise sequences from at least two panels, wherein the panels are selected from:
(a) panel 3: SEQ ID NOs:538-544;
(b) panel 41: SEQ ID NOs: 545-554;
(c) panel 42: SEQ ID NOs: 555-561;
(d) panel 43: SEQ ID NOs:562-570;
(e) panel 4452: SEQ ID NOs:571-594;
(f) panel 4553: SEQ ID NOs:595-615;
(g) panel 4654: SEQ ID NOs:616-635;
(h) panel 4755: SEQ ID NOs:636-657;
(i) panel 48: SEQ ID NOs:658-669;
(j) panel 4957: SEQ ID NOs:670-687;
(k) panel 5051: SEQ ID NOs:688-702; and
(l) panel 56A: SEQ ID NOs:703-711.

10. The plurality of primers of claim 9 comprising:
(a) 7 primers of panel 3;
(b) 10 primers of panel 41;
(c) 7 primers of panel 42;
(d) 9 primers of panel 43;
(e) 24 primers of panel 4452;
(f) 21 primers of panel 4553;
(g) 20 primers of panel 4654;
(h) 22 primers of panel 4755;
(i) 12 primers of panel 48;
(j) 18 primers of panel 4957;
(k) 15 primers of panel 5051; and
(l) 9 primers of panel 56A.

11. A kit, comprising the plurality of primer pairs of any of claims 1-5.

12. The kit of claim 11, further comprising at least one reagent for performing a DNA amplification reaction.

13. The kit of claim 12, wherein the reagent comprises at least one polymerase, deoxyribonucleotide triphosphate, deoxyribonucleotide triphosphate analog, or a combination thereof.

14. The kit of claim 13, wherein the deoxyribonucleotide triphosphate analog comprises a dideoxyribonucleotide triphosphate.

15. The kit of claim 13, which comprises dideoxyadenosine, dideoxycytidine, dideoxyguanidine, dideoxythymidine, or a combination thereof.

16. The kit of claim 13, wherein the deoxyribonucleotide triphosphate or deoxyribonucleotide triphosphate analog comprises a detectable label.

17. A kit, comprising the plurality of primers of any of claims 6-10.

18. The kit of claim 17, further comprising at least one reagent for performing a primer extension reaction.

19. The kit of claim 18, wherein the reagent comprises at least one polymerase, deoxyribonucleotide triphosphate, deoxyribonucleotide triphosphate analog, or a combination thereof.

20. The kit of claim 19, which comprises dideoxyadenosine, dideoxycytidine, dideoxyguanidine, dideoxythymidine, or a combination thereof.

21. The kit of claim 19, wherein the deoxyribonucleotide triphosphate or deoxyribonucleotide triphosphate analog comprises a detectable label.

22. A method of inferring, with a predetermined level of confidence, a trait of an individual, comprising:
(a) contacting at least a first sample comprising test nucleic acid molecules of an individual with at least a first plurality of primers as set forth in claim 7, under conditions suitable for single base extension of the primers, and
(b) detecting at least one single base extension product of a primer of the first plurality, wherein the single base extension product is informative of a nucleotide occurrence of a single nucleotide polymorphism (SNP) of an ancestry informative marker (AIM) indicative of a population structure correlated with a trait, thereby inferring, with a predetermined level of confidence, the trait of the individual.

23. The method of claim 22, further comprising detecting single base extension products of each primer of the plurality of claim 10.

24. The method of claim 22, further comprising
contacting at least a second sample comprising test nucleic acid molecules of the individual with at least a second plurality of primers as set forth in claim 7, under conditions suitable for single base extension of the primers, and
detecting at least one single base extension product of a primer of the second plurality, wherein the single base extension product is informative of a nucleotide occurrence of a SNP of an AIM indicative of a population structure correlated with the trait.

25. The method of claim 22, further comprising
contacting each of twelve samples comprising test nucleic acid molecules of the individual with each primer of the plurality as set forth in claim 10 under conditions suitable for single base extension of the primers, and
detecting single base extension product of the primers, wherein each single base extension product is informative of a nucleotide occurrence of a SNP of an AIM indicative of a population structure correlated with the trait.

26. The method of claim 22, wherein the single base extension product comprises a detectable label indicative of the single base.

27. The method of claim 22, wherein detecting the single base extension product is performed using electrophoresis, mass spectrometry, or gel chromatography.

28. The method of claim 27, wherein the electrophoresis comprises capillary gel electrophoresis.

29. The method of claim 22, wherein the test nucleic acid sample comprises a polymerase chain reaction amplification product.

30. The method of claim 22, wherein the trait comprises biogeographical ancestry (BGA).

31. The method of claim 30, wherein the BGA comprises a proportion of a sub-Saharan African, Native American, IndoEuropean, or East Asian ancestral group, or a combination of said ancestral groups.

32. The method of claim 22, wherein determination of BGA can be used to infer responsiveness of the individual to a drug.

33. The method of claim 32, wherein the drug is a cancer chemotherapeutic agent, or a statin.

34. The method of claim 22, wherein determination of BGA can be used to infer susceptibility to a disease.

35. The method of claim 34, wherein the disease has an ethnic predisposition.

36. The method of claim 22, wherein determination of BGA can be used to infer a pigmentation trait.

37. The method of claim 22, which comprises determining proportional ancestry of at least two ancestral groups of the individual.

38. The method of claim 37, which comprises determining proportional ancestry of three ancestral groups of the individual.

39. The method of claim 22, further comprising generating a graphical representation of the comparison of the three ancestral groups, said graphical representation comprising a triangle with each an ancestral group independently represented by a vertex of the triangle, wherein the maximum likelihood value of proportional affiliation for an individual comprises a point within the triangle.

40. The method of claim 39, wherein the graphical representation further comprises a confidence contour indicating a level of confidence associated with estimating the proportional ancestry.

41. A method of inferring, with a predetermined level of confidence, a trait of an individual, comprising:
(a) contacting each of 12 aliquots of a nucleic acid sample of test individual with one of each of the plurality of primer pairs of claim 5, under conditions suitable for polymerase chain reaction (PCR) amplification of target nucleotide sequences in the nucleic acid sample, thereby generating 12 PCR amplification samples;
(b) depleting each of the 12 PCR amplification samples of single stranded primers of the plurality of primer pairs and deoxyribonucleotide triphosphates;
(c) thereafter contacting each of the 12 PCR amplification samples with
(i) one of each plurality of primers as set forth in claim 10, and
(ii) dideoxyadenosine, dideoxycytidine, dideoxyguanidine, and dideoxythymidine, each of which comprises a different detectable label, under conditions suitable for single base extension of the primers, thereby generating 12 single base extension samples, each of which comprises a plurality of single base extension products; and
(d) detecting single base extension products in each of the 12 single base extension samples, wherein the single base extension products are informative of nucleotide occurrences of a single nucleotide polymorphisms of ancestry informative markers indicative of a population structure correlated with a trait,
thereby inferring, with a predetermined level of confidence, the trait of the individual.

42. The method of claim 37, wherein detecting the single base extension products is performed using capillary gel electrophoresis.

43. A method of inferring, with a predetermined level of confidence, a trait of an individual, comprising detecting a nucleotide occurrence of a single nucleotide polymorphism as set forth in any of SEQ ID NOS: 371 to 398, 400 to 408, 410 to 413, 415, 418, 420, 422, 423, 425, 431 to 433, 438 to 441, 443, 450 to 452, 455, 456, 461 to 463, 467 to 475, 477 to 485, 487, 495 to 498, 502 to 504, 506, 508 to 512, 514, 516, 519 to 521, 526, 529, and 533 to 537, wherein the nucleotide occurrence allows an inference as to the trait.
